SEQUENCE LISTING (552 sequences)

SEQ ID NO: 1 - RNA, 20 nt, synthetic construct
  misc_feature 1..20: note = Description of sequence: sgRNA: ACSF2_1
  source 1..20: mol_type = other RNA
atggtctccacgcccatcgg

SEQ ID NO: 2 - RNA, 20 nt, synthetic construct
  misc_feature 1..20: note = Description of sequence: sgRNA: ACSF2_2
  source 1..20: mol_type = other RNA
acgtagctgaggcctccgat

SEQ ID NO: 3 - RNA, 20 nt, synthetic construct
  misc_feature 1..20: note = Description of sequence: sgRNA: ACSF2_3
  source 1..20: mol_type = other RNA
cgaccaaggcctctcgttct

SEQ ID NO: 4 - RNA, 20 nt, synthetic construct
  misc_feature 1..20: note = Description of sequence: sgRNA: AIFM1_1
  source 1..20: mol_type = other RNA
ggaatactcaccagataacg

SEQ ID NO: 5 - RNA, 20 nt, synthetic construct
  misc_feature 1..20: note = Description of sequence: sgRNA: AIFM1_2
  source 1..20: mol_type = other RNA
tccatccgggctcgggatcc

SEQ ID NO: 6 - RNA, 20 nt, synthetic construct
  misc_feature 1..20: note = Description of sequence: sgRNA: AIFM1_3
  source 1..20: mol_type = other RNA
atcccgagcccggatggatc

SEQ ID NO: 7 - RNA, 20 nt, synthetic construct
  misc_feature 1..20: note = Description of sequence: sgRNA: AIM2_1
  source 1..20: mol_type = other RNA
ttgaagcgtgttgatcttcg

SEQ ID NO: 8 - RNA, 20 nt, synthetic construct
  misc_feature 1..20: note = Description of sequence: sgRNA: AIM2_2
  source 1..20: mol_type = other RNA
ttcacgtttgagacccaaga

SEQ ID NO: 9 - RNA, 20 nt, synthetic construct
  misc_feature 1..20: note = Description of sequence: sgRNA: AIM2_3
  source 1..20: mol_type = other RNA
tcagcgggacattaaccttt

SEQ ID NO: 10 - RNA, 20 nt, synthetic construct
  misc_feature 1..20: note = Description of sequence: sgRNA: APAF1_1
  source 1..20: mol_type = other RNA
agcattgtagaatgatacgt

SEQ ID NO: 11 - RNA, 20 nt, synthetic construct
  misc_feature 1..20: note = Description of sequence: sgRNA: APAF1_2
  source 1..20: mol_type = other RNA
gttagtggaataacttcgta

SEQ ID NO: 12 - RNA, 20 nt, synthetic construct
  misc_feature 1..20: note = Description of sequence: sgRNA: APAF1_3
  source 1..20: mol_type = other RNA
ccaggatgggtcaccataca

SEQ ID NO: 13 - RNA, 20 nt, synthetic construct
  misc_feature 1..20: note = Description of sequence: sgRNA: ATG10_1
  source 1..20: mol_type = other RNA
ctataagatgcgactgctac

SEQ ID NO: 14 - RNA, 20 nt, synthetic construct
  misc_feature 1..20: note = Description of sequence: sgRNA: ATG10_2
  source 1..20: mol_type = other RNA
gacactattacgcaacaggt

SEQ ID NO: 15 - RNA, 20 nt, synthetic construct
  misc_feature 1..20: note = Description of sequence: sgRNA: ATG10_3
  source 1..20: mol_type = other RNA
tgaattctgcacaataacgt

SEQ ID NO: 16 - RNA, 20 nt, synthetic construct
  misc_feature 1..20: note = Description of sequence: sgRNA: ATG101_1
  source 1..20: mol_type = other RNA
gcccacggtgccaatggagt

SEQ ID NO: 17 - RNA, 20 nt, synthetic construct
  misc_feature 1..20: note = Description of sequence: sgRNA: ATG101_2
  source 1..20: mol_type = other RNA
gtgccctgcgcaaggttgtt

SEQ ID NO: 18 - RNA, 20 nt, synthetic construct
  misc_feature 1..20: note = Description of sequence: sgRNA: ATG101_3
  source 1..20: mol_type = other RNA
tgcgcaactctggtggcgat

SEQ ID NO: 19 - RNA, 20 nt, synthetic construct
  misc_feature 1..20: note = Description of sequence: sgRNA: ATG12_1
  source 1..20: mol_type = other RNA
ctccccagaaacaaccaccc

SEQ ID NO: 20 - RNA, 20 nt, synthetic construct
  misc_feature 1..20: note = Description of sequence: sgRNA: ATG12_2
  source 1..20: mol_type = other RNA
cctccagcagcaattgaagt

SEQ ID NO: 21 - RNA, 20 nt, synthetic construct
  misc_feature 1..20: note = Description of sequence: sgRNA: ATG12_3
  source 1..20: mol_type = other RNA
agcaggttcctctgttcccg

SEQ ID NO: 22 - RNA, 20 nt, synthetic construct
  misc_feature 1..20: note = Description of sequence: sgRNA: ATG13_1
  source 1..20: mol_type = other RNA
gggtatatccaaactcgtca

SEQ ID NO: 23 - RNA, 20 nt, synthetic construct
  misc_feature 1..20: note = Description of sequence: sgRNA: ATG13_2
  source 1..20: mol_type = other RNA
agggtatatccaaactcgtc

SEQ ID NO: 24 - RNA, 20 nt, synthetic construct
  misc_feature 1..20: note = Description of sequence: sgRNA: ATG13_3
  source 1..20: mol_type = other RNA
tttacccaatctgaacccgt

SEQ ID NO: 25 - RNA, 20 nt, synthetic construct
  misc_feature 1..20: note = Description of sequence: sgRNA: ATG14_1
  source 1..20: mol_type = other RNA
tctacttcgacggccgcgac

SEQ ID NO: 26 - RNA, 20 nt, synthetic construct
  misc_feature 1..20: note = Description of sequence: sgRNA: ATG14_2
  source 1..20: mol_type = other RNA
ggcgatttcgtctacttcga

SEQ ID NO: 27 - RNA, 20 nt, synthetic construct
  misc_feature 1..20: note = Description of sequence: sgRNA: ATG14_3
  source 1..20: mol_type = other RNA
cgatgcggaggggctgtacg

SEQ ID NO: 28 - RNA, 20 nt, synthetic construct
  misc_feature 1..20: note = Description of sequence: sgRNA: ATG16L1_1
  source 1..20: mol_type = other RNA
catcatgtccgggactaaat

SEQ ID NO: 29 - RNA, 20 nt, synthetic construct
  misc_feature 1..20: note = Description of sequence: sgRNA: ATG16L1_2
  source 1..20: mol_type = other RNA
cgggactaaattggcaaacc

SEQ ID NO: 30 - RNA, 20 nt, synthetic construct
  misc_feature 1..20: note = Description of sequence: sgRNA: ATG16L1_3
  source 1..20: mol_type = other RNA
attaagccgattggcttcct

SEQ ID NO: 31 - RNA, 20 nt, synthetic construct
  misc_feature 1..20: note = Description of sequence: sgRNA: ATG16L2_1
  source 1..20: mol_type = other RNA
tacgcaaaaggcgcttttcc

SEQ ID NO: 32 - RNA, 20 nt, synthetic construct
  misc_feature 1..20: note = Description of sequence: sgRNA: ATG16L2_2
  source 1..20: mol_type = other RNA
cggacccacatacacttacc

SEQ ID NO: 33 - RNA, 20 nt, synthetic construct
  misc_feature 1..20: note = Description of sequence: sgRNA: ATG16L2_3
  source 1..20: mol_type = other RNA
gcaacctgcgcaacgagcgc

SEQ ID NO: 34 - RNA, 20 nt, synthetic construct
  misc_feature 1..20: note = Description of sequence: sgRNA: ATG2A_1
  source 1..20: mol_type = other RNA
cgtgacatctcggagaccgc

SEQ ID NO: 35 - RNA, 20 nt, synthetic construct
  misc_feature 1..20: note = Description of sequence: sgRNA: ATG2A_2
  source 1..20: mol_type = other RNA
gtgacatctcggagaccgcc

SEQ ID NO: 36 - RNA, 20 nt, synthetic construct
  misc_feature 1..20: note = Description of sequence: sgRNA: ATG2A_3
  source 1..20: mol_type = other RNA
atctcggagaccgccgggcc

SEQ ID NO: 37 - RNA, 20 nt, synthetic construct
  misc_feature 1..20: note = Description of sequence: sgRNA: ATG2B_1
  source 1..20: mol_type = other RNA
atggactccgaaaacggcca

SEQ ID NO: 38 - RNA, 20 nt, synthetic construct
  misc_feature 1..20: note = Description of sequence: sgRNA: ATG2B_2
  source 1..20: mol_type = other RNA
gaaactgctgacgaatcctc

SEQ ID NO: 39 - RNA, 20 nt, synthetic construct
  misc_feature 1..20: note = Description of sequence: sgRNA: ATG2B_3
  source 1..20: mol_type = other RNA
gtgcagattggacggttaat

SEQ ID NO: 40 - RNA, 20 nt, synthetic construct
  misc_feature 1..20: note = Description of sequence: sgRNA: ATG3_1
  source 1..20: mol_type = other RNA
ttatagtgccgtgctataag

SEQ ID NO: 41 - RNA, 20 nt, synthetic construct
  misc_feature 1..20: note = Description of sequence: sgRNA: ATG3_2
  source 1..20: mol_type = other RNA
tgtttgcaccgcttatagca

SEQ ID NO: 42 - RNA, 20 nt, synthetic construct
  misc_feature 1..20: note = Description of sequence: sgRNA: ATG3_3
  source 1..20: mol_type = other RNA
acaaccataatcgtggagtc

SEQ ID NO: 43 - RNA, 20 nt, synthetic construct
  misc_feature 1..20: note = Description of sequence: sgRNA: ATG4A_1
  source 1..20: mol_type = other RNA
attggtgggtattcgtaggt

SEQ ID NO: 44 - RNA, 20 nt, synthetic construct
  misc_feature 1..20: note = Description of sequence: sgRNA: ATG4A_2
  source 1..20: mol_type = other RNA
ggatccttcagttgcattgg

SEQ ID NO: 45 - RNA, 20 nt, synthetic construct
  misc_feature 1..20: note = Description of sequence: sgRNA: ATG4A_3
  source 1..20: mol_type = other RNA
ttaggatgttcattcgctgt

SEQ ID NO: 46 - RNA, 20 nt, synthetic construct
  misc_feature 1..20: note = Description of sequence: sgRNA: ATG4B_1
  source 1..20: mol_type = other RNA
agcaaaccggagagtgtcgt

SEQ ID NO: 47 - RNA, 20 nt, synthetic construct
  misc_feature 1..20: note = Description of sequence: sgRNA: ATG4B_2
  source 1..20: mol_type = other RNA
tcagagcccgtttggatact

SEQ ID NO: 48 - RNA, 20 nt, synthetic construct
  misc_feature 1..20: note = Description of sequence: sgRNA: ATG4B_3
  source 1..20: mol_type = other RNA
tcctgtcgatgaatgcgttg

SEQ ID NO: 49 - RNA, 20 nt, synthetic construct
  misc_feature 1..20: note = Description of sequence: sgRNA: ATG4C_1
  source 1..20: mol_type = other RNA
agagtcgggatgtacaatag

SEQ ID NO: 50 - RNA, 20 nt, synthetic construct
  misc_feature 1..20: note = Description of sequence: sgRNA: ATG4C_2
  source 1..20: mol_type = other RNA
atagaggatcacgtaattgc

SEQ ID NO: 51 - RNA, 20 nt, synthetic construct
  misc_feature 1..20: note = Description of sequence: sgRNA: ATG4C_3
  source 1..20: mol_type = other RNA
attgtacatcccgactctgc

SEQ ID NO: 52 - RNA, 20 nt, synthetic construct
  misc_feature 1..20: note = Description of sequence: sgRNA: ATG4D_1
  source 1..20: mol_type = other RNA
cctcgccctcgaaacggtag

SEQ ID NO: 53 - RNA, 20 nt, synthetic construct
  misc_feature 1..20: note = Description of sequence: sgRNA: ATG4D_2
  source 1..20: mol_type = other RNA
tggccgccgctaccgtttcg

SEQ ID NO: 54 - RNA, 20 nt, synthetic construct
  misc_feature 1..20: note = Description of sequence: sgRNA: ATG4D_3
  source 1..20: mol_type = other RNA
cccggcggtatgtgagccac

SEQ ID NO: 55 - RNA, 20 nt, synthetic construct
  misc_feature 1..20: note = Description of sequence: sgRNA: ATG5_1
  source 1..20: mol_type = other RNA
aacttgtttcacgctatatc

SEQ ID NO: 56 - RNA, 20 nt, synthetic construct
  misc_feature 1..20: note = Description of sequence: sgRNA: ATG5_2
  source 1..20: mol_type = other RNA
gagtgaacatctgagctacc

SEQ ID NO: 57 - RNA, 20 nt, synthetic construct
  misc_feature 1..20: note = Description of sequence: sgRNA: ATG5_3
  source 1..20: mol_type = other RNA
tccgattgatggcccaaaac

SEQ ID NO: 58 - RNA, 20 nt, synthetic construct
  misc_feature 1..20: note = Description of sequence: sgRNA: ATG7_1
  source 1..20: mol_type = other RNA
ctaggacgttgatggtaagt

SEQ ID NO: 59 - RNA, 20 nt, synthetic construct
  misc_feature 1..20: note = Description of sequence: sgRNA: ATG7_2
  source 1..20: mol_type = other RNA
gaagctgaacgagtatcggc

SEQ ID NO: 60 - RNA, 20 nt, synthetic construct
  misc_feature 1..20: note = Description of sequence: sgRNA: ATG7_3
  source 1..20: mol_type = other RNA
cttgaaagactcgagtgtgt

SEQ ID NO: 61 - RNA, 20 nt, synthetic construct
  misc_feature 1..20: note = Description of sequence: sgRNA: ATG9A_1
  source 1..20: mol_type = other RNA
ctgttggtgcacgtcgccga

SEQ ID NO: 62 - RNA, 20 nt, synthetic construct
  misc_feature 1..20: note = Description of sequence: sgRNA: ATG9A_2
  source 1..20: mol_type = other RNA
cctgttggtgcacgtcgccg

SEQ ID NO: 63 - RNA, 20 nt, synthetic construct
  misc_feature 1..20: note = Description of sequence: sgRNA: ATG9A_3
  source 1..20: mol_type = other RNA
cctcggcgacgtgcaccaac

SEQ ID NO: 64 - RNA, 20 nt, synthetic construct
  misc_feature 1..20: note = Description of sequence: sgRNA: ATG9B_1
  source 1..20: mol_type = other RNA
cgacgaggacgtgctagccg

SEQ ID NO: 65 - RNA, 20 nt, synthetic construct
  misc_feature 1..20: note = Description of sequence: sgRNA: ATG9B_2
  source 1..20: mol_type = other RNA
tagcacgtcctcgtcgtaga

SEQ ID NO: 66 - RNA, 20 nt, synthetic construct
  misc_feature 1..20: note = Description of sequence: sgRNA: ATG9B_3
  source 1..20: mol_type = other RNA
tgtgctcaccgtctacgacg

SEQ ID NO: 67 - RNA, 20 nt, synthetic construct
  misc_feature 1..20: note = Description of sequence: sgRNA: BAD_1
  source 1..20: mol_type = other RNA
ctttcggggccgctcgcgct

SEQ ID NO: 68 - RNA, 20 nt, synthetic construct
  misc_feature 1..20: note = Description of sequence: sgRNA: BAD_2
  source 1..20: mol_type = other RNA
gctatggccgcgagctccgg

SEQ ID NO: 69 - RNA, 20 nt, synthetic construct
  misc_feature 1..20: note = Description of sequence: sgRNA: BAD_3
  source 1..20: mol_type = other RNA
gagcgcgagcggccccgaaa

SEQ ID NO: 70 - RNA, 20 nt, synthetic construct
  misc_feature 1..20: note = Description of sequence: sgRNA: BAK1_1
  source 1..20: mol_type = other RNA
catgaagtcgaccacgaagc

SEQ ID NO: 71 - RNA, 20 nt, synthetic construct
  misc_feature 1..20: note = Description of sequence: sgRNA: BAK1_2
  source 1..20: mol_type = other RNA
gcatgaagtcgaccacgaag

SEQ ID NO: 72 - RNA, 20 nt, synthetic construct
  misc_feature 1..20: note = Description of sequence: sgRNA: BAK1_3
  source 1..20: mol_type = other RNA
gcaggtgagctacaaccgct

SEQ ID NO: 73 - RNA, 20 nt, synthetic construct
  misc_feature 1..20: note = Description of sequence: sgRNA: BAX_1
  source 1..20: mol_type = other RNA
gcgagtgtctcaagcgcatc

SEQ ID NO: 74 - RNA, 20 nt, synthetic construct
  misc_feature 1..20: note = Description of sequence: sgRNA: BAX_2
  source 1..20: mol_type = other RNA
caagcgcatcggggacgaac

SEQ ID NO: 75 - RNA, 20 nt, synthetic construct
  misc_feature 1..20: note = Description of sequence: sgRNA: BAX_3
  source 1..20: mol_type = other RNA
tcggaaaaagacctctcggg

SEQ ID NO: 76 - RNA, 20 nt, synthetic construct
  misc_feature 1..20: note = Description of sequence: sgRNA: BBC3_1
  source 1..20: mol_type = other RNA
tcaacgcacagtacgagcgg

SEQ ID NO: 77 - RNA, 20 nt, synthetic construct
  misc_feature 1..20: note = Description of sequence: sgRNA: BBC3_2
  source 1..20: mol_type = other RNA
gccgctcgtactgtgcgttg

SEQ ID NO: 78 - RNA, 20 nt, synthetic construct
  misc_feature 1..20: note = Description of sequence: sgRNA: BBC3_3
  source 1..20: mol_type = other RNA
caacgcacagtacgagcggc

SEQ ID NO: 79 - RNA, 20 nt, synthetic construct
  misc_feature 1..20: note = Description of sequence: sgRNA: BCL2_1
  source 1..20: mol_type = other RNA
ggggccgtacagttccacaa

SEQ ID NO: 80 - RNA, 20 nt, synthetic construct
  misc_feature 1..20: note = Description of sequence: sgRNA: BCL2_2
  source 1..20: mol_type = other RNA
tcaaacagaggccgcatgct

SEQ ID NO: 81 - RNA, 20 nt, synthetic construct
  misc_feature 1..20: note = Description of sequence: sgRNA: BCL2_3
  source 1..20: mol_type = other RNA
aagcgtccccgcgcggtgaa

SEQ ID NO: 82 - RNA, 20 nt, synthetic construct
  misc_feature 1..20: note = Description of sequence: sgRNA: BCL2A1_1
  source 1..20: mol_type = other RNA
cttataggtatccacatccg

SEQ ID NO: 83 - RNA, 20 nt, synthetic construct
  misc_feature 1..20: note = Description of sequence: sgRNA: BCL2A1_2
  source 1..20: mol_type = other RNA
ccttataggtatccacatcc

SEQ ID NO: 84 - RNA, 20 nt, synthetic construct
  misc_feature 1..20: note = Description of sequence: sgRNA: BCL2A1_3
  source 1..20: mol_type = other RNA
ttgaagacggcatcattaac

SEQ ID NO: 85 - RNA, 20 nt, synthetic construct
  misc_feature 1..20: note = Description of sequence: sgRNA: BCL2L1_1
  source 1..20: mol_type = other RNA
gtttgaactgcggtaccggc

SEQ ID NO: 86 - RNA, 20 nt, synthetic construct
  misc_feature 1..20: note = Description of sequence: sgRNA: BCL2L1_2
  source 1..20: mol_type = other RNA
agtttgaactgcggtaccgg

SEQ ID NO: 87 - RNA, 20 nt, synthetic construct
  misc_feature 1..20: note = Description of sequence: sgRNA: BCL2L1_3
  source 1..20: mol_type = other RNA
acgagtttgaactgcggtac

SEQ ID NO: 88 - RNA, 20 nt, synthetic construct
  misc_feature 1..20: note = Description of sequence: sgRNA: BCL2L10_1
  source 1..20: mol_type = other RNA
cgggaaccgcttcgagctgg

SEQ ID NO: 89 - RNA, 20 nt, synthetic construct
  misc_feature 1..20: note = Description of sequence: sgRNA: BCL2L10_2
  source 1..20: mol_type = other RNA
ccggtgaatctgccgtaacc

SEQ ID NO: 90 - RNA, 20 nt, synthetic construct
  misc_feature 1..20: note = Description of sequence: sgRNA: BCL2L10_3
  source 1..20: mol_type = other RNA
ctgccgtaacctggcggccg

SEQ ID NO: 91 - RNA, 20 nt, synthetic construct
  misc_feature 1..20: note = Description of sequence: sgRNA: BCL2L11_1
  source 1..20: mol_type = other RNA
gcccaagagttgcggcgtat

SEQ ID NO: 92 - RNA, 20 nt, synthetic construct
  misc_feature 1..20: note = Description of sequence: sgRNA: BCL2L11_2
  source 1..20: mol_type = other RNA
tccaatacgccgcaactctt

SEQ ID NO: 93 - RNA, 20 nt, synthetic construct
  misc_feature 1..20: note = Description of sequence: sgRNA: BCL2L11_3
  source 1..20: mol_type = other RNA
ctccaatacgccgcaactct

SEQ ID NO: 94 - RNA, 20 nt, synthetic construct
  misc_feature 1..20: note = Description of sequence: sgRNA: BCL2L12_1
  source 1..20: mol_type = other RNA
gggccgtcccatcggctttt

SEQ ID NO: 95 - RNA, 20 nt, synthetic construct
  misc_feature 1..20: note = Description of sequence: sgRNA: BCL2L12_2
  source 1..20: mol_type = other RNA
aagccgatgggacggcccgc

SEQ ID NO: 96 - RNA, 20 nt, synthetic construct
  misc_feature 1..20: note = Description of sequence: sgRNA: BCL2L12_3
  source 1..20: mol_type = other RNA
agccgatgggacggcccgct

SEQ ID NO: 97 - RNA, 20 nt, synthetic construct
  misc_feature 1..20: note = Description of sequence: sgRNA: BCL2L13_1
  source 1..20: mol_type = other RNA
gaggacgccattgaattggc

SEQ ID NO: 98 - RNA, 20 nt, synthetic construct
  misc_feature 1..20: note = Description of sequence: sgRNA: BCL2L13_2
  source 1..20: mol_type = other RNA
atccggaacggtatccggag

SEQ ID NO: 99 - RNA, 20 nt, synthetic construct
  misc_feature 1..20: note = Description of sequence: sgRNA: BCL2L13_3
  source 1..20: mol_type = other RNA
ctgacatccggaacggtatc

SEQ ID NO: 100 - RNA, 20 nt, synthetic construct
  misc_feature 1..20: note = Description of sequence: sgRNA: BCL2L14_1
  source 1..20: mol_type = other RNA
tggccgtgacgtctattaca

SEQ ID NO: 101 - RNA, 20 nt, synthetic construct
  misc_feature 1..20: note = Description of sequence: sgRNA: BCL2L14_2
  source 1..20: mol_type = other RNA
gatccagcagcacggtggat

SEQ ID NO: 102 - RNA, 20 nt, synthetic construct
  misc_feature 1..20: note = Description of sequence: sgRNA: BCL2L14_3
  source 1..20: mol_type = other RNA
ggccgtgacgtctattacaa

SEQ ID NO: 103 - RNA, 20 nt, synthetic construct
  misc_feature 1..20: note = Description of sequence: sgRNA: BCL2L2_1
  source 1..20: mol_type = other RNA
tctatacggggacggggccc

SEQ ID NO: 104 - RNA, 20 nt, synthetic construct
  misc_feature 1..20: note = Description of sequence: sgRNA: BCL2L2_2
  source 1..20: mol_type = other RNA
cacagctctatacggggacg

SEQ ID NO: 105 - RNA, 20 nt, synthetic construct
  misc_feature 1..20: note = Description of sequence: sgRNA: BCL2L2_3
  source 1..20: mol_type = other RNA
cggagttcacagctctatac

SEQ ID NO: 106 - RNA, 20 nt, synthetic construct
  misc_feature 1..20: note = Description of sequence: sgRNA: BECN1_1
  source 1..20: mol_type = other RNA
ttacggaaaccattcatatc

SEQ ID NO: 107 - RNA, 20 nt, synthetic construct
  misc_feature 1..20: note = Description of sequence: sgRNA: BECN1_2
  source 1..20: mol_type = other RNA
gcgttatgcccagacgcagc

SEQ ID NO: 108 - RNA, 20 nt, synthetic construct
  misc_feature 1..20: note = Description of sequence: sgRNA: BECN1_3
  source 1..20: mol_type = other RNA
gcgtctgggcataacgcatc

SEQ ID NO: 109 - RNA, 20 nt, synthetic construct
  misc_feature 1..20: note = Description of sequence: sgRNA: BID_1
  source 1..20: mol_type = other RNA
tggaaccgttgttgacctga

SEQ ID NO: 110 - RNA, 20 nt, synthetic construct
  misc_feature 1..20: note = Description of sequence: sgRNA: BID_2
  source 1..20: mol_type = other RNA
acatcatccggaatattgcc

SEQ ID NO: 111 - RNA, 20 nt, synthetic construct
  misc_feature 1..20: note = Description of sequence: sgRNA: BID_3
  source 1..20: mol_type = other RNA
agaacctacgcacctacgtg

SEQ ID NO: 112 - RNA, 20 nt, synthetic construct
  misc_feature 1..20: note = Description of sequence: sgRNA: BIK_1
  source 1..20: mol_type = other RNA
gccaagaacctccatggtcg

SEQ ID NO: 113 - RNA, 20 nt, synthetic construct
  misc_feature 1..20: note = Description of sequence: sgRNA: BIK_2
  source 1..20: mol_type = other RNA
tatggaggacttcgattctt

SEQ ID NO: 114 - RNA, 20 nt, synthetic construct
  misc_feature 1..20: note = Description of sequence: sgRNA: BIK_3
  source 1..20: mol_type = other RNA
cgtagatgaaagccagaccc

SEQ ID NO: 115 - RNA, 20 nt, synthetic construct
  misc_feature 1..20: note = Description of sequence: sgRNA: BIRC2_1
  source 1..20: mol_type = other RNA
atgttttgatacgagggacc

SEQ ID NO: 116 - RNA, 20 nt, synthetic construct
  misc_feature 1..20: note = Description of sequence: sgRNA: BIRC2_2
  source 1..20: mol_type = other RNA
tgttttgatacgagggacct

SEQ ID NO: 117 - RNA, 20 nt, synthetic construct
  misc_feature 1..20: note = Description of sequence: sgRNA: BIRC2_3
  source 1..20: mol_type = other RNA
atattcaactttccccgccg

SEQ ID NO: 118 - RNA, 20 nt, synthetic construct
  misc_feature 1..20: note = Description of sequence: sgRNA: BIRC3_1
  source 1..20: mol_type = other RNA
acccggaagtaatgagtgtg

SEQ ID NO: 119 - RNA, 20 nt, synthetic construct
  misc_feature 1..20: note = Description of sequence: sgRNA: BIRC3_2
  source 1..20: mol_type = other RNA
attgagcaattgggaaccga

SEQ ID NO: 120 - RNA, 20 nt, synthetic construct
  misc_feature 1..20: note = Description of sequence: sgRNA: BIRC3_3
  source 1..20: mol_type = other RNA
cggcagttagtagactatcc

SEQ ID NO: 121 - RNA, 20 nt, synthetic construct
  misc_feature 1..20: note = Description of sequence: sgRNA: BIRC5_1
  source 1..20: mol_type = other RNA
acttacatggggtcgtcatc

SEQ ID NO: 122 - RNA, 20 nt, synthetic construct
  misc_feature 1..20: note = Description of sequence: sgRNA: BIRC5_2
  source 1..20: mol_type = other RNA
ccggcccactgtaaggatta

SEQ ID NO: 123 - RNA, 20 nt, synthetic construct
  misc_feature 1..20: note = Description of sequence: sgRNA: BIRC5_3
  source 1..20: mol_type = other RNA
gcgccgtgccatcgagcagc

SEQ ID NO: 124 - RNA, 20 nt, synthetic construct
  misc_feature 1..20: note = Description of sequence: sgRNA: BIRC6_1
  source 1..20: mol_type = other RNA
gcatgcactgcgacgccgac

SEQ ID NO: 125 - RNA, 20 nt, synthetic construct
  misc_feature 1..20: note = Description of sequence: sgRNA: BIRC6_2
  source 1..20: mol_type = other RNA
gcctcatgtaggctataggt

SEQ ID NO: 126 - RNA, 20 nt, synthetic construct
  misc_feature 1..20: note = Description of sequence: sgRNA: BIRC6_3
  source 1..20: mol_type = other RNA
gcttgaattacccgttacag

SEQ ID NO: 127 - RNA, 20 nt, synthetic construct
  misc_feature 1..20: note = Description of sequence: sgRNA: BIRC7_1
  source 1..20: mol_type = other RNA
agtcagcggccagtcataga

SEQ ID NO: 128 - RNA, 20 nt, synthetic construct
  misc_feature 1..20: note = Description of sequence: sgRNA: BIRC7_2
  source 1..20: mol_type = other RNA
agccggtgatggtcccacgc

SEQ ID NO: 129 - RNA, 20 nt, synthetic construct
  misc_feature 1..20: note = Description of sequence: sgRNA: BIRC7_3
  source 1..20: mol_type = other RNA
gcgcggggacgacccctgga

SEQ ID NO: 130 - RNA, 20 nt, synthetic construct
  misc_feature 1..20: note = Description of sequence: sgRNA: BMF_1
  source 1..20: mol_type = other RNA
actcaccataaaagagtcgc

SEQ ID NO: 131 - RNA, 20 nt, synthetic construct
  misc_feature 1..20: note = Description of sequence: sgRNA: BMF_2
  source 1..20: mol_type = other RNA
cggcttcatgtgcagcaagt

SEQ ID NO: 132 - RNA, 20 nt, synthetic construct
  misc_feature 1..20: note = Description of sequence: sgRNA: BMF_3
  source 1..20: mol_type = other RNA
caagtaggcacgggggttta

SEQ ID NO: 133 - RNA, 20 nt, synthetic construct
  misc_feature 1..20: note = Description of sequence: sgRNA: BNIP3_1
  source 1..20: mol_type = other RNA
tctgcggccgcgtcgcccat

SEQ ID NO: 134 - RNA, 20 nt, synthetic construct
  misc_feature 1..20: note = Description of sequence: sgRNA: BNIP3_2
  source 1..20: mol_type = other RNA
cccggccggaggaactcaca

SEQ ID NO: 135 - RNA, 20 nt, synthetic construct
  misc_feature 1..20: note = Description of sequence: sgRNA: BNIP3_3
  source 1..20: mol_type = other RNA
gtcgcggccaatgggcgacg

SEQ ID NO: 136 - RNA, 20 nt, synthetic construct
  misc_feature 1..20: note = Description of sequence: sgRNA: BNIP3L_1
  source 1..20: mol_type = other RNA
actaggtgggacgacattgt

SEQ ID NO: 137 - RNA, 20 nt, synthetic construct
  misc_feature 1..20: note = Description of sequence: sgRNA: BNIP3L_2
  source 1..20: mol_type = other RNA
gcccttcgccacaagaagat

SEQ ID NO: 138 - RNA, 20 nt, synthetic construct
  misc_feature 1..20: note = Description of sequence: sgRNA: BNIP3L_3
  source 1..20: mol_type = other RNA
cccgacacacctgagagcta

SEQ ID NO: 139 - RNA, 20 nt, synthetic construct
  misc_feature 1..20: note = Description of sequence: sgRNA: BOK_1
  source 1..20: mol_type = other RNA
cgagcgtgccgcgccggtcc

SEQ ID NO: 140 - RNA, 20 nt, synthetic construct
  misc_feature 1..20: note = Description of sequence: sgRNA: BOK_2
  source 1..20: mol_type = other RNA
gatctcggcggcgaagaccg

SEQ ID NO: 141 - RNA, 20 nt, synthetic construct
  misc_feature 1..20: note = Description of sequence: sgRNA: BOK_3
  source 1..20: mol_type = other RNA
ggtcttcgccgccgagatca

SEQ ID NO: 142 - RNA, 20 nt, synthetic construct
  misc_feature 1..20: note = Description of sequence: sgRNA: CA9_1
  source 1..20: mol_type = other RNA
ggcggatatccaccggggac

SEQ ID NO: 143 - RNA, 20 nt, synthetic construct
  misc_feature 1..20: note = Description of sequence: sgRNA: CA9_2
  source 1..20: mol_type = other RNA
agctgggggcggatatccac

SEQ ID NO: 144 - RNA, 20 nt, synthetic construct
  misc_feature 1..20: note = Description of sequence: sgRNA: CA9_3
  source 1..20: mol_type = other RNA
ctgcgcaacaatggccacag

SEQ ID NO: 145 - RNA, 20 nt, synthetic construct
  misc_feature 1..20: note = Description of sequence: sgRNA: CARD6_1
  source 1..20: mol_type = other RNA
tagacacgttaacttctcgg

SEQ ID NO: 146 - RNA, 20 nt, synthetic construct
  misc_feature 1..20: note = Description of sequence: sgRNA: CARD6_2
  source 1..20: mol_type = other RNA
cagtacctcctcaatctatg

SEQ ID NO: 147 - RNA, 20 nt, synthetic construct
  misc_feature 1..20: note = Description of sequence: sgRNA: CARD6_3
  source 1..20: mol_type = other RNA
tatgatgacccagagcacgt

SEQ ID NO: 148 - RNA, 20 nt, synthetic construct
  misc_feature 1..20: note = Description of sequence: sgRNA: CARD8_1
  source 1..20: mol_type = other RNA
ttgttagcaaggcgtcgctg

SEQ ID NO: 149 - RNA, 20 nt, synthetic construct
  misc_feature 1..20: note = Description of sequence: sgRNA: CARD8_2
  source 1..20: mol_type = other RNA
tcctgctgcggatcgccagt

SEQ ID NO: 150 - RNA, 20 nt, synthetic construct
  misc_feature 1..20: note = Description of sequence: sgRNA: CARD8_3
  source 1..20: mol_type = other RNA
tcccactggcgatccgcagc

SEQ ID NO: 151 - RNA, 20 nt, synthetic construct
  misc_feature 1..20: note = Description of sequence: sgRNA: CARS_1
  source 1..20: mol_type = other RNA
gcttcgatggacattcacgg

SEQ ID NO: 152 - RNA, 20 nt, synthetic construct
  misc_feature 1..20: note = Description of sequence: sgRNA: CARS_2
  source 1..20: mol_type = other RNA
tgagatgcgtcatagacggt

SEQ ID NO: 153 - RNA, 20 nt, synthetic construct
  misc_feature 1..20: note = Description of sequence: sgRNA: CARS_3
  source 1..20: mol_type = other RNA
tgatgtcaacaggagcgcga

SEQ ID NO: 154 - RNA, 20 nt, synthetic construct
  misc_feature 1..20: note = Description of sequence: sgRNA: CASP1_1
  source 1..20: mol_type = other RNA
accgaaggtgatcatcatcc

SEQ ID NO: 155 - RNA, 20 nt, synthetic construct
  misc_feature 1..20: note = Description of sequence: sgRNA: CASP1_2
  source 1..20: mol_type = other RNA
gcattgagttgtagtatatc

SEQ ID NO: 156 - RNA, 20 nt, synthetic construct
  misc_feature 1..20: note = Description of sequence: sgRNA: CASP1_3
  source 1..20: mol_type = other RNA
tccgcaaggtaagatgctag

SEQ ID NO: 157 - RNA, 20 nt, synthetic construct
  misc_feature 1..20: note = Description of sequence: sgRNA: CASP10_1
  source 1..20: mol_type = other RNA
agccgagtcgtatcaaggag

SEQ ID NO: 158 - RNA, 20 nt, synthetic construct
  misc_feature 1..20: note = Description of sequence: sgRNA: CASP10_2
  source 1..20: mol_type = other RNA
ttcctctccttgatacgact

SEQ ID NO: 159 - RNA, 20 nt, synthetic construct
  misc_feature 1..20: note = Description of sequence: sgRNA: CASP10_3
  source 1..20: mol_type = other RNA
aaggaagccgagtcgtatca

SEQ ID NO: 160 - RNA, 20 nt, synthetic construct
  misc_feature 1..20: note = Description of sequence: sgRNA: CASP14_1
  source 1..20: mol_type = other RNA
aatgagcaatccgcggtctt

SEQ ID NO: 161 - RNA, 20 nt, synthetic construct
  misc_feature 1..20: note = Description of sequence: sgRNA: CASP14_2
  source 1..20: mol_type = other RNA
caatccgcggtctttggaag

SEQ ID NO: 162 - RNA, 20 nt, synthetic construct
  misc_feature 1..20: note = Description of sequence: sgRNA: CASP14_3
  source 1..20: mol_type = other RNA
accccactgccgaggtattg

SEQ ID NO: 163 - RNA, 20 nt, synthetic construct
  misc_feature 1..20: note = Description of sequence: sgRNA: CASP2_1
  source 1..20: mol_type = other RNA
tactaaagcttcataccggt

SEQ ID NO: 164 - RNA, 20 nt, synthetic construct
  misc_feature 1..20: note = Description of sequence: sgRNA: CASP2_2
  source 1..20: mol_type = other RNA
ttactaaagcttcataccgg

SEQ ID NO: 165 - RNA, 20 nt, synthetic construct
  misc_feature 1..20: note = Description of sequence: sgRNA: CASP2_3
  source 1..20: mol_type = other RNA
aagaagctccgcctgtcgac

SEQ ID NO: 166 - RNA, 20 nt, synthetic construct
  misc_feature 1..20: note = Description of sequence: sgRNA: CASP3_1
  source 1..20: mol_type = other RNA
tacccgggttaaccgaaagg

SEQ ID NO: 167 - RNA, 20 nt, synthetic construct
  misc_feature 1..20: note = Description of sequence: sgRNA: CASP3_2
  source 1..20: mol_type = other RNA
tcttacccgggttaaccgaa

SEQ ID NO: 168 - RNA, 20 nt, synthetic construct
  misc_feature 1..20: note = Description of sequence: sgRNA: CASP3_3
  source 1..20: mol_type = other RNA
tgccacctttcggttaaccc

SEQ ID NO: 169 - RNA, 20 nt, synthetic construct
  misc_feature 1..20: note = Description of sequence: sgRNA: CASP4_1
  source 1..20: mol_type = other RNA
tccatattcggatgagctgc

SEQ ID NO: 170 - RNA, 20 nt, synthetic construct
  misc_feature 1..20: note = Description of sequence: sgRNA: CASP4_2
  source 1..20: mol_type = other RNA
tgcagctcatccgaatatgg

SEQ ID NO: 171 - RNA, 20 nt, synthetic construct
  misc_feature 1..20: note = Description of sequence: sgRNA: CASP4_3
  source 1..20: mol_type = other RNA
gccactgaaagatacatacg

SEQ ID NO: 172 - RNA, 20 nt, synthetic construct
  misc_feature 1..20: note = Description of sequence: sgRNA: CASP5_1
  source 1..20: mol_type = other RNA
gtcgacttttgatccgtatt

SEQ ID NO: 173 - RNA, 20 nt, synthetic construct
  misc_feature 1..20: note = Description of sequence: sgRNA: CASP5_2
  source 1..20: mol_type = other RNA
ttgatccgtattaggtacta

SEQ ID NO: 174 - RNA, 20 nt, synthetic construct
  misc_feature 1..20: note = Description of sequence: sgRNA: CASP5_3
  source 1..20: mol_type = other RNA
ggggctcactatgacatcgt

SEQ ID NO: 175 - RNA, 20 nt, synthetic construct
  misc_feature 1..20: note = Description of sequence: sgRNA: CASP6_1
  source 1..20: mol_type = other RNA
aagattgtctctatctgcgc

SEQ ID NO: 176 - RNA, 20 nt, synthetic construct
  misc_feature 1..20: note = Description of sequence: sgRNA: CASP6_2
  source 1..20: mol_type = other RNA
ctgcaatgagctcggcctcg

SEQ ID NO: 177 - RNA, 20 nt, synthetic construct
  misc_feature 1..20: note = Description of sequence: sgRNA: CASP6_3
  source 1..20: mol_type = other RNA
caaagcaatcggcatctgcg

SEQ ID NO: 178 - RNA, 20 nt, synthetic construct
  misc_feature 1..20: note = Description of sequence: sgRNA: CASP7_1
  source 1..20: mol_type = other RNA
atatgtaggcactcggtccc

SEQ ID NO: 179 - RNA, 20 nt, synthetic construct
  misc_feature 1..20: note = Description of sequence: sgRNA: CASP7_2
  source 1..20: mol_type = other RNA
tgtaggcactcggtcccggg

SEQ ID NO: 180 - RNA, 20 nt, synthetic construct
  misc_feature 1..20: note = Description of sequence: sgRNA: CASP7_3
  source 1..20: mol_type = other RNA
ggtacaaacgaggaccggtc

SEQ ID NO: 181 - RNA, 20 nt, synthetic construct
  misc_feature 1..20: note = Description of sequence: sgRNA: CASP8_1
  source 1..20: mol_type = other RNA
tccgggatatatctcgtttg

SEQ ID NO: 182 - RNA, 20 nt, synthetic construct
  misc_feature 1..20: note = Description of sequence: sgRNA: CASP8_2
  source 1..20: mol_type = other RNA
gggtcgatcatctattaata

SEQ ID NO: 183 - RNA, 20 nt, synthetic construct
  misc_feature 1..20: note = Description of sequence: sgRNA: CASP8_3
  source 1..20: mol_type = other RNA
gcctggactacattccgcaa

SEQ ID NO: 184 - RNA, 20 nt, synthetic construct
  misc_feature 1..20: note = Description of sequence: sgRNA: CASP9_1
  source 1..20: mol_type = other RNA
ccgccgatccgcttcgtcca

SEQ ID NO: 185 - RNA, 20 nt, synthetic construct
  misc_feature 1..20: note = Description of sequence: sgRNA: CASP9_2
  source 1..20: mol_type = other RNA
gaacagctcgcggctcagca

SEQ ID NO: 186 - RNA, 20 nt, synthetic construct
  misc_feature 1..20: note = Description of sequence: sgRNA: CASP9_3
  source 1..20: mol_type = other RNA
cttcgtttctgcgaactaac

SEQ ID NO: 187 - RNA, 20 nt, synthetic construct
  misc_feature 1..20: note = Description of sequence: sgRNA: CD40_1
  source 1..20: mol_type = other RNA
accccagtgcgtgcgctgtt

SEQ ID NO: 188 - RNA, 20 nt, synthetic construct
  misc_feature 1..20: note = Description of sequence: sgRNA: CD40_2
  source 1..20: mol_type = other RNA
attcgctttcaccgcaagga

SEQ ID NO: 189 - RNA, 20 nt, synthetic construct
  misc_feature 1..20: note = Description of sequence: sgRNA: CD40_3
  source 1..20: mol_type = other RNA
aggaattcgctttcaccgca

SEQ ID NO: 190 - RNA, 20 nt, synthetic construct
  misc_feature 1..20: note = Description of sequence: sgRNA: CD40LG_1
  source 1..20: mol_type = other RNA
tctccccgatctgcggccac

SEQ ID NO: 191 - RNA, 20 nt, synthetic construct
  misc_feature 1..20: note = Description of sequence: sgRNA: CD40LG_2
  source 1..20: mol_type = other RNA
atgacatgtgccgcaatttg

SEQ ID NO: 192 - RNA, 20 nt, synthetic construct
  misc_feature 1..20: note = Description of sequence: sgRNA: CD40LG_3
  source 1..20: mol_type = other RNA
cgaatctaccgggggacttt

SEQ ID NO: 193 - RNA, 20 nt, synthetic construct
  misc_feature 1..20: note = Description of sequence: sgRNA: CD70_1
  source 1..20: mol_type = other RNA
tcaccaagcccgcgaccaat

SEQ ID NO: 194 - RNA, 20 nt, synthetic construct
  misc_feature 1..20: note = Description of sequence: sgRNA: CD70_2
  source 1..20: mol_type = other RNA
gctttggtcccattggtcgc

SEQ ID NO: 195 - RNA, 20 nt, synthetic construct
  misc_feature 1..20: note = Description of sequence: sgRNA: CD70_3
  source 1..20: mol_type = other RNA
agctgccgctcgagtcactt

SEQ ID NO: 196 - RNA, 20 nt, synthetic construct
  misc_feature 1..20: note = Description of sequence: sgRNA: CFLAR_1
  source 1..20: mol_type = other RNA
gacatcctactcttagacta

SEQ ID NO: 197 - RNA, 20 nt, synthetic construct
  misc_feature 1..20: note = Description of sequence: sgRNA: CFLAR_2
  source 1..20: mol_type = other RNA
ctcaccttgtttcggactat

SEQ ID NO: 198 - RNA, 20 nt, synthetic construct
  misc_feature 1..20: note = Description of sequence: sgRNA: CFLAR_3
  source 1..20: mol_type = other RNA
attacctatagtccgaaaca

SEQ ID NO: 199 - RNA, 20 nt, synthetic construct
  misc_feature 1..20: note = Description of sequence: sgRNA: COL4A3BP_1
  source 1..20: mol_type = other RNA
ccagctctgattatccgaca

SEQ ID NO: 200 - RNA, 20 nt, synthetic construct
  misc_feature 1..20: note = Description of sequence: sgRNA: COL4A3BP_2
  source 1..20: mol_type = other RNA
ccatgtcggataatcagagc

SEQ ID NO: 201 - RNA, 20 nt, synthetic construct
  misc_feature 1..20: note = Description of sequence: sgRNA: COL4A3BP_3
  source 1..20: mol_type = other RNA
cggttgtcgagcctccatgt

SEQ ID NO: 202 - RNA, 20 nt, synthetic construct
  misc_feature 1..20: note = Description of sequence: sgRNA: CS_1
  source 1..20: mol_type = other RNA
cgtcatgccataatactgta

SEQ ID NO: 203 - RNA, 20 nt, synthetic construct
  misc_feature 1..20: note = Description of sequence: sgRNA: CS_2
  source 1..20: mol_type = other RNA
ctcaagggcatccgtttccg

SEQ ID NO: 204 - RNA, 20 nt, synthetic construct
  misc_feature 1..20: note = Description of sequence: sgRNA: CS_3
  source 1..20: mol_type = other RNA
cagccgaaccaagtactggg

SEQ ID NO: 205 - RNA, 20 nt, synthetic construct
  misc_feature 1..20: note = Description of sequence: sgRNA: CYCS_1
  source 1..20: mol_type = other RNA
tcttgtttaggcatcatctg

SEQ ID NO: 206 - RNA, 20 nt, synthetic construct
  misc_feature 1..20: note = Description of sequence: sgRNA: CYCS_2
  source 1..20: mol_type = other RNA
tgccacaccgttgaaaaggg

SEQ ID NO: 207 - RNA, 20 nt, synthetic construct
  misc_feature 1..20: note = Description of sequence: sgRNA: CYCS_3
  source 1..20: mol_type = other RNA
atctccatggtctctttggg

SEQ ID NO: 208 - RNA, 20 nt, synthetic construct
  misc_feature 1..20: note = Description of sequence: sgRNA: CYLD_1
  source 1..20: mol_type = other RNA
ggataaccctattggcaact

SEQ ID NO: 209 - RNA, 20 nt, synthetic construct
  misc_feature 1..20: note = Description of sequence: sgRNA: CYLD_2
  source 1..20: mol_type = other RNA
tatggggtaatccgttggat

SEQ ID NO: 210 - RNA, 20 nt, synthetic construct
  misc_feature 1..20: note = Description of sequence: sgRNA: CYLD_3
  source 1..20: mol_type = other RNA
cgttggatcggtcagccacc

SEQ ID NO: 211 - RNA, 20 nt, synthetic construct
  misc_feature 1..20: note = Description of sequence: sgRNA: DDX58_1
  source 1..20: mol_type = other RNA
gcttacgtccacaagtgctc

SEQ ID NO: 212 - RNA, 20 nt, synthetic construct
  misc_feature 1..20: note = Description of sequence: sgRNA: DDX58_2
  source 1..20: mol_type = other RNA
tgactgcctcggttggtgtt

SEQ ID NO: 213 - RNA, 20 nt, synthetic construct
  misc_feature 1..20: note = Description of sequence: sgRNA: DDX58_3
  source 1..20: mol_type = other RNA
gactgcctcggttggtgttg

SEQ ID NO: 214 - RNA, 20 nt, synthetic construct
  misc_feature 1..20: note = Description of sequence: sgRNA: DIABLO_1
  source 1..20: mol_type = other RNA
ggcgtccgcgcgctgcacaa

SEQ ID NO: 215 - RNA, 20 nt, synthetic construct
  misc_feature 1..20: note = Description of sequence: sgRNA: DIABLO_2
  source 1..20: mol_type = other RNA
agccgccattgtgcagcgcg

SEQ ID NO: 216 - RNA, 20 nt, synthetic construct
  misc_feature 1..20: note = Description of sequence: sgRNA: DIABLO_3
  source 1..20: mol_type = other RNA
gtgtgcggttcctattgcac

SEQ ID NO: 217 - RNA, 20 nt, synthetic construct
  misc_feature 1..20: note = Description of sequence: sgRNA: DNM1L_1
  source 1..20: mol_type = other RNA
aatcgtcgtagtgggaacgc

SEQ ID NO: 218 - RNA, 20 nt, synthetic construct
  misc_feature 1..20: note = Description of sequence: sgRNA: DNM1L_2
  source 1..20: mol_type = other RNA
gttcccactacgacgatttg

SEQ ID NO: 219 - RNA, 20 nt, synthetic construct
  misc_feature 1..20: note = Description of sequence: sgRNA: DNM1L_3
  source 1..20: mol_type = other RNA
ctgcctcaaatcgtcgtagt

SEQ ID NO: 220 - RNA, 20 nt, synthetic construct
  misc_feature 1..20: note = Description of sequence: sgRNA: DSCC1_1
  source 1..20: mol_type = other RNA
ggtcctcttcatcgcagcgc

SEQ ID NO: 221 - RNA, 20 nt, synthetic construct
  misc_feature 1..20: note = Description of sequence: sgRNA: DSCC1_2
  source 1..20: mol_type = other RNA
cgtcgcatccacctcgtcgc

SEQ ID NO: 222 - RNA, 20 nt, synthetic construct
  misc_feature 1..20: note = Description of sequence: sgRNA: DSCC1_3
  source 1..20: mol_type = other RNA
tgctacagtcttgtgattcg

SEQ ID NO: 223 - RNA, 20 nt, synthetic construct
  misc_feature 1..20: note = Description of sequence: sgRNA: ENDOG_1
  source 1..20: mol_type = other RNA
cgggcgagctggccaagtac

SEQ ID NO: 224 - RNA, 20 nt, synthetic construct
  misc_feature 1..20: note = Description of sequence: sgRNA: ENDOG_2
  source 1..20: mol_type = other RNA
agcacagcacgtacgactcg

SEQ ID NO: 225 - RNA, 20 nt, synthetic construct
  misc_feature 1..20: note = Description of sequence: sgRNA: ENDOG_3
  source 1..20: mol_type = other RNA
cgcggaagtcgcactcgcgc

SEQ ID NO: 226 - RNA, 20 nt, synthetic construct
  misc_feature 1..20: note = Description of sequence: sgRNA: FADD_1
  source 1..20: mol_type = other RNA
gttcctatgcctcgggcgcg

SEQ ID NO: 227 - RNA, 20 nt, synthetic construct
  misc_feature 1..20: note = Description of sequence: sgRNA: FADD_2
  source 1..20: mol_type = other RNA
agtcgtcgacgcgccgcagc

SEQ ID NO: 228 - RNA, 20 nt, synthetic construct
  misc_feature 1..20: note = Description of sequence: sgRNA: FADD_3
  source 1..20: mol_type = other RNA
gcgtcgacgacttcgaggcg

SEQ ID NO: 229 - RNA, 20 nt, synthetic construct
  misc_feature 1..20: note = Description of sequence: sgRNA: FAS_1
  source 1..20: mol_type = other RNA
actgcgtgccctgccaagaa

SEQ ID NO: 230 - RNA, 20 nt, synthetic construct
  misc_feature 1..20: note = Description of sequence: sgRNA: FAS_2
  source 1..20: mol_type = other RNA
aagccaccccaagttagatc

SEQ ID NO: 231 - RNA, 20 nt, synthetic construct
  misc_feature 1..20: note = Description of sequence: sgRNA: FAS_3
  source 1..20: mol_type = other RNA
gatccagatctaacttgggg

SEQ ID NO: 232 - RNA, 20 nt, synthetic construct
  misc_feature 1..20: note = Description of sequence: sgRNA: FASLG_1
  source 1..20: mol_type = other RNA
actccgagaggtaagcctgc

SEQ ID NO: 233 - RNA, 20 nt, synthetic construct
  misc_feature 1..20: note = Description of sequence: sgRNA: FASLG_2
  source 1..20: mol_type = other RNA
ctggttgccttggtaggatt

SEQ ID NO: 234 - RNA, 20 nt, synthetic construct
  misc_feature 1..20: note = Description of sequence: sgRNA: FASLG_3
  source 1..20: mol_type = other RNA
tctggttgccttggtaggat

SEQ ID NO: 235 - RNA, 20 nt, synthetic construct
  misc_feature 1..20: note = Description of sequence: sgRNA: FOXI1_1
  source 1..20: mol_type = other RNA
atactcgccgcccccctcga

SEQ ID NO: 236 - RNA, 20 nt, synthetic construct
  misc_feature 1..20: note = Description of sequence: sgRNA: FOXI1_2
  source 1..20: mol_type = other RNA
tcgccgcccccctcgaagga

SEQ ID NO: 237 - RNA, 20 nt, synthetic construct
  misc_feature 1..20: note = Description of sequence: sgRNA: FOXI1_3
  source 1..20: mol_type = other RNA
tcgaggggggcggcgagtat

SEQ ID NO: 238 - RNA, 20 nt, synthetic construct
  misc_feature 1..20: note = Description of sequence: sgRNA: GPX4_1
  source 1..20: mol_type = other RNA
actcagcgtatcgggcgtgc

SEQ ID NO: 239 - RNA, 20 nt, synthetic construct
  misc_feature 1..20: note = Description of sequence: sgRNA: GPX4_2
  source 1..20: mol_type = other RNA
cacgcccgatacgctgagtg

SEQ ID NO: 240 - RNA, 20 nt, synthetic construct
  misc_feature 1..20: note = Description of sequence: sgRNA: GPX4_3
  source 1..20: mol_type = other RNA
cccgaactggttacacggga

SEQ ID NO: 241 - RNA, 20 nt, synthetic construct
  misc_feature 1..20: note = Description of sequence: sgRNA: GRB2_1
  source 1..20: mol_type = other RNA
acgacgagctgagcttcaaa

SEQ ID NO: 242 - RNA, 20 nt, synthetic construct
  misc_feature 1..20: note = Description of sequence: sgRNA: GRB2_2
  source 1..20: mol_type = other RNA
ttagacgttccggttcacgg

SEQ ID NO: 243 - RNA, 20 nt, synthetic construct
  misc_feature 1..20: note = Description of sequence: sgRNA: GRB2_3
  source 1..20: mol_type = other RNA
acgggggtgacataattgcg

SEQ ID NO: 244 - RNA, 20 nt, synthetic construct
  misc_feature 1..20: note = Description of sequence: sgRNA: GSDMD_1
  source 1..20: mol_type = other RNA
aggttgacacacttataacg

SEQ ID NO: 245 - RNA, 20 nt, synthetic construct
  misc_feature 1..20: note = Description of sequence: sgRNA: GSDMD_2
  source 1..20: mol_type = other RNA
ccacgtacacgttgtccccg

SEQ ID NO: 246 - RNA, 20 nt, synthetic construct
  misc_feature 1..20: note = Description of sequence: sgRNA: GSDMD_3
  source 1..20: mol_type = other RNA
tgagtgtggaccctaacacc

SEQ ID NO: 247 - RNA, 20 nt, synthetic construct
  misc_feature 1..20: note = Description of sequence: sgRNA: GZMB_1
  source 1..20: mol_type = other RNA
gcacgaagtcgtctcgtatc

SEQ ID NO: 248 - RNA, 20 nt, synthetic construct
  misc_feature 1..20: note = Description of sequence: sgRNA: GZMB_2
  source 1..20: mol_type = other RNA
agtaccattgagttgtgcgt

SEQ ID NO: 249 - RNA, 20 nt, synthetic construct
  misc_feature 1..20: note = Description of sequence: sgRNA: GZMB_3
  source 1..20: mol_type = other RNA
ggtgcatagtcttaccttaa

SEQ ID NO: 250 - RNA, 20 nt, synthetic construct
  misc_feature 1..20: note = Description of sequence: sgRNA: HRK_1
  source 1..20: mol_type = other RNA
gcggaacttgtaggaacgcg

SEQ ID NO: 251 - RNA, 20 nt, synthetic construct
  misc_feature 1..20: note = Description of sequence: sgRNA: HRK_2
  source 1..20: mol_type = other RNA
gcctagcgccttgagccggg

SEQ ID NO: 252 - RNA, 20 nt, synthetic construct
  misc_feature 1..20: note = Description of sequence: sgRNA: HRK_3
  source 1..20: mol_type = other RNA
gtcgcctagcgccttgagcc

SEQ ID NO: 253 - RNA, 20 nt, synthetic construct
  misc_feature 1..20: note = Description of sequence: sgRNA: HSPB1_1
  source 1..20: mol_type = other RNA
gtcttgaccgtcagctcgtc

SEQ ID NO: 254 - RNA, 20 nt, synthetic construct
  misc_feature 1..20: note = Description of sequence: sgRNA: HSPB1_2
  source 1..20: mol_type = other RNA
tggtcgaagaggcggctatg

SEQ ID NO: 255 - RNA, 20 nt, synthetic construct
  misc_feature 1..20: note = Description of sequence: sgRNA: HSPB1_3
  source 1..20: mol_type = other RNA
tgagttgccggctgagcgcg

SEQ ID NO: 256 - RNA, 20 nt, synthetic construct
  misc_feature 1..20: note = Description of sequence: sgRNA: HTRA2_1
  source 1..20: mol_type = other RNA
cgttcgagatagggacctcg

SEQ ID NO: 257 - RNA, 20 nt, synthetic construct
  misc_feature 1..20: note = Description of sequence: sgRNA: HTRA2_2
  source 1..20: mol_type = other RNA
cgcgaggtccctatctcgaa

SEQ ID NO: 258 - RNA, 20 nt, synthetic construct
  misc_feature 1..20: note = Description of sequence: sgRNA: HTRA2_3
  source 1..20: mol_type = other RNA
gtccctatctcgaacggctc

SEQ ID NO: 259 - RNA, 20 nt, synthetic construct
  misc_feature 1..20: note = Description of sequence: sgRNA: IFIH1_1
  source 1..20: mol_type = other RNA
tcatgtagcgggcggccaga

SEQ ID NO: 260 - RNA, 20 nt, synthetic construct
  misc_feature 1..20: note = Description of sequence: sgRNA: IFIH1_2
  source 1..20: mol_type = other RNA
cgaattcccgagtccaacca

SEQ ID NO: 261 - RNA, 20 nt, synthetic construct
  misc_feature 1..20: note = Description of sequence: sgRNA: IFIH1_3
  source 1..20: mol_type = other RNA
agacgtcttggataagtgca

SEQ ID NO: 262 - RNA, 20 nt, synthetic construct
  misc_feature 1..20: note = Description of sequence: sgRNA: IFNA1_1
  source 1..20: mol_type = other RNA
cccacagcctggataacagg

SEQ ID NO: 263 - RNA, 20 nt, synthetic construct
  misc_feature 1..20: note = Description of sequence: sgRNA: IFNA1_2
  source 1..20: mol_type = other RNA
atgtctgtccatcagacagg

SEQ ID NO: 264 - RNA, 20 nt, synthetic construct
  misc_feature 1..20: note = Description of sequence: sgRNA: IFNA1_3
  source 1..20: mol_type = other RNA
tgctttactgatggtcctgg

SEQ ID NO: 265 - RNA, 20 nt, synthetic construct
  misc_feature 1..20: note = Description of sequence: sgRNA: IFNA13_1
  source 1..20: mol_type = other RNA
cttgcagctgagcaccacca

SEQ ID NO: 266 - RNA, 20 nt, synthetic construct
  misc_feature 1..20: note = Description of sequence: sgRNA: IFNA13_2
  source 1..20: mol_type = other RNA
gttcggtgcagaatttgtct

SEQ ID NO: 267 - RNA, 20 nt, synthetic construct
  misc_feature 1..20: note = Description of sequence: sgRNA: IFNA13_3
  source 1..20: mol_type = other RNA
ctaccagcagctgaatgact

SEQ ID NO: 268 - RNA, 20 nt, synthetic construct
  misc_feature 1..20: note = Description of sequence: sgRNA: IFNA7_1
  source 1..20: mol_type = other RNA
acatgaattcagattcccag

SEQ ID NO: 269 - RNA, 20 nt, synthetic construct
  misc_feature 1..20: note = Description of sequence: sgRNA: IFNA7_2
  source 1..20: mol_type = other RNA
tcaaggccctcctattacgc

SEQ ID NO: 270 - RNA, 20 nt, synthetic construct
  misc_feature 1..20: note = Description of sequence: sgRNA: IFNA7_3
  source 1..20: mol_type = other RNA
cttcaatctcttcagcacag

SEQ ID NO: 271 - RNA, 20 nt, synthetic construct
  misc_feature 1..20: note = Description of sequence: sgRNA: IL18_1
  source 1..20: mol_type = other RNA
gacaatacgctttactttat

SEQ ID NO: 272 - RNA, 20 nt, synthetic construct
  misc_feature 1..20: note = Description of sequence: sgRNA: IL18_2
  source 1..20: mol_type = other RNA
tacgctttactttataggta

SEQ ID NO: 273 - RNA, 20 nt, synthetic construct
  misc_feature 1..20: note = Description of sequence: sgRNA: IL18_3
  source 1..20: mol_type = other RNA
tttgaatcttcatcatacga

SEQ ID NO: 274 - RNA, 20 nt, synthetic construct
  misc_feature 1..20: note = Description of sequence: sgRNA: IL1B_1
  source 1..20: mol_type = other RNA
tccgaccaccactacagcaa

SEQ ID NO: 275 - RNA, 20 nt, synthetic construct
  misc_feature 1..20: note = Description of sequence: sgRNA: IL1B_2
  source 1..20: mol_type = other RNA
cgcgtcagttgttgtggcca

SEQ ID NO: 276 - RNA, 20 nt, synthetic construct
  misc_feature 1..20: note = Description of sequence: sgRNA: IL1B_3
  source 1..20: mol_type = other RNA
catggccacaacaactgacg

SEQ ID NO: 277 - RNA, 20 nt, synthetic construct
  misc_feature 1..20: note = Description of sequence: sgRNA: IL4_1
  source 1..20: mol_type = other RNA
tgatatcgcacttgtgtccg

SEQ ID NO: 278 - RNA, 20 nt, synthetic construct
  misc_feature 1..20: note = Description of sequence: sgRNA: IL4_2
  source 1..20: mol_type = other RNA
gcagaaggtgagtacctatc

SEQ ID NO: 279 - RNA, 20 nt, synthetic construct
  misc_feature 1..20: note = Description of sequence: sgRNA: IL4_3
  source 1..20: mol_type = other RNA
tgcaaatcgacacctattaa

SEQ ID NO: 280 - RNA, 20 nt, synthetic construct
  misc_feature 1..20: note = Description of sequence: sgRNA: IREB2_1
  source 1..20: mol_type = other RNA
gcgatggacgccccaaaagc

SEQ ID NO: 281 - RNA, 20 nt, synthetic construct
  misc_feature 1..20: note = Description of sequence: sgRNA: IREB2_2
  source 1..20: mol_type = other RNA
ttactcaatacgggtcttgt

SEQ ID NO: 282 - RNA, 20 nt, synthetic construct
  misc_feature 1..20: note = Description of sequence: sgRNA: IREB2_3
  source 1..20: mol_type = other RNA
aacaagacccgtattgagta

SEQ ID NO: 283 - RNA, 20 nt, synthetic construct
  misc_feature 1..20: note = Description of sequence: sgRNA: IRGM_1
  source 1..20: mol_type = other RNA
attcacatacccgctccttc

SEQ ID NO: 284 - RNA, 20 nt, synthetic construct
  misc_feature 1..20: note = Description of sequence: sgRNA: IRGM_2
  source 1..20: mol_type = other RNA
gatgcttgccaaaaccgctg

SEQ ID NO: 285 - RNA, 20 nt, synthetic construct
  misc_feature 1..20: note = Description of sequence: sgRNA: IRGM_3
  source 1..20: mol_type = other RNA
caaccggtatgacttcatca

SEQ ID NO: 286 - RNA, 20 nt, synthetic construct
  misc_feature 1..20: note = Description of sequence: sgRNA: JUN_1
  source 1..20: mol_type = other RNA
tcggcggcgcagccggtcaa

SEQ ID NO: 287 - RNA, 20 nt, synthetic construct
  misc_feature 1..20: note = Description of sequence: sgRNA: JUN_2
  source 1..20: mol_type = other RNA
ccgtccgagagcggacctta

SEQ ID NO: 288 - RNA, 20 nt, synthetic construct
  misc_feature 1..20: note = Description of sequence: sgRNA: JUN_3
  source 1..20: mol_type = other RNA
cataaggtccgctctcggac

SEQ ID NO: 289 - RNA, 20 nt, synthetic construct
  misc_feature 1..20: note = Description of sequence: sgRNA: MAP1LC3A_1
  source 1..20: mol_type = other RNA
cgcagccgaccgctgtaagg

SEQ ID NO: 290 - RNA, 20 nt, synthetic construct
  misc_feature 1..20: note = Description of sequence: sgRNA: MAP1LC3A_2
  source 1..20: mol_type = other RNA
gtccgcagccgaccgctgta

SEQ ID NO: 291 - RNA, 20 nt, synthetic construct
  misc_feature 1..20: note = Description of sequence: sgRNA: MAP1LC3A_3
  source 1..20: mol_type = other RNA
ggactcaccgggattttgct

SEQ ID NO: 292 - RNA, 20 nt, synthetic construct
  misc_feature 1..20: note = Description of sequence: sgRNA: MAVS_1
  source 1..20: mol_type = other RNA
acagggtcagttgtatctac

SEQ ID NO: 293 - RNA, 20 nt, synthetic construct
  misc_feature 1..20: note = Description of sequence: sgRNA: MAVS_2
  source 1..20: mol_type = other RNA
ccagcacgggttgagttgat

SEQ ID NO: 294 - RNA, 20 nt, synthetic construct
  misc_feature 1..20: note = Description of sequence: sgRNA: MAVS_3
  source 1..20: mol_type = other RNA
caactcaacccgtgctggca

SEQ ID NO: 295 - RNA, 20 nt, synthetic construct
  misc_feature 1..20: note = Description of sequence: sgRNA: MCL1_1
  source 1..20: mol_type = other RNA
ttccatgtagaggacctaga

SEQ ID NO: 296 - RNA, 20 nt, synthetic construct
  misc_feature 1..20: note = Description of sequence: sgRNA: MCL1_2
  source 1..20: mol_type = other RNA
ctggagaccttacgacgggt

SEQ ID NO: 297 - RNA, 20 nt, synthetic construct
  misc_feature 1..20: note = Description of sequence: sgRNA: MCL1_3
  source 1..20: mol_type = other RNA
ggcgctggagaccttacgac

SEQ ID NO: 298 - RNA, 20 nt, synthetic construct
  misc_feature 1..20: note = Description of sequence: sgRNA: MLKL_1
  source 1..20: mol_type = other RNA
ttgaagcatattatcaccct

SEQ ID NO: 299 - RNA, 20 nt, synthetic construct
  misc_feature 1..20: note = Description of sequence: sgRNA: MLKL_2
  source 1..20: mol_type = other RNA
ttagctttggaatcgtcctc

SEQ ID NO: 300 - RNA, 20 nt, synthetic construct
  misc_feature 1..20: note = Description of sequence: sgRNA: MLKL_3
  source 1..20: mol_type = other RNA
actggagatatcccgtttca

SEQ ID NO: 301 - RNA, 20 nt, synthetic construct
  misc_feature 1..20: note = Description of sequence: sgRNA: MPG_1
  source 1..20: mol_type = other RNA
tacagttttgccgacggatg

SEQ ID NO: 302 - RNA, 20 nt, synthetic construct
  misc_feature 1..20: note = Description of sequence: sgRNA: MPG_2
  source 1..20: mol_type = other RNA
ttacagttttgccgacggat

SEQ ID NO: 303 - RNA, 20 nt, synthetic construct
  misc_feature 1..20: note = Description of sequence: sgRNA: MPG_3
  source 1..20: mol_type = other RNA
aaagggccaccttacccgac

SEQ ID NO: 304 - RNA, 20 nt, synthetic construct
  misc_feature 1..20: note = Description of sequence: sgRNA: NAIP_1
  source 1..20: mol_type = other RNA
agcatccacttcccaatacg

SEQ ID NO: 305 - RNA, 20 nt, synthetic construct
  misc_feature 1..20: note = Description of sequence: sgRNA: NAIP_2
  source 1..20: mol_type = other RNA
ctgttactgaaatgtgcgtg

SEQ ID NO: 306 - RNA, 20 nt, synthetic construct
  misc_feature 1..20: note = Description of sequence: sgRNA: NAIP_3
  source 1..20: mol_type = other RNA
gatgtcttatttcctcgtat

SEQ ID NO: 307 - RNA, 20 nt, synthetic construct
  misc_feature 1..20: note = Description of sequence: sgRNA: NFE2L2_1
  source 1..20: mol_type = other RNA
gcgacggaaagagtatgagc

SEQ ID NO: 308 - RNA, 20 nt, synthetic construct
  misc_feature 1..20: note = Description of sequence: sgRNA: NFE2L2_2
  source 1..20: mol_type = other RNA
cataccgtctaaatcaacag

SEQ ID NO: 309 - RNA, 20 nt, synthetic construct
  misc_feature 1..20: note = Description of sequence: sgRNA: NFE2L2_3
  source 1..20: mol_type = other RNA
gcataccgtctaaatcaaca

SEQ ID NO: 310 - RNA, 20 nt, synthetic construct
  misc_feature 1..20: note = Description of sequence: sgRNA: NLRC4_1
  source 1..20: mol_type = other RNA
cgtgttgatacactgctgcg

SEQ ID NO: 311 - RNA, 20 nt, synthetic construct
  misc_feature 1..20: note = Description of sequence: sgRNA: NLRC4_2
  source 1..20: mol_type = other RNA
caaactgccgtatgtgcctc

SEQ ID NO: 312 - RNA, 20 nt, synthetic construct
  misc_feature 1..20: note = Description of sequence: sgRNA: NLRC4_3
  source 1..20: mol_type = other RNA
ctacagaatcaacggctgcc

SEQ ID NO: 313 - RNA, 20 nt, synthetic construct
  misc_feature 1..20: note = Description of sequence: sgRNA: NLRP1_1
  source 1..20: mol_type = other RNA
tcgccaataaagcgcactcc

SEQ ID NO: 314 - RNA, 20 nt, synthetic construct
  misc_feature 1..20: note = Description of sequence: sgRNA: NLRP1_2
  source 1..20: mol_type = other RNA
gccggcaattcatggatcca

SEQ ID NO: 315 - RNA, 20 nt, synthetic construct
  misc_feature 1..20: note = Description of sequence: sgRNA: NLRP1_3
  source 1..20: mol_type = other RNA
cgccggcaattcatggatcc

SEQ ID NO: 316 - RNA, 20 nt, synthetic construct
  misc_feature 1..20: note = Description of sequence: sgRNA: NLRP3_1
  source 1..20: mol_type = other RNA
acgctaatgatcgacttcaa

SEQ ID NO: 317 - RNA, 20 nt, synthetic construct
  misc_feature 1..20: note = Description of sequence: sgRNA: NLRP3_2
  source 1..20: mol_type = other RNA
cgctaatgatcgacttcaat

SEQ ID NO: 318 - RNA, 20 nt, synthetic construct
  misc_feature 1..20: note = Description of sequence: sgRNA: NLRP3_3
  source 1..20: mol_type = other RNA
attgaagtcgatcattagcg

SEQ ID NO: 319 - RNA, 20 nt, synthetic construct
  misc_feature 1..20: note = Description of sequence: sgRNA: NonTargetingControlGuideForHuman_0001
  source 1..20: mol_type = other RNA
acggaggctaagcgtcgcaa

SEQ ID NO: 320 - RNA, 20 nt, synthetic construct
  misc_feature 1..20: note = Description of sequence: sgRNA: NonTargetingControlGuideForHuman_0002
  source 1..20: mol_type = other RNA
cgcttccgcggcccgttcaa

SEQ ID NO: 321 - RNA, 20 nt, synthetic construct
  misc_feature 1..20: note = Description of sequence: sgRNA: NonTargetingControlGuideForHuman_0003
  source 1..20: mol_type = other RNA
atcgtttccgcttaacggcg

SEQ ID NO: 322 - RNA, 20 nt, synthetic construct
  misc_feature 1..20: note = Description of sequence: sgRNA: NonTargetingControlGuideForHuman_0004
  source 1..20: mol_type = other RNA
gtaggcgcgccgctctctac

SEQ ID NO: 323 - RNA, 20 nt, synthetic construct
  misc_feature 1..20: note = Description of sequence: sgRNA: NonTargetingControlGuideForHuman_0005
  source 1..20: mol_type = other RNA
ccatatcggggcgagacatg

SEQ ID NO: 324 - RNA, 20 nt, synthetic construct
  misc_feature 1..20: note = Description of sequence: sgRNA: NonTargetingControlGuideForHuman_0006
  source 1..20: mol_type = other RNA
tactaacgccgctcctacag

SEQ ID NO: 325 - RNA, 20 nt, synthetic construct
  misc_feature 1..20: note = Description of sequence: sgRNA: NonTargetingControlGuideForHuman_0007
  source 1..20: mol_type = other RNA
tgaggatcatgtcgagcgcc

SEQ ID NO: 326 - RNA, 20 nt, synthetic construct
  misc_feature 1..20: note = Description of sequence: sgRNA: NonTargetingControlGuideForHuman_0008
  source 1..20: mol_type = other RNA
gggcccgcataggatatcgc

SEQ ID NO: 327 - RNA, 20 nt, synthetic construct
  misc_feature 1..20: note = Description of sequence: sgRNA: NonTargetingControlGuideForHuman_0009
  source 1..20: mol_type = other RNA
tagacaaccgcggagaatgc

SEQ ID NO: 328 - RNA, 20 nt, synthetic construct
  misc_feature 1..20: note = Description of sequence: sgRNA: NonTargetingControlGuideForHuman_0010
  source 1..20: mol_type = other RNA
acgggcggctatcgctgact

SEQ ID NO: 329 - RNA, 20 nt, synthetic construct
  misc_feature 1..20: note = Description of sequence: sgRNA: NonTargetingControlGuideForHuman_0011
  source 1..20: mol_type = other RNA
cgcggaaattttaccgacga

SEQ ID NO: 330 - RNA, 20 nt, synthetic construct
  misc_feature 1..20: note = Description of sequence: sgRNA: NonTargetingControlGuideForHuman_0012
  source 1..20: mol_type = other RNA
cttacaatcgtcggtccaat

SEQ ID NO: 331 - RNA, 20 nt, synthetic construct
  misc_feature 1..20: note = Description of sequence: sgRNA: NonTargetingControlGuideForHuman_0013
  source 1..20: mol_type = other RNA
gcgtgcgtcccgggttaccc

SEQ ID NO: 332 - RNA, 20 nt, synthetic construct
  misc_feature 1..20: note = Description of sequence: sgRNA: NonTargetingControlGuideForHuman_0014
  source 1..20: mol_type = other RNA
cggagtaacaagcggacgga

SEQ ID NO: 333 - RNA, 20 nt, synthetic construct
  misc_feature 1..20: note = Description of sequence: sgRNA: NonTargetingControlGuideForHuman_0015
  source 1..20: mol_type = other RNA
cgagtgttatacgcaccgtt

SEQ ID NO: 334 - RNA, 20 nt, synthetic construct
  misc_feature 1..20: note = Description of sequence: sgRNA: NonTargetingControlGuideForHuman_0016
  source 1..20: mol_type = other RNA
cgactaaccggaaacttttt

SEQ ID NO: 335 - RNA, 20 nt, synthetic construct
  misc_feature 1..20: note = Description of sequence: sgRNA: NonTargetingControlGuideForHuman_0017
  source 1..20: mol_type = other RNA
caacgggttctcccggctac

SEQ ID NO: 336 - RNA, 20 nt, synthetic construct
  misc_feature 1..20: note = Description of sequence: sgRNA: NonTargetingControlGuideForHuman_0018
  source 1..20: mol_type = other RNA
caggagtcgccgatacgcgt

SEQ ID NO: 337 - RNA, 20 nt, synthetic construct
  misc_feature 1..20: note = Description of sequence: sgRNA: NonTargetingControlGuideForHuman_0019
  source 1..20: mol_type = other RNA
ttcacgtcgtctcgcgacca

SEQ ID NO: 338 - RNA, 20 nt, synthetic construct
  misc_feature 1..20: note = Description of sequence: sgRNA: NonTargetingControlGuideForHuman_0020
  source 1..20: mol_type = other RNA
gtgtcggattccgccgctta

SEQ ID NO: 339 - RNA, 20 nt, synthetic construct
  misc_feature 1..20: note = Description of sequence: sgRNA: NonTargetingControlGuideForHuman_0021
  source 1..20: mol_type = other RNA
cacgaactcacaccgcgcga

SEQ ID NO: 340 - RNA, 20 nt, synthetic construct
  misc_feature 1..20: note = Description of sequence: sgRNA: NonTargetingControlGuideForHuman_0022
  source 1..20: mol_type = other RNA
cgctagtacgctcctctata

SEQ ID NO: 341 - RNA, 20 nt, synthetic construct
  misc_feature 1..20: note = Description of sequence: sgRNA: NonTargetingControlGuideForHuman_0023
  source 1..20: mol_type = other RNA
tcgcgcttgggttatacgct

SEQ ID NO: 342 - RNA, 20 nt, synthetic construct
  misc_feature 1..20: note = Description of sequence: sgRNA: NonTargetingControlGuideForHuman_0024
  source 1..20: mol_type = other RNA
ctatctcgagtggtaatgcg

SEQ ID NO: 343 - RNA, 20 nt, synthetic construct
  misc_feature 1..20: note = Description of sequence: sgRNA: NonTargetingControlGuideForHuman_0025
  source 1..20: mol_type = other RNA
aatcgactcgaacttcgtgt

SEQ ID NO: 344 - RNA, 20 nt, synthetic construct
  misc_feature 1..20: note = Description of sequence: sgRNA: NonTargetingControlGuideForHuman_0026
  source 1..20: mol_type = other RNA
cccgatggactataccgaac

SEQ ID NO: 345 - RNA, 20 nt, synthetic construct
  misc_feature 1..20: note = Description of sequence: sgRNA: NonTargetingControlGuideForHuman_0027
  source 1..20: mol_type = other RNA
acgttcgagtacgaccagct

SEQ ID NO: 346 - RNA, 20 nt, synthetic construct
  misc_feature 1..20: note = Description of sequence: sgRNA: NonTargetingControlGuideForHuman_0028
  source 1..20: mol_type = other RNA
cgcgacgactcaacctagtc

SEQ ID NO: 347 - RNA, 20 nt, synthetic construct
  misc_feature 1..20: note = Description of sequence: sgRNA: NonTargetingControlGuideForHuman_0029
  source 1..20: mol_type = other RNA
ggtcaccgatcgagagctag

SEQ ID NO: 348 - RNA, 20 nt, synthetic construct
  misc_feature 1..20: note = Description of sequence: sgRNA: NonTargetingControlGuideForHuman_0030
  source 1..20: mol_type = other RNA
ctcaaccgaccgtatggtca

SEQ ID NO: 349 - RNA, 20 nt, synthetic construct
  misc_feature 1..20: note = Description of sequence: sgRNA: NonTargetingControlGuideForHuman_0031
  source 1..20: mol_type = other RNA
cgtattcgactctcaacgcg

SEQ ID NO: 350 - RNA, 20 nt, synthetic construct
  misc_feature 1..20: note = Description of sequence: sgRNA: NonTargetingControlGuideForHuman_0032
  source 1..20: mol_type = other RNA
ctagccgcccagatcgagcc

SEQ ID NO: 351 - RNA, 20 nt, synthetic construct
  misc_feature 1..20: note = Description of sequence: sgRNA: NonTargetingControlGuideForHuman_0033
  source 1..20: mol_type = other RNA
gaatcgaccgacactaatgt

SEQ ID NO: 352 - RNA, 20 nt, synthetic construct
  misc_feature 1..20: note = Description of sequence: sgRNA: NonTargetingControlGuideForHuman_0034
  source 1..20: mol_type = other RNA
acttcagttcggcgtagtca

SEQ ID NO: 353 - RNA, 20 nt, synthetic construct
  misc_feature 1..20: note = Description of sequence: sgRNA: NonTargetingControlGuideForHuman_0035
  source 1..20: mol_type = other RNA
gtgcgatgtcgcttcaacgt

SEQ ID NO: 354 - RNA, 20 nt, synthetic construct
  misc_feature 1..20: note = Description of sequence: sgRNA: NonTargetingControlGuideForHuman_0036
  source 1..20: mol_type = other RNA
cgcctaatttccggatcaat

SEQ ID NO: 355 - RNA, 20 nt, synthetic construct
  misc_feature 1..20: note = Description of sequence: sgRNA: NonTargetingControlGuideForHuman_0037
  source 1..20: mol_type = other RNA
cgtggccggaaccgtcatag

SEQ ID NO: 356 - RNA, 20 nt, synthetic construct
  misc_feature 1..20: note = Description of sequence: sgRNA: NonTargetingControlGuideForHuman_0038
  source 1..20: mol_type = other RNA
accctccgaatcgtaacgga

SEQ ID NO: 357 - RNA, 20 nt, synthetic construct
  misc_feature 1..20: note = Description of sequence: sgRNA: NonTargetingControlGuideForHuman_0039
  source 1..20: mol_type = other RNA
aaacggtacgacagcgtgtg

SEQ ID NO: 358 - RNA, 20 nt, synthetic construct
  misc_feature 1..20: note = Description of sequence: sgRNA: NonTargetingControlGuideForHuman_0040
  source 1..20: mol_type = other RNA
acatagtcgacggctcgatt

SEQ ID NO: 359 - RNA, 20 nt, synthetic construct
  misc_feature 1..20: note = Description of sequence: sgRNA: NonTargetingControlGuideForHuman_0041
  source 1..20: mol_type = other RNA
gatggcgcttcagtcgtcgg

SEQ ID NO: 360 - RNA, 20 nt, synthetic construct
  misc_feature 1..20: note = Description of sequence: sgRNA: NonTargetingControlGuideForHuman_0042
  source 1..20: mol_type = other RNA
ataatccggaaacgctcgac

SEQ ID NO: 361 - RNA, 20 nt, synthetic construct
  misc_feature 1..20: note = Description of sequence: sgRNA: NonTargetingControlGuideForHuman_0043
  source 1..20: mol_type = other RNA
cgccgggctgacaattaacg

SEQ ID NO: 362 - RNA, 20 nt, synthetic construct
  misc_feature 1..20: note = Description of sequence: sgRNA: NonTargetingControlGuideForHuman_0044
  source 1..20: mol_type = other RNA
cgtcgccatatgccggtggc

SEQ ID NO: 363 - RNA, 20 nt, synthetic construct
  misc_feature 1..20: note = Description of sequence: sgRNA: NonTargetingControlGuideForHuman_0045
  source 1..20: mol_type = other RNA
cgggcctataacaccatcga

SEQ ID NO: 364 - RNA, 20 nt, synthetic construct
  misc_feature 1..20: note = Description of sequence: sgRNA: NonTargetingControlGuideForHuman_0046
  source 1..20: mol_type = other RNA
cgccgttccgagatacttga

SEQ ID NO: 365 - RNA, 20 nt, synthetic construct
  misc_feature 1..20: note = Description of sequence: sgRNA: NonTargetingControlGuideForHuman_0047
  source 1..20: mol_type = other RNA
cgggacgtcgcgaaaatgta

SEQ ID NO: 366 - RNA, 20 nt, synthetic construct
  misc_feature 1..20: note = Description of sequence: sgRNA: NonTargetingControlGuideForHuman_0048
  source 1..20: mol_type = other RNA
tcggcatacgggacacacgc

SEQ ID NO: 367 - RNA, 20 nt, synthetic construct
  misc_feature 1..20: note = Description of sequence: sgRNA: NonTargetingControlGuideForHuman_0049
  source 1..20: mol_type = other RNA
agctccatcgccgcgataat

SEQ ID NO: 368 - RNA, 20 nt, synthetic construct
  misc_feature 1..20: note = Description of sequence: sgRNA: NonTargetingControlGuideForHuman_0050
  source 1..20: mol_type = other RNA
atcgtatcatcagctagcgc

SEQ ID NO: 369 - RNA, 20 nt, synthetic construct
  misc_feature 1..20: note = Description of sequence: sgRNA: NOS3_1
  source 1..20: mol_type = other RNA
tgaccatctctaccgcgacg

SEQ ID NO: 370 - RNA, 20 nt, synthetic construct
  misc_feature 1..20: note = Description of sequence: sgRNA: NOS3_2
  source 1..20: mol_type = other RNA
gggcgttctgcacctcgtcg

SEQ ID NO: 371 - RNA, 20 nt, synthetic construct
  misc_feature 1..20: note = Description of sequence: sgRNA: NOS3_3
  source 1..20: mol_type = other RNA
caagcgcggggttgcttgca

SEQ ID NO: 372 - RNA, 20 nt, synthetic construct
  misc_feature 1..20: note = Description of sequence: sgRNA: NOX4_1
  source 1..20: mol_type = other RNA
gccaagagtgttcggcacat

SEQ ID NO: 373 - RNA, 20 nt, synthetic construct
  misc_feature 1..20: note = Description of sequence: sgRNA: NOX4_2
  source 1..20: mol_type = other RNA
acactcttggcttacctccg

SEQ ID NO: 374 - RNA, 20 nt, synthetic construct
  misc_feature 1..20: note = Description of sequence: sgRNA: NOX4_3
  source 1..20: mol_type = other RNA
acgcctacagaattacacca

SEQ ID NO: 375 - RNA, 20 nt, synthetic construct
  misc_feature 1..20: note = Description of sequence: sgRNA: NOX5_1
  source 1..20: mol_type = other RNA
gctcttcgaatcggccgacg

SEQ ID NO: 376 - RNA, 20 nt, synthetic construct
  misc_feature 1..20: note = Description of sequence: sgRNA: NOX5_2
  source 1..20: mol_type = other RNA
ttcgaatcggccgacgcgga

SEQ ID NO: 377 - RNA, 20 nt, synthetic construct
  misc_feature 1..20: note = Description of sequence: sgRNA: NOX5_3
  source 1..20: mol_type = other RNA
cggccgacgcggacggcaac

SEQ ID NO: 378 - RNA, 20 nt, synthetic construct
  misc_feature 1..20: note = Description of sequence: sgRNA: NPEPL1_1
  source 1..20: mol_type = other RNA
cggtggcgtagttcaggtag

SEQ ID NO: 379 - RNA, 20 nt, synthetic construct
  misc_feature 1..20: note = Description of sequence: sgRNA: NPEPL1_2
  source 1..20: mol_type = other RNA
cggccgcccacttcatcacg

SEQ ID NO: 380 - RNA, 20 nt, synthetic construct
  misc_feature 1..20: note = Description of sequence: sgRNA: NPEPL1_3
  source 1..20: mol_type = other RNA
cggagcgcatcgctgcattg

SEQ ID NO: 381 - RNA, 20 nt, synthetic construct
  misc_feature 1..20: note = Description of sequence: sgRNA: PARP1_1
  source 1..20: mol_type = other RNA
cgagtcgagtacgccaagag

SEQ ID NO: 382 - RNA, 20 nt, synthetic construct
  misc_feature 1..20: note = Description of sequence: sgRNA: PARP1_2
  source 1..20: mol_type = other RNA
gagtcgagtacgccaagagc

SEQ ID NO: 383 - RNA, 20 nt, synthetic construct
  misc_feature 1..20: note = Description of sequence: sgRNA: PARP1_3
  source 1..20: mol_type = other RNA
aagtacgtgcaaggggtgta

SEQ ID NO: 384 - RNA, 20 nt, synthetic construct
  misc_feature 1..20: note = Description of sequence: sgRNA: PARP2_1
  source 1..20: mol_type = other RNA
gttcgaattccatggcggcg

SEQ ID NO: 385 - RNA, 20 nt, synthetic construct
  misc_feature 1..20: note = Description of sequence: sgRNA: PARP2_2
  source 1..20: mol_type = other RNA
cgaattccatggcggcgcgg

SEQ ID NO: 386 - RNA, 20 nt, synthetic construct
  misc_feature 1..20: note = Description of sequence: sgRNA: PARP2_3
  source 1..20: mol_type = other RNA
acgtcagcgttcgaattcca

SEQ ID NO: 387 - RNA, 20 nt, synthetic construct
  misc_feature 1..20: note = Description of sequence: sgRNA: PGAM5_1
  source 1..20: mol_type = other RNA
aagcggcatggcgttccggc

SEQ ID NO: 388 - RNA, 20 nt, synthetic construct
  misc_feature 1..20: note = Description of sequence: sgRNA: PGAM5_2
  source 1..20: mol_type = other RNA
tggcgcgcgtcatagacgaa

SEQ ID NO: 389 - RNA, 20 nt, synthetic construct
  misc_feature 1..20: note = Description of sequence: sgRNA: PGAM5_3
  source 1..20: mol_type = other RNA
gtgccggctgatgatatcgg

SEQ ID NO: 390 - RNA, 20 nt, synthetic construct
  misc_feature 1..20: note = Description of sequence: sgRNA: PIK3C3_1
  source 1..20: mol_type = other RNA
taagctggacgttgatatcc

SEQ ID NO: 391 - RNA, 20 nt, synthetic construct
  misc_feature 1..20: note = Description of sequence: sgRNA: PIK3C3_2
  source 1..20: mol_type = other RNA
ctttgcagacggtgcgatgg

SEQ ID NO: 392 - RNA, 20 nt, synthetic construct
  misc_feature 1..20: note = Description of sequence: sgRNA: PIK3C3_3
  source 1..20: mol_type = other RNA
ggaacaacggtttcgctctt

SEQ ID NO: 393 - RNA, 20 nt, synthetic construct
  misc_feature 1..20: note = Description of sequence: sgRNA: PMAIP1_1
  source 1..20: mol_type = other RNA
tccagcaggtaccgacccgc

SEQ ID NO: 394 - RNA, 20 nt, synthetic construct
  misc_feature 1..20: note = Description of sequence: sgRNA: PMAIP1_2
  source 1..20: mol_type = other RNA
cccagcgggtcggtacctgc

SEQ ID NO: 395 - RNA, 20 nt, synthetic construct
  misc_feature 1..20: note = Description of sequence: sgRNA: PMAIP1_3
  source 1..20: mol_type = other RNA
cgctcaaccgagccccgcgc

SEQ ID NO: 396 - RNA, 20 nt, synthetic construct
  misc_feature 1..20: note = Description of sequence: sgRNA: PTPA_1
  source 1..20: mol_type = other RNA
taggcatacgctgactacat

SEQ ID NO: 397 - RNA, 20 nt, synthetic construct
  misc_feature 1..20: note = Description of sequence: sgRNA: PTPA_2
  source 1..20: mol_type = other RNA
taccctcaacgaaggtgtga

SEQ ID NO: 398 - RNA, 20 nt, synthetic construct
  misc_feature 1..20: note = Description of sequence: sgRNA: PTPA_3
  source 1..20: mol_type = other RNA
ttctcaacacgctggacagg

SEQ ID NO: 399 - RNA, 20 nt, synthetic construct
  misc_feature 1..20: note = Description of sequence: sgRNA: PYCARD_1
  source 1..20: mol_type = other RNA
cgctaacgtgctgcgcgaca

SEQ ID NO: 400 - RNA, 20 nt, synthetic construct
  misc_feature 1..20: note = Description of sequence: sgRNA: PYCARD_2
  source 1..20: mol_type = other RNA
catgtcgcgcagcacgttag

SEQ ID NO: 401 - RNA, 20 nt, synthetic construct
  misc_feature 1..20: note = Description of sequence: sgRNA: PYCARD_3
  source 1..20: mol_type = other RNA
gtgccgctgcgcgagggcta

SEQ ID NO: 402 - RNA, 20 nt, synthetic construct
  misc_feature 1..20: note = Description of sequence: sgRNA: RB1CC1_1
  source 1..20: mol_type = other RNA
atctttgattgcggaacaac

SEQ ID NO: 403 - RNA, 20 nt, synthetic construct
  misc_feature 1..20: note = Description of sequence: sgRNA: RB1CC1_2
  source 1..20: mol_type = other RNA
gcggatctatctgattcacc

SEQ ID NO: 404 - RNA, 20 nt, synthetic construct
  misc_feature 1..20: note = Description of sequence: sgRNA: RB1CC1_3
  source 1..20: mol_type = other RNA
tgaagatcggctctacgccc

SEQ ID NO: 405 - RNA, 20 nt, synthetic construct
  misc_feature 1..20: note = Description of sequence: sgRNA: RIPK1_1
  source 1..20: mol_type = other RNA
cggctttcagcacgtgcatc

SEQ ID NO: 406 - RNA, 20 nt, synthetic construct
  misc_feature 1..20: note = Description of sequence: sgRNA: RIPK1_2
  source 1..20: mol_type = other RNA
ctcgggcgccatgtagtaga

SEQ ID NO: 407 - RNA, 20 nt, synthetic construct
  misc_feature 1..20: note = Description of sequence: sgRNA: RIPK1_3
  source 1..20: mol_type = other RNA
gttgacgtcattcaggtgct

SEQ ID NO: 408 - RNA, 20 nt, synthetic construct
  misc_feature 1..20: note = Description of sequence: sgRNA: RIPK3_1
  source 1..20: mol_type = other RNA
cgggcgcaacataggaagtg

SEQ ID NO: 409 - RNA, 20 nt, synthetic construct
  misc_feature 1..20: note = Description of sequence: sgRNA: RIPK3_2
  source 1..20: mol_type = other RNA
cagtgttccgggcgcaacat

SEQ ID NO: 410 - RNA, 20 nt, synthetic construct
  misc_feature 1..20: note = Description of sequence: sgRNA: RIPK3_3
  source 1..20: mol_type = other RNA
cgggttcggcacagtgttcc

SEQ ID NO: 411 - RNA, 20 nt, synthetic construct
  misc_feature 1..20: note = Description of sequence: sgRNA: SafeHarborchr1_21
  source 1..20: mol_type = other RNA
gaaaacagaatatggaccta

SEQ ID NO: 412 - RNA, 20 nt, synthetic construct
  misc_feature 1..20: note = Description of sequence: sgRNA: SafeHarborchr10_45
  source 1..20: mol_type = other RNA
gaaaagtggatgtgtgtccc

SEQ ID NO: 413 - RNA, 20 nt, synthetic construct
  misc_feature 1..20: note = Description of sequence: sgRNA: SafeHarborchr11_28
  source 1..20: mol_type = other RNA
gaaaactgcattgtgcagac

SEQ ID NO: 414 - RNA, 20 nt, synthetic construct
  misc_feature 1..20: note = Description of sequence: sgRNA: SafeHarborchr11_33
  source 1..20: mol_type = other RNA
gaaaagacagtaaaccttgc

SEQ ID NO: 415 - RNA, 20 nt, synthetic construct
  misc_feature 1..20: note = Description of sequence: sgRNA: SafeHarborchr11_34
  source 1..20: mol_type = other RNA
gaaaagacagtaaacctttc

SEQ ID NO: 416 - RNA, 20 nt, synthetic construct
  misc_feature 1..20: note = Description of sequence: sgRNA: SafeHarborchr11_38
  source 1..20: mol_type = other RNA
gaaaagcctgtgtggaagag

SEQ ID NO: 417 - RNA, 20 nt, synthetic construct
  misc_feature 1..20: note = Description of sequence: sgRNA: SafeHarborchr11_41
  source 1..20: mol_type = other RNA
gaaaagtaaaacatgcagag

SEQ ID NO: 418 - RNA, 20 nt, synthetic construct
  misc_feature 1..20: note = Description of sequence: sgRNA: SafeHarborchr12_1
  source 1..20: mol_type = other RNA
gaaaaaactgtttctttggt

SEQ ID NO: 419 - RNA, 20 nt, synthetic construct
  misc_feature 1..20: note = Description of sequence: sgRNA: SafeHarborchr12_26
  source 1..20: mol_type = other RNA
gaaaactatacctcatgatt

SEQ ID NO: 420 - RNA, 20 nt, synthetic construct
  misc_feature 1..20: note = Description of sequence: sgRNA: SafeHarborchr12_29
  source 1..20: mol_type = other RNA
gaaaacttagagaaagcaca

SEQ ID NO: 421 - RNA, 20 nt, synthetic construct
  misc_feature 1..20: note = Description of sequence: sgRNA: SafeHarborchr12_30
  source 1..20: mol_type = other RNA
gaaaacttcagaaattgtgc

SEQ ID NO: 422 - RNA, 20 nt, synthetic construct
  misc_feature 1..20: note = Description of sequence: sgRNA: SafeHarborchr13_12
  source 1..20: mol_type = other RNA
gaaaaatcagacgagctcca

SEQ ID NO: 423 - RNA, 20 nt, synthetic construct
  misc_feature 1..20: note = Description of sequence: sgRNA: SafeHarborchr13_16
  source 1..20: mol_type = other RNA
gaaaaatgtctctacttgcc

SEQ ID NO: 424 - RNA, 20 nt, synthetic construct
  misc_feature 1..20: note = Description of sequence: sgRNA: SafeHarborchr13_25
  source 1..20: mol_type = other RNA
gaaaactagtagtggaaact

SEQ ID NO: 425 - RNA, 20 nt, synthetic construct
  misc_feature 1..20: note = Description of sequence: sgRNA: SafeHarborchr13_8
  source 1..20: mol_type = other RNA
gaaaaaggatacatttgcaa

SEQ ID NO: 426 - RNA, 20 nt, synthetic construct
  misc_feature 1..20: note = Description of sequence: sgRNA: SafeHarborchr15_19
  source 1..20: mol_type = other RNA
gaaaacacaccttatcacag

SEQ ID NO: 427 - RNA, 20 nt, synthetic construct
  misc_feature 1..20: note = Description of sequence: sgRNA: SafeHarborchr18_24
  source 1..20: mol_type = other RNA
gaaaaccctatatatatagg

SEQ ID NO: 428 - RNA, 20 nt, synthetic construct
  misc_feature 1..20: note = Description of sequence: sgRNA: SafeHarborchr18_3
  source 1..20: mol_type = other RNA
gaaaaaagttaacttagact

SEQ ID NO: 429 - RNA, 20 nt, synthetic construct
  misc_feature 1..20: note = Description of sequence: sgRNA: SafeHarborchr2_32
  source 1..20: mol_type = other RNA
gaaaagaataacagtagtaa

SEQ ID NO: 430 - RNA, 20 nt, synthetic construct
  misc_feature 1..20: note = Description of sequence: sgRNA: SafeHarborchr21_23
  source 1..20: mol_type = other RNA
gaaaaccagacaagttacct

SEQ ID NO: 431 - RNA, 20 nt, synthetic construct
  misc_feature 1..20: note = Description of sequence: sgRNA: SafeHarborchr21_31
  source 1..20: mol_type = other RNA
gaaaagaaatatcattggca

SEQ ID NO: 432 - RNA, 20 nt, synthetic construct
  misc_feature 1..20: note = Description of sequence: sgRNA: SafeHarborchr3_2
  source 1..20: mol_type = other RNA
gaaaaaagctggagagacac

SEQ ID NO: 433 - RNA, 20 nt, synthetic construct
  misc_feature 1..20: note = Description of sequence: sgRNA: SafeHarborchr3_22
  source 1..20: mol_type = other RNA
gaaaacagctattcggtgct

SEQ ID NO: 434 - RNA, 20 nt, synthetic construct
  misc_feature 1..20: note = Description of sequence: sgRNA: SafeHarborchr3_27
  source 1..20: mol_type = other RNA
gaaaactcccttgtcttggg

SEQ ID NO: 435 - RNA, 20 nt, synthetic construct
  misc_feature 1..20: note = Description of sequence: sgRNA: SafeHarborchr3_47
  source 1..20: mol_type = other RNA
gaaaagttctattcaatttg

SEQ ID NO: 436 - RNA, 20 nt, synthetic construct
  misc_feature 1..20: note = Description of sequence: sgRNA: SafeHarborchr3_6
  source 1..20: mol_type = other RNA
gaaaaagagacagagtatgt

SEQ ID NO: 437 - RNA, 20 nt, synthetic construct
  misc_feature 1..20: note = Description of sequence: sgRNA: SafeHarborchr4_17
  source 1..20: mol_type = other RNA
gaaaaattgtcccagtacac

SEQ ID NO: 438 - RNA, 20 nt, synthetic construct
  misc_feature 1..20: note = Description of sequence: sgRNA: SafeHarborchr4_35
  source 1..20: mol_type = other RNA
gaaaagacctcattagagtg

SEQ ID NO: 439 - RNA, 20 nt, synthetic construct
  misc_feature 1..20: note = Description of sequence: sgRNA: SafeHarborchr4_36
  source 1..20: mol_type = other RNA
gaaaagatctctaccaggaa

SEQ ID NO: 440 - RNA, 20 nt, synthetic construct
  misc_feature 1..20: note = Description of sequence: sgRNA: SafeHarborchr4_4
  source 1..20: mol_type = other RNA
gaaaaacagacttaggggag

SEQ ID NO: 441 - RNA, 20 nt, synthetic construct
  misc_feature 1..20: note = Description of sequence: sgRNA: SafeHarborchr4_42
  source 1..20: mol_type = other RNA
gaaaagtaaaaccctctcag

SEQ ID NO: 442 - RNA, 20 nt, synthetic construct
  misc_feature 1..20: note = Description of sequence: sgRNA: SafeHarborchr4_43
  source 1..20: mol_type = other RNA
gaaaagtatctatgatgaaa

SEQ ID NO: 443 - RNA, 20 nt, synthetic construct
  misc_feature 1..20: note = Description of sequence: sgRNA: SafeHarborchr4_44
  source 1..20: mol_type = other RNA
gaaaagtcacactaaagtgt

SEQ ID NO: 444 - RNA, 20 nt, synthetic construct
  misc_feature 1..20: note = Description of sequence: sgRNA: SafeHarborchr4_46
  source 1..20: mol_type = other RNA
gaaaagttagagtgaagtag

SEQ ID NO: 445 - RNA, 20 nt, synthetic construct
  misc_feature 1..20: note = Description of sequence: sgRNA: SafeHarborchr4_48
  source 1..20: mol_type = other RNA
gaaaataaaggtaatggctg

SEQ ID NO: 446 - RNA, 20 nt, synthetic construct
  misc_feature 1..20: note = Description of sequence: sgRNA: SafeHarborchr4_9
  source 1..20: mol_type = other RNA
gaaaaaggcaggcaatggtg

SEQ ID NO: 447 - RNA, 20 nt, synthetic construct
  misc_feature 1..20: note = Description of sequence: sgRNA: SafeHarborchr5_5
  source 1..20: mol_type = other RNA
gaaaaactcttagcttaatg

SEQ ID NO: 448 - RNA, 20 nt, synthetic construct
  misc_feature 1..20: note = Description of sequence: sgRNA: SafeHarborchr6_14
  source 1..20: mol_type = other RNA
gaaaaatgacccatgattaa

SEQ ID NO: 449 - RNA, 20 nt, synthetic construct
  misc_feature 1..20: note = Description of sequence: sgRNA: SafeHarborchr6_50
  source 1..20: mol_type = other RNA
gaaaataaggcatcaataac

SEQ ID NO: 450 - RNA, 20 nt, synthetic construct
  misc_feature 1..20: note = Description of sequence: sgRNA: SafeHarborchr7_11
  source 1..20: mol_type = other RNA
gaaaaatagctttctctcaa

SEQ ID NO: 451 - RNA, 20 nt, synthetic construct
  misc_feature 1..20: note = Description of sequence: sgRNA: SafeHarborchr8_13
  source 1..20: mol_type = other RNA
gaaaaatcgctaatatgaaa

SEQ ID NO: 452 - RNA, 20 nt, synthetic construct
  misc_feature 1..20: note = Description of sequence: sgRNA: SafeHarborchr8_37
  source 1..20: mol_type = other RNA
gaaaagatgagttatattgg

SEQ ID NO: 453 - RNA, 20 nt, synthetic construct
  misc_feature 1..20: note = Description of sequence: sgRNA: SafeHarborchr8_7
  source 1..20: mol_type = other RNA
gaaaaagcagcacttcttat

SEQ ID NO: 454 - RNA, 20 nt, synthetic construct
  misc_feature 1..20: note = Description of sequence: sgRNA: SafeHarborchr9_18
  source 1..20: mol_type = other RNA
gaaaaatttcagcagacctt

SEQ ID NO: 455 - RNA, 20 nt, synthetic construct
  misc_feature 1..20: note = Description of sequence: sgRNA: SafeHarborchr9_20
  source 1..20: mol_type = other RNA
gaaaacacagtcttaattat

SEQ ID NO: 456 - RNA, 20 nt, synthetic construct
  misc_feature 1..20: note = Description of sequence: sgRNA: SafeHarborchr9_39
  source 1..20: mol_type = other RNA
gaaaaggaagattctgctgt

SEQ ID NO: 457 - RNA, 20 nt, synthetic construct
  misc_feature 1..20: note = Description of sequence: sgRNA: SafeHarborchr9_40
  source 1..20: mol_type = other RNA
gaaaaggtgaggttaaggag

SEQ ID NO: 458 - RNA, 20 nt, synthetic construct
  misc_feature 1..20: note = Description of sequence: sgRNA: SafeHarborchr9_49
  source 1..20: mol_type = other RNA
gaaaataaccacaattatca

SEQ ID NO: 459 - RNA, 20 nt, synthetic construct
  misc_feature 1..20: note = Description of sequence: sgRNA: SafeHarborchrX_10
  source 1..20: mol_type = other RNA
gaaaaataaggtgaagatag

SEQ ID NO: 460 - RNA, 20 nt, synthetic construct
  misc_feature 1..20: note = Description of sequence: sgRNA: SafeHarborchrX_15
  source 1..20: mol_type = other RNA
gaaaaatgactggtcacatt

SEQ ID NO: 461 - RNA, 20 nt, synthetic construct
  misc_feature 1..20: note = Description of sequence: sgRNA: SIRT5_1
  source 1..20: mol_type = other RNA
aacttggccgagccattttc

SEQ ID NO: 462 - RNA, 20 nt, synthetic construct
  misc_feature 1..20: note = Description of sequence: sgRNA: SIRT5_2
  source 1..20: mol_type = other RNA
tcaatcgacttgggacaatc

SEQ ID NO: 463 - RNA, 20 nt, synthetic construct
  misc_feature 1..20: note = Description of sequence: sgRNA: SIRT5_3
  source 1..20: mol_type = other RNA
ccacacccgggacgggttgt

SEQ ID NO: 464 - RNA, 20 nt, synthetic construct
  misc_feature 1..20: note = Description of sequence: sgRNA: SLC11A2_1
  source 1..20: mol_type = other RNA
ccagcggcttgcagctagac

SEQ ID NO: 465 - RNA, 20 nt, synthetic construct
  misc_feature 1..20: note = Description of sequence: sgRNA: SLC11A2_2
  source 1..20: mol_type = other RNA
actattatggccctcacatt

SEQ ID NO: 466 - RNA, 20 nt, synthetic construct
  misc_feature 1..20: note = Description of sequence: sgRNA: SLC11A2_3
  source 1..20: mol_type = other RNA
cgaacatgcccttgagtacc

SEQ ID NO: 467 - RNA, 20 nt, synthetic construct
  misc_feature 1..20: note = Description of sequence: sgRNA: SLC25A15_1
  source 1..20: mol_type = other RNA
gcgatgttggcgattagtgc

SEQ ID NO: 468 - RNA, 20 nt, synthetic construct
  misc_feature 1..20: note = Description of sequence: sgRNA: SLC25A15_2
  source 1..20: mol_type = other RNA
gttggcgattagtgctggac

SEQ ID NO: 469 - RNA, 20 nt, synthetic construct
  misc_feature 1..20: note = Description of sequence: sgRNA: SLC25A15_3
  source 1..20: mol_type = other RNA
agtcggtgaggccccggtac

SEQ ID NO: 470 - RNA, 20 nt, synthetic construct
  misc_feature 1..20: note = Description of sequence: sgRNA: SLC7A11_1
  source 1..20: mol_type = other RNA
taccagcttttgtacgagtc

SEQ ID NO: 471 - RNA, 20 nt, synthetic construct
  misc_feature 1..20: note = Description of sequence: sgRNA: SLC7A11_2
  source 1..20: mol_type = other RNA
agcttttgtacgagtctggg

SEQ ID NO: 472 - RNA, 20 nt, synthetic construct
  misc_feature 1..20: note = Description of sequence: sgRNA: SLC7A11_3
  source 1..20: mol_type = other RNA
acccagactcgtacaaaagc

SEQ ID NO: 473 - RNA, 20 nt, synthetic construct
  misc_feature 1..20: note = Description of sequence: sgRNA: SNX30_1
  source 1..20: mol_type = other RNA
cccaacggtggtactccagc

SEQ ID NO: 474 - RNA, 20 nt, synthetic construct
  misc_feature 1..20: note = Description of sequence: sgRNA: SNX30_2
  source 1..20: mol_type = other RNA
acctgctggagtaccaccgt

SEQ ID NO: 475 - RNA, 20 nt, synthetic construct
  misc_feature 1..20: note = Description of sequence: sgRNA: SNX30_3
  source 1..20: mol_type = other RNA
cctgctggagtaccaccgtt

SEQ ID NO: 476 - RNA, 20 nt, synthetic construct
  misc_feature 1..20: note = Description of sequence: sgRNA: SNX4_1
  source 1..20: mol_type = other RNA
aggcacctccggaccccgag

SEQ ID NO: 477 - RNA, 20 nt, synthetic construct
  misc_feature 1..20: note = Description of sequence: sgRNA: SNX4_2
  source 1..20: mol_type = other RNA
gctctggggtcgacacggtg

SEQ ID NO: 478 - RNA, 20 nt, synthetic construct
  misc_feature 1..20: note = Description of sequence: sgRNA: SNX4_3
  source 1..20: mol_type = other RNA
aggtcagttgaacataccga

SEQ ID NO: 479 - RNA, 20 nt, synthetic construct
  misc_feature 1..20: note = Description of sequence: sgRNA: TFRC_1
  source 1..20: mol_type = other RNA
ctgcagcacgtcgcttatat

SEQ ID NO: 480 - RNA, 20 nt, synthetic construct
  misc_feature 1..20: note = Description of sequence: sgRNA: TFRC_2
  source 1..20: mol_type = other RNA
aatataagcgacgtgctgca

SEQ ID NO: 481 - RNA, 20 nt, synthetic construct
  misc_feature 1..20: note = Description of sequence: sgRNA: TFRC_3
  source 1..20: mol_type = other RNA
ctatacgccacataaccccc

SEQ ID NO: 482 - RNA, 20 nt, synthetic construct
  misc_feature 1..20: note = Description of sequence: sgRNA: TLR4_1
  source 1..20: mol_type = other RNA
caaagatacaccagcggctc

SEQ ID NO: 483 - RNA, 20 nt, synthetic construct
  misc_feature 1..20: note = Description of sequence: sgRNA: TLR4_2
  source 1..20: mol_type = other RNA
attctcccagaaccaaacga

SEQ ID NO: 484 - RNA, 20 nt, synthetic construct
  misc_feature 1..20: note = Description of sequence: sgRNA: TLR4_3
  source 1..20: mol_type = other RNA
caatcacctttcggctttta

SEQ ID NO: 485 - RNA, 20 nt, synthetic construct
  misc_feature 1..20: note = Description of sequence: sgRNA: TLR7_1
  source 1..20: mol_type = other RNA
gcgggtttgttggccactca

SEQ ID NO: 486 - RNA, 20 nt, synthetic construct
  misc_feature 1..20: note = Description of sequence: sgRNA: TLR7_2
  source 1..20: mol_type = other RNA
ttatttttacacggcgcaca

SEQ ID NO: 487 - RNA, 20 nt, synthetic construct
  misc_feature 1..20: note = Description of sequence: sgRNA: TLR7_3
  source 1..20: mol_type = other RNA
agcgcatcaaaagcatttac

SEQ ID NO: 488 - RNA, 20 nt, synthetic construct
  misc_feature 1..20: note = Description of sequence: sgRNA: TNF_1
  source 1..20: mol_type = other RNA
ttggagtgatcggcccccag

SEQ ID NO: 489 - RNA, 20 nt, synthetic construct
  misc_feature 1..20: note = Description of sequence: sgRNA: TNF_2
  source 1..20: mol_type = other RNA
agagctcttacctacaacat

SEQ ID NO: 490 - RNA, 20 nt, synthetic construct
  misc_feature 1..20: note = Description of sequence: sgRNA: TNF_3
  source 1..20: mol_type = other RNA
ggagctgagagataaccagc

SEQ ID NO: 491 - RNA, 20 nt, synthetic construct
  misc_feature 1..20: note = Description of sequence: sgRNA: TNFRSF10A_1
  source 1..20: mol_type = other RNA
cttcaagtttgtcgtcgtcg

SEQ ID NO: 492 - RNA, 20 nt, synthetic construct
  misc_feature 1..20: note = Description of sequence: sgRNA: TNFRSF10A_2
  source 1..20: mol_type = other RNA
tgagctaggtacgacctgtg

SEQ ID NO: 493 - RNA, 20 nt, synthetic construct
  misc_feature 1..20: note = Description of sequence: sgRNA: TNFRSF10A_3
  source 1..20: mol_type = other RNA
ctgagctaggtacgacctgt

SEQ ID NO: 494 - RNA, 20 nt, synthetic construct
  misc_feature 1..20: note = Description of sequence: sgRNA: TNFRSF10B_1
  source 1..20: mol_type = other RNA
ccgcggcgacaacgagcaca

SEQ ID NO: 495 - RNA, 20 nt, synthetic construct
  misc_feature 1..20: note = Description of sequence: sgRNA: TNFRSF10B_2
  source 1..20: mol_type = other RNA
gagcggccccacaacaaaag

SEQ ID NO: 496 - RNA, 20 nt, synthetic construct
  misc_feature 1..20: note = Description of sequence: sgRNA: TNFRSF10B_3
  source 1..20: mol_type = other RNA
atagtcctgtccatatttgc

SEQ ID NO: 497 - RNA, 20 nt, synthetic construct
  misc_feature 1..20: note = Description of sequence: sgRNA: TNFRSF10C_1
  source 1..20: mol_type = other RNA
gatgacgacgacgaacttta

SEQ ID NO: 498 - RNA, 20 nt, synthetic construct
  misc_feature 1..20: note = Description of sequence: sgRNA: TNFRSF10C_2
  source 1..20: mol_type = other RNA
cggcgtcgggaaccatacca

SEQ ID NO: 499 - RNA, 20 nt, synthetic construct
  misc_feature 1..20: note = Description of sequence: sgRNA: TNFRSF10C_3
  source 1..20: mol_type = other RNA
cgatgacgacgacgaacttt

SEQ ID NO: 500 - RNA, 20 nt, synthetic construct
  misc_feature 1..20: note = Description of sequence: sgRNA: TNFRSF10D_1
  source 1..20: mol_type = other RNA
gctcgagcagggcgctatcc

SEQ ID NO: 501 - RNA, 20 nt, synthetic construct
  misc_feature 1..20: note = Description of sequence: sgRNA: TNFRSF10D_2
  source 1..20: mol_type = other RNA
aacttcgtcctgccggggga

SEQ ID NO: 502 - RNA, 20 nt, synthetic construct
  misc_feature 1..20: note = Description of sequence: sgRNA: TNFRSF10D_3
  source 1..20: mol_type = other RNA
tggggaacttcgtcctgccg

SEQ ID NO: 503 - RNA, 20 nt, synthetic construct
  misc_feature 1..20: note = Description of sequence: sgRNA: TNFRSF11B_1
  source 1..20: mol_type = other RNA
cttccttgcattcgcacacg

SEQ ID NO: 504 - RNA, 20 nt, synthetic construct
  misc_feature 1..20: note = Description of sequence: sgRNA: TNFRSF11B_2
  source 1..20: mol_type = other RNA
caaccgcgtgtgcgaatgca

SEQ ID NO: 505 - RNA, 20 nt, synthetic construct
  misc_feature 1..20: note = Description of sequence: sgRNA: TNFRSF11B_3
  source 1..20: mol_type = other RNA
gcattcgcacacgcggttgt

SEQ ID NO: 506 - RNA, 20 nt, synthetic construct
  misc_feature 1..20: note = Description of sequence: sgRNA: TNFRSF1A_1
  source 1..20: mol_type = other RNA
atatacccctcaggggttat

SEQ ID NO: 507 - RNA, 20 nt, synthetic construct
  misc_feature 1..20: note = Description of sequence: sgRNA: TNFRSF1A_2
  source 1..20: mol_type = other RNA
attggactggtccctcacct

SEQ ID NO: 508 - RNA, 20 nt, synthetic construct
  misc_feature 1..20: note = Description of sequence: sgRNA: TNFRSF1A_3
  source 1..20: mol_type = other RNA
cactccaataatgccggtac

SEQ ID NO: 509 - RNA, 20 nt, synthetic construct
  misc_feature 1..20: note = Description of sequence: sgRNA: TNFSF10_1
  source 1..20: mol_type = other RNA
gaagatcacgatcagcacgc

SEQ ID NO: 510 - RNA, 20 nt, synthetic construct
  misc_feature 1..20: note = Description of sequence: sgRNA: TNFSF10_2
  source 1..20: mol_type = other RNA
actccgtcagctcgttagaa

SEQ ID NO: 511 - RNA, 20 nt, synthetic construct
  misc_feature 1..20: note = Description of sequence: sgRNA: TNFSF10_3
  source 1..20: mol_type = other RNA
cgtcagctcgttagaaaggt

SEQ ID NO: 512 - RNA, 20 nt, synthetic construct
  misc_feature 1..20: note = Description of sequence: sgRNA: TP53_1
  source 1..20: mol_type = other RNA
ccccggacgatattgaacaa

SEQ ID NO: 513 - RNA, 20 nt, synthetic construct
  misc_feature 1..20: note = Description of sequence: sgRNA: TP53_2
  source 1..20: mol_type = other RNA
ccattgttcaatatcgtccg

SEQ ID NO: 514 - RNA, 20 nt, synthetic construct
  misc_feature 1..20: note = Description of sequence: sgRNA: TP53_3
  source 1..20: mol_type = other RNA
ccattgcttgggacggcaag

SEQ ID NO: 515 - RNA, 20 nt, synthetic construct
  misc_feature 1..20: note = Description of sequence: sgRNA: UBR5_1
  source 1..20: mol_type = other RNA
cgggtgaaccacgaaatgga

SEQ ID NO: 516 - RNA, 20 nt, synthetic construct
  misc_feature 1..20: note = Description of sequence: sgRNA: UBR5_2
  source 1..20: mol_type = other RNA
ccaatacattcaaagggggg

SEQ ID NO: 517 - RNA, 20 nt, synthetic construct
  misc_feature 1..20: note = Description of sequence: sgRNA: UBR5_3
  source 1..20: mol_type = other RNA
ccacccccctttgaatgtat

SEQ ID NO: 518 - RNA, 20 nt, synthetic construct
  misc_feature 1..20: note = Description of sequence: sgRNA: ULK1_1
  source 1..20: mol_type = other RNA
cgaaggcgccgtggccgatc

SEQ ID NO: 519 - RNA, 20 nt, synthetic construct
  misc_feature 1..20: note = Description of sequence: sgRNA: ULK1_2
  source 1..20: mol_type = other RNA
gcccttgaagaccaccgcga

SEQ ID NO: 520 - RNA, 20 nt, synthetic construct
  misc_feature 1..20: note = Description of sequence: sgRNA: ULK1_3
  source 1..20: mol_type = other RNA
gtggccgatcaggtccttgc

SEQ ID NO: 521 - RNA, 20 nt, synthetic construct
  misc_feature 1..20: note = Description of sequence: sgRNA: ULK2_1
  source 1..20: mol_type = other RNA
tcgccgtggtcttccggggg

SEQ ID NO: 522 - RNA, 20 nt, synthetic construct
  misc_feature 1..20: note = Description of sequence: sgRNA: ULK2_2
  source 1..20: mol_type = other RNA
gacctcgcagattatttgca

SEQ ID NO: 523 - RNA, 20 nt, synthetic construct
  misc_feature 1..20: note = Description of sequence: sgRNA: ULK2_3
  source 1..20: mol_type = other RNA
gtccctttcgctggtacaaa

SEQ ID NO: 524 - RNA, 20 nt, synthetic construct
  misc_feature 1..20: note = Description of sequence: sgRNA: VDAC2_1
  source 1..20: mol_type = other RNA
cgcgcgtcgtaagtaaagct

SEQ ID NO: 525 - RNA, 20 nt, synthetic construct
  misc_feature 1..20: note = Description of sequence: sgRNA: VDAC2_2
  source 1..20: mol_type = other RNA
gcgcgcgtcgtaagtaaagc

SEQ ID NO: 526 - RNA, 20 nt, synthetic construct
  misc_feature 1..20: note = Description of sequence: sgRNA: VDAC2_3
  source 1..20: mol_type = other RNA
tgttaggaattttcaacgtc

SEQ ID NO: 527 - RNA, 20 nt, synthetic construct
  misc_feature 1..20: note = Description of sequence: sgRNA: VDAC3_1
  source 1..20: mol_type = other RNA
cctttacttacctggtcgaa

SEQ ID NO: 528 - RNA, 20 nt, synthetic construct
  misc_feature 1..20: note = Description of sequence: sgRNA: VDAC3_2
  source 1..20: mol_type = other RNA
cccttcgaccaggtaagtaa

SEQ ID NO: 529 - RNA, 20 nt, synthetic construct
  misc_feature 1..20: note = Description of sequence: sgRNA: VDAC3_3
  source 1..20: mol_type = other RNA
tcctttacttacctggtcga

SEQ ID NO: 530 - RNA, 20 nt, synthetic construct
  misc_feature 1..20: note = Description of sequence: sgRNA: WIPI1_1
  source 1..20: mol_type = other RNA
tgatacacgttcatctgccg

SEQ ID NO: 531 - RNA, 20 nt, synthetic construct
  misc_feature 1..20: note = Description of sequence: sgRNA: WIPI1_2
  source 1..20: mol_type = other RNA
tacttgccggttcagcctta

SEQ ID NO: 532 - RNA, 20 nt, synthetic construct
  misc_feature 1..20: note = Description of sequence: sgRNA: WIPI1_3
  source 1..20: mol_type = other RNA
ccttatggacaagatgttgc

SEQ ID NO: 533 - RNA, 20 nt, synthetic construct
  misc_feature 1..20: note = Description of sequence: sgRNA: WIPI2_1
  source 1..20: mol_type = other RNA
cccggccttacgtgttgtcc

SEQ ID NO: 534 - RNA, 20 nt, synthetic construct
  misc_feature 1..20: note = Description of sequence: sgRNA: WIPI2_2
  source 1..20: mol_type = other RNA
caggacaacacgtaaggccg

SEQ ID NO: 535 - RNA, 20 nt, synthetic construct
  misc_feature 1..20: note = Description of sequence: sgRNA: WIPI2_3
  source 1..20: mol_type = other RNA
accaggacaacacgtaaggc

SEQ ID NO: 536 - RNA, 20 nt, synthetic construct
  misc_feature 1..20: note = Description of sequence: sgRNA: XIAP_1
  source 1..20: mol_type = other RNA
ggactctactacacaggtat

SEQ ID NO: 537 - RNA, 20 nt, synthetic construct
  misc_feature 1..20: note = Description of sequence: sgRNA: XIAP_2
  source 1..20: mol_type = other RNA
catcaacactggcacgagca

SEQ ID NO: 538 - RNA, 20 nt, synthetic construct
  misc_feature 1..20: note = Description of sequence: sgRNA: XIAP_3
  source 1..20: mol_type = other RNA
tatcagacaccatatacccg

SEQ ID NO: 539 - DNA, 20 bp, synthetic construct
  misc_feature 1..20: note = Description of sequence: Primer: Human PTGFR2 primer 5'
  source 1..20: mol_type = other DNA
gctgcttctcattgtctcgg

SEQ ID NO: 540 - DNA, 20 bp, synthetic construct
  misc_feature 1..20: note = Description of sequence: Primer: Human PTGFR2 primer 3'
  source 1..20: mol_type = other DNA
gccaggagaatgaggtggtc

SEQ ID NO: 541 - DNA, 20 bp, synthetic construct
  misc_feature 1..20: note = Description of sequence: Primer: Mouse ptgfr2 primer 5'
  source 1..20: mol_type = other DNA
cctgctgcttatcgtggctg

SEQ ID NO: 542 - DNA, 20 bp, synthetic construct
  misc_feature 1..20: note = Description of sequence: Primer: Mouse ptgfr2 primer 3'
  source 1..20: mol_type = other DNA
gccaggagaatgaggtggtc

SEQ ID NO: 543 - DNA, 20 bp, synthetic construct
  misc_feature 1..20: note = Description of sequence: Primer: Human GAPDH primer 5'
  source 1..20: mol_type = other DNA
atgttcgtcatgggtgtgaa

SEQ ID NO: 544 - DNA, 20 bp, synthetic construct
  misc_feature 1..20: note = Description of sequence: Primer: Human GAPDH primer 3'
  source 1..20: mol_type = other DNA
aggggtgctaagcagttggt

SEQ ID NO: 545 - DNA, 21 bp, synthetic construct
  misc_feature 1..21: note = Description of sequence: Primer: Human FOXA2 primer 5'
  source 1..21: mol_type = other DNA
ccgactggagcagctactatg

SEQ ID NO: 546 - DNA, 20 bp, synthetic construct
  misc_feature 1..20: note = Description of sequence: Primer: Human FOXA2 primer 3'
  source 1..20: mol_type = other DNA
tacgtgttcatgccgttcat

SEQ ID NO: 547 - DNA, 22 bp, synthetic construct
  misc_feature 1..22: note = Description of sequence: Primer: Human NURR1 primer 5'
  source 1..22: mol_type = other DNA
cgcttctcagagctacagttac

SEQ ID NO: 548 - DNA, 20 bp, synthetic construct
  misc_feature 1..20: note = Description of sequence: Primer: Human NURR1 primer 3'
  source 1..20: mol_type = other DNA
tggtgaggtccatgctaaac

SEQ ID NO: 549 - DNA, 22 bp, synthetic construct
  misc_feature 1..22: note = Description of sequence: Primer: Human PUMA primer 5'
  source 1..22: mol_type = other DNA
cctggagggtcctgtacaatct

SEQ ID NO: 550 - DNA, 19 bp, synthetic construct
  misc_feature 1..19: note = Description of sequence: Primer: Human PUMA primer 3'
  source 1..19: mol_type = other DNA
tctgtggcccctgggtaag

SEQ ID NO: 551 - DNA, 23 bp, synthetic construct
  misc_feature 1..23: note = Description of sequence: Primer: Human TP53 primer 5'
  source 1..23: mol_type = other DNA
gtaccaccatccactacaactac

SEQ ID NO: 552 - DNA, 20 bp, synthetic construct
  misc_feature 1..20: note = Description of sequence: Primer: Human TP53 primer 3'
  source 1..20: mol_type = other DNA
cacaaacacgcacctcaaag

(19) United States
(12) Patent Application Publication
(10) Pub. No.: US 2025/0017979 A1
(43) Pub. Date: Jan. 16, 2025

(54) METHODS AND COMPOSITIONS FOR IMPROVING IN VIVO SURVIVAL OF MIDBRAIN DOPAMINE NEURONS

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Lorenz Studer, New York, NY (US); Taewan Kim, New York, NY (US); So Yeon Koo, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 18/889,101

(22) Filed: Sep. 18, 2024

Related U.S. Application Data
(63) Continuation of application No. PCT/US2023/015644, filed on Mar. 20, 2023.
(60) Provisional application No. 63/321,507, filed on Mar. 18, 2022.

(51) Int. Cl. A61K 35/50; A61P 25/28; C12N 5/0793; C12N 5/0797

(52) U.S. Cl. CPC A61K 35/50; A61P 25/28; C12N 5/0619; C12N 5/0623; C12N 2501/10; C12N 2506/03

(57) ABSTRACT

The present disclosure provides methods for improving in vivo survival of midbrain dopamine (mDA) neurons (e.g., in vitro differentiated mDA neurons) by suppressing p53-mediated apoptosis of mDA neurons. The present disclosure further provides methods for treating a subject (e.g., a subject suffering from neurodegeneration of midbrain dopamine neurons, and/or a neurodegenerative disease), comprising administering to the subject one or more mDAs, wherein p53-mediated apoptosis of the one or more mDA neurons is suppressed.

### PRIORITY

This patent application is a continuation of International Application No. PCT/US2023/015644 filed Mar. 20, 2023, which claims priority to U.S. provisional application 63/321,507 filed Mar. 18, 2022, the contents of both of which are incorporated herein by reference in their entireties.

### GRANT INFORMATION

The present disclosure was made with government support under Grant No. NS118067-01A1 awarded by the National Institute of Health. The government has certain rights in the disclosure.

### SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on May 12, 2023, is entitled “0727341657_ST26.xml”, and is 714,156 bytes in size.

### INTRODUCTION

The present disclosure provides methods and compositions for improving in vivo survival of midbrain dopamine (mDA) neurons (e.g., in vitro differentiated mDA neurons) by suppressing p53-mediated apoptosis of mDA neurons. The present disclosure further provides methods and compositions for treating a subject (e.g., a subject suffering from neurodegeneration of midbrain dopamine neurons and/or a neurodegenerative disease), comprising administering to the subject one or more mDAs, wherein p53-mediated apoptosis of the one or more mDA neurons is suppressed.

### BACKGROUND

Parkinson's disease (PD) remains a major scientific and therapeutic challenge. PD affects an estimated 10 million cases worldwide and brings enormous costs to affected individuals and the greater society in general (Dorsey et al., 2018, J Parkinsons Dis 8, S3-S8). The rapid increase in the number of PD cases globally has been referred to as a “Parkinson's Pandemic” by some stressing the urgent need for the development of disease-modifying therapies (Dorsey et al., 2018). Although existing therapies can relieve motor symptoms at early stages of the disease, these symptomatic treatments became gradually less effective, they do not prevent or slow down disease progression and can trigger side-effects such as dyskinesia, speech deterioration, or depression in some of the patients (Jenner, 2008, Nat Rev Neurosci 9, 665-677).

PD patients share a common pathological feature, which is the progressive degeneration of dopamine neurons in the substantia nigra para compacta (Poewe et al., 2017, Nat Rev Dis Primers 3, 17013). Cell-based therapy is being considered as a novel therapeutic strategy, as it has the potential to achieve circuit-level restoration of dopaminergic function. Current cell replacement therapy in PD patients was pursued using human fetal midbrain tissue transplantation. While the procedure has shown success in improving motor-related symptoms in a small subset of PD patients (Kefalopoulou et al., 2014, JAMA Neurol 71, 83-87; Li et al., 2016, Proc Natl Acad Sci USA 113, 6544-6549), there was huge variability in clinical responses across patients; a two placebo-controlled studies failed to reach their primary endpoints (Freed et al., 2001, N Engl J Med 344, 710-719; Olanow et al., 2003, Ann Neurol 54, 403-414). Given the challenges of using human fetal tissue as a routine, scalable, and defined source of dopamine neurons, alternative sources such as human pluripotent stem cell (hPSC)-derived dopamine neurons have become the focus for ongoing cell therapy efforts in PD (Piao et al., 2021, Cell Stem Cell 28, 217-229 e217; Schweitzer et al., 2020, N Engl J Med 382, 1926-1932).

One challenge that has been largely overlooked in all these efforts is the limited survival of grafted dopamine neurons following transplantation surgery. This is a critical factor in determining the success of the procedure given the need to reach a critical dose of dopamine neurons to achieve clinical improvement and given the challenges of predicting effective dose in the patient's brain if there is considerable variability in initial cell survival. Furthermore, injecting a large number of cells to overcome limited in vivo survival can pose a risk for triggering a host inflammatory response from the procedure and the associated cell death of the grafted cells (Kriks et al., 2011, Nature 480, 547-551.; Tao et al., 2021, Nat Med 27, 632-639.).

Therefore, there remains a need for improving the survival of midbrain dopamine neurons following transplantation.

### SUMMARY OF THE INVENTION

The present disclosure provides methods and compositions for improving in vivo survival of midbrain dopamine (mDA) neurons (e.g., in vitro differentiated mDA neurons) by suppressing p53-mediated apoptosis of mDA neurons. The present disclosure further provides methods and compositions for treating a subject (e.g., a subject suffering from neurodegeneration of midbrain dopamine neurons, and/or a neurodegenerative disease), comprising administering to the subject one or more mDAs, wherein p53-mediated apoptosis of the one or more mDA neurons is suppressed.

In certain embodiments, the present disclosure provides methods for treating a subject. In certain embodiments, the method comprises administering to the subject one or more midbrain dopamine (mDA) neurons, wherein p53-mediated apoptosis of the one or more mDA neurons is suppressed. In certain embodiments, the suppression of p53-mediated apoptosis comprises administering to the subject at least one compound selected from the group consisting of tumor necrosis factor alpha (TNFα) inhibitors, nuclear factor kappa B (NFκB) inhibitors, p53 inhibitors, and combinations thereof. In certain embodiments, the method comprises administering the one or more mDA neurons simultaneously with the administration of the at least one compound. In certain embodiments, the suppression of p53-mediated apoptosis comprises contacting the one or more mDA neurons with at least one compound selected from the group consisting of TNFα inhibitors, NFκB inhibitors, p53 inhibitors, and combinations thereof.

In certain embodiments, the subject suffers from a neurodegenerative disorder and/or neurodegeneration of midbrain dopamine neurons. In certain embodiments, the neurodegenerative disorder is selected from the group consisting of Parkinson's disease, Huntington's disease, Alzheimer's disease, multiple sclerosis, amyotrophic lateral sclerosis (ALS), frontotemporal dementia, and combinations thereof.

In certain embodiments, the present disclosure provides methods of improving in vivo survival of one or more midbrain dopamine (mDA) neurons. In certain embodiments, the method comprises suppressing p53-mediated apoptosis of the one or more mDA neurons. In certain embodiments, the suppression of p53-mediated apoptosis comprises contacting the one or more mDA neurons with a compound selected from the group consisting of TNFα inhibitors, NFκB inhibitors, p53 inhibitors, and combinations thereof.

In certain embodiments, the suppression of p53-mediated apoptosis comprises inhibition of tumor necrosis factor alpha (TNFα) signaling, inhibition of nuclear factor kappa B (NFκB) signaling, inhibition of p53 signaling, or a combination of the foregoing.

In certain embodiments, the TNFα inhibitor is selected from the group consisting of anti-TNFα antibodies, TNFα decoy receptors, chemical compounds, nucleic acid inhibitors, small molecule inhibitors, receptor biologic inhibitors, inactive TNF fragments, TNFα circulating receptor fusion protein, xanthine derivatives, 5-HT_{2A} agonists, and combinations thereof. In certain embodiments, the TNFα inhibitor is an anti-TNFα antibody. In certain embodiments, the anti-TNFα antibody is selected from the group consisting of adalimumab, adalimumab-adbm, adalimumab-adaz, adalimumab-atto, certolizumab pegol, golimumab, infliximab, infliximab-abda, infliximab-dyyb, remtolumab, afelimomab, nerelimomab, ozoralizumab, placulumab, and combinations thereof. In certain embodiments, the anti-TNFα antibody is adalimumab.

In certain embodiments, the NFκB inhibitor is selected from the group consisting of upstream inhibitors of NFκB, inhibitors of IKK activity, inhibitors of IκB phosphorylation, inhibitors of IκB degradation, proteasome inhibitors, protease inhibitors, IκB upregulators, inhibitors of NFκB nuclear translocation and expression, NFκB DNA-binding inhibitors, and NFκB transactivation inhibitors, inhibitors of NFκB directed gene transactivation, antioxidants, and combinations thereof.

In certain embodiments, the p53 inhibitor is selected from the group consisting of JNK inhibitors, p38 MAPK inhibitors, caspase inhibitors, puma/BBC3 inhibitors, BAX inhibitors, CDK inhibitors, MDM2 and MDMX activators, and combinations thereof.

In certain embodiments, the suppression of p53-mediated apoptosis comprises knocking out or knocking down TP53 gene in the one or more mDA neurons. In certain embodiments, the TP53 gene is knocked out or knocked down by a gene-engineering system. In certain embodiments, the gene-engineering system is a CRISPR-Cas system.

In certain embodiments, the one or more mDA neurons express a marker selected from the group consisting of EN1, OTX2, TH, NURR1, FOXA2, LMXIA, PITX3, LMO3, SNCA, ADCAP1, CHRNA4, ALDH1A1, SOX6, WNT1, DAT, VMAT2, GIRK2, SATB1, CALB1, CALB2, SNCG, PBX1, and combinations thereof. In certain embodiments, the one or more mDA neurons are post-mitotic mDA neurons.

In certain embodiments, the one or more mDA neurons are in vitro differentiated from one or more stem cells. In certain embodiments, the one or more stem cells are selected from the group consisting of human stem cells, nonhuman primate stem cells, rodent nonembryonic stem cells, human embryonic stem cells, nonhuman primate embryonic stem cells, rodent embryonic stem cells, human induced pluripotent stem cells, nonhuman primate induced pluripotent stem cells, rodent induced pluripotent stem cells, and human recombinant pluripotent cells, nonhuman primate recombinant pluripotent cells, and rodent recombinant pluripotent cells. In certain embodiments, the one or more stem cells are human stem cells. In certain embodiments, the one or more stem cells are one or more pluripotent stem cells or multipotent stem cell. In certain embodiments, the one or more stem cells are selected from the group consisting of embryonic stem cells, induced pluripotent stem cells, and combinations thereof. In certain embodiments, the one or more stem cells are one or more induced pluripotent stem cells.

In certain embodiments, the in vitro differentiation comprises contacting the one or more stem cells with at least one inhibitor of Small Mothers Against Decapentaplegic (SMAD) signaling, at least one activator of Sonic hedgehog (SHH) signaling, and at least one activator of wingless (Wnt) signaling.

In certain embodiments, the concentration of the at least one activator of Wnt signaling that is contacted with the cells is increased between about 2 days and about 6 days from the initial contact of the cells with the at least one activator of Wnt signaling. In certain embodiments, the concentration of the at least one activator of Wnt signaling that is contacted with the cells is increased by between about 250% and about 1800% of the initial concentration of the at least one activator of Wnt signaling contacted with the cells.

In certain embodiments, the at least one activator of Wnt signaling comprises an inhibitor of glycogen synthase kinase 3β (GSK3B) signaling. In certain embodiments, the at least one activator of Wnt signaling is selected from the group consisting of CHIR99021, CHIR98014, AMBMP hydrochloride, LP 922056, Lithium, deoxycholic acid, BIO, SB-216763, Wnt3A, Wnt1, Wnt5a, derivatives thereof, and combinations thereof. In certain embodiments, the at least one activator of Wnt signaling comprises CHIR99021.

In certain embodiments, the at least one inhibitor of SMAD signaling comprises an inhibitor of TGFβ/Activin-Nodal signaling, an inhibitor of bone morphogenetic protein (BMP) signaling, or a combination of the foregoing. In certain embodiments, the at least one inhibitor of TGFβ/Activin-Nodal signaling is selected from the group consisting of SB431542, derivatives of SB431542, and combinations thereof. In certain embodiments, derivative of SB431542 comprises A83-01. In certain embodiments, the at least one inhibitor of TGFβ/Activin-Nodal signaling comprises SB431542. In certain embodiments, the at least one inhibitor of BMP signaling is selected from the group consisting of LDN193189, Noggin, dorsomorphin, derivatives of LDN193189, derivatives of Noggin, derivatives of dorsomorphin, and combinations thereof. In certain embodiments, the at least one inhibitor of BMP comprises LDN-193189.

In certain embodiments, the at least one activator of SHH signaling is selected from the group consisting of SHH proteins, Smoothened agonists (SAG), and combinations thereof. In certain embodiments, the SHH protein is selected from the group consisting of recombinant SHHs, modified N-terminal SHHs, and combinations thereof. In certain embodiments, the modified N-terminal SHH comprises two isoleucines at the N-terminus. In certain embodiments, the modified N-terminal SHH has at least about 90% sequence identity to an un-modified N-terminal SHH. In certain embodiments, the un-modified N-terminal SHH is an un-modified mouse N-terminal SHH or an un-modified human N-terminal SHH. In certain embodiments, the modified N-terminal SHH comprises SHH C25II. In certain embodiments, the SAG comprises purmorphamine. In certain embodiments, the at least one activator of SHH signaling comprises SHH C25II.

In certain embodiments, the in vitro differentiation further comprises contacting the one or more stem cells with at least one activator of fibroblast growth factor (FGF) signaling. In certain embodiments, the at least one activator of FGF signaling is selected from the group consisting of FGF18, FGF17, FGF8a, FGF8b, FGF4, FGF2, and combination thereof. In certain embodiments, the at least one activator of FGF signaling comprises FGF18. In certain embodiments, the at least one activator of FGF signaling comprises FGF8.

In certain embodiments, the in vitro differentiation further comprises contacting the one or more stem cells with at least one inhibitor of Wnt signaling. In certain embodiments, the at least one inhibitor of Wnt signaling is selected from the group consisting of IWP2, IWR1-endo, XAV939, IWP-01, Wnt-C59, IWP-L6, and ICG-001, and combinations thereof. In certain embodiments, the at least one inhibitor of Wnt signaling comprises IWP2.

In certain embodiments, the one or more mDA neurons express a detectable level of CD184 and do not express a detectable level of CD49c.

In certain embodiment, the present disclosure provides compositions comprising: (a) one or more midbrain dopamine (mDA) neurons; and (b) at least one compound selected from the group consisting of TNFα inhibitors, NFκB inhibitors, p53 inhibitors, and combinations thereof. In certain embodiments, the composition is a pharmaceutical composition further comprising a pharmaceutically acceptable carrier.

In certain embodiments, the composition is for treating or ameliorating a neurodegenerative disorder, and/or neurodegeneration of midbrain dopamine neurons. In certain embodiments, the neurodegenerative disorder is selected from the group consisting of Parkinson's disease, Huntington's disease, Alzheimer's disease, multiple sclerosis, amyotrophic lateral sclerosis (ALS), frontotemporal dementia, and combinations thereof.

In certain embodiments, the TNFα inhibitor is selected from the group consisting of anti-TNFα antibodies, TNFα decoy receptors, chemical compounds, nucleic acid inhibitors, small molecule inhibitors, receptor biologic inhibitors, inactive TNF fragments, TNFα circulating receptor fusion protein, xanthine derivatives, 5-HT_{2A} agonist, and combinations thereof. In certain embodiments, the TNFα inhibitor is an anti-TNFα antibody. In certain embodiments, the anti-TNFα antibody is selected from the group consisting of adalimumab, adalimumab-adbm, adalimumab-adaz, adalimumab-atto, certolizumab pegol, golimumab, infliximab, infliximab-abda, infliximab-dyyb, remtolumab, afelimomab, nerelimomab, ozoralizumab, placulumab, and combinations thereof. In certain embodiments, the anti-TNFα antibody is adalimumab. In certain embodiments, the NFκB inhibitor is selected from the group consisting of upstream inhibitors of NFκB, inhibitors of IKK activity, inhibitors of IκB phosphorylation, inhibitors of IκB degradation, proteasome inhibitors, protease inhibitors, IκB upregulators, inhibitors of NFκB nuclear translocation and expression, NFκB DNA-binding inhibitors, and NFκB transactivation inhibitors, inhibitors of NFκB directed gene transactivation, antioxidants, and combinations thereof. In certain embodiments, the p53 inhibitor is selected from the group consisting of JNK inhibitors, p38 MAPK inhibitors, caspase inhibitors, BBC3/PUMA inhibitors, BAX inhibitors, CDK inhibitors, MDM2 and MDMX activators, and combinations thereof.

In certain embodiments, the one or more mDA neurons express a marker selected from the group consisting of EN1, OTX2, TH, NURR1, FOXA2, LMXIA, PITX3, LMO3, SNCA, ADCAP1, CHRNA4, ALDH1A1, SOX6, WNT1, DAT, VMAT2, GIRK2, SATB1, CALB1, CALB2, SNCG, PBX1, and combinations thereof. In certain embodiments, the one or more mDA neurons are post-mitotic mDA neurons.

In certain embodiments, the one or more mDA neurons are in vitro differentiated from one or more stem cells. In certain embodiments, the one or more stem cells are selected from the group consisting of human stem cells, nonhuman primate stem cells, rodent nonembryonic stem cells, human embryonic stem cells, nonhuman primate embryonic stem cells, rodent embryonic stem cells, human induced pluripotent stem cells, nonhuman primate induced pluripotent stem cells, rodent induced pluripotent stem cells, and human recombinant pluripotent cells, nonhuman primate recombinant pluripotent cells, and rodent recombinant pluripotent cells. In certain embodiments, the one or more stem cells are human stem cells. In certain embodiments, the one or more stem cells are one or more pluripotent stem cells or multipotent stem cell. In certain embodiments, the one or more stem cells are selected from the group consisting of embryonic stem cells, induced pluripotent stem cells, and combinations thereof. In certain embodiments, the one or more stem cells are one or more induced pluripotent stem cells.

In certain embodiments, the in vitro differentiation comprises contacting the one or more stem cells with at least one inhibitor of Small Mothers Against Decapentaplegic (SMAD) signaling, at least one activator of Sonic hedgehog (SHH) signaling, and at least one activator of wingless (Wnt) signaling.

In certain embodiments, the concentration of the at least one activator of Wnt signaling that is contacted with the cells is increased between about 2 days and about 6 days from the initial contact of the cells with the at least one activator of Wnt signaling. In certain embodiments, the concentration of the at least one activator of Wnt signaling that is contacted with the cells is increased by between about 250% and about 1800% of the initial concentration of the at least one activator of Wnt signaling contacted with the cells.

In certain embodiments, the at least one activator of Wnt signaling comprises an inhibitor of glycogen synthase kinase 3β (GSK3B) signaling. In certain embodiments, the at least one activator of Wnt signaling is selected from the group consisting of CHIR99021, CHIR98014, AMBMP hydrochloride, LP 922056, Lithium, deoxycholic acid, BIO, SB-216763, Wnt3A, Wnt1, Wnt5a, derivatives thereof, and combinations thereof. In certain embodiments, the at least one activator of Wnt signaling comprises CHIR99021.

In certain embodiments, the at least one inhibitor of SMAD signaling comprises an inhibitor of TGFβ/Activin-Nodal signaling, an inhibitor of bone morphogenetic protein (BMP) signaling, or a combination of the foregoing. In certain embodiments, the at least one inhibitor of TGFβ/Activin-Nodal signaling is selected from the group consisting of SB431542, derivatives of SB431542, and combinations thereof. In certain embodiments, derivative of SB431542 comprises A83-01. In certain embodiments, the at least one inhibitor of TGFβ/Activin-Nodal signaling comprises SB431542. In certain embodiments, the at least one inhibitor of BMP signaling is selected from the group consisting of LDN193189, Noggin, dorsomorphin, derivatives of LDN193189, derivatives of Noggin, derivatives of dorsomorphin, and combinations thereof. In certain embodiments, the at least one inhibitor of BMP comprises LDN-193189.

In certain embodiments, the at least one activator of SHH signaling is selected from the group consisting of SHH proteins, Smoothened agonists (SAG), and combinations thereof. In certain embodiments, the SHH protein is selected from the group consisting of recombinant SHHs, modified N-terminal SHHs, and combinations thereof. In certain embodiments, the modified N-terminal SHH comprises two isoleucines at the N-terminus. In certain embodiments, the modified N-terminal SHH has at least about 90% sequence identity to an un-modified N-terminal SHH. In certain embodiments, the un-modified N-terminal SHH is an un-modified mouse N-terminal SHH or an un-modified human N-terminal SHH. In certain embodiments, the modified N-terminal SHH comprises SHH C25II. In certain embodiments, the SAG comprises purmorphamine. In certain embodiments, the at least one activator of SHH signaling comprises SHH C25II.

In certain embodiments, the in vitro differentiation further comprises contacting the one or more stem cells with at least one activator of fibroblast growth factor (FGF) signaling. In certain embodiments, the at least one activator of FGF signaling is selected from the group consisting of FGF18, FGF17, FGF8a, FGF8b, FGF4, FGF2, and combination thereof. In certain embodiments, the at least one activator of FGF signaling comprises FGF18. In certain embodiments, the at least one activator of FGF signaling comprises FGF8.

In certain embodiments, the in vitro differentiation further comprises contacting the one or more stem cells with at least one inhibitor of Wnt signaling. In certain embodiments, the at least one inhibitor of Wnt signaling is selected from the group consisting of IWP2, IWR1-endo, XAV939, IWP-01, Wnt-C59, IWP-L6, and ICG-001, and combinations thereof. In certain embodiments, the at least one inhibitor of Wnt signaling comprises IWP2.

In certain embodiments, the one or more mDA neurons express a detectable level of CD184 and do not express a detectable level of CD49c.

### BRIEF DESCRIPTION OF THE DRAWINGS

FIGS. 1A-1I illustrate derivation and validation of NURR1:GFP sorted DA neurons for CRISPR/Cas9 screening in vitro and in vivo. FIG. 1A shows dopamine neurons population in culture. FIG. 1B shows microscopy images of endogenous NURR1::GFP and TH expression in graft 1 month post transplantation of day 25 NURR1:GFP sorted DA neurons. FIG. 1C shows flow-cytometry image to generate hPSC line containing pooled lentiviral sgRNAs with MOI 0.35, which indicates single copy sgRNA integration per cell. FIG. 1D shows day 40 dopamine neurons co-expressing NURR1::GFP and gRNA::tdTomato post sorting with GFP and Tomato at day 25 with/without dox treatment from day 16 to day 25. FIG. 1E shows ablation of the tdTomato signal in hPSC-derived post-mitotic dopamine neurons after dox exposure from day 16 to day 25. FIG. 1F shows graft fluorescence expression 1 month post transplantation. FIG. 1G shows PCR analysis for detecting a human PTGER2 gene from dissected tissue around graft region. FIG. 1H shows the overall representation of sgRNA across all condition by next-generation sequencing (NGS) from genomic DNA of in vitro cultured cells and an in vivo grafted cell. FIG. 1I shows the overall representations of sgRNAs across all conditions.

FIGS. 2A-2F illustrate CRISPR screens identifying TP53 as a limiting factor for the survival of post-mitotic dopamine neurons during transplantation. FIG. 2A shows a schematic of the in vivo CRISPR screen. FIG. 2B shows the correlation matrix of all the guide RNAs from different experimental conditions for large pool screen (day 16 in vitro vs. day 25 in vitro with no dox vs. day 25 in vitro with dox vs. day 25 in vivo with dox. Scale bar ranging from 0.9 to 1, 1 being the most correlated value. FIG. 2C shows volcano plots comparing each experimental condition. Depleted genes are labeled in red and enriched genes in blue. FIG. 2D shows enriched sgRNAs in in vivo grafted cells vs in vitro cultured day 25 cells. FIG. 2E shows correlation matrix plot for a small pool screen. A similar scale bar range is used. FIG. 2F shows heatmap of all the guide RNAs comparing day 16 progenitors vs. day 25 neurons with dox treatment vs. day 25 neurons without dox treatment (left). Heatmap of the same set of gRNAs for two independent small pool screens (right). Red coloring in the scale bar indicates enrichment of each guide RNAs in the surviving dopamine neurons in vivo.

FIGS. 3A-3F illustrate the characterization of p53-induced dopamine neuron death during transplantation. FIG. 3A shows FACS strategy to inject enriched NURR1::GFP and sgRNA-p53-tdTomato post-mitotic DA neuron. Dox treated (D16-D25) or non-treated dopamine neurons sorted by FACS are bilaterally injected into each striatum of the adult NSG mice. FIG. 3B shows a representative immunofluorescence image of sorted DA neurons in culture post sorting at day 25 expressing NURR1::GFP, gRNA::RFP, and TH at day 40. n=3 independent experiments. FIG. 3C shows representative confocal images of dopamine neuron graft at 1-month post-transplantation stained with antibodies against NURR1-GFP. Scale bar=100 μm. FIG. 3D shows stercological analysis for the number (using optical fractionator) and volume (cavalier estimator) of the surviving dopamine neurons at 1-month post-transplantation. * p<0.05 (paired t-test). FIG. 3E shows representative immunohistochemistry images of the grafted dopamine neurons at different time points (4 hpt, 24 hpt, 72 hpt, 7 dpt) for TP53, cleavage caspase 3 (CC3), and TUNEL assay. Scale bar=50 μm. FIG. 3F shows quantification of the percentage of TP53, CC3, and TUNEL among DA neurons upon engraftment. n=3 independent experiments.

FIGS. 4A-4D illustrate increased survival of p53 KO dopamine neuron in graft exhibiting dopamine neuron identity. FIGS. 4A-4C show immunofluorescence analysis of FOXA2, TH, and NURR1-GFP signal in surviving p53 WT and KO dopamine neurons in the graft (WT; −DOX, KO; +DOX). FIG. 4D shows RT-qPCR analysis of p53 and p53 downstream genes before and after needle injection of NURR1:GFP sorted DA neuron in culture.

FIGS. 5A-5E illustrate time-course analysis of neuroimmune cells' infiltration into the core of the graft. FIG. 5A shows H&E staining of the graft. FIG. 5B shows immunofluorescence analysis of IBA1. FIG. 5C shows immunofluorescence analysis of GFAP and FOXA2. FIG. 5D shows H&E staining of IBA1. FIG. 5E shows H&E staining of Ly6G.

FIG. 6 shows time-course analysis of fiber outgrowth pattern from the graft.

FIGS. 7A-7H illustrate TNFα-NFκB pathway is an upstream regulator triggering TP53-dependent DA neuron death in the graft. FIG. 7A shows a PCA plot of bulk RNAseq data set showing gene expression profiles from sorted dopamine neurons (day 0), in vitro cultured neurons for 1 day post sorting (day 1 culture), and in vivo grafted neurons for 1 day (day 1 graft). FIG. 7B shows differential expression gene analysis between day 1 culture and day 1 graft. FIG. 7C shows hallmark analysis on the upregulated categories in day 1 grafted neurons vs day 1 culture neuron. FIG. 7D shows NES analysis of enriched tumor necrosis factor-related genes in day 1 graft than day 1 culture neuron. FIG. 7E shows GSEA score from enriched genes in day 1 grafted vs day 1 culture neuron. FIG. 7F shows representative immunohistochemistry images of phosphorylated NF kappa B in grafted neurons at distinct time points post-transplantation. % phosphorylated NFκB positive cells from total grafts were quantified. n=2 independent experiments. FIG. 7G shows representative immunofluorescence images of in vitro dopamine neurons marked by NURR1::GFP for the induction of p53 and NFκB-p65 comparing mock vs. TNFα treatment group vs. TNFα and monoclonal antibodies against TNFα called adalimumab treated conditions. n=3 independent experiments. FIG. 7H shows qRT-PCR of gene expression profiles of the three groups listed in FIG. 7G for FOXA2, NURR1, P53, P21, and BBC3 (PUMA).

FIGS. 8A-8F illustrate TNF-NFκB pathway is an upstream regulator triggering TP53-dependent DA neuron death in the graft. FIG. 8A shows a dendrogram of the cells among sorted, in vitro cultured, and in vivo grafted DA neurons from total RNA-seq. FIG. 8B shows heat map analysis of TNFα-NFκB related genes enriched in the grafted DA neurons than sorted and in vitro cultured DA neurons from total RNA-seq. FIG. 8C shows the clustering distribution of wild-type and p53 knock-out (KO) of grafted DA neurons 1 day post transplantation from single cell RNA-seq. FIG. 8D shows histograms of clustering distribution of wild-type and p53 knock-out (KO). FIG. 8E shows clustering distribution of MAP2 expression for neurons. FIG. 8F shows violin plots of dopamine-specific marker and MAP2 expression for neurons (left). Percent of indicated genes from total population of single cell RNA-seq (right).

FIGS. 9A-9E illustrate TNF-NFκB pathway is an upstream regulator triggering TP53-dependent DA neuron death in the graft. FIG. 9A shows clustering analysis of PCA graphs indicating annotated neuroblasts and floor-plate progenitor. FIG. 9B shows heatmap from apoptotic cell-death related genes, enriched in clusters 3, 4, and 7. FIG. 9C shows TNFRSF12A positive cells in PCA. FIG. 9D shows violin plots of TNFRSF12A positive cells in the clusters. FIG. 9E shows violin plots of increased genes, such as BAX, BAD, TNFRSFIA, TNFRSF12A, and TNFRSF10B in p53 WT versus p53 KO DA neurons in each cluster.

FIGS. 10A-10H illustrate clinically relevant TNFα neutralizing antibodies and CD marker sorting strategies functionally improve the survival of post-mitotic dopamine neurons during transplantation. FIG. 10A shows a schematic of the flow-based cell surface marker screen to enrich post-mitotic DA neurons using genetic NURR1::GFP marker. FIG. 10B shows FACS plot of the % of NURR1::GFP population corresponding to each sorting strategy (CD49e depletion, CD49e depletion and CD171 enrichment double, CD49e depletion, and CD184 enrichment double), indicating CD49c depletion and CD184 enrichment double CD marker sorting lead to the most enriched DA neuron population expressing NURR1::GFP. FIG. 10C shows gene expression of NURR1 via qRT-PCR assay 2 days post sorting using each sorting strategy from FIG. 10B. FIG. 10D shows representative immunofluorescence images of CD49e−/CD184+ double sorted dopamine neurons at 40 DIV, giving rise to pure dopamine neuron cultures co-expressing NURR1::GFP, FOXA2, and TH. FIG. 10E shows 1-month short-term in vivo histology analysis of CD49e−/CD184+ double sorted graft compared with unsorted graft. The double CD marker sorted neuron graft exhibits highly compact surviving dopamine neurons than unsorted graft as shown by human NA and TH immunofluorescence-staining. FIG. 10F shows representative immunofluorescence images of CD49e−/CD184+ double sorted dopamine neurons either treated with PBS or TNFα blocking antibodies adalimumab. Stereological number and volume quantification of the surviving dopamine neurons using NURR1::GFP, n=3. FIG. 10G shows graphs representing data of FIG. 10F. FIG. 10H shows D-amphetamine induced rotation assay in 6-OHDA based PD mice model after transplantation of PBS, CD sorted cell, co-injection of CD sorted cell with adalimumab, frozen day 16 DA progenitor, and co-injection of frozen day 16 DA progenitor with adalimumab.

FIGS. 11A-11D illustrate high content cell surface marker screening finds a novel double sorting strategy matching NURR1-GFP+ dopamine neurons. FIG. 11A shows FACS analysis of NURR1:GFP neurons with indicated CD markers. FIG. 11B shows immunofluorescence analysis of CD49e−/CD184+ double sorted dopamine neurons and unsorted neuron with FOXA2 and a proliferation marker (Ki67). FIG. 11C shows analysis of survived DA neuron and their volume from CD49e−/CD184+ double sorted dopamine neurons in p53 WT and KO. FIG. 11D shows H&E analysis of the graft 1 day post transplantation either treated with PBS or TNFα blocking antibodies adalimumab.

FIGS. 12A-12F illustrate in vivo CRISPR/Cas9 screen for identifying TP53 as a limiting factor for the in vivo survival of hPSC-derived postmitotic dopamine neurons. FIG. 12A shows schematic illustration of the pooled CRISPR/Cas9 screen. FIG. 12B shows Pearson correlation abundance matrix of all guide RNAs across the different experimental conditions [day 16 in vitro (D16) vs. day 25 in vitro with no dox (−D25) vs. day 25 in vitro with dox (+D25) vs. day 25 in vivo with dox (D25)]. Scale bar range is from 0.9 to 1, with 1 being the most correlated value. FIG. 12C shows volcano plots comparing each experimental condition. Depleted sgRNAs are labeled in blue and enriched sgRNAs in red. FIG. 12D shows enriched sgRNAs for in vivo grafted cells versus day 25 in vitro cells, both treated with dox. Blue bar displays two sgRNAs and red bar show three sgRNAs targeting for an indicated gene are enriched in grafted cells than in vitro cells. FIG. 12E shows Pearson correlation matrix plot for the pooled validation screen (library #2). Scale bar range is from 0.8 to 1. FIG. 12F shows heatmap of all guide RNAs from pooled library #2 screen comparing day 16 (D16) vs. day 25 neurons without dox treatment (−D25) vs. day 25 with dox treatment (+D25) in culture (left). Heatmap of the same set of sgRNAs comparing+D25 in vivo vs+D25 in culture from two independent replicate screens (right). Red versus blue scale indicates enrichment of each sgRNAs in the surviving DA neurons in in vivo graft (+D25 in vivo) versus day 25 cultured cells with dox (+D25 in vitro).

FIGS. 13A-AG illustrate characterization of p53-induced dopamine neuron death during transplantation. FIG. 13A shows FACS strategy for injecting enriched postmitotic dopamine neurons expressing NURR1::GFP and sgRNA-TP53-tdTomato. Each dot in the scatterplot indicates a single dopamine neuron at 25 DIV. Dox treated from day 16 to 25 (+DOX, TP53 knock-out; KO) or non-treated (−DOX, isogenic TP53 wild-type; WT) dopamine neurons are isolated by FACS at day 25 based on NURR1::GFP signals (upper panel: P5), followed by sgRNA::tdTomato signal (bottom panel: P6). GFP+/tdTomato+dopamine neurons (P6 population: −DOX vs +DOX) are bilaterally injected into the striatum of adult NSG mice. FIGS. 13B-13C show representative confocal images of dopamine neuron grafts at 1 month post transplantation stained with antibodies against GFP (equivalent to NURR1), scale bar=100 μm. (FIG. 13B) and stereological analysis for the number (using optical fractionator, left) and volume (cavalier estimator, right) of the surviving dopamine neurons at 1 month post transplantation n=5 for each condition (FIG. 13C). FIGS. 13D and 13F show representative confocal images of ALDH1A1 (A9 type dopamine neuron marker) and CALB1 (A10 type dopamine neuron marker). Scale bar=100 μm. FIGS. 13E and 13G show quantification of the percentage of A9 (ALDH1A1, FIG. 13E) and A10 (CALB1, FIG. 13F) dopamine neurons per NURR1 expressing DA neurons at 1 month post transplantation. * p<0.05 (paired t-test). ns.=not significant.

FIGS. 14A-14F illustrate temporal kinetics of the p53-mediated dopamine neuron death-related pathways post implantation. FIGS. 14A-14F show representative immunohistochemistry image (FIGS. 14A-14C) and quantification of the percentages (FIGS. 14D-14F) of the grafted dopamine neurons at different time points (4 hpt, 24 hpt, 72 hpt, 7 dpt) for TP53, cleaved caspase 3 (CC3), and TUNEL as apoptosis markers in dopamine neuron grafts upon transplantation. All markers show robust induction at 24 hr. Scale bar=50 μm. n=4 independent experiments.

FIGS. 15A-15I illustrate TNFα-NFκB pathway is an upstream trigger of p53-dependent dopamine neuron death in the graft. FIG. 15A shows PCA plot of bulk RNAseq data for sorted dopamine neurons either immediately post FACS (day 0, DO), in vitro cultured for 1 day post sorting (day 1 culture, D1 culture) or in vivo grafted for 1 day (day 1 graft, D1 graft). FIG. 15B shows differentially expressed gene (DEG) analysis between D1 culture versus D1 graft. FIG. 15C shows hallmark pathway analysis on the upregulated genes for functional categories in D1 grafted vs D1 cultured neuron. FIG. 15D shows normalized enrichment score (NES) analysis of enriched TNF-related genes in D1 graft vs. D1 culture neuron. FIG. 15E shows unbiased gene set enrichment analysis (GSEA) identifies Apoptosis, TP53, and TNFα as the GO terms most frequently enriched in D1 graft versus D1 culture neuron. FIG. 15F (left panel) shows representative immunohistochemistry images of phosphorylated NFkappaB (p-NFκB) in grafted neurons at distinct time points post transplantation, scale bar=50 μm. FIG. 15F (right panel) shows quantification of the percentages of p-NFκB positive cells among total cells within the graft, n=3 independent experiments. FIG. 15G shows representative immunofluorescence images of NURR1::GFP sorted DA neurons in vitro for the induction of p53 and NFκB-p65 comparing mock vs. TNFα vs. TNFα and monoclonal antibody against TNFα (adalimumab), treated conditions for 1 day. FIGS. 15H and 15I show western blot and qRT-PCR of gene expression profiles of the three groups listed in FIG. 15G for TH and TP53 (FIG. 15H) and for the midbrain mDA markers FOXA2 and NURR1, TP53, and PUMA downstream target of TP53 (FIG. 15I). N>3 independent experiments.

FIGS. 16A-16H illustrate single cell RNA sequencing of grafted neurons identifies JUN-related survival signature and cell death associated dedifferentiation following transplantation. FIG. 16A-16C show UMAP plot of scRNA TP53 WT and KO grafted cells at 1 day post transplantation color coded by cell clusters (FIG. 16A), by TP53 WT and KO genotypes (FIG. 16B), and by annotated cell types including neuroblasts (hNbM), floor-plate progenitor (hProgFPL), and a very small portion of pericytes (hPeric) (FIG. 16C). FIG. 16D shows heatmap of top enriched gene-set in each cluster of TP53 WT versus TP53 KO cells at 1 day post transplantation, demonstrating highly increased cell death-related genes in clusters 3, 5, and 6 and survival related genes in clusters 2, 4. Red color indicates survival related genes. FIG. 16E volcano plots of differentially expressed genes, such as BAX, CDK1NA, CDKN2B, BBC3 (PUMA), and PHPT1 (in red) in TP53 WT versus TP53 KO grafted dopamine neurons from clusters 3, 5, and 6. FIG. 16F shows violin plots of BAX, TNFRSF12A, and JUN positive cells among the clusters. The cluster 7 is excluded due to a very small portion of cells. FIGS. 16G and 16H show HES5 positive cells specifically to clusters 3, 5, and 6 mark de-differentiated cells in UMAP from 1 day post graft (FIG. 16G) and is not expressed in the sorted cells prior to grafting (FIG. 16H).

FIGS. 17A-17E illustrate high-through flow-based cell surface marker screen identifies novel CD marker to purify NURR1 stage postmitotic dopamine neuron for translational use. FIG. 17A shows schematic illustration of the flow-based CD marker screen to enrich for postmitotic dopamine neurons matching genetic NURR1::GFP reporter expression. FIG. 17B shows FACS plot of % NURR1::GFP populations corresponding to each sorting strategy (Control, CD49e-low, CD49-low/CD171-high, CD49c-low/CD184-high), indicating CD49e-low/CD184-high double CD marker sorting leads to the most enriched dopamine neuron population expressing NURR1::GFP. FIG. 17C shows gene expression of NURR1 via qRT-PCR assay 2 days post sorting using each sorting strategy from FIG. 17B. FIG. 17D shows representative immunofluorescence image of CD49e-low/CD184-high double sorted dopamine neurons at day 40, giving rise to pure dopamine neuron culture co-expressing NURR1::GFP, FOXA2, and TH. Scale bar=100 μm. FIG. 17E shows short term in vivo histology analysis at 1-month post grafting of CD49e-low/CD184-high double sorted graft compared with unsorted cells. The double CD49e-low/CD184-high sorted neuron grafts are composed of densely packed dopamine neurons in contrast to unsorted grafts which yield a lower percentage of dopamine neurons as detected by human nuclear antigen (hNA) and TH immunofluorescence-staining. FIGS. 18A-18G illustrate clinically relevant TNFα neutralizing antibodies functionally improve the survival of postmitotic dopamine neuron during implantation. FIG. 18A shows representative immunofluorescence image of CD 49e-low/CD184-high double sorted dopamine neurons either co-injected with PBS or TNFα blocking antibody, adalimumab. Scale bar=100 μm. FIG. 18B shows stereological cell counts and volume quantification of the surviving dopamine neurons at 1 month post transplantation using NURR1::GFP, n=5. ** p<0.01, *p<0.05 (paired t-test). FIG. 18C shows D-amphetamine induced rotation assay in grafted PD mouse model carrying unilateral 6-OHDA lesion. The three treatment groups are: PBS injection (sham), CD sorted neurons, and CD sorted neurons but co-injected with adalimumab. FIG. 18D shows representative immunofluorescence images of human grafts that are highly enriched with floor-plated derived dopamine neurons marked by hNA, FOXA2, and TH for each group, scale bar=50 μm. FIG. 18E shows stereological analysis of the number (using optical fractionator) and volume (cavalier estimator) of the surviving dopamine neurons based on TH expression at 6 months post transplantation . . . * p<0.05 (paired t-test). FIGS. 18F and 18G show representative immunofluorescence image and quantification of portion of ALDH1A1 demarking A9 subtype (FIG. 18F) and CALB1 demarking A10 subtype (FIG. 18G) dopamine neurons population (TH+) in 6 months old graft.

FIGS. 19A-19H illustrates derivation and validation of NURR1:GFP sorted dopamine neurons for CRISPR/Cas9 screening in vitro and in vivo. FIG. 19A shows immunofluorescent staining of dopamine neuron markers, NURR1:GFP, FOXA2, and TH, in NURR1:GFP sorted cells two weeks post sorting (day 40). Scale bar=100 μm. FIG. 19B shows immunofluorescent staining of a dopamine neuron marker, TH, and NURR1::GFP in graft 1 month post transplantation of day 25 NURR1:GFP sorted DA neurons. Scale bar=100 μm. FIG. 19C shows transduction efficacy and isolate transduced hPSC containing pooled lentiviral sgRNAs expressing Tomato with MOI=0.35, which indicates single copy sgRNA integration per cell. FIG. 19D shows immunofluorescent staining of TH, NURR1::GFP and gRNA::tdTomato at day 40 dopamine neurons post sorting with GFP and Tomato at day 25 with/without dox treatment from day 16 to day 25. Scale bar=50 μm. FIG. 19E shows ablation of the tdTomato signal in hPSC-derived postmitotic dopamine neurons at day 25 after dox exposure from day 16 to day 25 (dox 1 μg/ml). Scale bar=200 μm. FIG. 19F shows graft fluorescence expression 1 month post transplantation (upper). PCR analysis for detecting a human PTGER2 gene from genomic DNA, isolated from dissected tissue around graft region (lower). Scale bar=500 μm. FIGS. 19G and 19H show overall representation of sgRNA across all condition by next-generation sequencing (NGS) from genomic DNA of in vitro cultured cells (day 16 and day 25) and an in vivo grafted cell from library (FIG. 19G) and more restricted library #2 (FIG. 19H).

FIGS. 20A-20F illustrate increased survival of TP53 KO dopamine neurons in graft exhibit dopamine neuron identity and needle injection of dopamine neuron does not induce TP53 and TP53 downstream genes. FIG. 20A shows immunofluorescent staining of NURR1:GFP, sgTP53RNA-Tomato, and FOXA2 and TH at day 40 dopamine neuron post sorting with GFP and Tomato at day 25 with/without dox exposure from day 16 to day 25. Scale bar=100 μm. FIGS. 20B-20E show representative immunofluorescence image for dopamine markers, such as FOXA2, TH, and NURR1-GFP (FIGS. 20B-20D) as well as a proliferation marker, hKi67 (FIG. 20E) expression in surviving p53 WT and KO dopamine neurons in the graft (WT; −DOX, KO; +DOX). FIG. 20F shows qRT-qPCR analysis of TP53 and TP53 downstream gene (p21 and PUMA) before and 1 hour after needle injection of NURR1:GFP sorted dopamine neuron.

FIGS. 21A-21D illustrate time-course analysis of host neuroimmune cells after transplantation near the graft site. FIG. 21A shows immunofluorescence analysis of IBA1 (upper) and GFAP (lower) following transplantation (4 hrs, 24 hrs, 72 hrs, 7 days). Scale bar=50 μm. FIG. 21B shows H&E staining of the graft. Scale bar=100 μm. FIG. 21C shows immunofluorescence and immunohistochemistry analysis for Ly6G to examine neutrophils at the graft site. Left panels are examined at 12 hpt and yellow arrows indicate Ly6G positive cells nearby the grafted cells positive for FOXA2. Right panels are examined at 3dpt. Ly6G positive cells marked by dark brown staining infiltrate within the graft. Scale bar=50 μm. FIG. 21D shows immunofluorescence analysis of fiber outgrowth pattern from the graft after implantation using STEM 121. Fiber extension begins at 24 hpt. Scale bar=50 μm.

FIGS. 22A-22E illustrate analysis of bulk RNA-seq from sorted cells vs. 1 day cultured cells post sorting vs. 1 day grafted cells post sorting, and characterization of 1 day cultured cells post sorting. FIG. 22A shows dendrogram of the cells among sorted (Day 0), in vitro cultured (Day 1 Culture), and in vivo grafted dopamine neurons (Day 1 Graft) from bulk RNA-seq demonstrating agreement among the replicate samples and distinct signature oof the day 1 grafted samples. FIG. 22B shows pathway enrichment analysis identified mTORC1 signaling as upregulated categories in day 1 cultured samples. FIG. 22C shows heatmap analysis from total RNA-seq for enriched TNFα-NFκB related genes in the grafted dopamine neurons versus the sorted and cultured dopamine neurons. FIG. 22D shows immunofluorescence staining of NURR1::GFP, TP53, and NFκB-p65 at day 26 dopamine neuron in culture 1 day post sorting with GFP shows no nuclear induction of NFκB. Scale bar=100 μm. FIG. 22E shows immunofluorescence staining of TNFα ligand at 1 day grafted neurons, confirming the protein expression of TNFα ligand.

FIG. 23A-23D illustrate scRNA-seq analysis from p53 WT and KO dopamine neuron grafts 1 day post implantation. FIG. 23A shows clustering distribution of p53 WT and p53 KO of grafted dopamine neurons 1 day post transplantation from scRNA-seq. FIG. 23B shows histograms of fraction of cells expressing MAP2, Ki67, and TH positive cells in p53 WT and KO. FIG. 23C shows UMAP plots of MAP2, PBX1, and MKI67 in p53 WT and p53 KO neurons from scRNA-seq. FIG. 23D shows volcano plot of differentially expressed genes in p53 WT versus p53 KO from scRNA-seq.

FIGS. 24A-24B illustrate characterization of cell surface (CD) marker sorted cells matching NURR1::GFP in vitro and in vivo. FIG. 24A shows FACS analysis of NURR1::GFP neuron population with indicated CD markers, demonstrating 3 CD markers (49c, 99, and 340) are negatively whereas 2 CD markers (171 and 184) are positively enriched to NURR1::GFP populations. FIG. 24B shows immunofluorescence analysis of grafted cells from CD49-low/CD184-high double sorted and unsorted cells with FOXA2 and a human proliferation marker (hKi67) at 1 month post transplantation (left) and quantification of Ki67 positive cells within the grafts (right).

FIGS. 25A-25C illustrate innervation of grafts from CD marker sorted dopamine neurons co-injected either PBS or TNFα inhibitor, adalimumab, at 6 months. FIGS. 25A-25B show representative image of extensive innervation of CD marker sorted dopamine neuron grafts co-injected with PBS or adalimumab towards the host striatum as indicated by TH (FIG. 25A) and hNCAM immunofluorescent staining (FIG. 25B). Scale bar=500 μm. FIG. 25C shows immunofluorescence assay to examine the spread of adalimumab within the brain. Anti-human IgG1 Alexa fluorophore 555 was probed to detect the presence of adalimumab comparing adalimumab co-injected neurons vs. PBS injected neurons at 24 hpt, and red signal indicates a specific detection of the human monoclonal antibodies only present in adalimumab.

### DETAILED DESCRIPTION

The present disclosure provides methods and compositions for improving in vivo survival of midbrain dopamine (mDA) neurons (e.g., in vitro differentiated mDA neurons) by suppressing p53-mediated apoptosis of mDA neurons. The present disclosure further provides methods and compositions for treating a subject (e.g., a subject suffering from a neurodegenerative disease and/or neurodegeneration of midbrain dopamine neurons), comprising administering to the subject one or more mDAs, wherein p53-mediated apoptosis of the one or more mDA neurons is suppressed. In certain embodiments, the suppression of p53-mediated apoptosis comprises inhibition of TNFα signaling, inhibition of NFκB signaling, inhibition of p53 signaling, or a combination of foregoing. In certain embodiments, the suppression of p53-mediated apoptosis comprises administering to the subject a TNFα inhibitor (e.g., an antagonistic anti-TNFα antibody). In certain embodiments, the suppression of p53-mediated apoptosis comprises contacting the one or more mDA neurons with a TNFα inhibitor (e.g., an antagonistic anti-TNFα antibody).

The present disclosure is at least based on the discovery that the expression of p53 restricted in vivo postmitotic dopamine neuron survival following transplantation. Moreover, transcriptomic analysis revealed that TNFα-mediated activation of NFκB is a main upstream regulator of p53-mediated dopamine neuron death. The inventors discovered that knocking out TP53 gene in midbrain dopamine neurons significantly improved in vivo survival of post-mitotic midbrain dopamine neurons. The inventors also discovered that in vivo survival of post-mitotic midbrain dopamine neurons after transplantation can be significantly improved by an TNFα antagonist, e.g., adalimumab. Non-limiting embodiments of the present disclosure are described by the present specification and Examples.

For purposes of clarity of disclosure and not by way of limitation, the detailed description is divided into the following subsections:
5.1. Definitions;
5.2. Methods of Improving In Vivo Survival of Dopamine Neurons;
5.3. Compositions; and
5.4. Methods of Treatment.

### 5.1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, tumor necrosis factor alpha (TNFα), nuclear factor kappa B (NFκB), p53. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors.

“Inhibitor” as used herein, refers to a compound or molecule (e.g., small molecules, antibodies, peptides, peptidomimetic, natural compounds, siRNA, anti-sense nucleic acids, or aptamers) that interferes with (e.g., antagonizes, reduces, decreases, suppresses, eliminates, or blocks) the function of the target molecule or pathway. An inhibitor can be any compound or molecule that changes the activity of a named protein (signaling molecule, any molecule involved with the named signaling molecule, a named associated molecule) (e.g., including, but not limited to, the signaling molecules described herein), for one example, via directly contacting TNFα, contacting TNFα mRNA, causing conformational changes of TNFα, decreasing TNFα protein levels, or interfering with TNFα interactions with signaling partners/receptors (e.g., TNFRSF11B, TNFRSF10B, and TNFRSF12A), and affecting the expression of TNFα target genes.

Inhibitors also include molecules that indirectly regulate biological activity, for example, SMAD biological activity, by intercepting upstream signaling molecules (e.g., within the extracellular domain, examples of a signaling molecule and an effect include: Noggin which sequesters bone morphogenic proteins, inhibiting activation of ALK receptors 1,2,3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules). Antibodies that block upstream or downstream proteins are contemplated for use to neutralize extracellular activators of protein signaling, and the like. Although the foregoing example relates to SMAD signaling inhibition, similar or analogous mechanisms can be used to inhibit other signaling molecules. Examples of inhibitors include, but are not limited to: LDN193189 (LDN) and SB431542 (SB) (LSB) for SMAD signaling inhibition, and IWP2 for Wnt inhibition. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators,” as used herein, refer to compounds that increase, induce, stimulate, activate, facilitate, or enhance activation the signaling function of the molecule or pathway, e.g., Wnt signaling, SHH signaling, FGF signaling, etc.

As used herein, the term “Wnt” or “wingless” in reference to a ligand refers to a group of secreted proteins (e.g., integration 1 in humans) that are capable of interacting with a Wnt receptor, such as a receptor in the Frizzled and LRPDerailed/RYK receptor family. As used herein, the term “a Wnt or wingless signaling pathway refers to a signaling pathway composed of Wnt family ligands and Wnt family receptors, such as Frizzled and LRPDerailed/RYK receptors, mediated with or without β-catenin. The Wnt signaling pathway include canonical Wnt signaling (e.g., mediation by β-catenin) and non-canonical Wnt signaling (mediation without β-catenin).

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of midbrain DA precursors, or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells that have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “neuron” refers to a nerve cell, the principal functional units of the nervous system. A neuron consists of a cell body and its processes—an axon and at least one dendrite. Neurons transmit information to other neurons or cells by releasing neurotransmitters at synapses.

As used herein, the term “differentiation” refers to a process whereby an unspecialized embryonic cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as midbrain dopamine neurons or precursors thereof. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein marker of mDA neurons, such as EN1, OTX2, TH, NURR1, FOXA2, LMXIA, PITX3, LMO3, SNCA, ADCAP1, CHRNA4, ALDH1A1, SOX6, WNT1, DAT, VMAT2, GIRK2, PBX1, SNCG, SATB1, CALB1, and CALB2.

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed cither for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “negative”, “weak”, or “−” when used in reference to any surface marker disclosed herein refer to that the surface marker (e.g., CD49e) is not expressed at a detectable level, or is expressed at a reduced level in a cell as compared to the mean expression of the surface marker in a population of cells of which the cell is selected or sorted from. As used herein, the term “high”, “strong”, “+”, or “positive” when used in reference to any surface marker disclosed herein refer to that the surface marker (e.g., CD184) is expressed at a detectable level or expressed at an increased level as compared to the mean expression of the surface marker in a population of cells.

In certain embodiments, the cells are distinguished according to their surface marker expression levels based on a readily discernible differences in staining intensity as is known to one or ordinary skill in the art. In certain embodiments, the cut off for designating a cell as a surface marker “weak”, “negative”, or “−” cell can be set in terms of the staining intensity distribution (e.g., fluorescence intensity distribution) observed for all the cells, with those cells falling below about 50%, about 40%, about 30%, about 20%, about 10%, or about 5% of staining intensity being designated as the surface marker “weak”, “negative”, or “−” cell. In certain embodiments, the cut off for designating a cell as a surface marker “strong”, “high”, “+”, or “positive” cell can be set in terms of the staining intensity distribution (e.g., fluorescence intensity distribution) observed for all the cells, with those cells falling above about 50%, about 60%, about 70%, about 80%, about 90%, or about 95% of staining intensity being designated as the surface marker “strong”, “high”, “+”, or “positive” cell. In certain embodiments, the frequency distribution of the surface marker staining is obtained for all the cells and the population curve fit to a higher staining and lower staining population, and cells assigned to the population to which they most statistically are likely to belong in view of a statistical analysis of the respective population distributions.

### 5.2. Methods of Improving In Vivo Survival of Midbrain Dopamine Neurons

The present disclosure provides methods of improving in vivo survival of one or more midbrain dopamine (mDA) neurons. In certain embodiments, the methods comprise suppressing p53-mediated apoptosis of the one or more mDA neurons. In certain embodiments, the suppression of p53-mediated apoptosis comprises inhibition of tumor necrosis factor alpha (TNFα) signaling, inhibition of nuclear factor kappa B (NFκB) signaling, inhibition of p53 signaling, or a combination of the foregoing. In certain embodiments, the suppression of p53-mediated apoptosis comprises contacting the one or more mDA neurons with at least one compound selected from the group consisting of TNFα inhibitors, NFκB inhibitors, p53 inhibitors, and combinations thereof. In certain embodiments, the suppression of p53-mediated apoptosis comprises contacting the one or more mDA neurons with a TNFα inhibitor.

In certain embodiments, the one or more mDA neurons express a marker selected from the group consisting of EN1, OTX2, TH, NURR1, FOXA2, LMXIA, PITX3, LMO3, SNCA, ADCAP1, CHRNA4, ALDH1A1, SOX6, WNT1, DAT, VMAT2, GIRK2, SATB1, CALB1, CALB2, SNCG, PBX1, and combinations thereof. In certain embodiments, the one or more mDA neurons are post-mitotic mDA neurons. In certain embodiments, the one or more mDA neurons are in vitro differentiated from one or more stem cells. In certain embodiments, the one or more stem cells are human stem cells. In certain embodiments, the one or more stem cells are one or more induced pluripotent stem cells.

### 5.2.1. Inhibition of TNFα Signaling

In certain embodiments, the suppression of p53-mediated apoptosis comprises inhibition of TNFα signaling. The TNFα signaling pathway plays an important role in various physiological and pathological processes, including cell proliferation, differentiation, apoptosis, and modulation of immune responses and induction of inflammation. TNFα is a multifunctional proinflammatory cytokines, with effects on lipid metabolism, coagulation, insulin resistance, and endothelial function. TNFα can be produced by many cell types (e.g., macrophages, lymphocytes, fibroblasts, and keratinocytes) in response to inflammation, infection, and other environmental stresses. TNFα acts by binding to its receptors (e.g., TNFR1, TNFR2, TNFRSF11B, TNFRSF10B, and TNFRSF12A) which in turn recruit and activate complex signaling cascades and networks.

In certain embodiments, inhibition of TNFα signaling is achieved by an inhibitor of TNFα signaling. In certain embodiments, the inhibitor of TNFα signaling can be a molecule (e.g., a chemical compound or an antibody) that interferes with (e.g., antagonizes, reduces, decreases, suppresses, eliminates, or blocks) the function of TNFα and/or its signaling. In certain embodiments, the inhibitor of TNFα signaling is a TNFα inhibitor.

In certain embodiments, the inhibitor of TNFα signaling and/or the TNFα inhibitor can include, without any limitation, interfering ribonucleic acids (e.g., siRNA, shRNA), aptamers, or peptidomimetics.

Non-limiting examples of inhibitors of TNFα signaling and/or the TNFα inhibitors include anti-TNFα antibodies, TNFα decoy receptors, chemical compounds, In certain embodiments, the TNFα inhibitor is selected from the group consisting of anti-TNFα antibodies, TNFα decoy receptors, chemical compounds, nucleic acid inhibitors, small molecule inhibitors, receptor biologic inhibitors, inactive TNF fragments, TNFα circulating receptor fusion protein (e.g. etanercept, etanercept-szzs), xanthine derivatives (e.g. pentoxifylline), and 5-HT_{2A} agonist (e.g., (R)-DOI, TCB-2, LSD, LA-SS-Ac).

In certain embodiments, the inhibitor of TNFα signaling and/or the TNFα inhibitor is an antibody. In certain embodiments, the antibody is an anti-TNFα antibody. In certain embodiments, the antibody is an antagonistic anti-TNFα antibody. In certain embodiments, the anti-TNFα antibody is selected from the group consisting of adalimumab (Humira®), adalimumab-adbm (Cyltezo®), adalimumab-adaz (Hyrimoz®), adalimumab-atto (Amgevita®), certolizumab pegol (Cimzia®), golimumab (Simponi®, Simponi Aria®), infliximab (Remicade®), infliximab-abda (Renflexis®), infliximab-dyyb (Inflectra®), remtolumab, afelimomab, nerelimomab, ozoralizumab, placulumab, and combinations thereof. In certain embodiments, the TNFα inhibitor is adalimumab.

In certain embodiments, the inhibitor of TNFα signaling and/or the TNFα inhibitor is a polypeptide. In certain embodiments, the polypeptide is a TNFα decoy receptor. In certain embodiments, the TNFα decoy receptor is selected from the group consisting of etanercept (Enbrel®), etanercept-szzs (Ereizi®), pegsunercept, onercept, and lenercept.

In certain embodiments, the inhibitor of TNFα signaling and/or the TNFα inhibitor is a chemical compound. Non-limiting examples of chemical compounds that can be used with the present disclosure include apremilast (Otezla®), bupropion (Zyban®), cathechin, cannabinoids, curcumin, lysergic acid 2,4-dimethylazetidide (LA-SS-Az, LSZ), apigenin-7-O-glucuronide, JTE-607 dihydrochloride, MD2-TLR4-IN-1, AUDA, isuzinaxib (APX-115 free base), IQ 3, 3-deazaadenosine hydrochloride, cucurbitacin IIb, lenalidomide (CC-5013), aprepitant (MK-0869), thalidomide (K17), amarogentin, pomalidomide (CC-4047), acetylcysteine (N-acetylcysteine), butoconazole nitrate, CPI-1189, methylthiouracil, UCB-9260, mesaconitine, myrislignan, falcarindiol, gardenoside, demethyleneberberine, stylopine, benpyrine racemate, muscone, ginsenoside Rb1, cepharanthine, QNZ (EVP4593), AX-024 HCl, NE 52-QQ57, resatorvid (TAK-242), apremilast (CC-10004), necrostatin-1, PF-3644022, GSK583, shikonin (C.I. 75535), GSK2982772, mulberroside A, corilagin, 20 (S)-ginsenoside Rh1, forsythoside B, 2′,5′-dihydroxyacetophenone, geraniin, homoplantaginin, SPD-304, UCB-6876, UCB-5307, UCB-9260, PF-3644022, R-7050, citronellol, and madecassic acid.

### 5.2.2. Inhibition of NFκB Signaling

In certain embodiments, the suppression of p53-mediated apoptosis comprises inhibition of NFκB signaling. NFκB represents a family of inducible transcription factors, which regulates a large array of genes involved in different processes of the immune and inflammatory responses. This family is composed of five structurally related members, including NFκB1, NFκB2, RelA, RelB and c-Rel, which mediates transcription of target genes by binding to a specific DNA element (e.g., KB enhancer). NFκB can regulate inflammatory responses by mediating induction of various proinflammatory genes in innate immune cells. NFκB can also regulate the activation, differentiation and effector function of inflammatory T cells.

In certain embodiments, inhibition of NFκB signaling is achieved by an inhibitor of NFκB signaling. In certain embodiments, the inhibitor of NFκB signaling can be a molecule (e.g., a chemical compound) that interferes with (e.g., antagonizes, reduces, decreases, suppresses, eliminates, or blocks) the transcription activity of NFκB and its signaling. In certain embodiments, the inhibitor of NFκB signaling is a NFκB inhibitor.

Non-limiting examples of inhibitors of NFκB signaling and NFκB inhibitors that can be used with the present disclosure include upstream inhibitors of NFκB, inhibitors of IKK activity, inhibitors of IκB phosphorylation, inhibitors of IκB degradation, proteasome inhibitors, protease inhibitors, IκB upregulators, inhibitors of NFκB nuclear translocation and expression, NFκB DNA-binding inhibitors, and NFκB transactivation inhibitors, inhibitors of NFκB directed gene transactivation, and antioxidants. Additional examples of inhibitors of NFκB signaling and NFκB inhibitors that can be used with the present disclosure include, without any limitation, antioxidants, interfering ribonucleic acids (e.g., siRNA, shRNA), antibodies, aptamers, or peptidomimetics.

In certain embodiments, inhibition of NFκB signaling is achieved by an upstream inhibitor of NFκB. Non-limiting examples of upstream inhibitors of NFκB that can be used with the present disclosure include rituximab, pigment epithelium derived factor, betaine, desloratadine, LY29, LY30, MOL 294, pefabloc, rhein, salmeterol, and fluticasone propionate.

In certain embodiments, inhibition of NFκB signaling is achieved by an inhibitor of IKK activity of IκB phosphorylation. Non-limiting examples of inhibitors of IKK activity of IκB phosphorylation that can be used with the present disclosure include heparin-binding epidermal growth factor-like growth factor, hepatocyte growth factor, interleukin-10, anti-thrombin III, chorionic gonadotropin, interferon-α, 2-amino-3-cyano-4-aryl-6-(2-hydroxy-phenyl) pyridine derivatives, acrolein, AS602868, aspirin, dihydroxyphenylethanol, epoxyquinone A monomer, MLB120, BMS-345541, CYL-19s, CYL-26z, 2-amino-6-[2-(cyclopropylmethoxy)-6-hydroxyphenyl]-4-piperidin-4-yl nicotinonitrile, compound A, compound 5, cyclopentenones, jesterone dimer, PS-1145 (MLN1145), 2-[(aminocarbonyl)amino]-5-acetylenyl-3-thionphenecarboxamides, aminopyrimidine, benzoimidazole derivative, CDDO-Me (synthetic triterpenoid), CHS 828 (anticancer drug), diaylpyridine derivative, imidazolylquinoline-carboxaldehyde derivatives, indolecarboxamide, LF15-0195 (analog of 15-deoxyspergualine), ML120B, MX781 (retinoid antagonist), N-(4-hydroxyphenyl) retinamide, pyrazolo[4,3-c]quinoline derivatives, pyridooxazinone derivative, scytonemin, sulfasalazine, thalidomide, azidothymidine (AZT), BAY-11-7082 (E3 ((4-methylphenyl)-sulfonyl)-2-propenenitrile), BAY-11-7083 (E3 ((4-t-butylphenyl)-sulfonyl)-2-propenenitrile), benzyl isothiocyanate, carboplatin, gabexate mesylate, Gleevec (Imatanib), hydroquinone, ibuprofen, methotrexate, monochloramine, nafamostat mesylate, statins, and THI 52 (1-naphthylethyl-6,7-dihydroxy-1,2,3,4-tetrahydroisoquinoline).

In certain embodiments, inhibition of NFκB signaling is achieved by an inhibitor of IκB degradation. Non-limiting examples of inhibitors of IκB degradation that can be used with the present disclosure include penetratin, vasoactive intestinal peptide, α-melanocyte-stimulating hormone (a-MSH), IL-13, intravenous immunoglobulin, pituitary adenylate cyclase-activating polypeptide (PACAP), SAIF (Saccharomyces boulardii anti-inflammatory factor), acetaminophen, 1-Bromopropane, diamide (tyrosine phosphatase inhibitor), dobutamine, E-73 (cycloheximide analog), ccabet sodium, gabexate mesylate, glimepiride, losartan, pervanadate, phenylarsine oxide, phenytoin, sabaeksan, U0126 (MEK inhibitor), and Ro106-9920 (small molecule).

In certain embodiments, inhibition of NFκB signaling is achieved by a proteasome or protease inhibitor. Non-limiting examples of proteasomes or protease inhibitors that can be used with the present disclosure include N-acetyl-leucinyl-leucynil-norleucynal, MG101, N-acetyl-leucinyl-leucynil-methional, carbobenzoxyl-leucinyl-leucynil-norvalinal, MG115, N-carbobenzoxyl-L-leucinyl-L-leucinyl-L-norleucinal, MG132, ubiquitin ligase inhibitors, boronic acid peptide, bortezomib, salinosporamide A, tacrolimus, deoxyspergualin, disulfiram, N-acetyl-DL-phenylalanine-b-naphthylester, N-benzoyl L-tyrosine-ethylester, 3,4-dichloroisocoumarin, diisopropyl fluorophosphate, N-α-tosyl-L-phenylalanine chloromethyl ketone, and N-α-tosyl-L-lysine chloromethyl ketonc.

In certain embodiments, inhibition of NFκB signaling is achieved by an inhibitor of NFκB nuclear translocation and expression. Non-limiting examples of inhibitors of NFκB nuclear translocation and expression for use with the present disclosure include atorvastatin, phallacidin, piperine, pitavastatin, selenomethionine, clarithromycin, cantharidin, neomycin, paconiflorin, rapamycin, ranpirnase, BMD (N (1)-Benzyl-4-methylbenzene-1,2-diamine), carbaryl, indole-3-carbinol, dehydroxymethylepoxyquinomicin, dipyridamole, disulfiram, diltiazem, fluvastatin, levamisole, rolipram, SC236, omapatrilat, enalapril, and CGS 25462.

In certain embodiments, inhibition of NFκB signaling is achieved by an NFκB DNA-binding or transactivation inhibitor. Non-limiting examples of inhibitors of NFκB DNA-binding or transactivation inhibitors that can be used with the present disclosure include 7-amino-4-methylcoumarin, amrinone, atrovastat (HMG-COA reductase inhibitor), benfotiamine (thiamine derivative), bisphenol A, caprofen, carbocisteine, celecoxib, germcitabine, flurbiprofen, lovastatinm mercaptopyrazine, monomethylfumarate, moxifloxacin, nicorandil, nilvadipine, pioglitazone, pirfenidone, pyridine N-oxide derivatives, quinadril, raloxifene, raxofelast, ribavirin, rifamides, ritonavir, rosiglitazone, roxithromycin, simvastatin, SM-7368, sulfasalazine, verapamil, dimethylfumarate (DMF), ethyl 2-[(3-methyl-2,5-dioxo (3-pyrrolinyl)) pyrimidine-5-carboxylate, nelfinavir, ritonavir, saquinavir, RO31-8220, SB203580, and troglitazone.

### 5.2.3. Inhibition of p53 Signaling

In certain embodiments, the suppression of p53-mediated apoptosis comprises inhibition of p53 signaling. p53 is a transcriptional factor often associated with cancers. Physiologically, p53 can be disabled either by mutations or by upstream negative regulators, including, but not limited to, MDM2 and MDMX.

In certain embodiments, inhibition of p53 signaling is achieved by reducing the expression of p53. In certain embodiments, reducing the expression of p53 comprises knocking out or knocking down TP53 gene in the one or more mDAs. In certain embodiments, the TP53 gene is knocked out or knocked down in the one or more mDA neurons by a gene-editing system.

Non-limiting examples of gene-editing systems for use with the present disclosure include systems utilizing a non-naturally occurring or engineered nuclease (including, but not limited to, Zinc-finger nuclease (ZNFs), meganuclease, transcription activator-like effector nuclease (TALEN)), or a CRISPR-Cas system. Details on the gene-editing systems for use with the present disclosure can be found in Adli et al., Nat Commun. 2018 May 15; 9 (1): 1911 and Maeder & Gersbach, Mol Ther. 2016 March; 24 (3): 430-46, the content of each of which is incorporated by reference in its entirety.

In certain embodiments, a CRISPR-Cas system is used for knocking out or knocking down the TP53 gene. The CRISPR (Clustered Regularly Interspaced Short Palindromic Repeats)-Cas (CRISPR Associated) system is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide a CRISPR-Cas nuclease to a region homologous to the crRNA in the target DNA called a “proto spacer”. The CRISPR-Cas nuclease cleaves the DNA to generate blunt ends at the DSB at sites specified by a 20-nucleotide guide sequence contained within the crRNA transcript. The CRISPR-Cas nuclease requires both the crRNA and the tracrRNA for site specific DNA recognition and cleavage. This system has been engineered such that the crRNA and tracrRNA can be combined into one molecule (the “single guide RNA”); and the crRNA equivalent portion of the single guide RNA can be engineered to guide the CRISPR-Cas nuclease to target any desired sequence (see Jinek et al., Science (2012); 337:816-821). Thus, the CRISPR-Cas system can be engineered to create a DSB at a desired target in a genome. In certain embodiments, the CRISPR-Cas system comprises a CRISPR-Cas nuclease and a single-guide RNA. Suitable examples of CRISPR-Cas nucleases include, but are not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx 14, Csx 10, Csx16, CsaX, Csx3, Csx1, Csx15, Csf1, Csf2, Csf3, Csf4, homologs thereof, or modified versions thereof. These CRISPR-Cas nucleases are known; for example, the amino acid sequence of S. pyogenes Cas9 protein may be found in the SwissProt database under accession number Q99ZW2. In some embodiments, the CRISPR-Cas nuclease has DNA cleavage activity, e.g., Cas9. In certain embodiments, the CRISPR-Cas nuclease is Cas9. The CRISPR-Cas nuclease can direct cleavage of one or both strands at the location of a target sequence (e.g., a genomic safe harbor site). Additionally, the CRISPR-Cas nuclease can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

In certain embodiments, inhibition of p53 signaling is achieved by an inhibitor of p53 signaling. In certain embodiments, the inhibitor of p53 signaling can be a molecule (e.g., a chemical compound) that interferes with (e.g., antagonizes, reduces, decreases, suppresses, eliminates, or blocks) the transcription activity of p53 and its signaling. In certain embodiments, the inhibitor of p53 signaling is a p53 inhibitor.

Non-limiting examples of inhibitors of p53 signaling and p53 inhibitors that can be used with the present disclosure include JNK inhibitors, p38 MAPK inhibitors, caspase inhibitors, puma/BBC3 inhibitors, BAX inhibitors, CDK inhibitors, MDM2 and MDMX activators, and combinations thereof. Additional examples of p53 signaling and p53 inhibitors for use with the present disclosure include, without any limitation, interfering ribonucleic acids (e.g., siRNA, shRNA), antibodies, aptamers, or peptidomimetics.

In certain embodiments, inhibition of p53 signaling is achieved by a JNK inhibitor. c-Jun N-terminal protein kinase (JNK) is a subfamily of the mitogen activated protein kinase (MAPK) superfamily. JNK is a key regulator of many cellular events, including programmed cell death (apoptosis). In addition, JNK activates p53, which regulates apoptosis processes. Non-limiting examples of JNK inhibitors that can be used with the present disclosure include SP600125, AS601245, AS602801, JNK-IN-1, JNK-IN-8, ginsenoside Rg1, AV7, BI-78D3, pyridopyrimidione derivates, CC-930, quinazoline, triazolothione 1, XG-102 (D-JNKI-1), 4-fluorophenyl isoxazoles, 4-quinolone analogs, and 4-phenylisoquinolone.

In certain embodiments, inhibition of p53 signaling is achieved by a p38 MAPK inhibitor. p38 mitogen-activated protein kinases are a class of mitogen-activated protein kinases (MAPKs) that are responsive to stress stimuli, such as cytokines, ultraviolet irradiation, heat shock, and osmotic shock, and are involved in cell differentiation, apoptosis and autophagy. p38 MAPK can activate p53. Non-limiting examples of p38 MAPK inhibitors that can be used with the present disclosure include doramapimod, skepinone, ralimetinib, TAK-715, losmapimod, neflamapimod, R1487, VX-702, pamapimod, and adezmapimod.

In certain embodiments, inhibition of p53 signaling is achieved by a caspase inhibitor. Non-limiting examples of caspase inhibitors that can be used with the present disclosure include Ac-IETD-CHO, Ac-YVAD-CHO, Ac-DEVD-CMK, Z-VAD-FMK, Z-YVAD-FMK, Boc-D-FMK, TRP-601, Q-VD-OPh, VX-765 (belnacasan), VRT-043198, VX-740 (pralnacasan), IDN-6556 (emricasan, PF-034911390), VX-166, M826, M867, QPI-1007 (cosdosiran), NCX-1000, and Isatin sulfonamides.

In certain embodiments, inhibition of p53 signaling is achieved by a nucleic acid targeting a protein regulating the p53 pathway. In certain embodiments, the nucleic acid targets p53. Non-limiting examples of nucleic acids that can be used with the present disclosure include siRNAs and shRNAs. siRNA molecules are polynucleotides that are generally about 20 to about 25 nucleotides long and are designed to bind specific RNA sequence (e.g., p53 mRNA). siRNAs silence gene expression in a sequence-specific manner, binding to a target RNA (e.g., an RNA having the complementary sequence) and causing the RNA to be degraded by endoribonucleases. siRNA molecules able to inhibit the expression of p53 can be produced by suitable methods. There are several algorithms that can be used to design siRNA molecules that bind the sequence of a gene of interest (see e.g., Huesken et al., Nat. Biotechnol. 23:995-1001; Jagla et al., RNA 11:864-872, 2005; Shabalinea, BMC Bioinformatics 7:65, 2005). Additionally or alternatively, expression vectors expressing siRNA or shRNA can be used (see e.g., Brummelkamp, Science 296:550-553, 2002; Lee et al., Nature Biotechnol. 20:500-505, 2002; Elbashir et al., Nature 411:494-498, 2001).

In certain embodiments, inhibition of p53 signaling is achieved by a ribozyme that inhibits the expression of p53. Ribozymes are RNA molecules possessing enzymatic activity. One class of ribozymes is capable of repeatedly cleaving other separate RNA molecules into two or more pieces in a nucleotide base sequence specific manner (see Kim et al., Proc Natl Acad Sci USA, 84:8788 (1987); Haseloff & Gerlach, Nature, 334:585 (1988); and Jefferies et al., Nucleic Acid Res, 17:1371 (1989). Such ribozymes typically have two functional domains: a catalytic domain and a binding sequence that guides the binding of ribozymes to a target RNA through complementary base-pairing. Once a specifically-designed ribozyme is bound to a target mRNA, it enzymatically cleaves the target mRNA, reducing its stability and destroying its ability to directly translate an encoded protein. Methods for selecting a ribozyme target sequence and designing and making ribozymes are generally known in the art.

### 5.2.4. Midbrain Dopamine (mDA) Neurons

In certain embodiments, the one or more mDA neurons used with the presently disclosed methods express a marker indicating a mDA neuron. Non-limiting examples of markers indicating a mDA neuron include engrailed-1 (EN1), orthodenticle homeobox 2 (OTX2), tyrosine hydroxylase (TH), nuclear receptor related-1 protein (NURR1), forkhead box protein A2 (FOXA2), and LIM homeobox transcription factor 1 alpha (LMX1A), PITX3, LMO3, SNCA, ADCAP1, CHRNA4, ALDH1A1, DAT, VMAT1, SOX6, WNT1, GIRK2, SATB1, CALB1, CALB2, and PBX1. In certain embodiments, the mDA neurons do not express a detectable level of at least one marker selected from the group consisting of PAX6, EMX2, LHX2, SMA, SIX1, PITX2, SIMI, POU4F1, PHOX2A, BARHL1, BARHL2, GBX2, HOXA1, HOXA2, HOXB1, HOXB2, POU5F1, NANOG, and combinations thereof. In certain embodiments, the one or more mDA neurons express at least one of A9 subtype mDA neuron markers, A10 subtype mDA neuron markers, and mDA neuron maturity markers. In certain embodiments, the one or more mDA neurons express at least one marker selected from the group consisting of TH, EN1, NURR1, and ALDH1A1. In certain embodiments, the one or more mDA neurons express ALDH1A1.

In certain embodiments, the one or more mDA neurons are one or more post-mitotic mDA neurons. A “post-mitotic” cell is a terminally differentiated cell that is no longer able to undergo mitosis and proliferation. In certain embodiments, the one or more post-mitotic mDA neurons do not express a detectable level of CD49e and express a detectable level of CD184. In certain embodiments, the one or more mDA neurons are sorted by not expressing a detectable level of CD49e and expressing a detectable level of CD184.

In certain embodiments, the one or more mDA neurons are in vitro differentiated from stem cells. In certain embodiments, the one or more mDA neurons are in vitro differentiated from one or more stem cells in accordance to the methods disclosed in International Patent Publication Nos. WO2013067362, WO2016196661, WO2021042027, and WO2021203009, the contents of each of which are incorporated by reference in their entireties.

In certain embodiments, the in vitro differentiation comprises contacting stem cells with at least one inhibitor of Small Mothers Against Decapentaplegic (SMAD) signaling (referred to as “SMAD inhibitor”), at least one activator of Sonic hedgehog (SHH) signaling (referred to as “SHH activator”), and at least one activator of wingless (Wnt) signaling (referred to as “Wnt activator”). In certain embodiments, the in vitro differentiation further comprises contacting the cells with at least one activator of fibroblast growth factor (FGF) signaling (referred to as “FGF activator”). In certain embodiments, the in vitro differentiation further comprises contacting the cells with at least one inhibitor of Wnt signaling. In certain embodiments, the cells are further contacted with DA neuron lineage specific activators and inhibitors.

### 5.2.4.1. Stem Cells

In certain embodiments, the stem cells are pluripotent stem cells. In certain embodiments, the pluripotent stem cells are selected from embryonic stem cells (ESCs), induced pluripotent stem cells (iPSCs), and combinations thereof. In certain embodiments, the stem cells are multipotent stem cells. Non-limiting examples of stem cells that can be used with the presently disclosed methods include nonembryonic stem cells, embryonic stem cells, induced nonembryonic pluripotent cells, and engineered pluripotent cells. In certain embodiments, the stem cells are human stem cells. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell is a human embryonic stem cell (hESC). In certain embodiments, the stem cell is a human induced pluripotent stem cell (hiPSC). In certain embodiments, the stem cells are non-human stem cells. In certain embodiments, the stem cell is a nonhuman primate stem cell. In certain embodiments, the stem cell is a rodent stem cell.

### 5.2.4.2. SMAD Inhibitors

Non-limiting examples of SMAD inhibitors include inhibitors of transforming growth factor beta (TGFβ)/Activin-Nodal signaling (referred to as “TGFβ/Activin-Nodal inhibitor”), and inhibitors of bone morphogenetic proteins (BMP) signaling. In certain embodiments, the TGFβ/Activin-Nodal inhibitor can neutralize the ligands including TGFβs, BMPs, Nodal, and activins, and/or block their signal pathways through blocking the receptors and downstream effectors. Non-limiting examples of TGFβ/Activin-Nodal inhibitors include those disclosed in WO/2010/096496, WO/2011/149762, WO/2013/067362, WO/2014/176606, WO/2015/077648, Chambers et al., Nat Biotechnol. 2009 March; 27 (3): 275-80, Kriks et al., Nature. 2011 Nov. 6; 480 (7378): 547-51, and Chambers et al., Nat Biotechnol. 2012 Jul. 1; 30 (7): 715-20 (2012), all of which are incorporated by reference in their entireties herein for all purposes. In certain embodiments, the at least one TGFβ/Activin-Nodal inhibitor is selected from inhibitors of ALK5, inhibitors of ALK4, inhibitors of ALK7, and combinations thereof). In certain embodiments, the TGFβ/Activin-Nodal inhibitor comprises an inhibitor of ALK5. In certain embodiments, the TGFβ/Activin-Nodal inhibitor is a small molecule selected from SB431542, derivatives thereof, and mixtures thereof. “SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

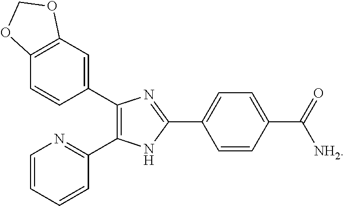 [SMILES: NC(=O)c1ccc(-c2nc(-c3ccc4c(c3)OCO4)c(-c3ccccn3)[nH]2)cc1]

In certain embodiments, the TGFβ/Activin-Nodal inhibitor comprises SB431542. In certain embodiments, the TGFβ/Activin-Nodal inhibitor comprises a derivative of SB431542. In certain embodiments, the derivative of SB431542 is A83-01.

In certain embodiments, the at least one SMAD inhibitor comprises an inhibitor of BMP signaling (referred to as “BMP inhibitor”). Non-limiting examples of BMP inhibitors include those disclosed in WO2011/149762, Chambers et al., Nat Biotechnol. 2009 March; 27 (3): 275-80, Kriks et al., Nature. 2011 Nov. 6; 480 (7378): 547-51, and Chambers et al., Nat Biotechnol. 2012 Jul. 1; 30 (7): 715-20, all of which are incorporated by reference in their entireties. In certain embodiments, the BMP inhibitor is a small molecule selected from LDN193189, Noggin, dorsomorphin, derivatives thereof, and mixtures thereof. “LDN193189” refers to a small molecule DM-3189, IUPAC name 4-(6-(4-(piperazin-1-yl)phenyl) pyrazolo[1,5-a]pyrimidin-3-yl) quinoline, with a chemical formula of C₂₅H₂₂N₆ with the following formula.

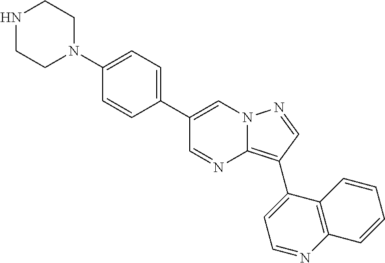 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18:4388-4392, herein incorporated by reference).

In certain embodiments, the BMP inhibitor comprises LDN193189. In certain embodiments, the BMP inhibitor comprises Noggin.

In certain embodiments, the stem cells are exposed to one SMAD inhibitor, e.g., one TGFβ/Activin-Nodal inhibitor. In certain embodiments, the TGFβ/Activin-Nodal inhibitor is SB431542. In certain embodiments, the TGFβ/Activin-Nodal inhibitor is a derivative of SB431542. In certain embodiments, the TGFβ/Activin-Nodal inhibitor is A83-01.

In certain embodiments, the stem cells are exposed to two SMAD inhibitors. In certain embodiments, the two SMAD inhibitors are a TGFβ/Activin-Nodal inhibitor and a BMP inhibitor. In certain embodiments, the stem cells are exposed to SB431542 or A83-01, and LDN193189 or Noggin. In certain embodiments, the stem cells are exposed to SB431542 and LDN193189. In certain embodiments, the stem cells are exposed to A83-01 and LDN193189. In certain embodiments, the stem cells are exposed to SB431542 and Noggin. In certain embodiments, the stem cells are exposed to A83-01 and Noggin.

In certain embodiments, the stem cells are contacted with or exposed to the at least one SMAD inhibitor for about 5 days, for 6 days or for 7 days. In certain embodiments, the cells are contacted with or exposed to the at least one SMAD inhibitor from day 0 through day 6.

In certain embodiments, the cells are contacted with or exposed to a TGFβ/Activin-Nodal inhibitor. In certain embodiments, the concentration of the TGFβ/Activin-Nodal inhibitor contacted with or exposed to the cells is about 5 μM, or about 10 μM. In certain embodiments, the TGFβ/Activin-Nodal inhibitor comprises SB431542 or a derivative thereof (e.g., A83-01). In certain embodiments, the TGFβ/Activin-Nodal inhibitor comprises SB431542.

In certain embodiments, the cells are contacted with or exposed to a BMP inhibitor. In certain embodiments, the concentration of the BMP inhibitor contacted with or exposed to the cells is about 250 nM. In certain embodiments, the BMP inhibitor comprises LDN193189 or a derivative thereof. In certain embodiments, the BMP inhibitor comprises LDN193189.

In certain embodiments, the cells are contacted with or exposed to the TGFβ/Activin-Nodal inhibitor and the BMP inhibitor simultaneously. In certain embodiments, the stem cells are contacted with or exposed to the TGFβ/Activin-Nodal inhibitor and the BMP inhibitor for about 5 days, for 6 days, or for 7 days. In certain embodiments, the cells are contacted with or exposed to the TGFβ/Activin-Nodal inhibitor and the BMP inhibitor from day 0 through day 6.

### 5.2.4.3. Wnt Activators

In certain embodiments, the at least one Wnt activator lowers GSK3β for activation of Wnt signaling. Thus, in certain embodiments, the Wnt activator is a GSK3B inhibitor. A GSK3B inhibitor is capable of activating a WNT signaling pathway, see e.g., Cadigan, et al., J Cell Sci. 2006; 119:395-402; Kikuchi, et al., Cell Signaling. 2007; 19:659-671, which are incorporated by reference herein in their entireties. As used herein, the term “glycogen synthase kinase 3β inhibitor” or “GSK3β inhibitor” refers to a compound that inhibits a glycogen synthase kinase 3β enzyme, for example, see Doble, et al., J Cell Sci. 2003; 116:1175-1186, which is incorporated by reference herein in its entirety. Non-limiting examples of GSK3B inhibitors include CHIR99021, BIO ((3E)-6-bromo-3-[3-(hydroxyamino) indol-2-ylidene]-1H-indol-2-one), AMBMP hydrochloride, LP 922056, SB-216763, CHIR98014, Lithium, 3F8, deoxycholic acid, and those disclosed in WO2011/149762, WO13/067362, Chambers et al., Nat Biotechnol. 2012 Jul. 1; 30 (7): 715-20, Kriks et al., Nature. 2011 Nov. 6; 480 (7378): 547-51, and Calder et al., J Neurosci. 2015 Aug. 19; 35 (33): 11462-81, all of which are incorporated by reference in their entireties.

Non-limiting examples of Wnt activators include CHIR99021, Wnt3A, Wnt1, Wnt5a, BIO ((3E)-6-bromo-3-[3-(hydroxyamino) indol-2-ylidene]-1H-indol-2-one), AMBMP hydrochloride, LP 922056, SB-216763, CHIR98014, Lithium, 3F8, deoxycholic acid, and those disclosed in WO2011/149762, WO13/067362, Chambers et al., Nat Biotechnol. 2012 Jul. 1; 30 (7): 715-20, Kriks et al., Nature. 2011 Nov. 6; 480 (7378): 547-51, and Calder et al., J Neurosci. 2015 Aug. 19; 35 (33): 11462-81, all of which are incorporated by reference in their entireties. In certain embodiments, the at least one Wnt activator is a small molecule selected from CHIR99021, Wnt3A, Wnt1, Wnt5a, BIO, CHIR98014, Lithium, 3F8, deoxycholic acid, derivatives thereof, and mixtures thereof. In certain embodiments, the at least one Wnt activator comprises CHIR99021 or a derivative thereof. In certain embodiments, the at least one Wnt activator comprises CHIR99021. “CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl)pyrimidin-2-ylamino) ethylamino) nicotinonitrile with the following formula.

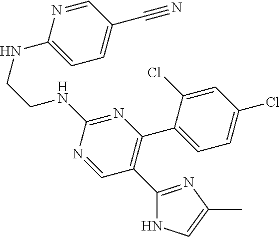 [SMILES: Cc1c[nH]c(-c2cnc(NCCNc3ccc(C#N)cn3)nc2-c2ccc(Cl)cc2Cl)n1]

CHIR99021 is highly selective, showing nearly thousand-fold selectivity against a panel of related and unrelated kinases, with an IC50=6.7 nM against human GSK3B and nanomolar IC50 values against rodent GSK3β homologs.

In certain embodiments, the cells are contacted with the at least one Wnt activator for about 15 days, for 16 days, or for 17 days. In certain embodiments, the cells are contacted with the at least one Wnt activator from day 0 through day 16.

In certain embodiments, the concentration of the at least Wnt activator is increased during its exposure to the cells (also referred to as “Wnt Boost”). In certain embodiments, the increase or Wnt Boost is initiated at least about 2 days, at least about 4 days, or at least about 5 days from the initial exposure of the cells to the at least one Wnt activator. In certain embodiments, the increase or Wnt Boost is initiated about 4 days from the initial exposure of the cells to the at least one Wnt activator. In certain embodiments, the concentration of the at least one activator of Wnt signaling that is contacted with the cells is increased between about 2 days and about 6 days from the initial contact of the cells with the at least one activator of Wnt signaling.

In certain embodiments, the cells are contacted with or exposed to the increased concentration of the at least one Wnt activator for at least about 5 days, or at least about 10 days. In certain embodiments, the cells are contacted with the increased concentration of the at least one Wnt activator for up to about 10 days.

In certain embodiments, the cells are contacted with or exposed to the increased concentration of the at least one Wnt activator for about 5 days, for 5 days or for 6 days. In certain embodiments, the cells are contacted with or exposed to the increased concentration of the at least one Wnt activator from day 4 through day 9. In certain embodiments, the cells are contacted with or exposed to the increased concentration of the at least one Wnt activator for about 10 days, for 12 days, or for 13 days. In certain embodiments, the cells are contacted with or exposed to the increased concentration of the at least one Wnt activator from day 4 through day 16.

In certain embodiments, the initial concentration of the at least one Wnt activator contacted with or exposed to the cells prior to the Wnt boost is about 1 μM or about 0.5 μM. In certain embodiments, the initial concentration of the at least one Wnt activator contacted with or exposed to the cells prior to the Wnt boost is about 0.7 μM.

In certain embodiments, the increased concentration of the at least one Wnt activator post the Wnt Boost is about 3 μM or about 6 μM. In certain embodiments, the increased concentration of the at least one Wnt activator post the Wnt boost is about 7 μM or about 7.5 μM.

In certain embodiments, the concentration of the at least one Wnt activator is increased from the initial concentration contacted with or exposed to the cells by about 200%, about 250%, about 300%, about 350%, about 400%, about 450%, about 500%, about 550%, about 600%, about 650%, about 700%, about 750%, about 800%, about 850%, about 900%, about 950%, about 1000%, about 1050%, or about 1100%. In certain embodiments, the concentration of the at least one activator of Wnt signaling that is contacted with the cells is increased by between about 250% and about 1800% of the initial concentration of the at least one activator of Wnt signaling contacted with the cells.

In certain embodiments, the concentration of the at least one Wnt activator is increased from about 1 μM to about 6 μM. In certain embodiments, the concentration of the at least one Wnt activator is increased from about 1 μM to between about 3 μM and about 5 μM. In certain embodiments, the concentration of the at least one Wnt activator is increased from about 1 μM to about 3 μM.

In certain embodiments, the at least one Wnt activator comprises a GSK3B inhibitor. In certain embodiments, the at least one Wnt activator comprises CHIR99021 or a derivative thereof. In certain embodiments, the at least one Wnt activator comprises CHIR99021.

### 5.2.4.4. SHH Activators

As used herein, the term “Sonic hedgehog,” “SHH,” or “Shh” refers to a protein that is one of at least three proteins in the mammalian signaling pathway family called hedgehog, another is desert hedgehog (DHH) wile a third is Indian hedgehog (IHH). SHH interacts with at least two transmembrane proteins by interacting with transmembrane molecules Patched (PTC) and Smoothened (SMO). SHH typically binds to PTC, which then allows the activation of SMO as a signal transducer. In the absence of SHH, PTC typically inhibits SMO, which in turn activates a transcriptional repressor so transcription of certain genes does not occur. When SHH is present and binds to PTC, PTC cannot interfere with the functioning of SMO. With SMO uninhibited, certain proteins are able to enter the nucleus and act as transcription factors allowing certain genes to be activated (see Gilbert, 2000 Developmental Biology (Sunderland, Mass., Sinauer Associates, Inc., Publishers). In certain embodiments, an SHH activator refers to any molecule or compound that is capable of activating a SHH signaling pathway, including a molecule or compound that is capable of binding to PTC or a SMO. In certain embodiments, the at least one SHH activator is selected from the group consisting of molecules that bind to PCT, molecules that bind to SMO, and combinations thereof. Non-limiting examples of SHH activators include those disclosed in WO10/096496, WO13/067362, Chambers et al., Nat Biotechnol. 2009 March; 27 (3): 275-80, and Kriks et al., Nature. 2011 Nov. 6; 480 (7378): 547-51. In certain embodiments, the at least one SHH activator is selected from the group consisting of a SHH protein, a SMO agonist, or a combination thereof. In certain embodiments, the SHH protein is selected from the group consisting of a recombinant SHH, a modified N-terminal SHH, or a combination thereof. In certain embodiments, the recombinant SHH comprises a N-terminal fragment and a C-terminal fragment. In certain embodiments, the modified N-terminal SHH comprises two Isoleucines at the N-terminus. In certain embodiments, the modified N-terminal SHH has at least about 80%, about 85%, about 90%, about 95%, or about 99% sequence identity to an un-modified N-terminal SHH. In certain embodiments, the modified N-terminal SHH has at least about 80%, about 85%, about 90%, about 95%, or about 99% sequence identity to an un-modified human N-terminal SHH. In certain embodiments, the modified N-terminal SHH has at least about 80%, about 85%, about 90%, about 95%, or about 99% sequence identity to an un-modified mouse N-terminal SHH. In certain embodiments, the modified N-terminal SHH comprises SHH C25II. In certain embodiments, the modified N-terminal SHH comprises SHH C24II.

Non-limiting examples of SMO agonists (SAGs) include purmorphamine, GSA10, and 20 (S)-hydroxy Cholesterol. In certain embodiments, the SAG comprises purmorphamine.

In certain embodiments, the cells are contacted with or exposed to the at least one SHH activator for about 5 days, for 6 days, or for 7 days. In certain embodiments, the cells are contacted with or exposed to the at least one SHH activator from day 0 through day 6.

In certain embodiments, the concentration of the at least one SHH activator contacted with or exposed to the cells is about 400 ng/ml, about 450 ng/ml, about 500 ng/ml, about 550 ng/mL, or about 600 ng/mL.

In certain embodiments, the at least one activator of SHH signaling comprises SHH C25II.

### 5.2.4.5. FGF Activators

FGF family includes secreted signaling proteins (secreted FGFs) that signal to receptor tyrosine kinases. Phylogenetic analysis suggests that 22 Fgf genes can be arranged into seven subfamilies containing two to four members each. Branch lengths are proportional to the evolutionary distance between each gene.

In certain embodiments, the at least one FGF activator is selected from the group consisting of FGF8a, FGF17, FGF18, FGF8b, FGF2, FGF4, and derivatives thereof. In certain embodiments, the at least one FGF activator is selected from the group consisting of FGF8a, FGF17, FGF18, FGF2, FGF4, and derivatives thereof. In certain embodiments, the at least one FGF activator is selected from the group consisting of FGF8a, FGF17, and FGF18.

The FGF8 subfamily is comprised of FGF8a, FGF8b, FGF17, and FGF18. Early patterning of the vertebrate midbrain and cerebellum is regulated by a mid/hindbrain organizer that produces FGF8a, FGF8b, FGF17 and FGF18. It has been shown that FGF8b functions differently from FGF8a, FGF17, and FGF18 (Liu et al., Development. 2003 December; 130 (25): 6175-85). FGF8b is the only protein that can induce the rl gene Gbx2 and strongly activate the pathway inhibitors Spry 1/2, as well as repress the midbrain gene Otx2 (Liu 2003). Moreover, FGF8b extends the organizer along the junction between the induced Gbx2 domain and the remaining Otx2 region in the midbrain, correlating with cerebellum development (Liu 2003). By contrast, FGF8a, FGF17, and FGF18 cause expansion of the midbrain and upregulating midbrain gene expression (Liu 2003).

In certain embodiments, the at least one FGF activator is capable of causing expansion of the midbrain and upregulating midbrain gene expression. In certain embodiments, the at least one FGF activator is capable of promoting midbrain development. In certain embodiments, the at least one FGF activator is selected from the group consisting of FGF8a, FGF17, FGF18, FGF2, FGF4, derivatives thereof, and combinations thereof. In certain embodiments, the at least one FGF activator is selected from the group consisting of FGF8a, FGF17, FGF18, and combinations thereof. In certain embodiments, the at least one FGF activator comprises or is FGF18.

In certain embodiments, the cells are contacted with or exposed to the at least one FGF activator for about 3 days, about 5 days, or about 8 days. In certain embodiments, the cells are contacted with or exposed to the at least one FGF activator for about 4 days. In certain embodiments, the cells are contacted with or exposed to the at least one FGF activator for 5 days.

In certain embodiments, the contact of the cells with or the exposure of the cells to the at least one FGF activator is initiated about 10 days from the initial contact of the cells with or the initial exposure of the cells to the at least one SMAD inhibitor, and the cells are contacted with the at least FGF activator for about 5 days. In certain embodiments, the contact of the cells with or the exposure of the cells to the at least one FGF activator is initiated 12 days or 13 days from the initial contact of the cells with or the initial exposure of the cells to the at least one SMAD inhibitor, and the cells are contacted with the at least one FGF activator for 4 days or 5 days. In certain embodiments, the cells are contacted with or exposed to the at least one FGF activator from day 12 through day 16.

In certain embodiments, the concentration of the at least one FGF activator contacted with or exposed to the cells is about 100 ng/ml or about 200 ng/ml. In certain embodiments, concentration of the at least one FGF activator contacted with or exposed to the cells is about 200 ng/mL.

In certain embodiments, the at least one FGF activator comprises FGF18.

### 5.2.4.6. Wnt Inhibitors

Wnt signaling includes canonical Wnt signaling and non-canonical Wnt signaling. In certain embodiments, the at least one Wnt inhibitor is capable of inhibiting canonical Wnt signaling. In certain embodiments, the at least one Wnt inhibitor is capable of inhibiting both canonical Wnt signaling and non-canonical Wnt signaling. Non-limiting examples of Wnt inhibitors that are capable of inhibiting both canonical Wnt signaling and non-canonical Wnt signaling include IWP2, IWR1-endo, IWP-01, Wnt-C59, IWP-L6, IWP12, LGK-974, IWR-1, ETC-159, iCRT3, IWP-4, salinomycin, Pyrvinium Pamoate, iCRT14, FH535, CCT251545, Wogonin, NCB-0846, hexachrorophene, KY02111, SO3031 (KY01-I), SO2031 (KY02-I), BC2059, PKF115-584, Quercetin, NSC668036, G007-LK, and derivatives thereof. In certain embodiments, the at least one Wnt inhibitor is selected from the group consisting of IWP2, IWR1-endo, XAV939, IWP-01, Wnt-C59, IWP-L6, LGK-974, IWR-1, Wnt-C59, ETC-159, iCRT3, IWP-4, ICG-001, Salinomycin, Pyrvinium Pamoate, iCRT14, FH535, CCT251545, KYA1797K, Wogonin, NCB-0846, Hexachrorophene, PNU-74654, KY02111, SO3031 (KY01-I), SO2031 (KY02-I), triptonide, IWP12, BC2059, PKF115-584, Quercetin, NSC668036, G007-LK, MSAB, LF3, JW55, isoquercitrin, WIKI4 (Wnt Inhibitor Kinase Inhibitor 4), derivatives thereof, and combinations thereof. In certain embodiments, the at least one inhibitor of Wnt signaling is selected from the group consisting of IWP2, IWR1-endo, IWP-01, IWP12, Wnt-C59, IWP-L6, LGK-974, IWR-1, ETC-159, iCRT3, IWP-4, Salinomycin, Pyrvinium Pamoate, iCRT14, FH535, CCT251545, Wogonin, NCB-0846, Hexachrorophene, KY02111, SO3031 (KY01-I), SO2031 (KY02-I), BC2059, PKF115-584, Quercetin, NSC668036, G007-LK, derivatives thereof, and combinations thereof. In certain embodiments, the at least one inhibitor of Wnt signaling is selected from the group consisting of XAV939, ICG-001, PNU-74654, Triptonide, KYA1797K, MSAB, LF3, JW55, Isoquercitrin, WIKI4, derivatives thereof, and combinations thereof. In certain embodiments, the at least one Wnt inhibitor comprises IWP2 or a derivative thereof.

In certain embodiments, the cells are contacted with or exposed to the at least one Wnt inhibitor for about 15 days or about 20 days. In certain embodiments, the cells are contacted with or exposed to the at least one Wnt inhibitor for 4 days, 5 days, 6 days, 7 days, 14 days, 15 days, 16 days, 17 days, 18 days, 19 days, 20 days, 21 days, 22 days, or 23 days.

In certain embodiments, the cells that are contacted with the at least one Wnt inhibitor comprise mDA neuron precursors and mDA neurons.

In certain embodiments, the contact of the cells with or the exposure of the cells to the at least one Wnt inhibitor is initiated about 10 days from the initial contact of the cells with or the initial exposure of the cells to the at least one SMAD inhibitor, and the cells are contacted with the at least Wnt inhibitor for about 5 days. In certain embodiments, the contact of the cells with or the exposure of the cells to the at least one Wnt inhibitor is initiated 12 days or 13 days from the initial contact of the cells with or the initial exposure of the cells to the at least one SMAD inhibitor, and the cells are contacted with the at least one Wnt inhibitor for 4 days or 5 days. In certain embodiments, the cells are contacted with or exposed to the at least one Wnt inhibitor from day 12 through day 16. In certain embodiments, the cells are contacted with or exposed to the at least one Wnt inhibitor from day 12 through day 25. In certain embodiments, the at least one Wnt inhibitor is added every day or every other day to a cell culture medium comprising the cells from day 12 through day 25. In certain embodiments, the at least one Wnt inhibitor is added every day or every other day to a cell culture medium comprising the cells from day 12 through day 30.

In certain embodiments, the concentration of the at least one Wnt inhibitor contacted with or exposed to the cells is about 1 μM.

In certain embodiments, the at least one Wnt inhibitor comprises IWP2.

### 5.2.4.7. DA Neuron Lineage Specific Activators and Inhibitors

In certain embodiments, the cells are further contacted with DA neuron lineage specific activators and inhibitors to obtain the mDA neurons (e.g., post-mitotic mDA neurons). Non-limiting examples of DA neuron lineage specific activators and inhibitors include L-glutamine, brain-derived neurotrophic factor (BDNF), glial cell-derived neurotrophic factor (GDNF), Cyclic adenosine monophosphate (cAMP), Transforming growth factor beta (TGFβ, for example, TGFβ3), ascorbic acid (AA), and DAPT (which is also known as, N-[(3,5-Difluorophenyl) acetyl]-L-alanyl-2-phenyl]glycine-1,1-dimethylethyl ester; LY-374973, N-[N-(3,5-Difluorophenacetyl)-L-alanyl]-S-phenylglycine t-butyl ester; or N-[N-(3,5-difluorophenacetyl)-L-alanyl]-S-phenylglycine t-butyl ester).

In certain embodiments, the cells are contacted with the foregoing DA neuron lineage specific activators and inhibitors for about 4 days, about 5 days, about 6 days, about 7 days, or about 8 days.

In certain embodiments, the cells are contacted with L-glutamine at a concentration of about 2 mM. In certain embodiments, the cells are contacted with BDNF at a concentration of about 20 ng/mL. In certain embodiments, the cells are contacted with AA at a concentration of about 200 nM. In certain embodiments, the cells are contacted with GDNF at a concentration of about 20 ng/ml. In certain embodiments, the cells are contacted with cAMP at a concentration of about 500 nM. In certain embodiments, the cells are contacted with TGFβ3 at a concentration of about 1 ng/mL. In certain embodiments, the cells are contacted with DAPT at a concentration of about 10 nM.

### 5.3. Compositions

The present disclosure provides compositions for treating neurodegeneration of midbrain dopamine neurons and/or for treating neurodegenerative disorders. In certain embodiments, the composition comprises (i) one or more mDA neurons (e.g., mDA neurons disclosed in Section 5.2.4.) and (ii) at least one compound selected from the group consisting of TNFα inhibitors (e.g., the TNFα inhibitors disclosed in Section 5.2.1), NFκB inhibitors (e.g., the NFκB inhibitors disclosed in Section 5.2.2), p53 inhibitors (e.g., the p53 inhibitors disclosed in Section 5.2.3), and combinations thereof.

In certain embodiments, the one or more mDA neurons and the at least one compound are comprised in a composition that further comprises a biocompatible scaffold or matrix, for example, a biocompatible three-dimensional scaffold that facilitates tissue regeneration when the one or more mDA neurons are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties). In certain embodiments, the composition further comprises growth factors for promoting maturation of the implanted/grafted cells.

In certain embodiments, the composition comprises from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the mDA neurons. In certain embodiments, the composition comprises from about 1×10⁵ to about 1×10⁷ the mDA neurons.

In certain embodiments, the composition is frozen. In certain embodiments, the composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition is a pharmaceutical composition that comprises a pharmaceutically acceptable carrier. In certain embodiments, the compositions can be used for preventing and/or treating a neurodegenerative disorder include Parkinson's disease, Huntington's disease, Alzheimer's disease, multiple sclerosis, amyotrophic lateral sclerosis (ALS), and frontotemporal dementia. In certain embodiments, the compositions can be used for ameliorating neurodegeneration of mDA neurons.

The present disclosure also provides devices comprising the presently disclosed compositions. The devices can be used for administering the presently disclosed compositions. Any suitable administration devices can be used for the present disclosure. Non-limiting examples of devices include syringes, fine glass tubes, stereotactic needles and cannulas.

### 5.4 Methods of Treatment

The present disclosure provides methods of treating a subject. In certain embodiments, the subject suffers from neurodegeneration of midbrain dopamine neurons. The present disclosure also provides methods for ameliorating neurodegeneration of mDA neurons. In certain embodiments, the subject has a neurodegenerative disorder. Non-limiting examples of neurodegenerative disorders include Parkinson's disease, Huntington's disease, Alzheimer's disease, multiple sclerosis, amyotrophic lateral sclerosis (ALS), and frontotemporal dementia. The present disclosure further provides methods of preventing and/or treating at least a symptom in a subject having a neurological disorder, and/or suffering from neurodegeneration of midbrain dopamine neurons.

In certain embodiments, the methods comprise administering to the subject one or more mDA neurons, wherein p53-mediated apoptosis of the one or more mDA neurons is suppressed (e.g., those mDA neurons disclosed in Section 5.2.4.). In certain embodiments, the suppression of p53-mediated apoptosis comprises inhibition of tumor necrosis factor alpha (TNFα) signaling (e.g., inhibition of TNFα signaling disclosed in Section 5.2.1), inhibition of nuclear factor kappa B (NFκB) signaling (e.g., inhibition of NFκB signaling disclosed in Section 5.2.2), inhibition of p53 signaling (e.g., inhibition of p53 signaling disclosed in Section 5.2.3), or a combination of the foregoing. In certain embodiments, the suppression of p53-mediated apoptosis comprises administering to the subject at least one compound selected from the group consisting of TNFα inhibitors, NFκB inhibitors, p53 inhibitors, and combinations thereof. In certain embodiments, the method comprises administering the one or more mDA neurons simultaneously with the administration of the compound. In certain embodiments, the one or more mDA neurons are contacted with the at least one compound prior to the administration of the one or more mDA neurons to the subject. In certain embodiments, the method comprises administering the at least one compound shortly after administration of the one or more mDA neurons. In certain embodiments, the method comprises administering the at least one compound before the administration of the one or more mDA neurons.

In certain embodiments, the methods comprise administering to the subject a composition disclosed in Section 5.3.

In certain embodiments, the neurodegenerative disorder is Parkinson's disease. Primary motor signs of Parkinson's disease include, for example, but not limited to, tremor of the hands, arms, legs, jaw and face, bradykinesia or slowness of movement, rigidity or stiffness of the limbs and trunk and postural instability or impaired balance and coordination.

In certain embodiments, the neurodegenerative disorder is a parkinsonism disease, which refers to diseases that are linked to an insufficiency of dopamine in the basal ganglia, which is a part of the brain that controls movement. Symptoms include tremor, bradykinesia (extreme slowness of movement), flexed posture, postural instability, and rigidity. Non-limiting examples of parkinsonism diseases include corticobasal degeneration, Lewy body dementia, multiple systematrophy, and progressive supranuclear palsy.

In certain embodiments, the one or more mDA neurons and the means to suppress p53-mediated apoptosis (e.g., a TNFα inhibitor, a NFκB inhibitor, a p53 inhibitor, or a combination of the foregoing) can be administered or provided systemically or directly to a subject suffering from a neurodegenerative disorder and/or neurodegeneration of midbrain dopamine neurons. In certain embodiments, the one or more mDA neurons and the means to suppress p53-mediated apoptosis (e.g., a TNFα inhibitor, a NFκB inhibitor, a p53 inhibitor, or a combination of the foregoing) are directly injected into an organ of interest (e.g., the central nervous system (CNS) or peripheral nervous system (PNS)). In certain embodiments, the one or more mDA neurons and the means to suppress p53-mediated apoptosis (e.g., a TNFα inhibitor, a NFκB inhibitor, a p53 inhibitor, or a combination of the foregoing) are directly injected into the striatum.

In certain embodiments, the one or more mDA neurons and the means to suppress p53-mediated apoptosis (e.g., a TNFα inhibitor, a NFκB inhibitor, a p53 inhibitor, or a combination of the foregoing) can be administered in any physiologically acceptable vehicle. In certain embodiments, the one or more mDA neurons and the means to suppress p53-mediated apoptosis (e.g., a TNFα inhibitor, a NFκB inhibitor, a p53 inhibitor, or a combination of the foregoing) can be administered via localized injection, orthotopic (OT) injection, systemic injection, intravenous injection, or parenteral administration. In certain embodiments, the one or more mDA neurons and the means to suppress p53-mediated apoptosis (e.g., a TNFα inhibitor, a NFκB inhibitor, a p53 inhibitor, or a combination of the foregoing) are administered to a subject suffering from a neurodegenerative disorder and/or neurodegeneration of midbrain dopamine neurons via orthotopic (OT) injection.

In certain embodiments, the one or more mDA neurons and the means to suppress p53-mediated apoptosis (e.g., a TNFα inhibitor, a NFκB inhibitor, a p53 inhibitor, or a combination of the foregoing) can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter (e.g., a composition of Section 5.4), in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived precursors. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the cells and the means to suppress p53-mediated apoptosis (e.g., a TNFα inhibitor, a NFκB inhibitor, a p53 inhibitor, or a combination of the foregoing) is the quantity of cells and inhibitor(s) necessary to achieve an optimal effect. An optimal effect includes, but is not limited to, repopulation of CNS and/or PNS regions of a subject suffering from a neurodegenerative disorder and/or neurodegeneration of midbrain dopamine neurons, improved function of midbrain dopamine neurons of the subject, the subject's CNS and/or PNS, and improving the in vivo survival of the cells.

An “effective amount” (or “therapeutically effective amount”) is an amount sufficient to affect a beneficial or desired clinical result upon treatment. An effective amount can be administered to a subject in at least one doses. In terms of treatment, an effective amount is an amount that is sufficient to palliate, ameliorate, stabilize, reverse or slow the progression of the neurodegenerative disorder or pituitary disorder, or otherwise reduce the pathological consequences of the neurodegenerative disorder. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the cells is an amount that is sufficient to ameliorate neurodegeneration of midbrain dopamine neurons. In certain embodiments, an effective amount of the cells is an amount that is sufficient to improve the function of midbrain dopamine neurons of a subject suffering from a neurodegenerative disorder and/or neurodegeneration of midbrain dopamine neurons, e.g., the improved function can be about 1%, about 5%, about 10%, about 20%, about 30%, about 40%, about 50%, about 60%, about 70%, about 80%, about 90%, about 95%, about 98%, about 99% or about 100% of the function of midbrain dopamine neurons of a normal person.

In certain embodiments, an effective amount of the means to suppress p53-mediated apoptosis (e.g., a TNFα inhibitor, a NFκB inhibitor, a p53 inhibitor, or a combination of the foregoing) is an amount that is sufficient to improve the in vivo survival of transplanted mDA neurons.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the cells are administered to a subject. In certain embodiments, from about 1×10⁵ to about 1×10⁷ of the cells are administered to a subject suffering from a neurodegenerative disorder and/or neurodegeneration of midbrain dopamine neurons. In certain embodiments, from about 1×10⁶ to about 1×10⁷ of the cells are administered to a subject suffering from a neurodegenerative disorder and/or neurodegeneration of midbrain dopamine neurons. In certain embodiments, from about 1×10⁶ to about 4×10⁶ of the cells are administered to a subject suffering from a neurodegenerative disorder and/or neurodegeneration of midbrain dopamine neurons.

The precise determination of what would be considered an effective dose may be based on factors individual to each subject, including their size, age, sex, weight, and condition of the particular subject. Dosages can be readily ascertained by those skilled in the art from this disclosure and the knowledge in the art.

### Exemplary Embodiments

A. In certain non-limiting embodiments, the presently disclosed subject matter provides for a method for treating a subject, comprising administering to the subject one or more midbrain dopamine (mDA) neurons, wherein p53-mediated apoptosis of the one or more mDA neurons is suppressed.

A1. The foregoing method of A, wherein the subject suffers from a neurodegenerative disorder and/or neurodegeneration of midbrain dopamine neurons.

A2. The foregoing method of A-A1, wherein the neurodegenerative disorder is selected from the group consisting of Parkinson's disease, Huntington's disease, Alzheimer's disease, multiple sclerosis, amyotrophic lateral sclerosis (ALS), frontotemporal dementia, and combinations thereof.

A3. The foregoing method of A-A2, wherein the suppression of p53-mediated apoptosis comprises a) administering to the subject at least one compound selected from the group consisting of tumor necrosis factor alpha (TNFα) inhibitors, nuclear factor kappa B (NFκB) inhibitors, p53 inhibitors, and combinations thereof; or b) contacting the one or more mDA neurons with at least one compound selected from the group consisting of TNFα inhibitors, NFκB inhibitors, p53 inhibitors, and combinations thereof.

A4. The foregoing method of A-A3, wherein the suppression of p53-mediated apoptosis comprises administering to the subject a TNFα inhibitor.

A5. The foregoing method of A-A4, comprising administering the one or more mDA neurons simultaneously with the administration of the at least one compound.

B. In certain non-limiting embodiments, the presently disclose subject matter provides for a method of improving in vivo survival of one or more midbrain dopamine (mDA) neurons, comprising suppressing p53-mediated apoptosis of the one or more mDA neurons.

B1. The foregoing method of B, wherein the suppression of p53-mediated apoptosis comprises contacting the one or more mDA neurons with a compound selected from the group consisting of TNFα inhibitors, NFκB inhibitors, p53 inhibitors, and combinations thereof.

B2. The foregoing method of B-B1, wherein the suppression of p53-mediated apoptosis comprises contacting the one or more mDA neurons with a TNFα inhibitor.

C. The foregoing method of A-A5 or B-B2, wherein the suppression of p53-mediated apoptosis comprises inhibition of TNFα signaling, inhibition of NFκB signaling, inhibition of p53 signaling, or a combination of the foregoing.

C1. The foregoing method of any one of claims A3, A4, B1, and B2, wherein the TNFα inhibitor is selected from the group consisting of anti-TNFα antibodies, TNFα decoy receptors, chemical compounds, nucleic acid inhibitors, small molecule inhibitors, receptor biologic inhibitors, inactive TNF fragments, TNFα circulating receptor fusion protein, xanthine derivatives, 5-HT_{2A} agonist, and combinations thereof.

C2. The foregoing method of C1, wherein the TNFα inhibitor is an anti-TNFα antibody.

C3. The foregoing method of C2, wherein the anti-TNFα antibody is selected from the group consisting of adalimumab, adalimumab-adbm, adalimumab-adaz, adalimumab-atto, certolizumab pegol, golimumab, infliximab, infliximab-abda, infliximab-dyyb, remtolumab, afelimomab, nerelimomab, ozoralizumab, placulumab, and combinations thereof.

C4. The foregoing method of C3, wherein the anti-TNFα antibody is adalimumab.

C5. The foregoing method of A3 or B1, wherein the NFκB inhibitor is selected from the group consisting of upstream inhibitors of NFκB, inhibitors of IKK activity, inhibitors of IκB phosphorylation, inhibitors of IκB degradation, proteasome inhibitors, protease inhibitors, inhibitors of NFκB nuclear translocation and expression, NFκB DNA-binding inhibitors, and NFκB transactivation inhibitors, inhibitors of NFκB directed gene transactivation, antioxidants, and combinations thereof.

C6. The foregoing method of claim A3 or B1, wherein the p53 inhibitor is selected from the group consisting of JNK inhibitors, p38 MAPK inhibitors, caspase inhibitors, puma/BBC3 inhibitors, BAX inhibitors, CDK inhibitors, MDM2 and MDMX activators, and combinations thereof.

C7. The foregoing method of A-A5, B-B2, or C-C6, wherein the suppression of p53-mediated apoptosis comprises knocking out or knocking down TP53 gene in the one or more mDA.

C8. The foregoing method of C7, wherein the TP53 gene is knocked out or knocked down by a gene-engineering system.

C9. The foregoing method of C8, wherein the gene-engineering system is a CRISPR-Cas system.

C10. The foregoing method of A-A5, B-B2, or C-C9, wherein the one or more mDA neurons express a marker selected from the group consisting of EN1, OTX2, TH, NURR1, FOXA2, LMX1A, PITX3, LMO3, SNCA, ADCAP1, CHRNA4, ALDH1A1, SOX6, WNT1, DAT, VMAT2, GIRK2, SATB1, CALB1, CALB2, SNCG, PBX1, and combinations thereof.

C11. The foregoing method of A-A5, B-B2, or C-C10, wherein the one or more mDA neurons are post-mitotic mDA neurons.

C12. The foregoing method of A-A5, B-B2, or C-C11, wherein the one or more mDA neurons are in vitro differentiated from one or more stem cells.

C13. The foregoing method of C12, wherein the one or more stem cells are selected from the group consisting of human stem cells, nonhuman primate stem cells, rodent nonembryonic stem cells, human embryonic stem cells, nonhuman primate embryonic stem cells, rodent embryonic stem cells, human induced pluripotent stem cells, nonhuman primate induced pluripotent stem cells, rodent induced pluripotent stem cells, and human recombinant pluripotent cells, nonhuman primate recombinant pluripotent cells, and rodent recombinant pluripotent cells.

C14. The foregoing method of C12 or C13, wherein the one or more stem cells are human stem cells.

C15. The foregoing method of C12-C14, wherein the one or more stem cells are one or more pluripotent stem cells or multipotent stem cell.

C16. The foregoing method of C12-C15, wherein the one or more stem cells are selected from the group consisting of embryonic stem cells, induced pluripotent stem cells, and combinations thereof.

C17. The foregoing method of C16, wherein the one or more stem cells are one or more induced pluripotent stem cells.

C18. The foregoing method of C12-C17, wherein the in vitro differentiation comprises contacting the one or more stem cells with at least one inhibitor of Small Mothers Against Decapentaplegic (SMAD) signaling, at least one activator of Sonic hedgehog (SHH) signaling, and at least one activator of wingless (Wnt) signaling.

C19. The foregoing method of C18, wherein the concentration of the at least one activator of Wnt signaling that is contacted with the cells is increased between about 2 days and about 6 days from the initial contact of the cells with the at least one activator of Wnt signaling.

C20. The foregoing method of C18 or C19, wherein the concentration of the at least one activator of Wnt signaling that is contacted with the cells is increased by between about 250% and about 1800% of the initial concentration of the at least one activator of Wnt signaling contacted with the cells.

C21. The foregoing method of C18-C20, wherein the at least one activator of Wnt signaling comprises an inhibitor of glycogen synthase kinase 3β (GSK3B) signaling.

C22. The foregoing method of any one of claims C18-C21, wherein the at least one activator of Wnt signaling is selected from the group consisting of CHIR99021, CHIR98014, AMBMP hydrochloride, LP 922056, Lithium, deoxycholic acid, BIO, SB-216763, Wnt3A, Wnt1, Wnt5a, derivatives thereof, and combinations thereof.

C23. The foregoing method of C22, wherein the at least one activator of Wnt signaling comprises CHIR99021.

C24. The foregoing method of C18-C23, wherein the at least one inhibitor of SMAD signaling comprises an inhibitor of TGFβ/Activin-Nodal signaling, an inhibitor of bone morphogenetic protein (BMP) signaling, or a combination of the foregoing.

C25. The foregoing method of C24, wherein the at least one inhibitor of TGFβ/Activin-Nodal signaling is selected from the group consisting of SB431542, derivatives of SB431542, and combinations thereof.

C26. The foregoing method of C25, wherein the derivative of SB431542 comprises A83-01.

C27. The foregoing method of C24-C26, wherein the at least one inhibitor of TGFβ/Activin-Nodal signaling comprises SB431542.

C28. The foregoing method of C24, wherein the at least one inhibitor of BMP signaling is selected from the group consisting of LDN193189, Noggin, dorsomorphin, derivatives of LDN193189, derivatives of Noggin, derivatives of dorsomorphin, and combinations thereof.

C29. The foregoing method of C28, wherein the at least one inhibitor of BMP comprises LDN-193189.

C30. The foregoing method of any one of C18-C29, wherein the at least one activator of SHH signaling is selected from the group consisting of SHH proteins, Smoothened agonists (SAG), and combinations thereof.

C31. The foregoing method of C30, wherein the SHH protein is selected from the group consisting of recombinant SHHs, modified N-terminal SHHs, and combinations thereof.

C32. The foregoing method of C31, wherein the modified N-terminal SHH comprises two isoleucines at the N-terminus.

C33. The foregoing method of C31 or C32, wherein the modified N-terminal SHH has at least about 90% sequence identity to an un-modified N-terminal SHH.

C34. The foregoing method of C33, wherein the un-modified N-terminal SHH is an un-modified mouse N-terminal SHH or an un-modified human N-terminal SHH.

C35. The foregoing method of C31-C33, wherein the modified N-terminal SHH comprises SHH C25II.

C36. The foregoing method of C30, wherein the SAG comprises purmorphamine.

C37. The foregoing method of C18-C36, wherein the in vitro differentiation further comprises contacting the one or more stem cells with at least one activator of fibroblast growth factor (FGF) signaling.

C38. The foregoing method of C37, wherein the at least one activator of FGF signaling is selected from the group consisting of FGF18, FGF17, FGF8a, FGF8b, FGF4, FGF2, and combination thereof.

C39. The foregoing method of C37 or C38, wherein the at least one activator of FGF signaling comprises FGF18 or FGF8.

C40. The foregoing method of C18-C39, wherein the in vitro differentiation further comprises contacting the one or more stem cells with at least one inhibitor of Wnt signaling.

C41. The foregoing method of C40, wherein the at least one inhibitor of Wnt signaling is selected from the group consisting of IWP2, IWR1-endo, XAV939, IWP-01, Wnt-C59, IWP-L6, and ICG-001, and combinations thereof.

C42. The foregoing method of C40 or C41, wherein the at least one inhibitor of Wnt signaling comprises IWP2.

C42. The foregoing method of any one of claims A-A5, B-B2, or C-C42, wherein the one or more mDA neurons express a detectable level of CD184 and do not express a detectable level of CD49e.

D. In certain non-limiting embodiments, the presently disclose subject matter provides for a composition comprising: (a) one or more midbrain dopamine (mDA) neurons; and (b) at least one compound selected from the group consisting of TNFα inhibitors, NFκB inhibitors, p53 inhibitors, and combinations thereof.

D1. The foregoing composition of D, wherein the composition is a pharmaceutical composition further comprising a pharmaceutically acceptable carrier.

D2. The foregoing composition of D or D1, wherein the composition is for treating or ameliorating a neurodegenerative disorder and/or neurodegeneration of midbrain dopamine neurons.

D3. The foregoing composition of D2, wherein the neurodegenerative disorder is selected from the group consisting of Parkinson's disease, Huntington's disease, Alzheimer's disease, multiple sclerosis, amyotrophic lateral sclerosis (ALS), frontotemporal dementia, and combinations thereof.

D4. The foregoing composition of D-D3, wherein the TNFα inhibitor is selected from the group consisting of anti-TNFα antibodies, TNFα decoy receptors, chemical compounds, nucleic acid inhibitors, small molecule inhibitors, receptor biologic inhibitors, inactive TNF fragments, TNFα circulating receptor fusion protein, xanthine derivatives, 5-HT_{2A} agonist, and combinations thereof.

D5. The foregoing composition of D4, wherein the TNFα inhibitor is an anti-TNFα antibody.

D6. The foregoing composition of D5, wherein the anti-TNFα antibody is selected from the group consisting of adalimumab, adalimumab-adbm, adalimumab-adaz, adalimumab-atto, certolizumab pegol, golimumab, infliximab, infliximab-abda, infliximab-dyyb, remtolumab, afelimomab, nerelimomab, ozoralizumab, placulumab, and combinations thereof.

D7. The foregoing composition of D5, wherein the anti-TNFα antibody is adalimumab.

D8. The foregoing composition of any one of D-D7, wherein the NFκB inhibitor is selected from the group consisting of upstream inhibitors of NFκB, inhibitors of IKK activity, inhibitors of IκB phosphorylation, inhibitors of IκB degradation, proteasome inhibitors, protease inhibitors, inhibitors of NFκB nuclear translocation and expression, NFκB DNA-binding inhibitors, and NFκB transactivation inhibitors, inhibitors of NFκB directed gene transactivation, antioxidants, and combinations thereof.

D9. The foregoing composition of D-D8, wherein the p53 inhibitor is selected from the group consisting of JNK inhibitors, p38 MAPK inhibitors, caspase inhibitors, puma/BBC3 inhibitors, BAX inhibitors, CDK inhibitors, MDM2 and MDMX activators, and combinations thereof.

D10. The foregoing composition of D-D9, wherein the one or more mDA neurons express a marker selected from the group consisting of EN1, OTX2, TH, NURR1, FOXA2, LMXIA, PITX3, LMO3, SNCA, ADCAP1, CHRNA4, ALDH1A1, SOX6, WNT1, DAT, VMAT2, GIRK2, SATB1, CALB1, CALB2, SNCG, PBX1, and combinations thereof.

D11. The foregoing composition of D-D10, wherein the one or more mDA neurons are post-mitotic mDA neurons.

D12. The foregoing composition of D-D11, wherein the one or more mDA neurons are in vitro differentiated from one or more stem cells.

D13. The foregoing composition of D12, wherein the one or more stem cells are selected from the group consisting of human stem cells, nonhuman primate stem cells, rodent nonembryonic stem cells, human embryonic stem cells, nonhuman primate embryonic stem cells, rodent embryonic stem cells, human induced pluripotent stem cells, nonhuman primate induced pluripotent stem cells, rodent induced pluripotent stem cells, and human recombinant pluripotent cells, nonhuman primate recombinant pluripotent cells, and rodent recombinant pluripotent cells.

D14. The foregoing composition of D12 or D13, wherein the one or more stem cells are human stem cells.

D15. The foregoing composition of D12-D14, wherein the one or more stem cells are one or more pluripotent stem cells or multipotent stem cell.

D16. The foregoing composition of D12-D14, wherein the one or more stem cells are selected from the group consisting of embryonic stem cells, induced pluripotent stem cells, and combinations thereof.

D17. The foregoing composition of D16, wherein the one or more stem cells are one or more induced pluripotent stem cells.

D18. The foregoing composition of D12-D17, wherein the in vitro differentiation comprises contacting the one or more stem cells with at least one inhibitor of Small Mothers Against Decapentaplegic (SMAD) signaling, at least one activator of Sonic hedgehog (SHH) signaling, and at least one activator of wingless (Wnt) signaling.

D19. The forgoing composition of D18, wherein the concentration of the at least one activator of Wnt signaling that is contacted with the cells is increased between about 2 days and about 6 days from the initial contact of the cells with the at least one activator of Wnt signaling.

D20. The foregoing composition of D18 or D19, wherein the concentration of the at least one activator of Wnt signaling that is contacted with the cells is increased by between about 250% and about 1800% of the initial concentration of the at least one activator of Wnt signaling contacted with the cells.

D21. The foregoing composition of any one of claims D18-D20, wherein the at least one activator of Wnt signaling comprises an inhibitor of glycogen synthase kinase 3B (GSK3B) signaling.

D22. The foregoing composition of D18-D21, wherein the at least one activator of Wnt signaling is selected from the group consisting of CHIR99021, CHIR98014, AMBMP hydrochloride, LP 922056, Lithium, deoxycholic acid, BIO, SB-216763, Wnt3A, Wnt1, Wnt5a, derivatives thereof, and combinations thereof.

D23. The foregoing composition of D22, wherein the at least one activator of Wnt signaling comprises CHIR99021.

D24. The foregoing composition of D18-D23, wherein the at least one inhibitor of SMAD signaling comprises an inhibitor of TGFβ/Activin-Nodal signaling, an inhibitor of bone morphogenetic protein (BMP) signaling, or a combination of the foregoing.

D25. The foregoing composition of D24, wherein the at least one inhibitor of TGFβ/Activin-Nodal signaling is selected from the group consisting of SB431542, derivatives of SB431542, and combinations thereof.

D26. The foregoing composition of D25, wherein the derivative of SB431542 comprises A83-01.

D27. The foregoing composition of D25, wherein the at least one inhibitor of TGFβ/Activin-Nodal signaling comprises SB431542.

D28. The foregoing composition of D24-D27, wherein the at least one inhibitor of BMP signaling is selected from the group consisting of LDN193189, Noggin, dorsomorphin, derivatives of LDN193189, derivatives of Noggin, derivatives of dorsomorphin, and combinations thereof.

D29. The foregoing composition of D28, wherein the at least one inhibitor of BMP comprises LDN-193189.

D30. The foregoing composition of D19-D29, wherein the at least one activator of SHH signaling is selected from the group consisting of SHH proteins, Smoothened agonists (SAG), and combinations thereof.

D31. The foregoing composition of D30, wherein the SHH protein is selected from the group consisting of recombinant SHHs, modified N-terminal SHHs, and combinations thereof.

D32. The foregoing composition of D31, wherein the modified N-terminal SHH comprises two isoleucines at the N-terminus.

D33. The foregoing composition of D31 or D32, wherein the modified N-terminal SHH has at least about 90% sequence identity to an un-modified N-terminal SHH.

D34. The foregoing composition of D33, wherein the un-modified N-terminal SHH is an un-modified mouse N-terminal SHH or an un-modified human N-terminal SHH.

D35. The foregoing composition of D31-D34, wherein the modified N-terminal SHH comprises SHH C25II.

D36. The foregoing composition of D30, wherein the SAG comprises purmorphamine.

D37. The foregoing composition of D18-D36, wherein the in vitro differentiation further comprises contacting the one or more stem cells with at least one activator of fibroblast growth factor (FGF) signaling.

D38. The foregoing composition of D37, wherein the at least one activator of FGF signaling is selected from the group consisting of FGF18, FGF17, FGF8a, FGF8b, FGF4, FGF2, and combination thereof.

D39. The foregoing composition of D37 or D38, wherein the at least one activator of FGF signaling comprises FGF18 or FGF8.

D40. The foregoing composition of D18-D39, wherein the in vitro differentiation further comprises contacting the one or more stem cells with at least one inhibitor of Wnt signaling.

D41. The foregoing composition of D40, wherein the at least one inhibitor of Wnt signaling is selected from the group consisting of IWP2, IWR1-endo, XAV939, IWP-O1, Wnt-C59, IWP-L6, and ICG-001, and combinations thereof.

D42. The foregoing composition of D40 or D41, wherein the at least one inhibitor of Wnt signaling comprises IWP2.

D43. The foregoing composition of D-D42, wherein the one or more mDA neurons express a detectable level of CD184 and do not express a detectable level of CD49e.

### EXAMPLES

The present disclosure will be better understood by reference to the following Example, which is provided as exemplary of the presently disclosed subject matter, and not by way of limitation.

### Example 1—Manipulation of TNF-NF KB-p53 Axis for the Survival of Enriched hPSC-Derived Post-Mitotic Dopamine Neuron In Vivo

One challenge in developing hPSC-based cell therapies in PD is to eliminate contaminating cell types such as non-dopaminergic neurons and a range of non-neuronal lineages within the graft. Studies using fetal tissue transplantation resulted in graft-induced dyskinesia (GID) as an unexpected side effect in some of the patients (Dorsey et al., 2018, J Parkinsons Dis 8, S3-S8), and it has been suggested that GID may be caused by contaminating serotonergic neurons in fetal grafts (Politis et al., 2010, Sci Transl Med 2, 38ra46). Recent hPSC-based studies highlighted the presence of potential other contaminants in dopamine neuron grafts, such as in rare instances, contamination with TTR+ positive choroid plexus epithelial cells (Doi et al., 2020, Nat Commun 11, 3369) or the presence of perivascular fibroblast-like populations.

Surface marker sorting strategies have been proposed to enrich floor-plate intermediate or late stage of dopamine precursor for transplantation, but none of these markers are specific for dopamine neuron lineage. The first-in-human clinical trials of using hPSC derived dopamine neuron grafts are based on grafting dopamine neuron precursors, rather than postmitotic dopamine neurons as cell type of choice (Doi et al., 2020, Nat Commun 11, 3369; Kirkeby et al., 2017, Cell Stem Cell 20, 135-148.; Piao et al., 2021, Cell Stem Cell 28, 217-229 e217; Schweitzer et al., 2020, N Engl J Med 382, 1926-1932). However, given the limited understanding of long-term risks and potential side-effects that could come from “off-target” neuronal or non-neuronal populations, it can be ideal to engraft homogenous post-mitotic dopamine neuron populations to minimize any safety-related concerns.

Pooled genetic screening using CRISPR/Cas9 technology has increasingly become a technology of choice to uncover causal genes driving specific phenotypes in an unbiased manner (Shalem et al., 2015, Nat Rev Genet 16, 299-311). This approach has been widely adopted for in vitro studies but has also been vital to discover essential genes for in vivo tumor growth and metastasis (Chen et al., 2015, Cell 160, 1246-1260.), modulators for macrophage infiltration, and cancer immunotherapy targets (Wang et al., 2021, Cell 184, 5357-5374 e5322) and in vivo colon tumor suppressors (Michels et al., 2020, Cell Stem Cell 26, 782-792 e787.). Here, the presently disclosed subject matter sets out to systematically identify candidate mechanisms driving the death of the grafted cell dopamine neurons using CRISPR-Cas9 technology. Purified Nurr1::H2B-GFP+ postmitotic dopamine neurons were used for this screen to avoid confounding factors such as dopamine neuron precursor proliferation and to identify conditions that may eventually enable the efficient grafting of postmitotic neurons in a translational setting. Barcode sequencing identified a key role for TP53 in restricting postmitotic dopamine neuron survival following transplantation. Further, the kinetics of p53 induction upon grafting and the subsequent recruitment of host neuroimmune cells to the dying neurons were mapped and examined. Transcriptomic analysis revealed TNFα-mediated activation of NFκB as one of the main upstream regulators of p53-mediated dopamine neuron death. To further exploit those insights towards translational use, a set of two cell surface markers was identified to reliably enrich post-mitotic dopamine neurons and thereby avoiding the need for a genetic reporter system. Furthermore, it was demonstrated that the use of an FDA-approved monoclonal antibody blocking TNFα (adalimumab) was capable of dramatically improving post-mitotic dopamine neuron survival, mimicking the results observed in TP53 null dopamine neurons. The present example offers a better understanding of the mechanisms that drive postmitotic dopamine neuron death upon transplantation and establishes a clinically relevant strategy for future implementation in cell-based therapy approaches for PD.

### Methods

Cell line. H9 (WA-09, passage 40-60) human pluripotent stem cell (hPSC) line was employed throughout the study, which engineer to generate Nurr1::GFP reporter hPSC and doxycycline-inducible CRISPR/Cas9 expression in the Nurr1::GFP hPSC line (iCas9/NURR1::GFP hPSC) as well as iCas9/NURR1::GFP hPSC lines containing sgRNA-pool libraries and sgRNA for dTomato and p53. hPSCs were grown in feed-free conditions on vitronectin (VTN-N; Thermo Fisher Scientific)-coated dishes in E8-essential medium and maintained at 37° C., 5% CO₂. Tri-I (MSKCC, Weil-Cornell, Rockefeller University) Embryonic Stem Cell Oversight (ESCRO) approved this study.

Construction of Nurr1:GFP and inducible expression of CRISPR/Cas9 in Nurr1:GFP hESC lines. Generation of Nurr1::GFP hESC line was previously described (Riessland et al., 2019, Cell Stem Cell. October 3; 25 (4): 514-530.c8). Briefly, the stop codon of endogenous NR4A2 (Nurr1) was replaced by EGFP expression cassette (P2A-H2B-PgkPuro) by using a CRISPR/CAS9-mediated knock-in approach. The resulting NURR1:GFP+ cells almost express TH (a mature mDA marker; 98%) based on single-cell qRT-PCR. To generate doxycycline-inducible CRISPR/Cas9 expression in the Nurr1::GFP hPSC line (iCas9/NURR1::GFP hPSC line), a pair of TALEN, Neo-M2rtTA donor, and Hygro-Cas9 donor (Addgene #86883), targeting to an AAVS locus, were transfected into the Nurr1::GFP hPSC using Nucleofector (Lonza, B-016 program) in AMAXA machine and stable cell line was generated following a published protocol. Briefly, 2 days after transfection, neomycin and hygromycin (100 μg/ml) were treated for 1 week and picked clonal expanded hPSC. Inducible expression of Cas9 in each clone was confirmed by immunofluorescent staining with a Cas9 antibody after Dox (1 μg/ml) exposure for 3 days.

Single-strand guide RNA (sgRNA) Design and Cloning. sgRNA sequences for pool library were identified by Guidescan (MSKCC) and sgRNA oligos were synthesized on-Chip (Agilent). Synthesized oligos were PCR amplified and amplicons were restriction cloned into SGL40C.EFS.dTomato (Addgene #89395). Library representation was assessed by NGS (Illumina). Individual sgRNA for dTomato and p53, designed by web-based tool (http://crispor.tefor.net) and using Guidescan (MSKCC) subsequently, were restriction cloned into SGL40C.EFS.dTomato plasmid vector (Addgene #89395).

Lentiviral production and transduction. SGL40C.EFS.dTomato vector containing sgRNA for libraries, dTomato, and p53 was co-transfected with packing vectors, psPAX2 (Addgene #12260) and pMD2.G (Addgene #12259) into HEK293T cell using Xtream Gene 9 transfection reagent (Sigma). The virus was collected after 2 days of transfection, and infected into the iCas9/NURR1::GFP hPSC. 2 days post infection, dTomato expressed hPSCs were sorted using flow cytometry associated cell sorting (FACS) in Flow Cytometry Core Facility at MSKCC. The sorted hPSCs were cultured and maintained for subsequent experiments until use.

hESC differentiation toward DA neurons. Midbrain dopaminergic neuron differentiation was performed using H9 hESCs, which include Nurr1::GFP. hESCs were grown on VTN-N (Thermo Fisher Scientific)-coated 6-well plates in E8-essential medium. Cells were maintained at 37° C., 5% CO₂. hESCs were differentiated with an optimized protocol from a previously reported study (Kim et al., 2021, Cell Stem Cell February 4; 28 (2): 343-355.c5; Riessland et al., 2019, Cell Stem Cell. October 3; 25 (4): 514-530.c8). At day 25 of differentiation from hPSCs iCas9/NURR1::GFP hPSC lines containing sgRNA-pool libraries and sgRNA for dTomato and p53 hPSCs, GFP and dTomato expressed dopamine neurons were sorted using flow cytometry associated cell sorting (FACS). The sorted hPSCs were either injected into mice or cultured for subsequent experiments until use. Furthermore, double sorting approach with CD49e-low and CD184-high was applied to enrich post-mitotic dopamine neuron at day 25 differentiation derived from hPSC using FACS, and sorted cells were used for transplantation and in vitro culture. TNF-α neutralizing antibody was employed either to co-injection (1 mg/ml) or in vitro cultured dopamine neuron (10 μg/ml).

sgRNA barcode Sequencing and Analysis to identify targets. Cell Pellets at each desired time point were lysed, and genomic DNA was extracted (Qiagen) and quantified by Qubit (Thermo Scientific). A quantity of gDNA covering 1000× representation of sgRNAs was PCR amplified to add Illumina adapters and multiplexing barcodes. Amplicons were quantified by Qubit and Bioanalyzer (Agilent) and sequenced on Illumina HiSeq 2500. Sequencing reads were aligned to the screened library and counts were obtained for each gRNA. The resulting single end reads were checked for quality (FastQC v0.11.5) and processed using the Digital Expression Explorer 2 (DEE2) workflow. Adapter trimming was performed with Skewer (v0.2.2). Further quality control done with Minion, part of the Kraken package. Differential gRNA hits were identified using EdgeR, a Bioconductor package, to identify the primary hits. We used the Trimmed Mean of M-values for normalization and the glmQLFTest/F test for statistic tests. Additionally, we used the camera analysis function from EdgeR for gene-level analysis as previously described. To calculate the correlation between the screen samples, quantifications were normalized by the estimateSizeFactorsForMatrix of DESeq2 using only the non-targeting control and safe harbor probes. Pearson correlation of the pairwise comparison was plotted using an R package pheatmap (https://CRAN.R-project.org/package=pheatmap).

Intracranial transplantation. hESCs-derived DA neurons that were sorted based on either NURR1::H2B-GFP or CD49e low and CD184 high were resuspended in 100,000+ cells/μL in neurobasal medium with 200 mM L-glutamine and 100 mM ascorbic acid (AA) transplantation medium (without human albumin or kedbumin 25%). Unless specified, 3-4 μL of sorted neurons were injected at the rate of 0.5-1 μL per deposit) into the striatum of wild-type (unlesioned) 6 to 8-week-old male NSG mice ([AP]+0.5 mm, [ML]+/−1.8 mm, [DV] −3.4 to −3.3 mm from dura). Each surgery did not exceed more than 30 minutes per animal and the entire surgery time was within 8-10 hours post cell preparation. For the short-term 1-month survival study, p53 WT (−dox) vs. KO (+dox) NURR1::GFP+ dopamine neurons or CD+PBS vs. CD+adalimumab was bilaterally engrafted into the striatum of the same mouse brain to reduce variability between animals. For other studies including time course or behavior studies, cells are engrafted unilaterally. For the time-course experiments, the mice were euthanized and used for immunohistochemistry analysis at designated time points (4 hrs, 1 day, 3 days, and 7 days post engraftment). For in vivo CRISPR screen, maximum loadable amount of the cells is implanted with cell density of 200,000±10,000 cells/μL (4 μL total in each striatum) in order to have enough representations of the guide RNAs.

DNA extraction from the xenograft sample and detection of human DNA. Grafted dopamine neurons at 1 month were isolated from thick tissue slices based on NURR1::GFP and sgRNA::Tomato expressions and extracted genomic DNAs using the DNeasy Tissue kit according to the manufactural protocol (Qiagen #69556). To detect human DNA from the extracted genomic DNA, PCRs were performed to detect human and mouse cells with human and mouse specific primers for PTGFR2 and ptgfr2 subsequently using the Q5 High-Fidelity PCR kit (NEB #M0493S). Primer sequences are below.

| | Human PTGFR2 primer 5′: |
|---|---|
| (SEQ ID NO.: 539) | |
| | GCTGCTTCTCATTGTCTCGG |
| | Human PTGFR2 primer 3′: |
| (SEQ ID NO.: 540) | |
| | GCCAGGAGAATGAGGTGGTC |
| | Mouse ptgfr2 primer 5′: |
| (SEQ ID NO.: 541) | |
| | CCTGCTGCTTATCGTGGCTG |
| | Mouse ptgfr2 primer 3′: |
| (SEQ ID NO.: 542) | |
| | GCCAGGAGAATGAGGTGGTC |

6-OHDA mouse model. Briefly, adult female and male NSG mice (6-12 weeks) were anesthetized with 1%-2% isoflurane mixed in oxygen. 1 μL 6-OHDA (3 mg/ml, in saline with 1% ascorbic acid) was directly injected into the right side of substantial nigra (anterior-posterior [AP]=−2.9 mm, lateral [ML]=1.1 mm, vertical [DV]=4.5 mm, from dura) with rate of 0.5-1 μL per minute to generate unilateral toxin Parkinsonian mouse model. Animals with amphetamine-induced rotation at >6 rotations per min were selected for cell transplantation 4 weeks after 6-OHDA-lesion surgery. Animals were randomly grouped and transplanted with CD sorted neurons vs. CD sorted neurons+adalimumab vs. PBS (sham surgery control).

d-amphetamine induced rotation behavior assays. Amphetamine-induced rotation tests were performed twice before transplantation, and 1, 2, 3, 4, 5, 6 months after transplantation. The mice were injected intraperitoneally with a d-amphetamine in saline (Sigma, 10 mg/kg). After 10 minutes, the rotation

Immunohistochemistry. Histology on tissues from mice was performed on frozen sections from xenografts. Mice were anesthetized with pentobarbital and transcardically perfused using heparinized (10 U/mL) PBS (pH 7.4), followed by 4% paraformaldehyde in PBS. The liquid is administered using peristaltic pump to control the rate of the solution delivery to the system. Tissues were post-fixed in ice cold 4% paraformaldehyde for 18 hours sharp and transferred to 30% sucrose until the tissue sink (typically 3-6 days post-), followed by snap freezing in O.C.T (Fisher Scientific, Pittsburgh, PA) or Neg-50 (Thermo Scientific). Brain tissues are all sectioned in 30 μm thick coronal sections using a cryostat and mounted onto a Superfrost plus microscope slides (Fisher Scientific). All the slides are stored at −80° C. for long-term until use. To process for immunolabeling, tissues are washed twice with 1×PBS, followed by permeabilization in in 0.5% Triton X-100 in PBS for 10 minutes. Living cells in culture were directly fixed in 4% paraformaldehyde for 18 min, followed by 10 min permeabilization in 0.5% Triton X-100 in PBS. For labeling, cells or tissue sections were immunostained with primary antibodies of interest in 2% BSA in 0.25% Triton X-100 in PBS at 4° C. overnight. Next day appropriate Alexa Fluor secondary antibodies are conjugated at room temperature for 1 hr at a dilution of 1:1,000. Nuclei were counterstained by DAPI.

Tissue immunohistochemistry (IHC), TUNEL, and H&E stain. H&E and IHC on tissues from mice was performed on FFPE (formalin fixed paraffin embedded) sections from xenografts. Mice were anesthetized with pentobarbital and transcardically perfused using heparinized (10 U/mL) PBS (pH 7.4), followed by 10% formalin. The liquid is administered using peristaltic pump to control the rate of the solution delivery to the system. Tissues were post-fixed in 10% formalin 24-48 hours at room temperature (can stay up to a week in 10% formalin) and directly transferred to 70% ethanol. Histology was performed by HistoWiz Inc. (histowiz.com) using a Standard Operating Procedure and fully automated workflow. Samples were embedded in paraffin, and sectioned at 4 μm. Immunohistochemistry was performed on a Bond Rx autostainer (Leica Biosystems) with heat-mediated antigen retrieval using Epitope Retrieval Solution 1 (Leica Biosystems). Primary antibodies used were rabbit polyclonal total NFκB (Cell Signaling, CST8242, 1:300), Cleaved Caspase-3 (Cell signaling, CST9661, 1:300), p53 (CM5P, 1:500), and p-NFκB (GTX55113, 1:5000) followed by anti-rabbit HRP conjugated polymer system. Bond Polymer Refine Detection (Leica Biosystems) was used according to the manufacturer's protocol. After staining, sections were dehydrated and film coverslipped using a TissueTek-Prisma and Coverslipper (Sakura). Whole slide scanning (40×) was performed on an Aperio AT2 (Leica Biosystems). For TUNEL: Standardized conditions using the Promega DeadEnd Colorimetric Detection System (G3250), Enzyme Digestion for 10 minutes, using the Leica Bond Polymer Refine Detection Kit (DS9800). For H&E, staining was performed on Sakura Autostainer. Briefly, deparaffinize the slides in 2 changes of xylene, 2 changes of 100% alcohol, 1 change in 95% alcohol, then wash with water. Place slides in this sequence: hematoxylin, a rinse with water, define solution, a rinse with water, bluing agent solution, rinse with water, 95% alcohol, eosin, and 95% alcohol. Finish with two changes of 100% alcohol and two with xylene.

RNA extraction and Real time quantitative reverse transcription-PCR (gRT-PCR). Total RNA samples were prepared from cells and DNase I treated using TRIzol according to the manufacturer's instructions. Delta-delta-cycle threshold (AACT) was determined relative to GAPDH levels and normalized to control samples. Error bars indicate the standard deviation of the mean from three biological replicates. The sequences of qRT-PCR primers are shown below.

| | Human GAPDH primer 5′: |
|---|---|
| (SEQ ID NO.: 543) | |
| | ATGTTCGTCATGGGTGTGAA |
| | Human GAPDH primer 3′: |
| (SEQ ID NO.: 544) | |
| | AGGGGTGCTAAGCAGTTGGT |
| | Human FOXA2 primer 5′: |
| (SEQ ID NO.: 545) | |
| | CCGACTGGAGCAGCTACTATG |
| | Human FOXA2 primer 3′: |
| (SEQ ID NO.: 546) | |
| | TACGTGTTCATGCCGTTCAT |
| | Human NURR1 primer 5′: |
| (SEQ ID NO.: 547) | |
| | CGCTTCTCAGAGCTACAGTTAC |
| | Human NURR1 primer 3′: |
| (SEQ ID NO.: 548) | |
| | TGGTGAGGTCCATGCTAAAC |
| | Human PUMA primer 5′: |
| (SEQ ID NO.: 549) | |
| | CCTGGAGGGTCCTGTACAATCT |
| | Human PUMA primer 3′: |
| (SEQ ID NO.: 550) | |
| | TCTGTGGCCCCTGGGTAAG |
| | Human TP53 primer 5′: |
| (SEQ ID NO.: 551) | |
| | GTACCACCATCCACTACAACTAC |
| | Human TP53 primer 3′: |
| (SEQ ID NO.: 552) | |
| | CACAAACACGCACCTCAAAG |

Protein isolation and western-blot analysis. Cells treated with PBS or monoclonal antibodies against TNFα were harvested and lysed in the following lysis buffer (RIPA buffer, 1:1000 Halt™ Protease, and Phosphatase Inhibitor cocktail (Thermo Fisher Scientific)). After cells are resuspended in a lysis buffer, cells are sonicated for 3×30 seconds at 4° C. Supernatant was collected upon 15 minutes centrifugation >15,000 rpm at 4° C. and protein concentration was measured using Precision Red Advanced Protein Assay (Cytoskeleton). Equal amounts of protein (20 micrograms) were boiled in NuPAGE LSD sample buffer (Invitrogen) at 95° C. for 5 minutes and separated using NuPAGE 4%-12% Bis-Tris Protein Gel (Invitrogen) in NuPAGE MES SDS Running Buffer (Invitrogen). Proteins were electrophoretically transferred to a nitrocellulose membrane (Thermo Fisher Scientific) with NuPAGE Transfer Buffer (Invitrogen). Blots were blocked for 60 minutes at RT in 5% nonfat milk (Cell Signaling) in TBS-T+ and incubated with respective primary antibody at 4° C. overnight. The following primary antibodies were used: mouse-anti-GAPDH (6C5) (Santa Cruz, 1:1,000); mouse anti-p53 (DO-1) (Santa Cruz, 1:1,000); rabbit anti-tyrosine hydroxylase (Pel Freeze, 1:1000). Primary antibodies were detected using the secondary anti-rabbit IgG HRP-linked (Cell Signaling, 1:1,000) or anti-mouse IgG HRP-linked (Cell Signaling, 1:1,000) with the SuperSignal™ West Femto Chemiluminescent substrate (Thermo Fisher Scientific).

RNA-Seg 1 day post transplantation. One day after the intracranial injection of mDA neurons sorted by NURR1-GFP⁺ signal, the mice were euthanized, and the injection site was grossly dissected and processed for papain dissociation (Worthington). Dissociated xenograft samples along with in vitro cultured neurons (day 1 in vitro) were simultaneously subject to FACS for re-isolating dopamine neurons based on the endogenous reporter signal. Total RNA was extracted in TRIzol (Invitrogen) according to the manufacturer's instructions. RNAseq libraries of polyadenylated RNA were prepared using the TruSeq Stranded mRNA Library Prep Kit (Illumina) according to the manufacturer's instructions and sequenced on an Illumina NextSeq 500 platform. The resulting single-end reads were checked for quality (FastQC v0.11.5) and processed RNAseq libraries of polyadenylated RNA were prepared using the TruSeq Stranded mRNA Library Prep Kit (Illumina) according to the manufacturer's instructions and sequenced on an Illumina NextSeq 500 platform. The filtered reads were mapped to human reference genome hg19 using STAR aligner (version 2.5.0a). The expression data at the coding sequence were quantified using the GENCODE version 38 transcriptome and HTSeq version 1.99.2 (Anders et al., 2015, Bioinformatics, 31 (2): 166-9). Differential expression analysis was done with the negative binomial statistical model using DESeq2 version 1.30.1 (Love et al., 2014, Genome Biol 15 (12): 550). Enrichment analysis with the various reference data was done using clusterProfiler version 4.2.1 (Yu et al., 2012, OMICS 16 (5): 284-7; Wu et al., 2021, Innovation (N Y) 2 (3): 100141).

Single-cell transcriptome sequencing. Single cell suspensions were stained with Trypan blue, and the Countess II Automated Cell Counter (ThermoFisher) was used to assess both cell number and viability. Following QC, the single cell suspension was loaded onto Chromium Chip B (10× Genomics PN 2000060) and GEM generation, cDNA synthesis, cDNA amplification, and library preparation of 3,900-5,300 cells proceeded using the Chromium Single Cell 3′ Reagent Kit v3 (10× Genomics PN 1000075) according to the manufacturer's protocol. cDNA amplification included 12 cycles and 88-99 ng of the material was used to prepare sequencing libraries with 12 cycles of PCR. Indexed libraries were pooled equimolar and sequenced on a NovaSeq 6000 in a PE28/91 paired end run using the NovaSeq 6000 SI Reagent Kit (100 cycles) (Illumina). An average of 94 million paired reads was generated per sample.

Single-cell analysis. The samples underwent 10× chromium Single Cell 3′ v3 processing. The reads were aligned to human GRCh38 (GENCODE v32/Ensembl 98) using Cell Ranger 5.0.0. The resulting filtered count matrix was further filtered for cells with i) minimum 1000 UMI counts, ii) 500≤gene counts≤7000, iii) and mitochondrial gene percentage of less than 25%. Normalization by deconvolution in scran version 1.22.1 was performed and the signal from the gene expression related to the cell cycle was regressed out as directed by Seurat version 4.1. The default 2000 highly variable genes were selected, and the first 50 principal components were extracted from the cell cycle-regressed matrix. Subsequently, the shared nearest neighbors were calculated from the principal components using buildSNNGraph of R software scran using the k parameter of 40. Seven clusters were identified and using the walktrap algorithm, with the function cluster_walktrap of R implementation of the igraph package version 1.3.5. The uniform manifold approximation and projection (UMAP) was performed. Differential gene expression was performed via the Seurat package using MAST. Cluster annotation was performed via clusterProfiler package version 4.2.2, and differential expression visualization using EnhancedVolcano version 1.12.0.

Stereological analysis. For short-term survival studies, unbiased stercological counts of NURR1::H2B-GFP DA neurons within the striatum (AP+0.5, ML+/−1.8, DV −3.4 to −3.3 from dura) were performed using stereological principles and analyzed with StereoInvestigator software (Microbrightfield, Williston, VT, USA), as previously described. For long-term behavioral studies, unbiased stereological counts of TH positive DA neurons within the striatum were performed. The tissue was embedded in O.C.T. or Neg-50 and sections are sliced at 30 μm. Optical fractionator sampling was carried out with an Olympus BX61 microscope equipped with a motorized stage and Olympus 40× objective lens objective. Graft region was outlined on the basis of NURR1::GFP immunolabeling, with reference to a coronal atlas of the mouse brain. Every 3rd-10th section (depending on the total thickness of the graft) from the beginning of the graft to the end of the graft was randomly and systematically selected for analysis. For each tissue section analyzed, section thickness was assessed in each sampling site and guard zones of 1 μm were used at the top and bottom of each section. Pilot studies were used to determine suitable counting frame and sampling grid dimensions prior to counting to achieve enough statistical power and low Gunderson coefficient variance. The following stereological parameters were used in the final study: for optical fractionator probe, 65 μm×65 μm optical dissector, 100 μm×100 μm (or 10% of ROI) SRS, 20 μm optical dissector height and 1 μm guard zone; for cavalier estimator probe, 50 μm×50 μm grid spacing, 0-degree grid rotation, and 30 μm section cut thickness. For analysis, at least 2-8 sections were evaluated for analysis. Gundersen coefficients of error for all conditions were less than 0.1. Stereological estimations were performed with the same parameters for all experimental conditions, p53 KO vs. WT (NURR1::GFP sort) or TNFα monoclonal antibodies treatment vs. PBS (CD sort).

High throughput cell surface marker screen and enrichment of dopamine neuron with using cell surface markers. Dopamine neuron differentiated cells at day 25 from the NURR1::GFP reporter hESC were single cell suspended in flow cytometer staining buffer (PBS containing 2% bovine serum albumin). The cells were stained with 387 cell surface (CD) markers (0.2M cells per a CD marker) for 30 min on ice in the dark. After 3 times washing with PBS, cells were co-stained with DAPI. All staining for the screen was done in 96 well plates. Data collection using a flow cytometer to identify CD markers to enrich GFP positive population was performed by the MSKCC Flow Cytometry core facility. For enrichment of dopamine neuron with using CD markers, 49c and 184, day 25 cells were

stained with 49e and/or 184, followed by isolation of 49e weak, 49e weak and 174 high, and 49e weak and 184 high expressed cells via FACS at the MSKCC Flow Cytometry core facility.

Transcriptome sequencing. After RiboGreen quantification and quality control by Agilent BioAnalyzer, 0.5-1 ng total RNA with RNA integrity numbers ranging from 6.1 to 10 underwent amplification using the SMART-Seq v4 Ultra Low Input RNA Kit (Clonetech catalog #63488), with 12 cycles of amplification. Subsequently, 1.6-10 ng of amplified cDNA was used to prepare libraries with the KAPA Hyper Prep Kit (Kapa Biosystems KK8504) using 8 cycles of PCR. Samples were barcoded and run on a HiSeq 4000 or NovaSeq 6000 in a PE50 run, using the HiSeq 3000/4000 SBS Kit or NovaSeq 6000 SI Reagent Kit (100 Cycles) (Illumina). An average of 56 million paired reads were generated per sample and the percent of mRNA bases per sample ranged from 48% to 76%.

Quantification and Statistical analysis. N=3 independent biological replicates were used for all experiments unless otherwise indicated. n.s. indicates a non-significant difference. P-values were calculated by unpaired two-tailed Student's t-test unless otherwise indicated. * p<0.05, ** p<0.01 and *** p<0.001.

### Results

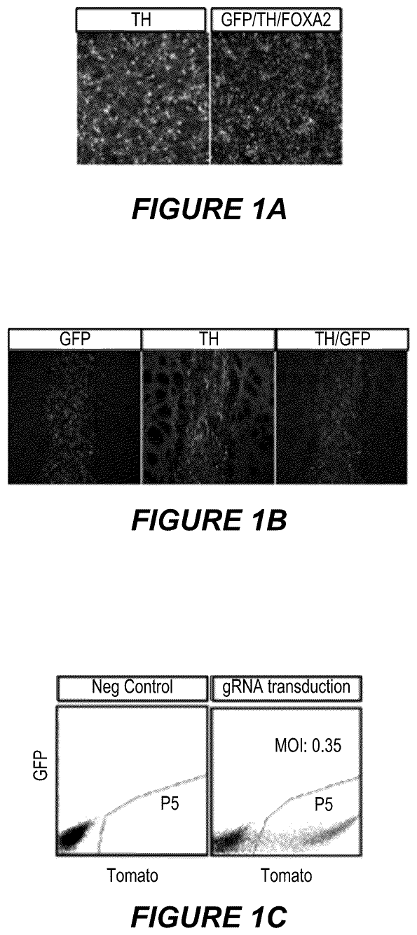

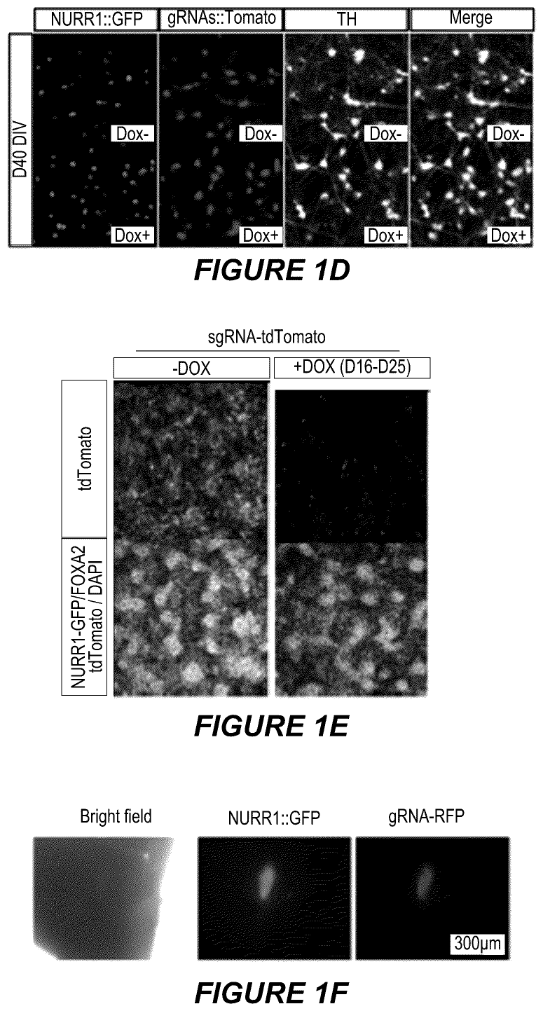

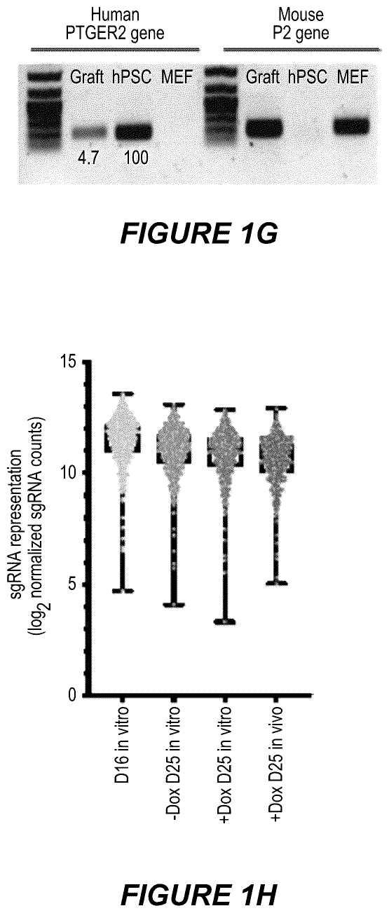

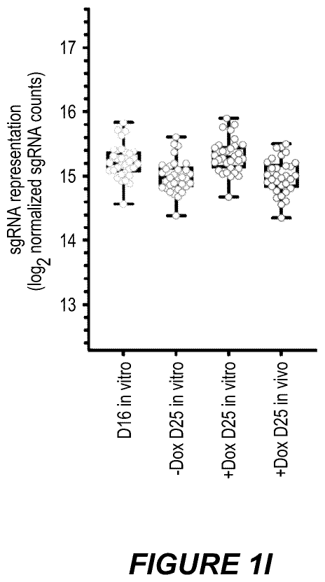

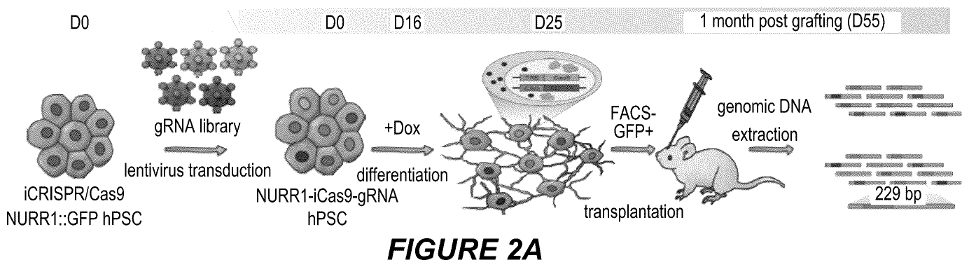

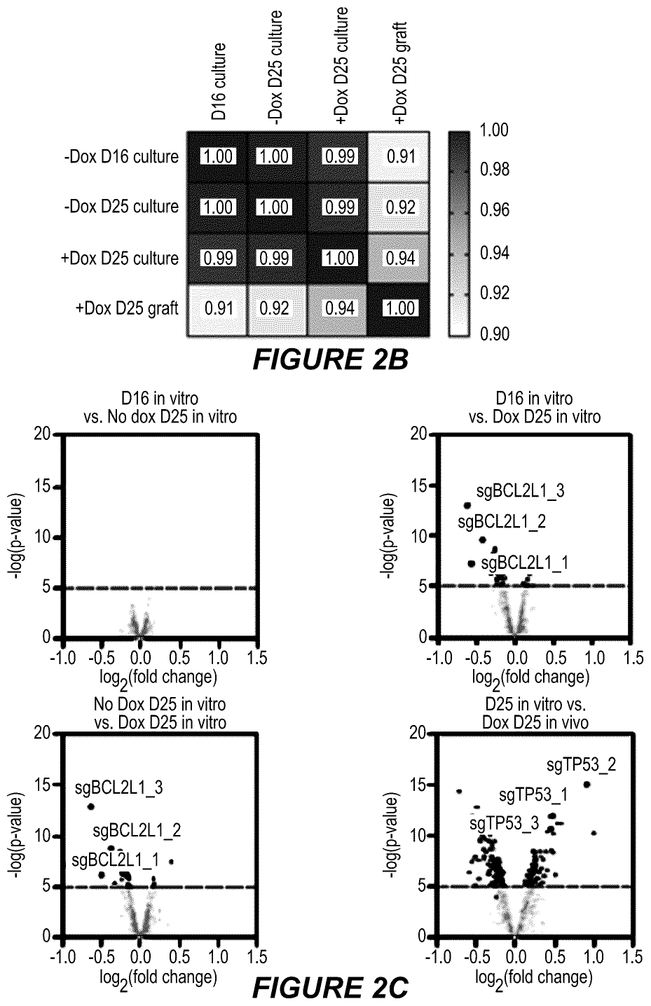

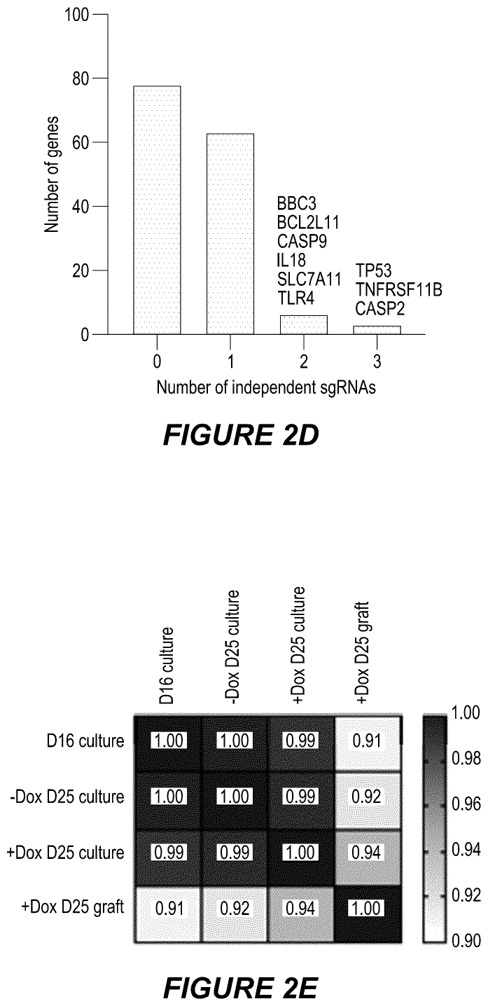

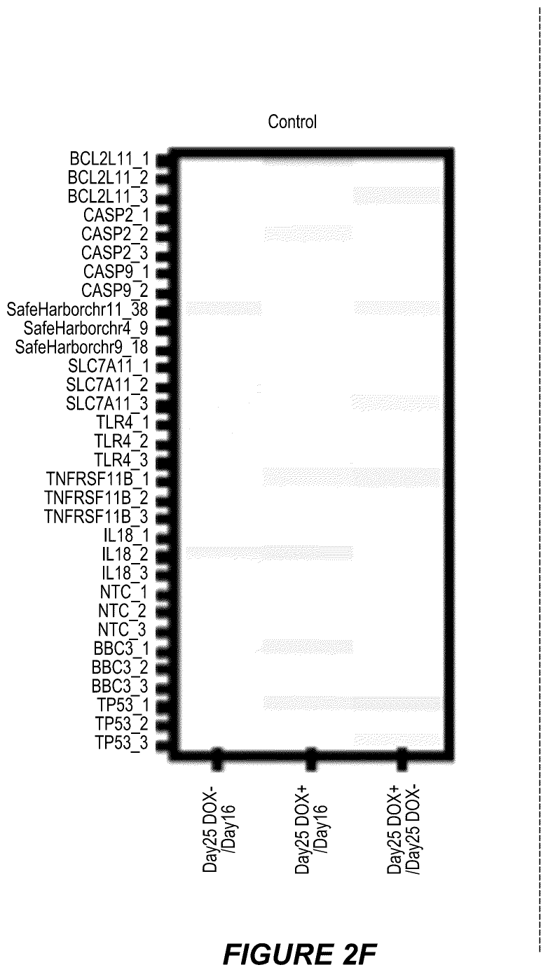

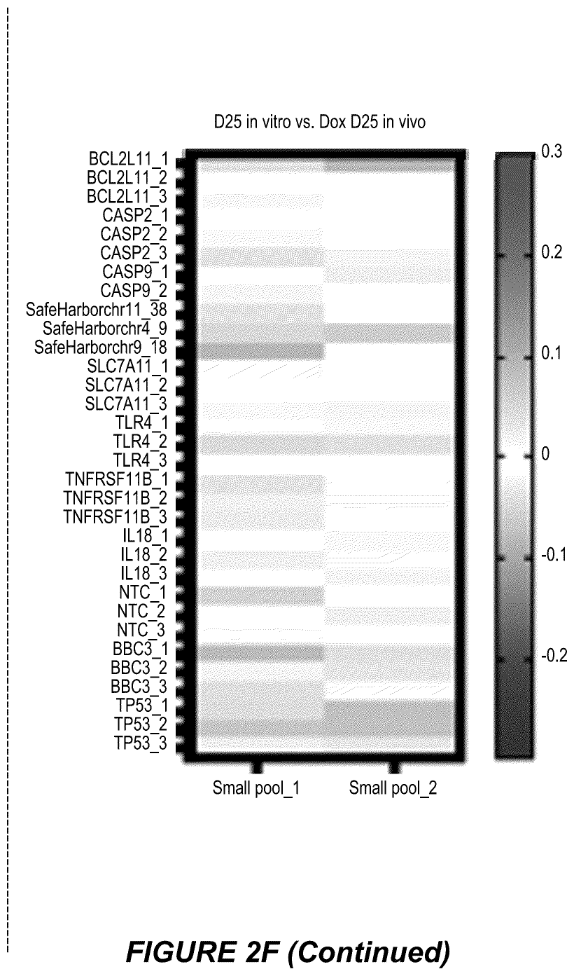

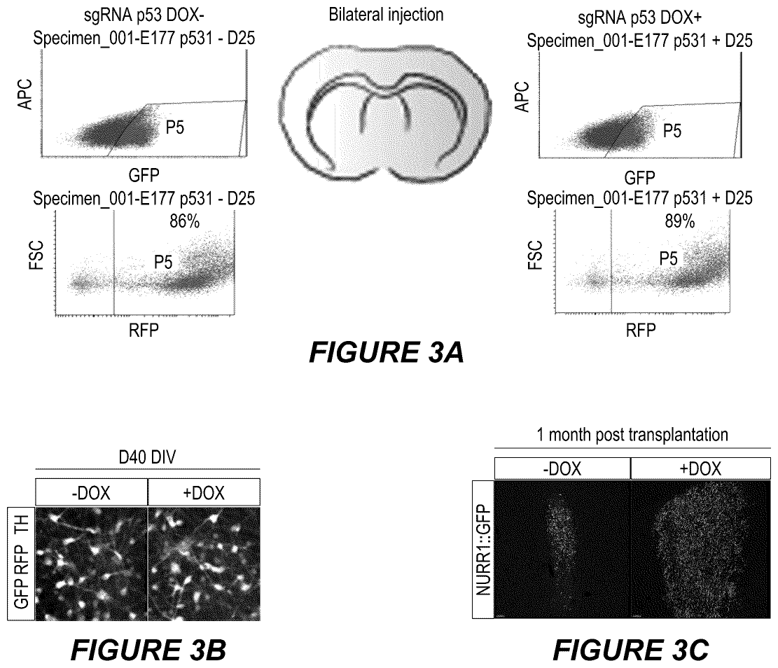

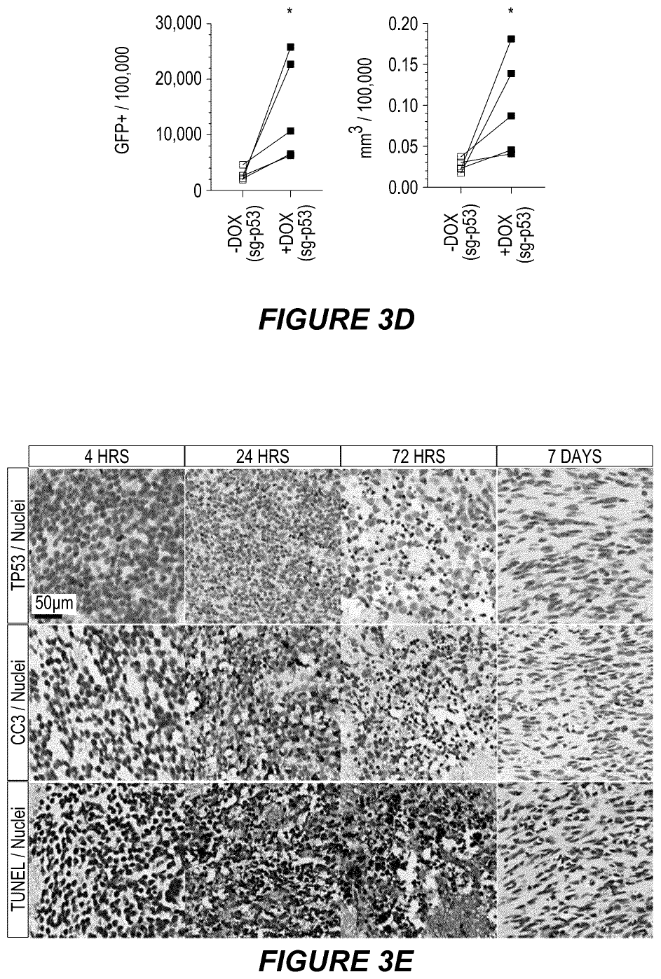

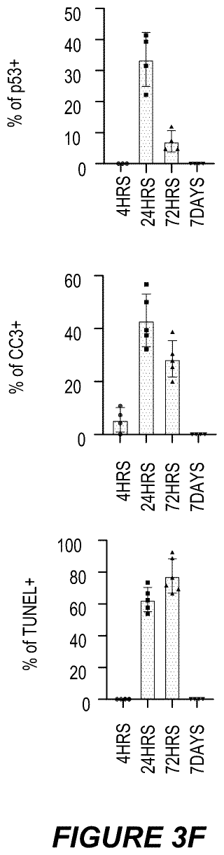

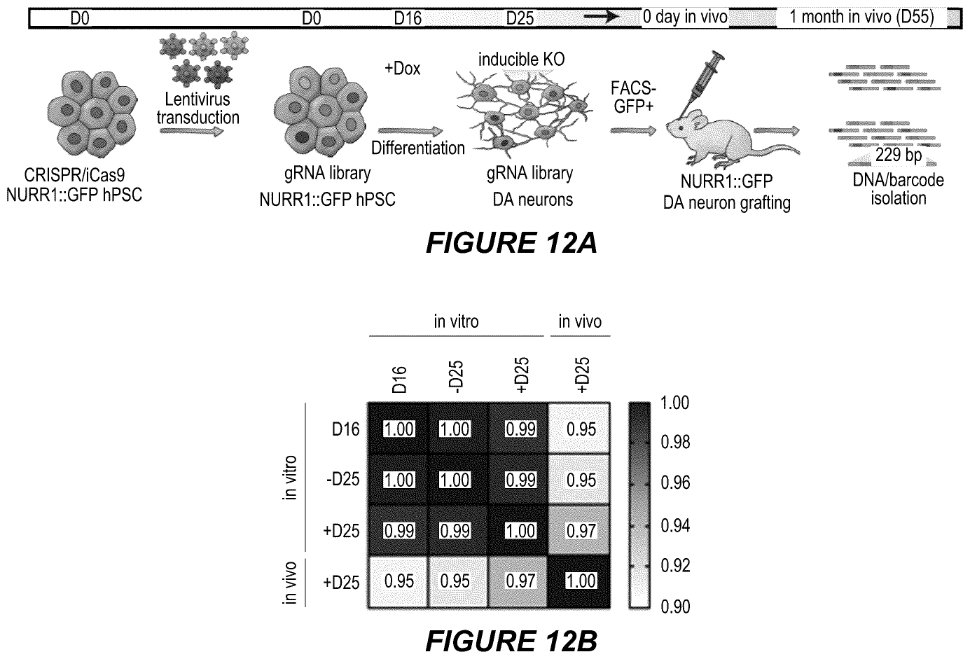

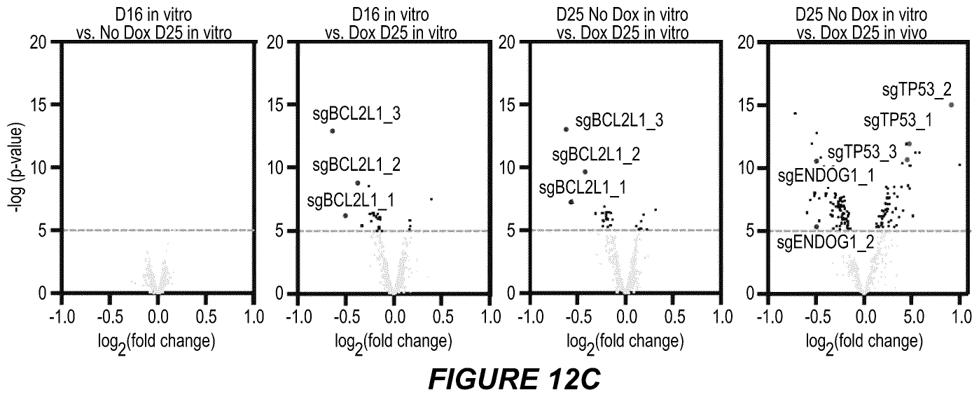

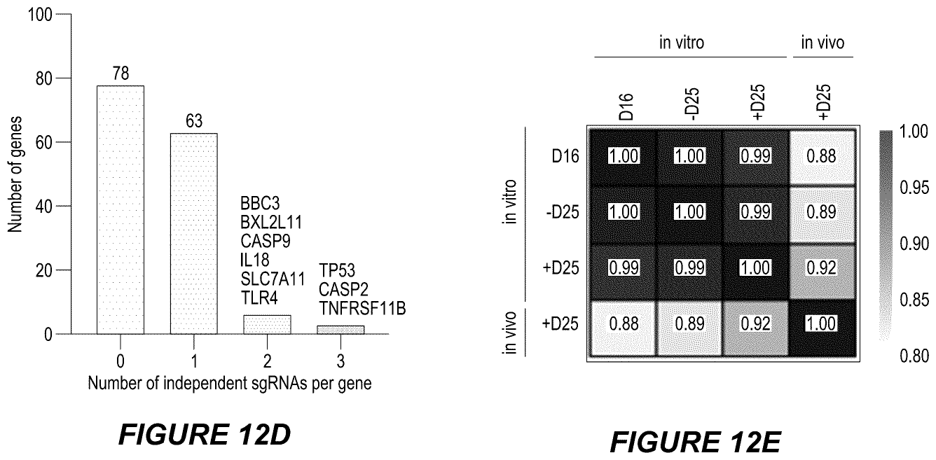

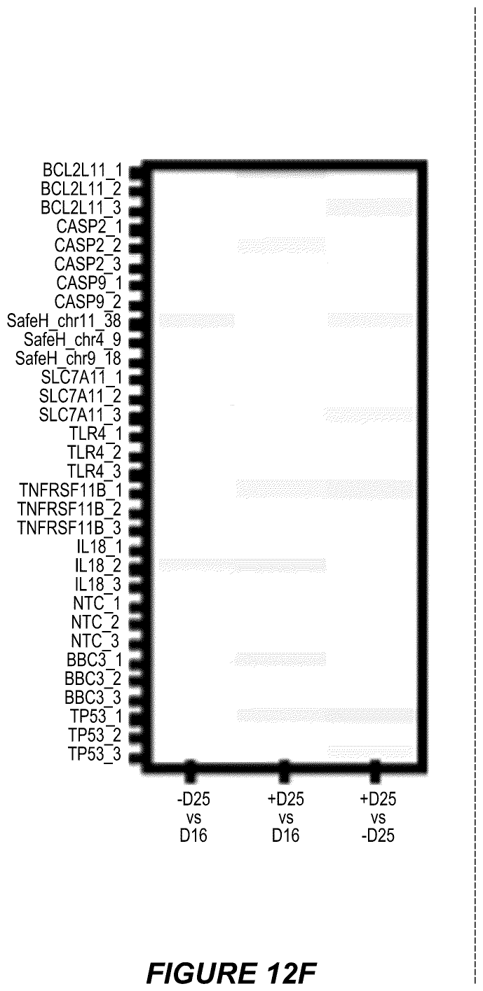

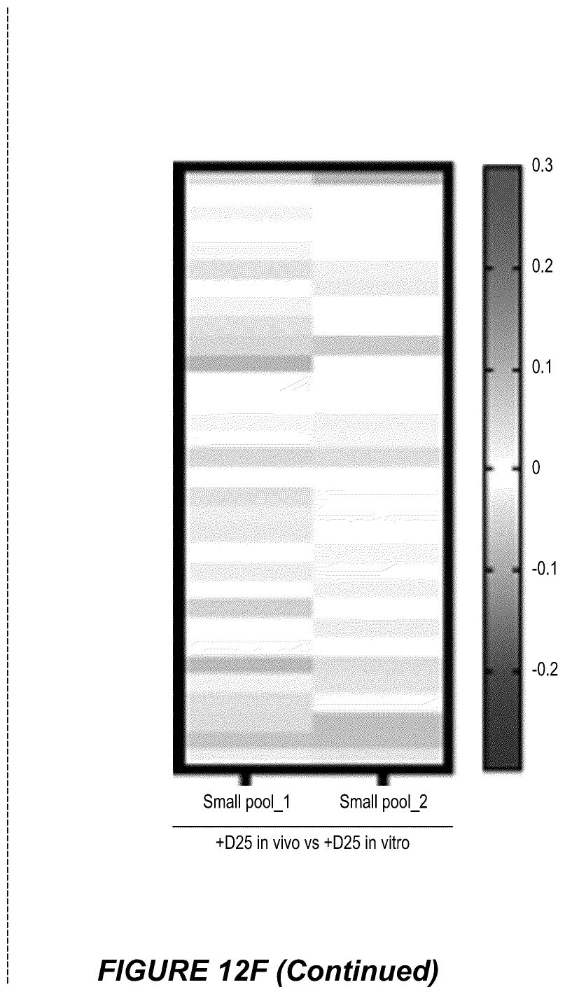

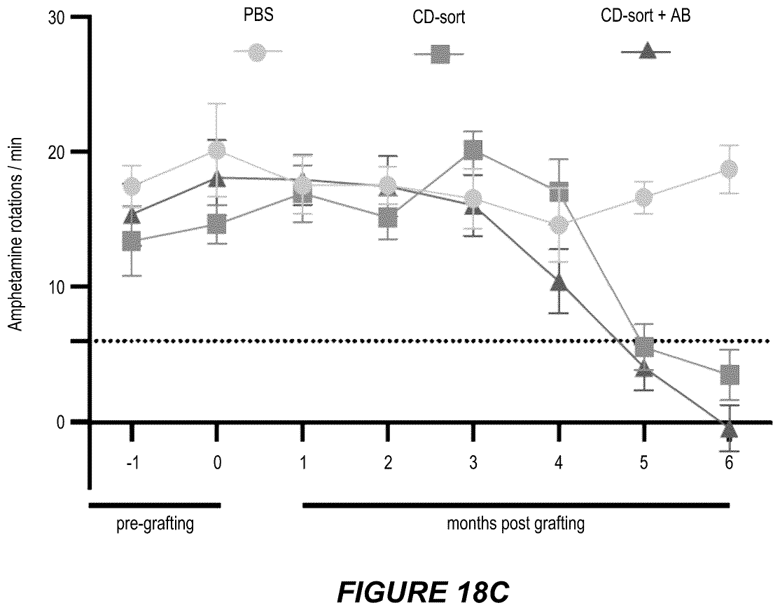

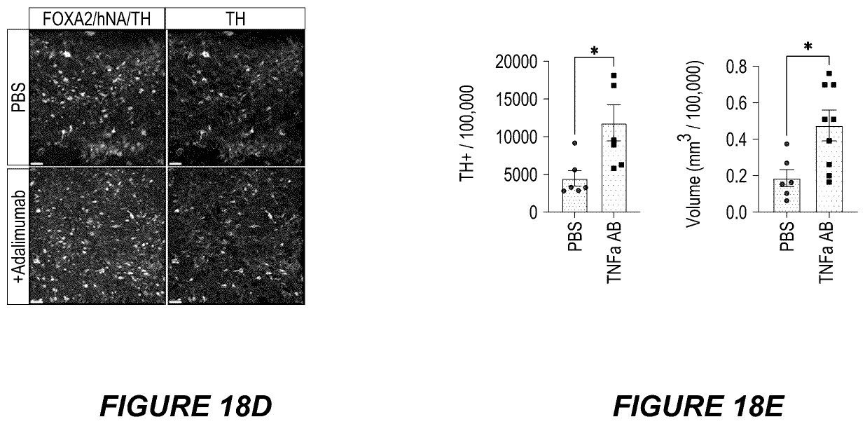

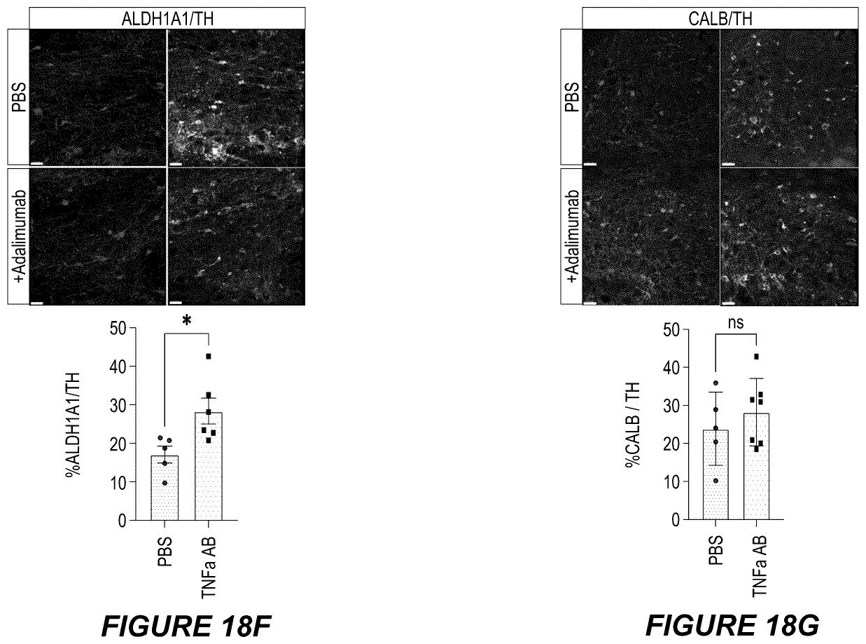

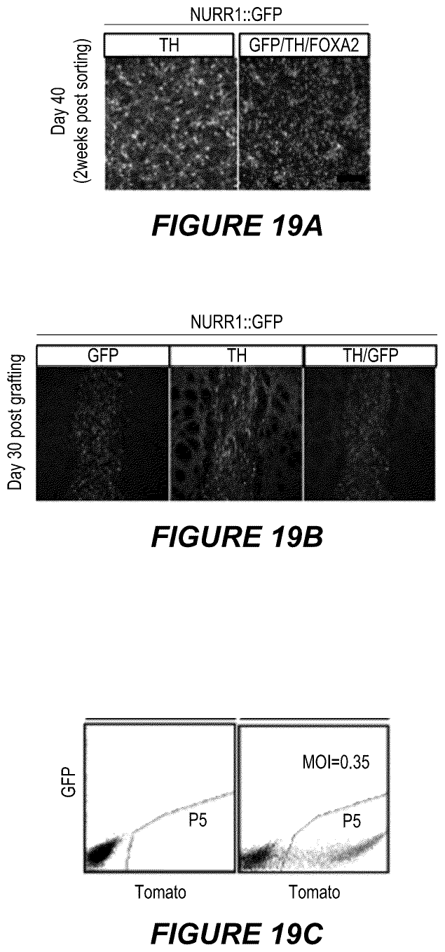

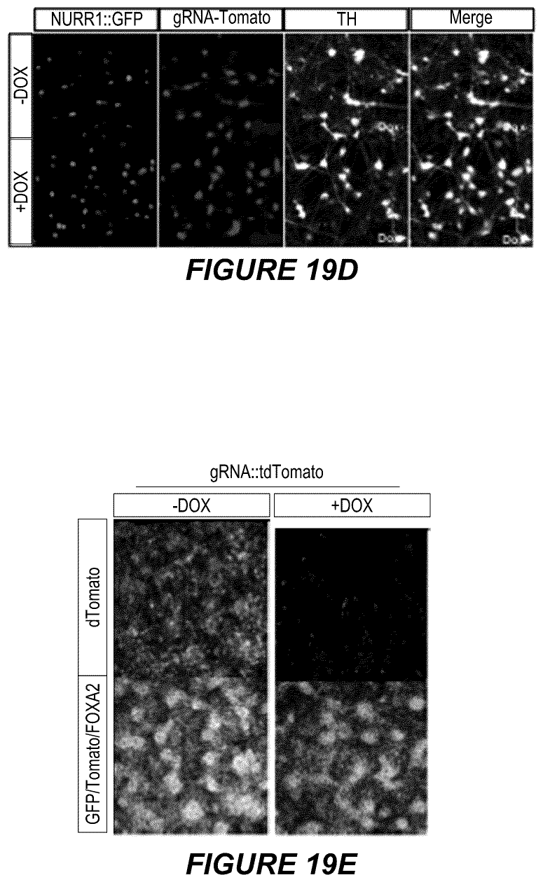

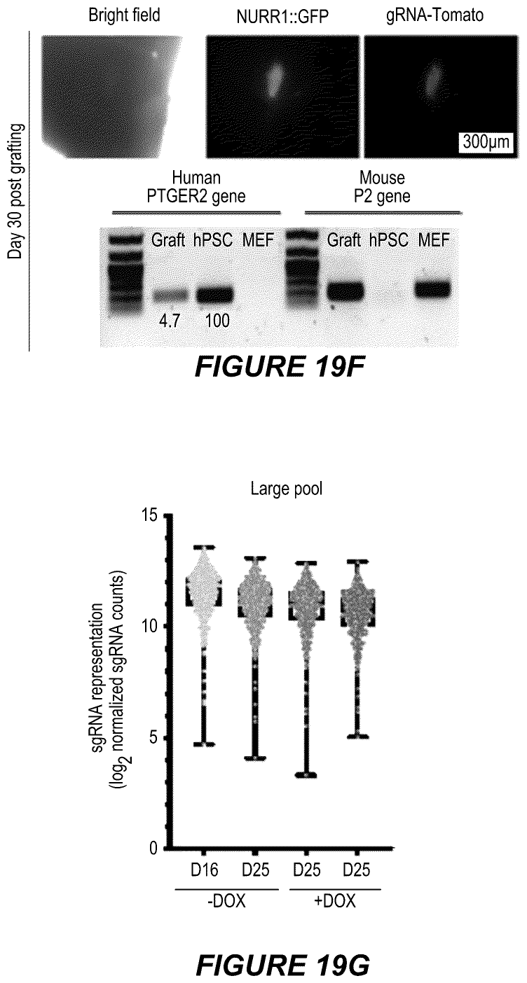

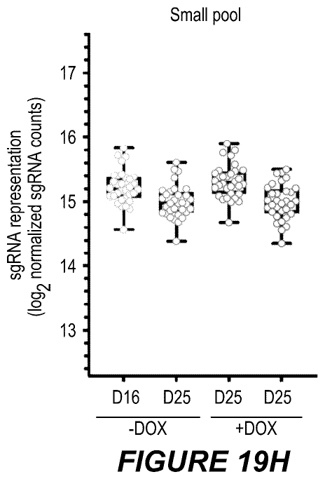

In vivo CRISPR/Cas9 screens discovered a regulator of post-mitotic dopamine neuron death. An in vivo CRISPR-Cas9 screen was developed to systematically address intrinsic factors that restrict the survival of hPSC derived post-mitotic dopamine neurons. Previously, it was demonstrated that NURR1 can serve as a reliable marker to denote early post-mitotic dopamine neurons derived from hPSCs under the floor-plate differentiation paradigm (Riessland et al., 2019, Cell Stem Cell 25, 514-530 e518). FACS-based purification of NURR1::GFP positive cells yielded nearly pure dopamine neurons populations in culture (Riessland et al., 2019, Cell Stem Cell 25, 514-530 e518) (FIG. 1A; FIG. 19A), and gave rise to a highly homogenous and dense dopamine neuron graft in vivo, marked by TH, albeit overall poor survival rates less than ˜5% derived from an endogenous NURR1::GFP hPSC reporter line (FIGS. 1B and 1G; FIG. 3D; FIGS. 19B and 19F). Using this established NURR1::H2B-GFP reporter hPSC, the presently disclosed subject matter further engineered inducible Cas9 (iCas9) hPSC lines, such as doxycycline-inducible expression of Cas9, integrated into a safe harbor locus, an AAVS locus, through TALEN-mediated gene targeting, (iCas9/NURR1::H2B-GFP hPSC line) (FIG. 2A; FIG. 12A). To determine the efficiency of iCas9 for gene knock-out in the presently disclosed culture system, a sgRNA targeting tdTomato was stably incorporated in the iCas9/NURR1::H2B-GFP hPSC line. Upon doxycycline treatment from day 16 to day 25 during dopamine neuron differentiation using clinical-grade protocol, efficient ablation of the tdTomato signal (FIG. 1E; FIG. 19E) was observed without disrupting dopamine neuron induction as shown by NURR1 and FOXA2 expressions, which allows a CRISPR/Cas9 loss-of-function pool screen in hPSC-derived post-mitotic dopamine neurons.

Next, a pooled-lentiviral custom-design library of 550 sgRNAs targeting a total of 150 genes (3 sgRNAs per gene) related to cell death pathways, such as apoptosis, necroptosis, pyroptosis, ferroptosis, and autophagy as well as 50 non-targeting and 50 safe harbor control guides were stably introduced into the iCas9/NURR1::H2B-GFP hPSC line (iCas9/NURR1::H2B-GFP/library) (Table 1 and Table 2).

TABLE 1

| | |
|---|---|
| APOPTOSIS | FAS |
| | FASLG |
| | TNFRSF10A |
| | TNFRSF10B |
| | TNFRSF10C |
| | TNFRSF10D |
| | TNFRSF11B |
| | TNFSF10 |
| | TNFRSF1A |
| | TNF |
| | FADD |
| | CFLAR |
| | CASP1 |
| | CASP2 |
| | CASP3 |
| | CASP4 |
| | CASP5 |
| | CASP6 |
| | CASP7 |
| | CASP8 |
| | CASP9 |
| | CASP10 |
| | CASP14 |
| | NAIP |
| | BIRC2 |
| | BIRC3 |
| | XIAP |
| | BIRC5 |
| | BIRC6 |
| | BIRC7 |
| | BCL2 |
| | MCL1 |
| | BCL2L1 |
| | BCL2L2 |
| | BCL2A1 |
| | BCL2L10 |
| | BAX |
| | BAK1 |
| | BOK |
| | BID |
| | BCL2L11 |
| | BMF |
| | BAD |
| | BIK |
| | HRK |
| | PMAIP1 |
| | BNIP3 |
| | BNIP3L |
| | BCL2L14 |
| | BBC3 |
| | BCL2L12 |
| | BCL2L13 |
| | APAF1 |
| | CYCS |
| | DIABLO |
| | HTRA2 |
| | AIFM1 |
| | ENDOG |
| | CARD8 |
| | GZMB |
| | CARD6 |
| | NOX5 |
| NECROSIS | RIPK1 |
| | RIPK3 |
| | MLKL |
| | CD40LG |
| | CD70 |
| | PARP2 |
| | FOXI1 |
| | UBR5 |
| | MPG |
| | CA9 |
| | SLC25A15 |
| | SIRTS |
| | NPEPL1 |
| | DSC1 |
| | CD40 |
| | COL4A3BP |
| | DNM1L |
| | PGAM5 |
| | PARP1 |
| | PTPA(PP2A) |
| | CYLD |
| | JUN |
| | GRB2 |
| | IFNA1 |
| | IFNA7 |
| | IFNA13 |
| | IRGM |
| PYROPTOSIS | NAIP |
| | NLRP1 |
| | NLRC4 |
| | AIM2 |
| | NLRP1 |
| | NLRP3 |
| | PYCARD |
| | GASDMD |
| FERROPTOSIS | VDAC2 |
| | VDAC3 |
| | NOX4 |
| | TFRC |
| | CARS |
| | IREB2 |
| | SLC11A2 |
| | CS |
| | ACSF2 |
| | TP53 |
| | GPX4 |
| | SLC7A11 |
| | HSPB1 |
| | NEF212 |
| AUTOPHAGY | ULK1 |
| | ULK2 |
| | ATG2A |
| | ATG2B |
| | ATG3 |
| | ATG4A |
| | ATG4B |
| | ATG4C |
| | ATG4D |
| | ATG5 |
| | BECN1 |
| | ATG7 |
| | ATG9A |
| | ATG9B |
| | ATG10 |
| | ATG12 |
| | ATG13 |
| | ATG14 |
| | ATG16L1 |
| | ATG16L2 |
| | RB1CC1 |
| | WIPI1 |
| | WIPI2 |
| | SNX30 |
| | SNX4 |
| | ATG101 |
| | MAP1LC3A |
| | PIK3C3 |
| | TLR7 |
| | TLR4 |
| | DDX58 |
| | IFIH1 |
| | MAVS |
| | IL1B |
| | IL18 |
| | IL4 |

TABLE 2

| | | SEQ |
|---|---|---|
| sgRNA Target | | ID |
| Sequence | Target Gene | NO. |
| ATGGTCTCCACGCCCATCGG | ACSF2_1 | 1 |
| ACGTAGCTGAGGCCTCCGAT | ACSF2_2 | 2 |
| CGACCAAGGCCTCTCGTTCT | ACSF2_3 | 3 |
| GGAATACTCACCAGATAACG | AIFM1_1 | 4 |
| TCCATCCGGGCTCGGGATCC | AIFM1_2 | 5 |
| ATCCCGAGCCCGGATGGATC | AIFM1_3 | 6 |
| TTGAAGCGTGTTGATCTTCG | AIM2_1 | 7 |
| TTCACGTTTGAGACCCAAGA | AIM2_2 | 8 |
| TCAGCGGGACATTAACCTTT | AIM2_3 | 9 |
| AGCATTGTAGAATGATACGT | APAF1_1 | 10 |
| GTTAGTGGAATAACTTCGTA | APAF1_2 | 11 |
| CCAGGATGGGTCACCATACA | APAF1_3 | 12 |
| CTATAAGATGCGACTGCTAC | ATG10_1 | 13 |
| GACACTATTACGCAACAGGT | ATG10_2 | 14 |
| TGAATTCTGCACAATAACGT | ATG10_3 | 15 |
| GCCCACGGTGCCAATGGAGT | ATG101_1 | 16 |
| GTGCCCTGCGCAAGGTTGTT | ATG101_2 | 17 |
| TGCGCAACTCTGGTGGCGAT | ATG101_3 | 18 |
| CTCCCCAGAAACAACCACCC | ATG12_1 | 19 |
| CCTCCAGCAGCAATTGAAGT | ATG12_2 | 20 |
| AGCAGGTTCCTCTGTTCCCG | ATG12_3 | 21 |
| GGGTATATCCAAACTCGTCA | ATG13_1 | 22 |
| AGGGTATATCCAAACTCGTC | ATG13_2 | 23 |
| TTTACCCAATCTGAACCCGT | ATG13_3 | 24 |
| TCTACTTCGACGGCCGCGAC | ATG14_1 | 25 |
| GGCGATTTCGTCTACTTCGA | ATG14_2 | 26 |
| CGATGCGGAGGGGCTGTACG | ATG14_3 | 27 |
| CATCATGTCCGGGACTAAAT | ATG16L1_1 | 28 |
| CGGGACTAAATTGGCAAACC | ATG16L1_2 | 29 |
| ATTAAGCCGATTGGCTTCCT | ATG16L1_3 | 30 |
| TACGCAAAAGGCGCTTTTCC | ATG16L2_1 | 31 |
| CGGACCCACATACACTTACC | ATG16L2_2 | 32 |
| GCAACCTGCGCAACGAGCGC | ATG16L2_3 | 33 |
| CGTGACATCTCGGAGACCGC | ATG2A_1 | 34 |
| GTGACATCTCGGAGACCGCC | ATG2A_2 | 35 |
| ATCTCGGAGACCGCCGGGCC | ATG2A_3 | 36 |
| ATGGACTCCGAAAACGGCCA | ATG2B_1 | 37 |
| GAAACTGCTGACGAATCCTC | ATG2B_2 | 38 |
| GTGCAGATTGGACGGTTAAT | ATG2B_3 | 39 |
| TTATAGTGCCGTGCTATAAG | ATG3_1 | 40 |
| TGTTTGCACCGCTTATAGCA | ATG3_2 | 41 |
| ACAACCATAATCGTGGAGTC | ATG3_3 | 42 |
| ATTGGTGGGTATTCGTAGGT | ATG4A_1 | 43 |
| GGATCCTTCAGTTGCATTGG | ATG4A_2 | 44 |
| TTAGGATGTTCATTCGCTGT | ATG4A_3 | 45 |
| AGCAAACCGGAGAGTGTCGT | ATG4B_1 | 46 |
| TCAGAGCCCGTTTGGATACT | ATG4B_2 | 47 |
| TCCTGTCGATGAATGCGTTG | ATG4B_3 | 48 |
| AGAGTCGGGATGTACAATAG | ATG4C_1 | 49 |
| ATAGAGGATCACGTAATTGC | ATG4C_2 | 50 |
| ATTGTACATCCCGACTCTGC | ATG4C_3 | 51 |
| CCTCGCCCTCGAAACGGTAG | ATG4D_1 | 52 |
| TGGCCGCCGCTACCGTTTCG | ATG4D_2 | 53 |
| CCCGGCGGTATGTGAGCCAC | ATG4D_3 | 54 |
| AACTTGTTTCACGCTATATC | ATG5_1 | 55 |
| GAGTGAACATCTGAGCTACC | ATG5_2 | 56 |
| TCCGATTGATGGCCCAAAAC | ATG5_3 | 57 |
| CTAGGACGTTGATGGTAAGT | ATG7_1 | 58 |
| GAAGCTGAACGAGTATCGGC | ATG7_2 | 59 |
| CTTGAAAGACTCGAGTGTGT | ATG7_3 | 60 |
| CTGTTGGTGCACGTCGCCGA | ATG9A_1 | 61 |
| CCTGTTGGTGCACGTCGCCG | ATG9A_2 | 62 |
| CCTCGGCGACGTGCACCAAC | ATG9A_3 | 63 |
| CGACGAGGACGTGCTAGCCG | ATG9B_1 | 64 |
| TAGCACGTCCTCGTCGTAGA | ATG9B_2 | 65 |
| TGTGCTCACCGTCTACGACG | ATG9B_3 | 66 |
| CTTTCGGGGCCGCTCGCGCT | BAD_1 | 67 |
| GCTATGGCCGCGAGCTCCGG | BAD_2 | 68 |
| GAGCGCGAGCGGCCCCGAAA | BAD_3 | 69 |
| CATGAAGTCGACCACGAAGC | BAK1_1 | 70 |
| GCATGAAGTCGACCACGAAG | BAK1_2 | 71 |
| GCAGGTGAGCTACAACCGCT | BAK1_3 | 72 |
| GCGAGTGTCTCAAGCGCATC | BAX_1 | 73 |
| CAAGCGCATCGGGGACGAAC | BAX_2 | 74 |
| TCGGAAAAAGACCTCTCGGG | BAX_3 | 75 |
| TCAACGCACAGTACGAGCGG | BBC3_1 | 76 |
| GCCGCTCGTACTGTGCGTTG | BBC3_2 | 77 |
| CAACGCACAGTACGAGCGGC | BBC3_3 | 78 |
| GGGGCCGTACAGTTCCACAA | BCL2_1 | 79 |
| TCAAACAGAGGCCGCATGCT | BCL2_2 | 80 |
| AAGCGTCCCCGCGCGGTGAA | BCL2_3 | 81 |
| CTTATAGGTATCCACATCCG | BCL2A1_1 | 82 |
| CCTTATAGGTATCCACATCC | BCL2A1_2 | 83 |
| TTGAAGACGGCATCATTAAC | BCL2A1_3 | 84 |
| GTTTGAACTGCGGTACCGGC | BCL2L1_1 | 85 |
| AGTTTGAACTGCGGTACCGG | BCL2L1_2 | 86 |
| ACGAGTTTGAACTGCGGTAC | BCL2L1_3 | 87 |
| CGGGAACCGCTTCGAGCTGG | BCL2L10_1 | 88 |
| CCGGTGAATCTGCCGTAACC | BCL2L10_2 | 89 |
| CTGCCGTAACCTGGCGGCCG | BCL2L10_3 | 90 |
| GCCCAAGAGTTGCGGCGTAT | BCL2L11_1 | 91 |
| TCCAATACGCCGCAACTCTT | BCL2L11_2 | 92 |
| CTCCAATACGCCGCAACTCT | BCL2L11_3 | 93 |
| GGGCCGTCCCATCGGCTTTT | BCL2L12_1 | 94 |
| AAGCCGATGGGACGGCCCGC | BCL2L12_2 | 95 |
| AGCCGATGGGACGGCCCGCT | BCL2L12_3 | 96 |
| GAGGACGCCATTGAATTGGC | BCL2L13_1 | 97 |
| ATCCGGAACGGTATCCGGAG | BCL2L13_2 | 98 |
| CTGACATCCGGAACGGTATC | BCL2L13_3 | 99 |
| TGGCCGTGACGTCTATTACA | BCL2L14_1 | 100 |
| GATCCAGCAGCACGGTGGAT | BCL2L14_2 | 101 |
| GGCCGTGACGTCTATTACAA | BCL2L14_3 | 102 |
| TCTATACGGGGACGGGGCCC | BCL2L2_1 | 103 |
| CACAGCTCTATACGGGGACG | BCL2L2_2 | 104 |
| CGGAGTTCACAGCTCTATAC | BCL2L2_3 | 105 |
| TTACGGAAACCATTCATATC | BECN1_1 | 106 |
| GCGTTATGCCCAGACGCAGC | BECN1_2 | 107 |
| GCGTCTGGGCATAACGCATC | BECN1_3 | 108 |
| TGGAACCGTTGTTGACCTGA | BID_1 | 109 |
| ACATCATCCGGAATATTGCC | BID_2 | 110 |
| AGAACCTACGCACCTACGTG | BID_3 | 111 |
| GCCAAGAACCTCCATGGTCG | BIK_1 | 112 |
| TATGGAGGACTTCGATTCTT | BIK_2 | 113 |
| CGTAGATGAAAGCCAGACCC | BIK_3 | 114 |
| ATGTTTTGATACGAGGGACC | BIRC2_1 | 115 |
| TGTTTTGATACGAGGGACCT | BIRC2_2 | 116 |
| ATATTCAACTTTCCCCGCCG | BIRC2_3 | 117 |
| ACCCGGAAGTAATGAGTGTG | BIRC3_1 | 118 |
| ATTGAGCAATTGGGAACCGA | BIRC3_2 | 119 |
| CGGCAGTTAGTAGACTATCC | BIRC3_3 | 120 |
| ACTTACATGGGGTCGTCATC | BIRC5_1 | 121 |
| CCGGCCCACTGTAAGGATTA | BIRC5_2 | 122 |
| GCGCCGTGCCATCGAGCAGC | BIRC5_3 | 123 |
| GCATGCACTGCGACGCCGAC | BIRC6_1 | 124 |
| GCCTCATGTAGGCTATAGGT | BIRC6_2 | 125 |
| GCTTGAATTACCCGTTACAG | BIRC6_3 | 126 |
| AGTCAGCGGCCAGTCATAGA | BIRC7_1 | 127 |
| AGCCGGTGATGGTCCCACGC | BIRC7_2 | 128 |
| GCGCGGGGACGACCCCTGGA | BIRC7_3 | 129 |
| ACTCACCATAAAAGAGTCGC | BMF_1 | 130 |
| CGGCTTCATGTGCAGCAAGT | BMF_2 | 131 |
| CAAGTAGGCACGGGGGTTTA | BMF_3 | 132 |
| TCTGCGGCCGCGTCGCCCAT | BNIP3_1 | 133 |
| CCCGGCCGGAGGAACTCACA | BNIP3_2 | 134 |
| GTCGCGGCCAATGGGCGACG | BNIP3_3 | 135 |
| ACTAGGTGGGACGACATTGT | BNIP3L_1 | 136 |
| GCCCTTCGCCACAAGAAGAT | BNIP3L_2 | 137 |
| CCCGACACACCTGAGAGCTA | BNIP3L_3 | 138 |
| CGAGCGTGCCGCGCCGGTCC | BOK_1 | 139 |
| GATCTCGGCGGCGAAGACCG | BOK_2 | 140 |
| GGTCTTCGCCGCCGAGATCA | BOK_3 | 141 |
| GGCGGATATCCACCGGGGAC | CA9_1 | 142 |
| AGCTGGGGGCGGATATCCAC | CA9_2 | 143 |
| CTGCGCAACAATGGCCACAG | CA9_3 | 144 |
| TAGACACGTTAACTTCTCGG | CARD6_1 | 145 |
| CAGTACCTCCTCAATCTATG | CARD6_2 | 146 |
| TATGATGACCCAGAGCACGT | CARD6_3 | 147 |
| TTGTTAGCAAGGCGTCGCTG | CARD8_1 | 148 |
| TCCTGCTGCGGATCGCCAGT | CARD8_2 | 149 |
| TCCCACTGGCGATCCGCAGC | CARD8_3 | 150 |
| GCTTCGATGGACATTCACGG | CARS_1 | 151 |
| TGAGATGCGTCATAGACGGT | CARS_2 | 152 |
| TGATGTCAACAGGAGCGCGA | CARS_3 | 153 |
| ACCGAAGGTGATCATCATCC | CASP1_1 | 154 |
| GCATTGAGTTGTAGTATATC | CASP1_2 | 155 |
| TCCGCAAGGTAAGATGCTAG | CASP1_3 | 156 |
| AGCCGAGTCGTATCAAGGAG | CASP10_1 | 157 |
| TTCCTCTCCTTGATACGACT | CASP10_2 | 158 |
| AAGGAAGCCGAGTCGTATCA | CASP10_3 | 159 |
| AATGAGCAATCCGCGGTCTT | CASP14_1 | 160 |
| CAATCCGCGGTCTTTGGAAG | CASP14_2 | 161 |
| ACCCCACTGCCGAGGTATTG | CASP14_3 | 162 |
| TACTAAAGCTTCATACCGGT | CASP2_1 | 163 |
| TTACTAAAGCTTCATACCGG | CASP2_2 | 164 |
| AAGAAGCTCCGCCTGTCGAC | CASP2_3 | 165 |
| TACCCGGGTTAACCGAAAGG | CASP3_1 | 166 |
| TCTTACCCGGGTTAACCGAA | CASP3_2 | 167 |
| TGCCACCTTTCGGTTAACCC | CASP3_3 | 168 |
| TCCATATTCGGATGAGCTGC | CASP4_1 | 169 |
| TGCAGCTCATCCGAATATGG | CASP4_2 | 170 |
| GCCACTGAAAGATACATACG | CASP4_3 | 171 |
| GTCGACTTTTGATCCGTATT | CASP5_1 | 172 |
| TTGATCCGTATTAGGTACTA | CASP5_2 | 173 |
| GGGGCTCACTATGACATCGT | CASP5_3 | 174 |
| AAGATTGTCTCTATCTGCGC | CASP6_1 | 175 |
| CTGCAATGAGCTCGGCCTCG | CASP6_2 | 176 |
| CAAAGCAATCGGCATCTGCG | CASP6_3 | 177 |
| ATATGTAGGCACTCGGTCCC | CASP7_1 | 178 |
| TGTAGGCACTCGGTCCCGGG | CASP7_2 | 179 |
| GGTACAAACGAGGACCGGTC | CASP7_3 | 180 |
| TCCGGGATATATCTCGTTTG | CASP8_1 | 181 |
| GGGTCGATCATCTATTAATA | CASP8_2 | 182 |
| GCCTGGACTACATTCCGCAA | CASP8_3 | 183 |
| CCGCCGATCCGCTTCGTCCA | CASP9_1 | 184 |
| GAACAGCTCGCGGCTCAGCA | CASP9_2 | 185 |
| CTTCGTTTCTGCGAACTAAC | CASP9_3 | 186 |
| ACCCCAGTGCGTGCGCTGTT | CD40_1 | 187 |
| ATTCGCTTTCACCGCAAGGA | CD40_2 | 188 |
| AGGAATTCGCTTTCACCGCA | CD40_3 | 189 |
| TCTCCCCGATCTGCGGCCAC | CD40LG_1 | 190 |
| ATGACATGTGCCGCAATTTG | CD40LG_2 | 191 |
| CGAATCTACCGGGGGACTTT | CD40LG_3 | 192 |
| TCACCAAGCCCGCGACCAAT | CD70_1 | 193 |
| GCTTTGGTCCCATTGGTCGC | CD70_2 | 194 |
| AGCTGCCGCTCGAGTCACTT | CD70_3 | 195 |
| GACATCCTACTCTTAGACTA | CFLAR_1 | 196 |
| CTCACCTTGTTTCGGACTAT | CFLAR_2 | 197 |
| ATTACCTATAGTCCGAAACA | CFLAR_3 | 198 |
| CCAGCTCTGATTATCCGACA | COL4A3BP_1 | 199 |
| CCATGTCGGATAATCAGAGC | COL4A3BP_2 | 200 |
| CGGTTGTCGAGCCTCCATGT | COL4A3BP_3 | 201 |
| CGTCATGCCATAATACTGTA | CS_1 | 202 |
| CTCAAGGGCATCCGTTTCCG | CS_2 | 203 |
| CAGCCGAACCAAGTACTGGG | CS_3 | 204 |
| TCTTGTTTAGGCATCATCTG | CYCS_1 | 205 |
| TGCCACACCGTTGAAAAGGG | CYCS_2 | 206 |
| ATCTCCATGGTCTCTTTGGG | CYCS_3 | 207 |
| GGATAACCCTATTGGCAACT | CYLD_1 | 208 |
| TATGGGGTAATCCGTTGGAT | CYLD_2 | 209 |
| CGTTGGATCGGTCAGCCACC | CYLD_3 | 210 |
| GCTTACGTCCACAAGTGCTC | DDX58_1 | 211 |
| TGACTGCCTCGGTTGGTGTT | DDX58_2 | 212 |
| GACTGCCTCGGTTGGTGTTG | DDX58_3 | 213 |
| GGCGTCCGCGCGCTGCACAA | DIABLO_1 | 214 |
| AGCCGCCATTGTGCAGCGCG | DIABLO_2 | 215 |
| GTGTGCGGTTCCTATTGCAC | DIABLO_3 | 216 |
| AATCGTCGTAGTGGGAACGC | DNM1L_1 | 217 |
| GTTCCCACTACGACGATTTG | DNMIL_2 | 218 |
| CTGCCTCAAATCGTCGTAGT | DNM1L_3 | 219 |
| GGTCCTCTTCATCGCAGCGC | DSCC1_1 | 220 |
| CGTCGCATCCACCTCGTCGC | DSCC1_2 | 221 |
| TGCTACAGTCTTGTGATTCG | DSCC1_3 | 222 |
| CGGGCGAGCTGGCCAAGTAC | ENDOG_1 | 223 |
| AGCACAGCACGTACGACTCG | ENDOG_2 | 224 |
| CGCGGAAGTCGCACTCGCGC | ENDOG_3 | 225 |
| GTTCCTATGCCTCGGGCGCG | FADD_1 | 226 |
| AGTCGTCGACGCGCCGCAGC | FADD_2 | 227 |
| GCGTCGACGACTTCGAGGCG | FADD_3 | 228 |
| ACTGCGTGCCCTGCCAAGAA | FAS_1 | 229 |
| AAGCCACCCCAAGTTAGATC | FAS_2 | 230 |
| GATCCAGATCTAACTTGGGG | FAS_3 | 231 |
| ACTCCGAGAGGTAAGCCTGC | FASLG_1 | 232 |
| CTGGTTGCCTTGGTAGGATT | FASLG_2 | 233 |
| TCTGGTTGCCTTGGTAGGAT | FASLG_3 | 234 |
| ATACTCGCCGCCCCCCTCGA | FOXI1_1 | 235 |
| TCGCCGCCCCCCTCGAAGGA | FOXI1_2 | 236 |
| TCGAGGGGGGCGGCGAGTAT | FOXI1_3 | 237 |
| ACTCAGCGTATCGGGCGTGC | GPX4_1 | 238 |
| CACGCCCGATACGCTGAGTG | GPX4_2 | 239 |
| CCCGAACTGGTTACACGGGA | GPX4_3 | 240 |
| ACGACGAGCTGAGCTTCAAA | GRB2_1 | 241 |
| TTAGACGTTCCGGTTCACGG | GRB2_2 | 242 |
| ACGGGGGTGACATAATTGCG | GRB2_3 | 243 |
| AGGTTGACACACTTATAACG | GSDMD_1 | 244 |
| CCACGTACACGTTGTCCCCG | GSDMD_2 | 245 |
| TGAGTGTGGACCCTAACACC | GSDMD_3 | 246 |
| GCACGAAGTCGTCTCGTATC | GZMB_1 | 247 |
| AGTACCATTGAGTTGTGCGT | GZMB_2 | 248 |
| GGTGCATAGTCTTACCTTAA | GZMB_3 | 249 |
| GCGGAACTTGTAGGAACGCG | HRK_1 | 250 |
| GCCTAGCGCCTTGAGCCGGG | HRK_2 | 251 |
| GTCGCCTAGCGCCTTGAGCC | HRK_3 | 252 |
| GTCTTGACCGTCAGCTCGTC | HSPB1_1 | 253 |
| TGGTCGAAGAGGCGGCTATG | HSPB1_2 | 254 |
| TGAGTTGCCGGCTGAGCGCG | HSPB1_3 | 255 |
| CGTTCGAGATAGGGACCTCG | HTRA2_1 | 256 |
| CGCGAGGTCCCTATCTCGAA | HTRA2_2 | 257 |
| GTCCCTATCTCGAACGGCTC | HTRA2_3 | 258 |
| TCATGTAGCGGGCGGCCAGA | IFIH1_1 | 259 |
| CGAATTCCCGAGTCCAACCA | IFIH1_2 | 260 |
| AGACGTCTTGGATAAGTGCA | IFIH1_3 | 261 |
| CCCACAGCCTGGATAACAGG | IFNA1_1 | 262 |
| ATGTCTGTCCATCAGACAGG | IFNA1_2 | 263 |
| TGCTTTACTGATGGTCCTGG | IFNA1_3 | 264 |
| CTTGCAGCTGAGCACCACCA | IFNA13_1 | 265 |
| GTTCGGTGCAGAATTTGTCT | IFNA13_2 | 266 |
| CTACCAGCAGCTGAATGACT | IFNA13_3 | 267 |
| ACATGAATTCAGATTCCCAG | IFNA7_1 | 268 |
| TCAAGGCCCTCCTATTACGC | IFNA7_2 | 269 |
| CTTCAATCTCTTCAGCACAG | IFNA7_3 | 270 |
| GACAATACGCTTTACTTTAT | IL18_1 | 271 |
| TACGCTTTACTTTATAGGTA | IL18_2 | 272 |
| TTTGAATCTTCATCATACGA | IL18_3 | 273 |
| TCCGACCACCACTACAGCAA | IL1B_1 | 274 |
| CGCGTCAGTTGTTGTGGCCA | IL1B_2 | 275 |
| CATGGCCACAACAACTGACG | IL1B_3 | 276 |
| TGATATCGCACTTGTGTCCG | IL4_1 | 277 |
| GCAGAAGGTGAGTACCTATC | IL4_2 | 278 |
| TGCAAATCGACACCTATTAA | IL4_3 | 279 |
| GCGATGGACGCCCCAAAAGC | IREB2_1 | 280 |
| TTACTCAATACGGGTCTTGT | IREB2_2 | 281 |
| AACAAGACCCGTATTGAGTA | IREB2_3 | 282 |
| ATTCACATACCCGCTCCTTC | IRGM_1 | 283 |
| GATGCTTGCCAAAACCGCTG | IRGM_2 | 284 |
| CAACCGGTATGACTTCATCA | IRGM_3 | 285 |
| TCGGCGGCGCAGCCGGTCAA | JUN_1 | 286 |
| CCGTCCGAGAGCGGACCTTA | JUN_2 | 287 |
| CATAAGGTCCGCTCTCGGAC | JUN_3 | 288 |
| CGCAGCCGACCGCTGTAAGG | MAPILC3A_1 | 289 |
| GTCCGCAGCCGACCGCTGTA | MAPILC3A_2 | 290 |
| GGACTCACCGGGATTTTGCT | MAPILC3A_3 | 291 |
| ACAGGGTCAGTTGTATCTAC | MAVS_1 | 292 |
| CCAGCACGGGTTGAGTTGAT | MAVS_2 | 293 |
| CAACTCAACCCGTGCTGGCA | MAVS_3 | 294 |
| TTCCATGTAGAGGACCTAGA | MCL1_1 | 295 |
| CTGGAGACCTTACGACGGGT | MCL1_2 | 296 |
| GGCGCTGGAGACCTTACGAC | MCL1_3 | 297 |
| TTGAAGCATATTATCACCCT | MLKL_1 | 298 |
| TTAGCTTTGGAATCGTCCTC | MLKL_2 | 299 |
| ACTGGAGATATCCCGTTTCA | MLKL_3 | 300 |
| TACAGTTTTGCCGACGGATG | MPG_1 | 301 |
| TTACAGTTTTGCCGACGGAT | MPG_2 | 302 |
| AAAGGGCCACCTTACCCGAC | MPG_3 | 303 |
| AGCATCCACTTCCCAATACG | NAIP_1 | 304 |
| CTGTTACTGAAATGTGCGTG | NAIP_2 | 305 |
| GATGTCTTATTTCCTCGTAT | NAIP_3 | 306 |
| GCGACGGAAAGAGTATGAGC | NFE2L2_1 | 307 |
| CATACCGTCTAAATCAACAG | NFE2L2_2 | 308 |
| GCATACCGTCTAAATCAACA | NFE2L2_3 | 309 |
| CGTGTTGATACACTGCTGCG | NLRC4_1 | 310 |
| CAAACTGCCGTATGTGCCTC | NLRC4_2 | 311 |
| CTACAGAATCAACGGCTGCC | NLRC4_3 | 312 |
| TCGCCAATAAAGCGCACTCC | NLRP1_1 | 313 |
| GCCGGCAATTCATGGATCCA | NLRP1_2 | 314 |
| CGCCGGCAATTCATGGATCC | NLRP1_3 | 315 |
| ACGCTAATGATCGACTTCAA | NLRP3_1 | 316 |
| CGCTAATGATCGACTTCAAT | NLRP3_2 | 317 |
| ATTGAAGTCGATCATTAGCG | NLRP3_3 | 318 |
| ACGGAGGCTAAGCGTCGCAA | NonTargetingControlGuide- | 319 |
| | ForHuman_0001 | |
| CGCTTCCGCGGCCCGTTCAA | NonTargetingControlGuide- | 320 |
| | ForHuman_0002 | |
| ATCGTTTCCGCTTAACGGCG | NonTargetingControlGuide- | 321 |
| | ForHuman_0003 | |
| GTAGGCGCGCCGCTCTCTAC | NonTargetingControlGuide- | 322 |
| | ForHuman_0004 | |
| CCATATCGGGGCGAGACATG | NonTargetingControlGuide- | 323 |
| | ForHuman_0005 | |
| TACTAACGCCGCTCCTACAG | NonTargetingControlGuide- | 324 |
| | ForHuman_0006 | |
| TGAGGATCATGTCGAGCGCC | NonTargetingControlGuide- | 325 |
| | ForHuman_0007 | |
| GGGCCCGCATAGGATATCGC | NonTargetingControlGuide- | 326 |
| | ForHuman_0008 | |
| TAGACAACCGCGGAGAATGC | NonTargetingControlGuide- | 327 |
| | ForHuman_0009 | |
| ACGGGCGGCTATCGCTGACT | NonTargetingControlGuide- | 328 |
| | ForHuman_0010 | |
| CGCGGAAATTTTACCGACGA | NonTargetingControlGuide- | 329 |
| | ForHuman_0011 | |
| CTTACAATCGTCGGTCCAAT | NonTargetingControlGuide- | 330 |
| | ForHuman_0012 | |
| GCGTGCGTCCCGGGTTACCC | NonTargetingControlGuide- | 331 |
| | ForHuman_0013 | |
| CGGAGTAACAAGCGGACGGA | NonTargetingControlGuide- | 332 |
| | ForHuman_0014 | |
| CGAGTGTTATACGCACCGTT | NonTargetingControlGuide- | 333 |
| | ForHuman_0015 | |
| CGACTAACCGGAAACTTTTT | NonTargetingControlGuide- | 334 |
| | ForHuman_0016 | |
| CAACGGGTTCTCCCGGCTAC | NonTargetingControlGuide- | 335 |
| | ForHuman_0017 | |
| CAGGAGTCGCCGATACGCGT | NonTargetingControlGuide- | 336 |
| | ForHuman_0018 | |
| TTCACGTCGTCTCGCGACCA | NonTargetingControlGuide- | 337 |
| | ForHuman_0019 | |
| GTGTCGGATTCCGCCGCTTA | NonTargetingControlGuide- | 338 |
| | ForHuman_0020 | |
| CACGAACTCACACCGCGCGA | NonTargetingControlGuide- | 339 |
| | ForHuman_0021 | |
| CGCTAGTACGCTCCTCTATA | NonTargetingControlGuide- | 340 |
| | ForHuman_0022 | |
| TCGCGCTTGGGTTATACGCT | NonTargetingControlGuide- | 341 |
| | ForHuman_0023 | |
| CTATCTCGAGTGGTAATGCG | NonTargetingControlGuide- | 342 |
| | ForHuman_0024 | |
| AATCGACTCGAACTTCGTGT | NonTargetingControlGuide- | 343 |
| | ForHuman_0025 | |
| CCCGATGGACTATACCGAAC | NonTargetingControlGuide- | 344 |
| | ForHuman_0026 | |
| ACGTTCGAGTACGACCAGCT | NonTargetingControlGuide- | 345 |
| | ForHuman_0027 | |
| CGCGACGACTCAACCTAGTC | NonTargetingControlGuide- | 346 |
| | ForHuman_0028 | |
| GGTCACCGATCGAGAGCTAG | NonTargetingControlGuide- | 347 |
| | ForHuman_0029 | |
| CTCAACCGACCGTATGGTCA | NonTargetingControlGuide- | 348 |
| | ForHuman_0030 | |
| CGTATTCGACTCTCAACGCG | NonTargetingControlGuide- | 349 |
| | ForHuman_0031 | |
| CTAGCCGCCCAGATCGAGCC | NonTargetingControlGuide- | 350 |
| | ForHuman_0032 | |
| GAATCGACCGACACTAATGT | NonTargetingControlGuide- | 351 |
| | ForHuman_0033 | |
| ACTTCAGTTCGGCGTAGTCA | NonTargetingControlGuide- | 352 |
| | ForHuman_0034 | |
| GTGCGATGTCGCTTCAACGT | NonTargetingControlGuide- | 353 |
| | ForHuman_0035 | |
| CGCCTAATTTCCGGATCAAT | NonTargetingControlGuide- | 354 |
| | ForHuman_0036 | |
| CGTGGCCGGAACCGTCATAG | NonTargetingControlGuide- | 355 |
| | ForHuman_0037 | |
| ACCCTCCGAATCGTAACGGA | NonTargetingControlGuide- | 356 |
| | ForHuman_0038 | |
| AAACGGTACGACAGCGTGTG | NonTargetingControlGuide- | 357 |
| | ForHuman_0039 | |
| ACATAGTCGACGGCTCGATT | NonTargetingControlGuide- | 358 |
| | ForHuman_0040 | |
| GATGGCGCTTCAGTCGTCGG | NonTargetingControlGuide- | 359 |
| | ForHuman_0041 | |
| ATAATCCGGAAACGCTCGAC | NonTargetingControlGuide- | 360 |
| | ForHuman_0042 | |
| CGCCGGGCTGACAATTAACG | NonTargetingControlGuide- | 361 |
| | ForHuman_0043 | |
| CGTCGCCATATGCCGGTGGC | NonTargetingControlGuide- | 362 |
| | ForHuman_0044 | |
| CGGGCCTATAACACCATCGA | NonTargetingControlGuide- | 363 |
| | ForHuman_0045 | |
| CGCCGTTCCGAGATACTTGA | NonTargetingControlGuide- | 364 |
| | ForHuman_0046 | |
| CGGGACGTCGCGAAAATGTA | NonTargetingControlGuide- | 365 |
| | ForHuman_0047 | |
| TCGGCATACGGGACACACGC | NonTargetingControlGuide- | 366 |
| | ForHuman_0048 | |
| AGCTCCATCGCCGCGATAAT | NonTargetingControlGuide- | 367 |
| | ForHuman_0049 | |
| ATCGTATCATCAGCTAGCGC | NonTargetingControlGuide- | 368 |
| | ForHuman_0050 | |
| TGACCATCTCTACCGCGACG | NOS3_1 | 369 |
| GGGCGTTCTGCACCTCGTCG | NOS3_2 | 370 |
| CAAGCGCGGGGTTGCTTGCA | NOS3_3 | 371 |
| GCCAAGAGTGTTCGGCACAT | NOX4_1 | 372 |
| ACACTCTTGGCTTACCTCCG | NOX4_2 | 373 |
| ACGCCTACAGAATTACACCA | NOX4_3 | 374 |
| GCTCTTCGAATCGGCCGACG | NOX5_1 | 375 |
| TTCGAATCGGCCGACGCGGA | NOX5_2 | 376 |
| CGGCCGACGCGGACGGCAAC | NOX5_3 | 377 |
| CGGTGGCGTAGTTCAGGTAG | NPEPL1_1 | 378 |
| CGGCCGCCCACTTCATCACG | NPEPL1_2 | 379 |
| CGGAGCGCATCGCTGCATTG | NPEPL1_3 | 380 |
| CGAGTCGAGTACGCCAAGAG | PARP1_1 | 381 |
| GAGTCGAGTACGCCAAGAGC | PARP1_2 | 382 |
| AAGTACGTGCAAGGGGTGTA | PARP1_3 | 383 |
| GTTCGAATTCCATGGCGGCG | PARP2_1 | 384 |
| CGAATTCCATGGCGGCGCGG | PARP2_2 | 385 |
| ACGTCAGCGTTCGAATTCCA | PARP2_3 | 386 |
| AAGCGGCATGGCGTTCCGGC | PGAM5_1 | 387 |
| TGGCGCGCGTCATAGACGAA | PGAM5_2 | 388 |
| GTGCCGGCTGATGATATCGG | PGAM5_3 | 389 |
| TAAGCTGGACGTTGATATCC | PIK3C3_1 | 390 |
| CTTTGCAGACGGTGCGATGG | PIK3C3_2 | 391 |
| GGAACAACGGTTTCGCTCTT | PIK3C3_3 | 392 |
| TCCAGCAGGTACCGACCCGC | PMAIP1_1 | 393 |
| CCCAGCGGGTCGGTACCTGC | PMAIP1_2 | 394 |
| CGCTCAACCGAGCCCCGCGC | PMAIP1_3 | 395 |
| TAGGCATACGCTGACTACAT | PTPA_1 | 396 |
| TACCCTCAACGAAGGTGTGA | PTPA_2 | 397 |
| TTCTCAACACGCTGGACAGG | PTPA_3 | 398 |
| CGCTAACGTGCTGCGCGACA | PYCARD_1 | 399 |
| CATGTCGCGCAGCACGTTAG | PYCARD_2 | 400 |
| GTGCCGCTGCGCGAGGGCTA | PYCARD_3 | 401 |
| ATCTTTGATTGCGGAACAAC | RB1CC1_1 | 402 |
| GCGGATCTATCTGATTCACC | RB1CC1_2 | 403 |
| TGAAGATCGGCTCTACGCCC | RB1CC1_3 | 404 |
| CGGCTTTCAGCACGTGCATC | RIPK1_1 | 405 |
| CTCGGGCGCCATGTAGTAGA | RIPK1_2 | 406 |
| GTTGACGTCATTCAGGTGCT | RIPK1_3 | 407 |
| CGGGCGCAACATAGGAAGTG | RIPK3_1 | 408 |
| CAGTGTTCCGGGCGCAACAT | RIPK3_2 | 409 |
| CGGGTTCGGCACAGTGTTCC | RIPK3_3 | 410 |
| GAAAACAGAATATGGACCTA | SafeHarborchr1_21 | 411 |
| GAAAAGTGGATGTGTGTCCC | SafeHarborchr10_45 | 412 |
| GAAAACTGCATTGTGCAGAC | SafeHarborchr11_28 | 413 |
| GAAAAGACAGTAAACCTTGC | SafeHarborchr11_33 | 414 |
| GAAAAGACAGTAAACCTTTC | SafeHarborchr11_34 | 415 |
| GAAAAGCCTGTGTGGAAGAG | SafeHarborchr11_38 | 416 |
| GAAAAGTAAAACATGCAGAG | SafeHarborchr11_41 | 417 |
| GAAAAAACTGTTTCTTTGGT | SafeHarborchr12_1 | 418 |
| GAAAACTATACCTCATGATT | SafeHarborchr12_26 | 419 |
| GAAAACTTAGAGAAAGCACA | SafeHarborchr12_29 | 420 |
| GAAAACTTCAGAAATTGTGC | SafeHarborchr12_30 | 421 |
| GAAAAATCAGACGAGCTCCA | SafeHarborchr13_12 | 422 |
| GAAAAATGTCTCTACTTGCC | SafeHarborchr13_16 | 423 |
| GAAAACTAGTAGTGGAAACT | SafeHarborchr13_25 | 424 |
| GAAAAAGGATACATTTGCAA | SafeHarborchr13_8 | 425 |
| GAAAACACACCTTATCACAG | SafeHarborchr15_19 | 426 |
| GAAAACCCTATATATATAGG | SafeHarborchr18_24 | 427 |
| GAAAAAAGTTAACTTAGACT | SafeHarborchr18_3 | 428 |
| GAAAAGAATAACAGTAGTAA | SafeHarborchr2_32 | 429 |
| GAAAACCAGACAAGTTACCT | SafeHarborchr21_23 | 430 |
| GAAAAGAAATATCATTGGCA | SafeHarborchr21_31 | 431 |
| GAAAAAAGCTGGAGAGACAC | SafeHarborchr3_2 | 432 |
| GAAAACAGCTATTCGGTGCT | SafeHarborchr3_22 | 433 |
| GAAAACTCCCTTGTCTTGGG | SafeHarborchr3_27 | 434 |
| GAAAAGTTCTATTCAATTTG | SafeHarborchr3_47 | 435 |
| GAAAAAGAGACAGAGTATGT | SafeHarborchr3_6 | 436 |
| GAAAAATTGTCCCAGTACAC | SafeHarborchr4_17 | 437 |
| GAAAAGACCTCATTAGAGTG | SafeHarborchr4_35 | 438 |
| GAAAAGATCTCTACCAGGAA | SafeHarborchr4_36 | 439 |
| GAAAAACAGACTTAGGGGAG | SafeHarborchr4_4 | 440 |
| GAAAAGTAAAACCCTCTCAG | SafeHarborchr4_42 | 441 |
| GAAAAGTATCTATGATGAAA | SafeHarborchr4_43 | 442 |
| GAAAAGTCACACTAAAGTGT | SafeHarborchr4_44 | 443 |
| GAAAAGTTAGAGTGAAGTAG | SafeHarborchr4_46 | 444 |
| GAAAATAAAGGTAATGGCTG | SafeHarborchr4_48 | 445 |
| GAAAAAGGCAGGCAATGGTG | SafeHarborchr4_9 | 446 |
| GAAAAACTCTTAGCTTAATG | SafeHarborchr5_5 | 447 |
| GAAAAATGACCCATGATTAA | SafeHarborchr6_14 | 448 |
| GAAAATAAGGCATCAATAAC | SafeHarborchr6_50 | 449 |
| GAAAAATAGCTTTCTCTCAA | SafeHarborchr7_11 | 450 |
| GAAAAATCGCTAATATGAAA | SafeHarborchr8_13 | 451 |
| GAAAAGATGAGTTATATTGG | SafeHarborchr8_37 | 452 |
| GAAAAAGCAGCACTTCTTAT | SafeHarborchr8_7 | 453 |
| GAAAAATTTCAGCAGACCTT | SafeHarborchr9_18 | 454 |
| GAAAACACAGTCTTAATTAT | SafeHarborchr9_20 | 455 |
| GAAAAGGAAGATTCTGCTGT | SafeHarborchr9_39 | 456 |
| GAAAAGGTGAGGTTAAGGAG | SafeHarborchr9_40 | 457 |
| GAAAATAACCACAATTATCA | SafeHarborchr9_49 | 458 |
| GAAAAATAAGGTGAAGATAG | SafeHarborchrX_10 | 459 |
| GAAAAATGACTGGTCACATT | SafeHarborchrX_15 | 460 |
| AACTTGGCCGAGCCATTTTC | SIRT5_1 | 461 |
| TCAATCGACTTGGGACAATC | SIRT5_2 | 462 |
| CCACACCCGGGACGGGTTGT | SIRT5_3 | 463 |
| CCAGCGGCTTGCAGCTAGAC | SLC11A2_1 | 464 |
| ACTATTATGGCCCTCACATT | SLC11A2_2 | 465 |
| CGAACATGCCCTTGAGTACC | SLC11A2_3 | 466 |
| GCGATGTTGGCGATTAGTGC | SLC25A15_1 | 467 |
| GTTGGCGATTAGTGCTGGAC | SLC25A15_2 | 468 |
| AGTCGGTGAGGCCCCGGTAC | SLC25A15_3 | 469 |
| TACCAGCTTTTGTACGAGTC | SLC7A11_1 | 470 |
| AGCTTTTGTACGAGTCTGGG | SLC7A11_2 | 471 |
| ACCCAGACTCGTACAAAAGC | SLC7A11_3 | 472 |
| CCCAACGGTGGTACTCCAGC | SNX30_1 | 473 |
| ACCTGCTGGAGTACCACCGT | SNX30_2 | 474 |
| CCTGCTGGAGTACCACCGTT | SNX30_3 | 475 |
| AGGCACCTCCGGACCCCGAG | SNX4_1 | 476 |
| GCTCTGGGGTCGACACGGTG | SNX4_2 | 477 |
| AGGTCAGTTGAACATACCGA | SNX4_3 | 478 |
| CTGCAGCACGTCGCTTATAT | TFRC_1 | 479 |
| AATATAAGCGACGTGCTGCA | TFRC_2 | 480 |
| CTATACGCCACATAACCCCC | TFRC_3 | 481 |
| CAAAGATACACCAGCGGCTC | TLR4_1 | 482 |
| ATTCTCCCAGAACCAAACGA | TLR4_2 | 483 |
| CAATCACCTTTCGGCTTTTA | TLR4_3 | 484 |
| GCGGGTTTGTTGGCCACTCA | TLR7_1 | 485 |
| TTATTTTTACACGGCGCACA | TLR7_2 | 486 |
| AGCGCATCAAAAGCATTTAC | TLR7_3 | 487 |
| TTGGAGTGATCGGCCCCCAG | TNF_1 | 488 |
| AGAGCTCTTACCTACAACAT | TNF_2 | 489 |
| GGAGCTGAGAGATAACCAGC | TNF_3 | 490 |
| CTTCAAGTTTGTCGTCGTCG | TNFRSF10A_1 | 491 |
| TGAGCTAGGTACGACCTGTG | TNFRSF10A_2 | 492 |
| CTGAGCTAGGTACGACCTGT | TNFRSF10A_3 | 493 |
| CCGCGGCGACAACGAGCACA | TNFRSF10B_1 | 494 |
| GAGCGGCCCCACAACAAAAG | TNFRSF10B_2 | 495 |
| ATAGTCCTGTCCATATTTGC | TNFRSF10B_3 | 496 |
| GATGACGACGACGAACTTTA | TNFRSF10C_1 | 497 |
| CGGCGTCGGGAACCATACCA | TNFRSF10C_2 | 498 |
| CGATGACGACGACGAACTTT | TNFRSF10C_3 | 499 |
| GCTCGAGCAGGGCGCTATCC | TNFRSF10D_1 | 500 |
| AACTTCGTCCTGCCGGGGGA | TNFRSF10D_2 | 501 |
| TGGGGAACTTCGTCCTGCCG | TNFRSF10D_3 | 502 |
| CTTCCTTGCATTCGCACACG | TNFRSF11B_1 | 503 |
| CAACCGCGTGTGCGAATGCA | TNFRSF11B_2 | 504 |
| GCATTCGCACACGCGGTTGT | TNFRSF11B_3 | 505 |
| ATATACCCCTCAGGGGTTAT | TNFRSF1A_1 | 506 |
| ATTGGACTGGTCCCTCACCT | TNFRSF1A_2 | 507 |
| CACTCCAATAATGCCGGTAC | TNFRSF1A_3 | 508 |
| GAAGATCACGATCAGCACGC | TNFSF10_1 | 509 |
| ACTCCGTCAGCTCGTTAGAA | TNFSF10_2 | 510 |
| CGTCAGCTCGTTAGAAAGGT | TNFSF10_3 | 511 |
| CCCCGGACGATATTGAACAA | TP53_1 | 512 |
| CCATTGTTCAATATCGTCCG | TP53_2 | 513 |
| CCATTGCTTGGGACGGCAAG | TP53_3 | 514 |
| CGGGTGAACCACGAAATGGA | UBR5_1 | 515 |
| CCAATACATTCAAAGGGGGG | UBR5_2 | 516 |
| CCACCCCCCTTTGAATGTAT | UBR5_3 | 517 |
| CGAAGGCGCCGTGGCCGATC | ULK1_1 | 518 |
| GCCCTTGAAGACCACCGCGA | ULK1_2 | 519 |
| GTGGCCGATCAGGTCCTTGC | ULK1_3 | 520 |
| TCGCCGTGGTCTTCCGGGGG | ULK2_1 | 521 |
| GACCTCGCAGATTATTTGCA | ULK2_2 | 522 |
| GTCCCTTTCGCTGGTACAAA | ULK2_3 | 523 |
| CGCGCGTCGTAAGTAAAGCT | VDAC2_1 | 524 |
| GCGCGCGTCGTAAGTAAAGC | VDAC2_2 | 525 |
| TGTTAGGAATTTTCAACGTC | VDAC2_3 | 526 |
| CCTTTACTTACCTGGTCGAA | VDAC3_1 | 527 |
| CCCTTCGACCAGGTAAGTAA | VDAC3_2 | 528 |
| TCCTTTACTTACCTGGTCGA | VDAC3_3 | 529 |
| TGATACACGTTCATCTGCCG | WIPI1_1 | 530 |
| TACTTGCCGGTTCAGCCTTA | WIPI1_2 | 531 |
| CCTTATGGACAAGATGTTGC | WIPI1_3 | 532 |
| CCCGGCCTTACGTGTTGTCC | WIPI2_1 | 533 |
| CAGGACAACACGTAAGGCCG | WIPI2_2 | 534 |
| ACCAGGACAACACGTAAGGC | WIPI2_3 | 535 |
| GGACTCTACTACACAGGTAT | XIAP_1 | 536 |
| CATCAACACTGGCACGAGCA | XIAP_2 | 537 |
| TATCAGACACCATATACCCG | XIAP_3 | 538 |

Pooled lentiviral screening requires a multiplicity of infection (MOI) less than 1 (FIG. 1C; FIG. 19C) for single-copy sgRNA integration per cell. Then, the iCas9/NURR1::H2B-GFP/library hPSC was directed differentiated into dopamine neurons using an established protocol. At day 25 of differentiation, 800,000 homogenous post-mitotic dopamine neurons were grafted into the bilateral striatum of the eight NOD/SCID IL2Rgnull (NSG) mice using FACS with NURR1-GFP and gRNA-tdTomato (FIG. 2A; FIG. 12A). Additional 2 weeks in vitro culture post sorting gave rise to highly homogenous dopamine neuron identity co-expressing NURR1::GFP and gRNA::tdTomato with a mature dopamine marker, TH, regardless of doxycycline treatment (FIG. 1D; FIG. 19D). The use of sorted NURR1 positive cell population allowed to cleanly examine the survival rate of post-mitotic dopamine neurons in vivo without any confounding factors from proliferating cells in the graft. After 1-month post-transplantation, the implanted cells were re-isolated by dissecting the graft based on fluorescence expression, which genomic DNA was extracted (FIG. 1F; FIG. 19F). Human-specific PCR reaction for detecting a human PTGER2 gene indicated the presence of the human cells in a xenograft sample (FIG. 1G; FIG. 19F). Next-generation sequencing (NGS) from genomic DNA of in vitro cultured cells and an in vivo grafted cell to detect sgRNA barcode indicated consistent sgRNA library representation in vitro and in vivo independent of Dox treatment (FIG. 1H; FIG. 19G).

Next, the correlation of each sgRNA incorporation ratio from all samples was compared (in vitro cells; day 16, day 25 no dox, day 25 plus dox, and in vivo grafted cells; 1-month post grafted cells from day 25 plus dox). Clustering dendrogram showed comparable sgRNA incorporation ratio regardless of dox treatment and cell stage in culture, except the grafted cell (FIG. 2B; FIG. 12B). In-depth analysis of each sgRNA ratio in the conditions indicated that, without dox treatment in culture, enriched or depleted sgRNAs could not be detected. However, in dox treatment conditions, all 3 sgRNAs for BCL2L (BCLXL) were significantly depleted during the directed differentiation (FIG. 2C; FIG. 12C), demonstrating that BCLX is an essential gene for proper dopamine neuron induction from hPSC in the culture system. Previously, it was noted that Bcl-x knock-out mice had less dopamine neuron generation and overexpression of BCLX enhanced the derivation of dopamine neurons from neural stem cells, emphasizing hPSC-based iCas9 system with pooled sgRNA library clearly worked in dopamine neuron differentiation. Most importantly, several significantly enriched sgRNAs were identified in surviving dopamine neurons compared to the cells before grafting, based on two criteria: 1) sgRNAs above a threshold [fold change; more than 0.3 (log 2 value), and p-value; p<0.05], 2) multiple different guide RNA hits targeting the same gene [fold change; more than 0.25 (log 2 value), and p-value; p<0.05]. Using these criteria, surviving dopamine neurons in the graft had enriched sgRNAs corresponding to 9 genes, implying apoptosis and inflammation were major limiting pathways for the survival of post-mitotic dopamine neurons in the graft (FIGS. 2C and 2D; FIGS. 12C and 12D).

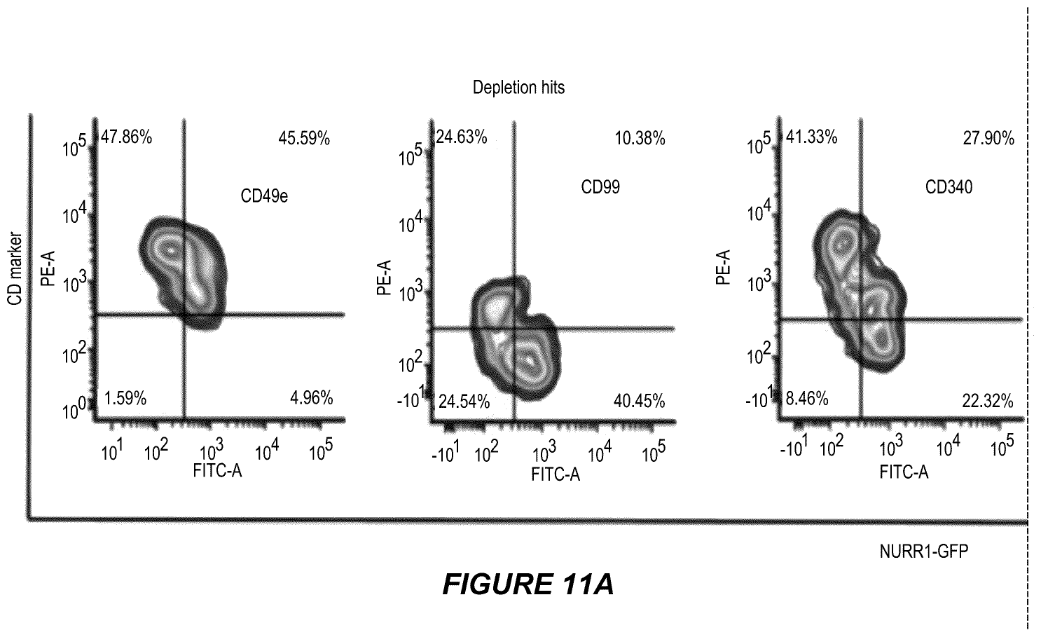

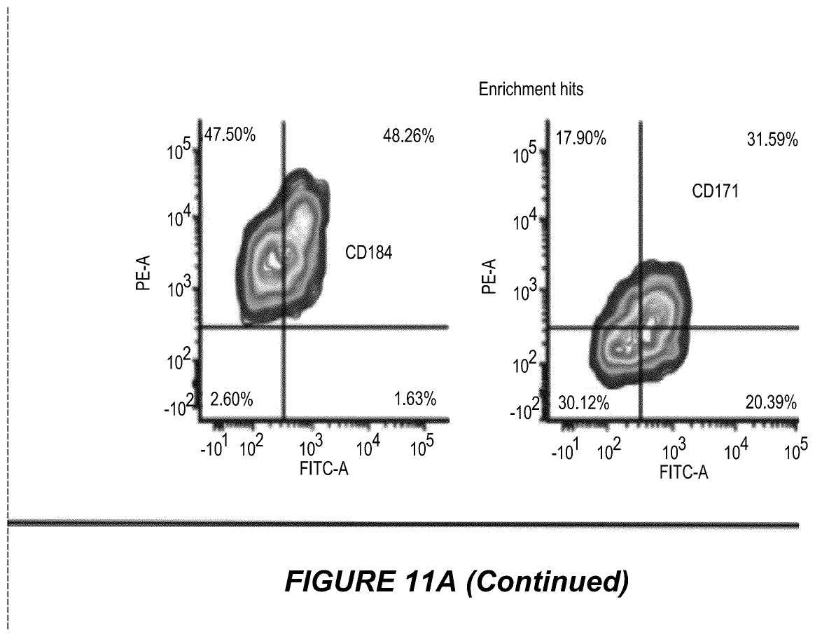

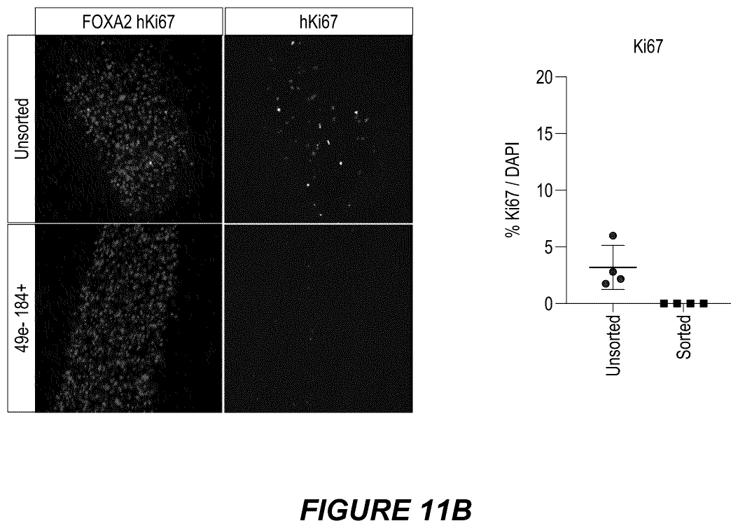

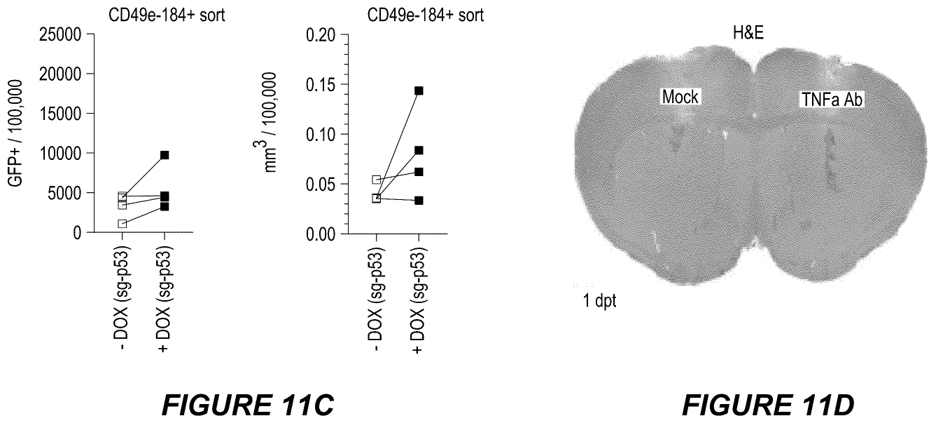

To further validate the hits, 9 genes, from the screen, stable hPSC line were generated containing only 33 sgRNAs targeting a sum of 9 genes (3 sgRNAs per a gene) with 6 non-targeting and safe harbor control guides in the iCas9/NURR1::H2B-GFP hPSC line (iCas9/NURR1::H2B-GFP/library #2). Similarly, the overall representations of sgRNAs were comparable across all conditions (FIG. 11; FIG. 19H), and sgRNA incorporation was only the most distinct in the grafted cells than in vitro cultured cells as indicated by clustering dendrogram (FIG. 2E; FIG. 12E). Two-independent pooled screens with small-pooled sgRNA library using the iCas9/NURR1::H2B-GFP/library #2 hPSC line consistently identified p53 as the most significant hit with two or more independent sgRNAs enriched in the surviving dopamine neurons in the graft (FIG. 2F; FIG. 12F). Overall, these results demonstrate that post-mitotic dopamine neuron death upon transplantation would be driven by the p53-mediated cell death pathway.

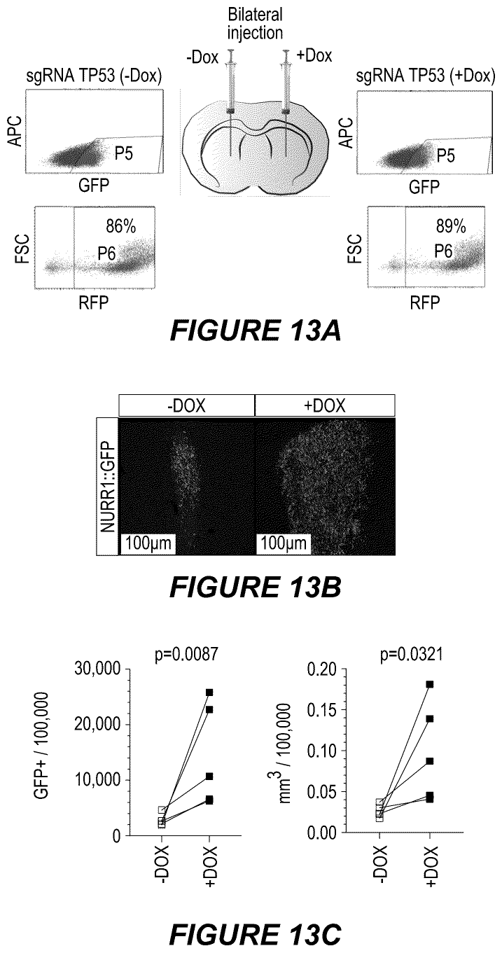

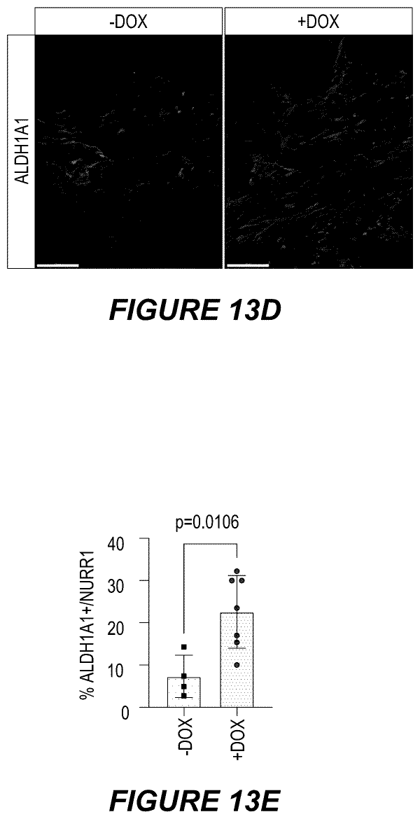

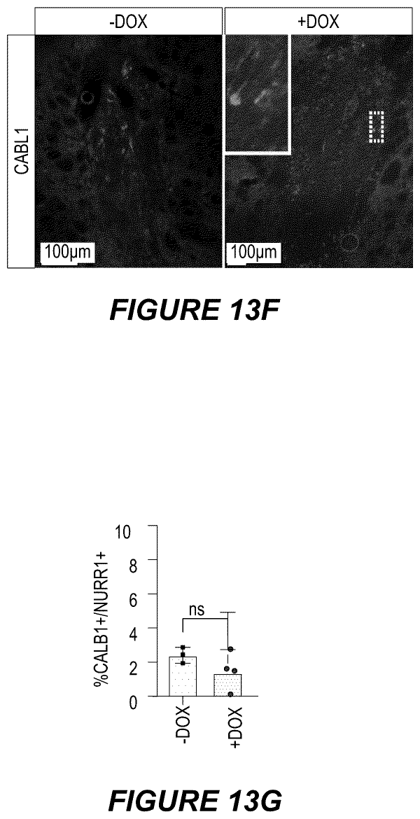

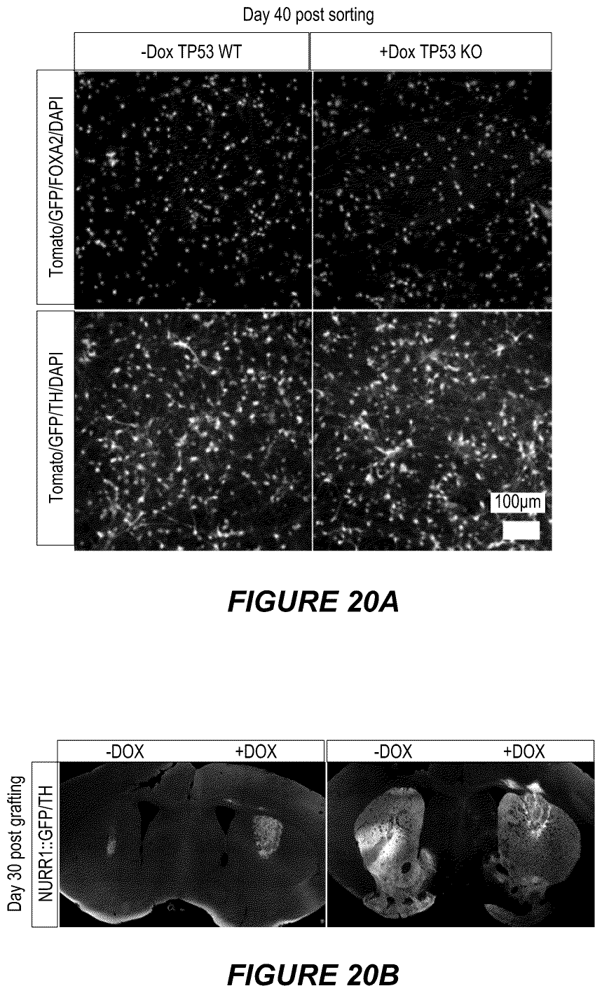

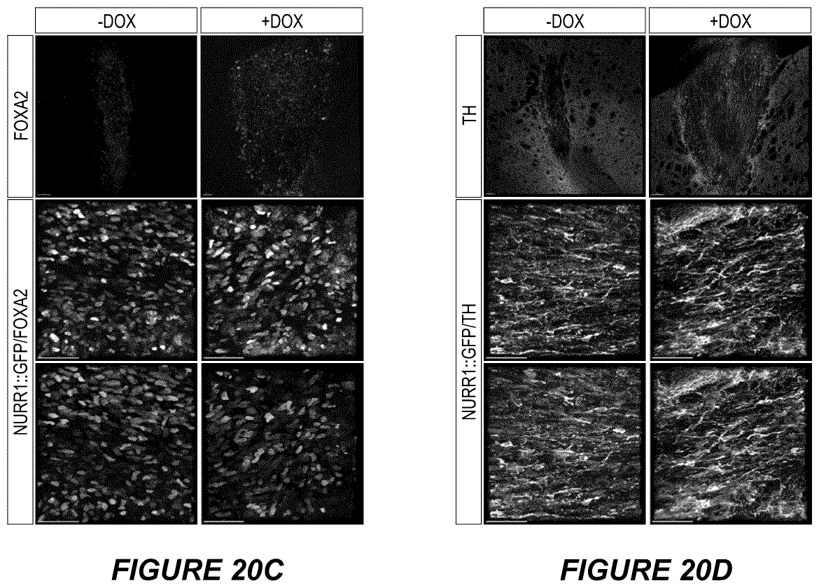

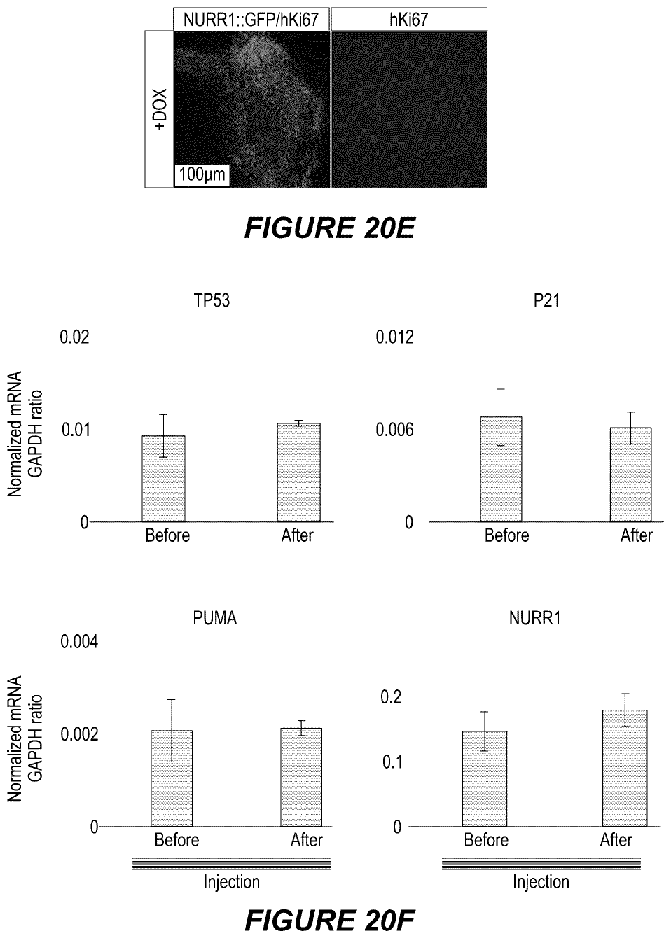

p53 knockout (KO) resulted in improved dopamine neuron survival. To examine the p53 role on dopamine neuron survival in vivo graft, iCas9/NURR1::H2B-GFP hPSC line were generated with a stably incorporated sgRNA targeting p53 gene. While p53 pathway has been previously implicated in the death of exogenous fetal ventral-mesencephalic graft in rats, the p53 effect on the survival of human post-mitotic dopamine neuron in in vivo engraft remains unknown. Furthermore, kinetics of p53-mediated graft death during the time-course of the transplantation is poorly investigated. Directed differentiation towards midbrain dopamine neuron lineages and purification strategy via FACS was similarly performed as the pooled-sgRNA screens for this individual p53 KO studies. Either dox treated (p53 KO) or non-treated dopamine (isogenic p53 WT) neurons were engrafted bilaterally into striatum to reduce any variability between mice (FIG. 3A; FIG. 13A). Notably, dopamine neurons in the p53 KO had no obvious survival benefit or excessive proliferation in culture but rather gave rise to highly pure dopamine neuron identity expressing TH and FOXA2 when examined 2 weeks post-sorting with NURR1::GFP and sgRNA::tdTomato (FIG. 3B; FIG. 20A).

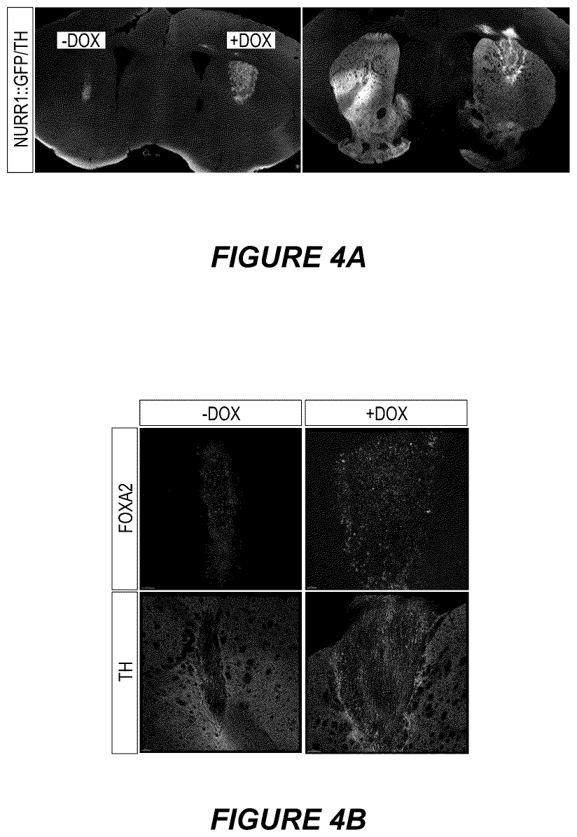

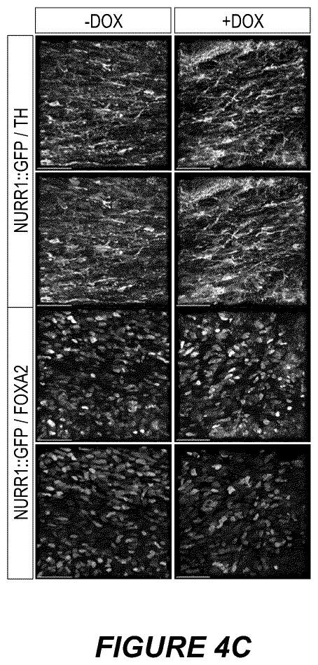

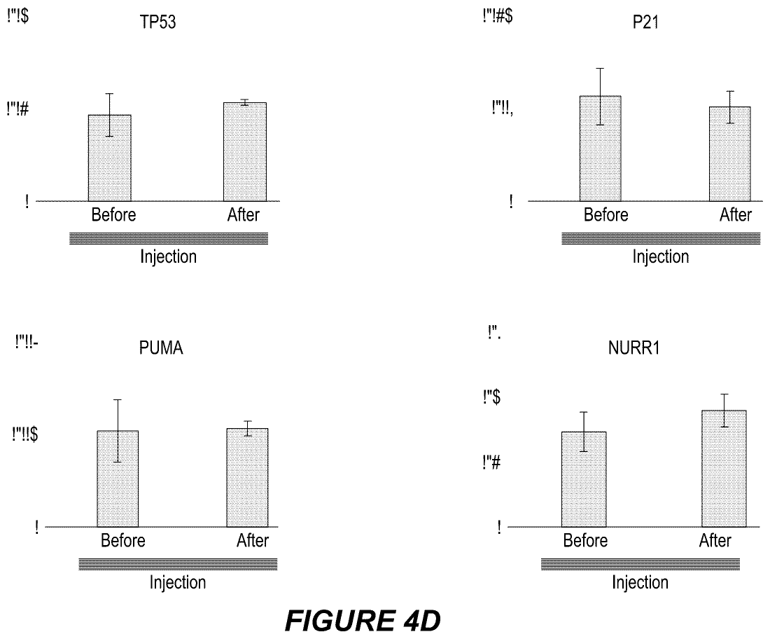

After 1-month post-transplantation, the overall graft composition and size were determined using immunofluorescence assay followed by stercological quantification (FIG. 3C-3D; FIGS. 4A-4C; FIGS. 13B-13C; FIGS. 20A-20D). All the surviving p53 KO dopamine neurons expressed floor-plate identity expressing FOXA2, a mature dopamine neuron marker TH, and an endogenous NURR1-GFP signal (FIGS. 4A-4C;

FIGS. 20A-20D) without obvious detection of a hKi67, which marks proliferating cells, in TP53 KO or in isogenic control grafts (FIG. 20E). The stercological method measured the total number and volume of surviving NURR1::GFP positive neurons per 100,000 cells at 1 month after grafting, and found that p53 KO graft contains 13,666.78 (mean)+7,797.59 (stdev) NURR1::GFP dopamine neurons compared to 2,775.99 (mean)+1,178.21 (stdev) in wild-type neurons (FIG. 3D; FIG. 13C). The volume of the p53 KO graft was 0.086±0.060 mm³ while the wild-type graft was 0.022±0.010 mm³ per 100,000 cells (FIG. 3D; FIG. 13C). Taken together, the single p53 gene KO had augmented a significant (p<0.05, paired t-test, n=5) survival benefits for post-mitotic dopamine neurons in the transplant, emphasizing the functional role of p53 in neuronal survival in in vivo implant. In addition to the increased volume and increased numbers of NURR1+ neurons, p53 KO grafts also showed an increased proportion of ALDH1A1+A9-type dopamine neurons. In contrast, the proportion of CALB1 positive (A10) neuron was comparable between p53 KO and p53 WT grafts (FIGS. 2D-2G) suggesting a particular vulnerability of A9 but not A10 dopamine neuron subtype upon grafting, which is alleviated in p53 KO grafts.

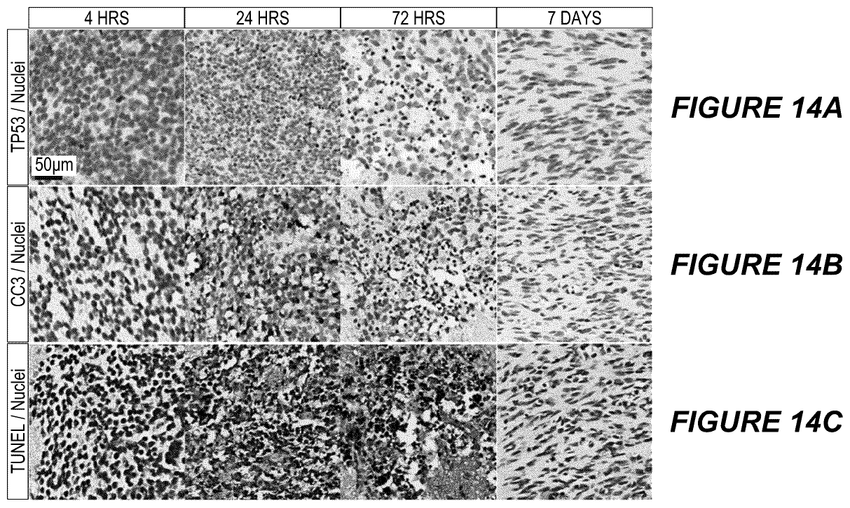

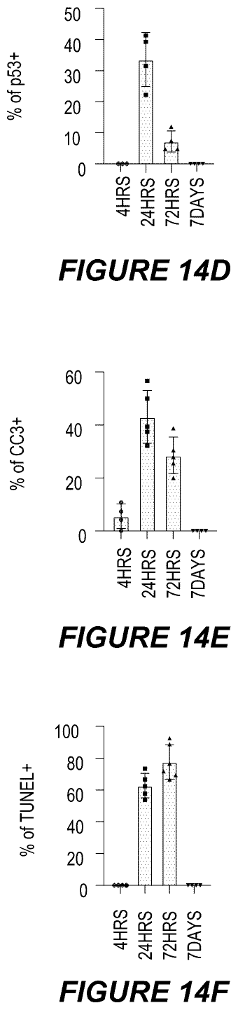

Temporal analysis of p53-mediated dopamine neuron death and host response. Next. experiments were performed to address the kinetics of the p53-dependent neuronal cell death during the transplantation procedure. There would be three main stages of cell death in the grafting paradigm: 1) Mechanical damage stress during loading cells onto the needle 2) short-term survival factors immediately post-transplantation 3) longer-term effects in established graft. First, p53 and p53 downstream genes were not induced using RT-qPCR assay during cell preparation and loading cells into the needle (FIG. 4D; FIG. 20F), which was consistent with a previous report demonstrating that there were no signs of apparent cell death after passaging through the needle. Next, p53 and cleaved caspase 3 (CC3) inductions were mapped using immunofluorescence assays at different time points immediately post grafting, observing a strong induction of p53 and CC3 in 30-40% of the dopamine neurons at 1-day post-transplantation (dpt) (FIGS. 3E and 3F; FIGS. 14A-14F). Such inductions were diminished at 3 dpt with signs of many apoptotic pyknotic nuclei within the graft, implying that p53-induced neurons at 1 dpt already triggered intrinsic apoptotic “suicide” program. Additionally, the rapid engagement of cell death resulted in a dramatic decrease in the grafted cell density from 1 to 3 dpt (FIGS. 3E and 3F; FIGS. 14A-14F). At 7 dpt and beyond, this dynamic programmed cell death cascade appeared to be completely absent. Furthermore, a TUNEL assay to probe for any apoptotic DNA fragmentation and detect the last phase of apoptosis showed a strong TUNEL positive expression among 60% of grafted neurons starting at 1 dpt and the peak TUNEL positive signals at 3 dpt in the graft (FIGS. 3E and 3F; FIGS. 14A-14F). Again, the TUNEL signal was completely abolished at 7 dpt, implying this apoptotic program might no longer continue beyond 7 dpt.

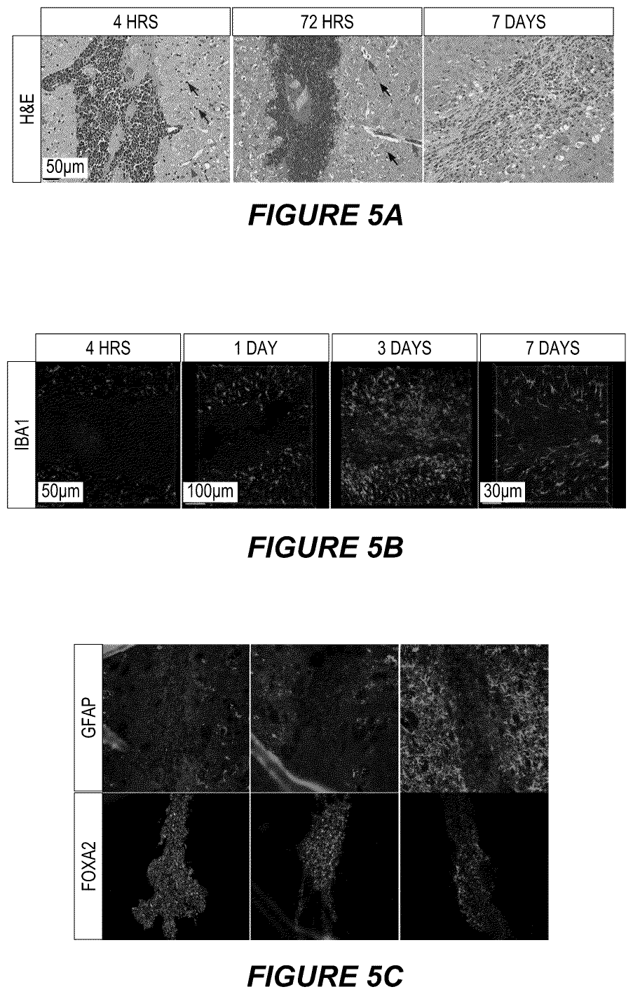

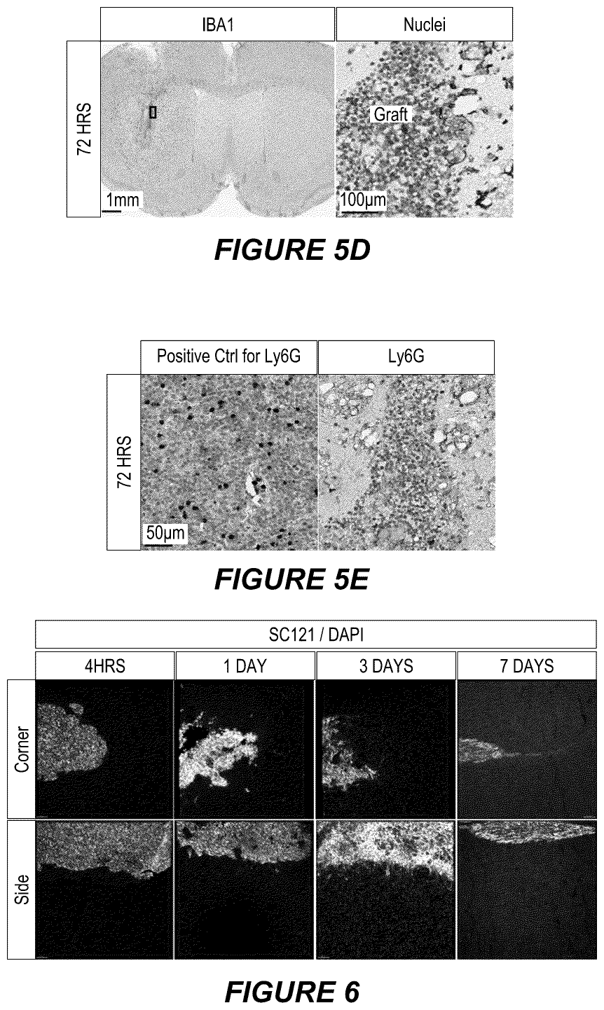

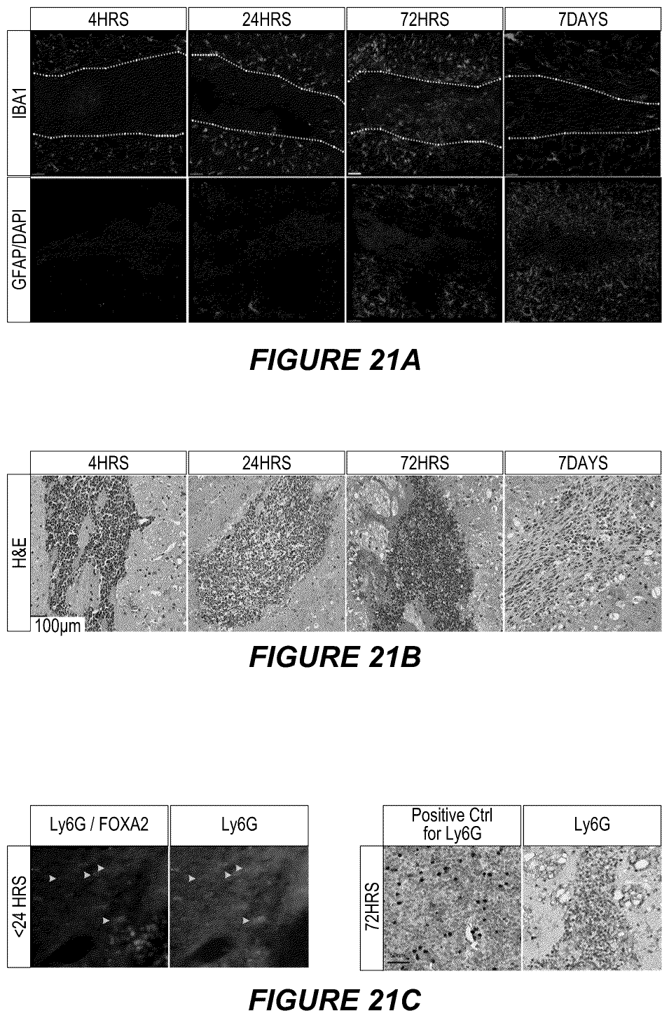

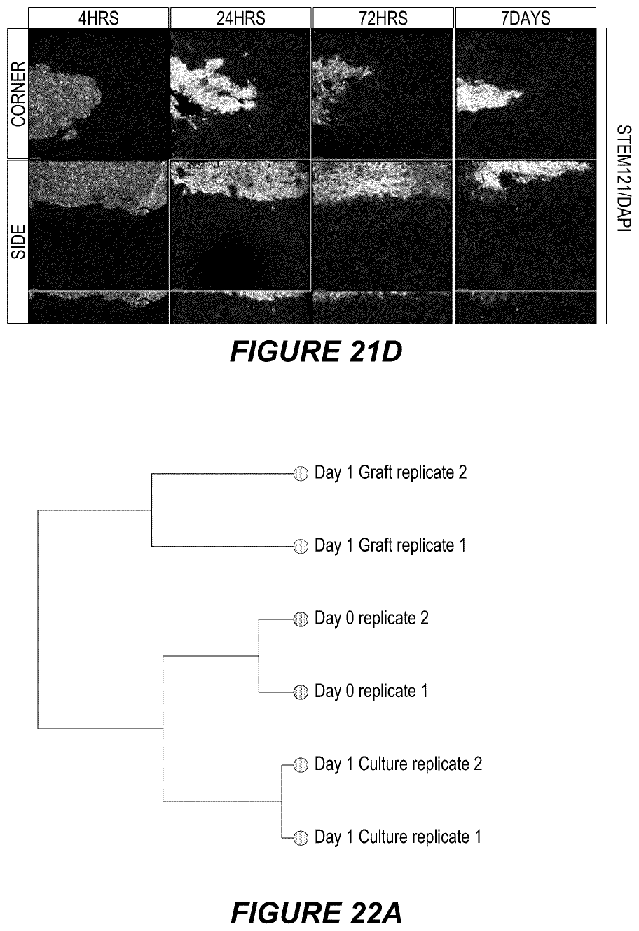

Next, neuroimmune cells near the graft site were characterized since published studies indicated apoptotic neurons recruit neighboring macrophages which clear apoptotic cells. Together with the finding that high induction of apoptosis appears in the graft a 1 dpt, both astrocyte and microglia rapidly and concurrently polarized their processes towards the transplants (FIGS. 5B and 5C). At 4 hours and at 1 dpt, most glial cells were located outside the graft core, suggesting that they are respond to rather than driving the initial wave of dopamine neuron cell death (FIG. 21A). At 3 dpt, immune response peaked, but the extent of polarization was more obvious in microglia than in astrocyte where microglia migrated further and fully encapsulated apoptotic cell bodies by infiltrating into the graft core by day 3 dpt (FIG. 21A, upper panels), which was further corroborated by existing literature, reporting the coordinated behaviors of astrocytes and microglia (FIGS. 5B-5D). GFAP staining, more specific to A1-activated type of astrocytes, were also more strongly induced in the host cells at 3 dpt, suggesting that activated microglia could further recruit neurotoxic reactive astrocytes (FIG. 5C). Strong GFAP staining persisted by 7 dpt creating an “astrocyte border” at the graft host interface (FIG. 21A, lower panels). Also, evidence of vascular recruitment to the graft at 3 dpt by H&E staining (FIG. 5A; FIG. 21B) and neutrophil invasion marked by Ly6G present prior to 24 hours post grafting (FIG. 5E; FIG. 21C). Despite the transplanted neurons undergoing apoptosis and engulfed by immune cells, simultaneously surviving neurons began to extend axons at 1 dpt as indicated by SC121 positive fiber outgrowth from the implants (FIG. 6; FIG. 21D). Overall, these data revealed that p53-dependent apoptotic cell death of hPSC-derived post-mitotic dopamine neurons in vivo post grafting occurred rapidly in a very narrow window during the first 3 dpt in which brain immune cells were heavily recruited towards the graft.

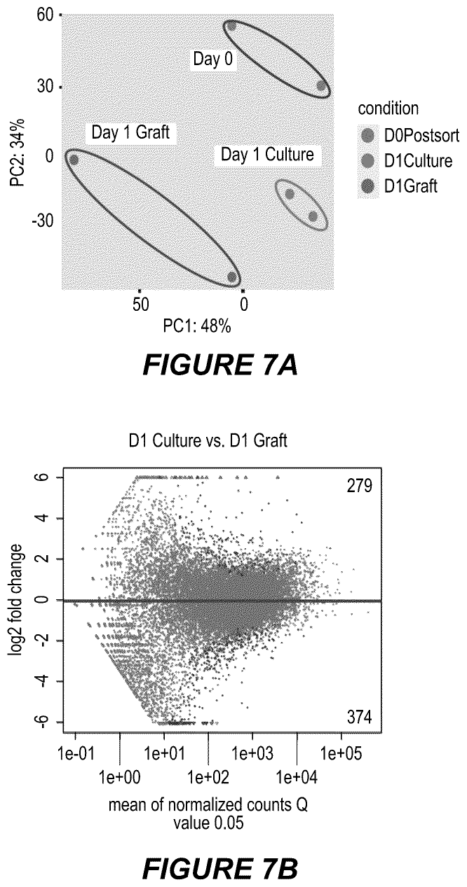

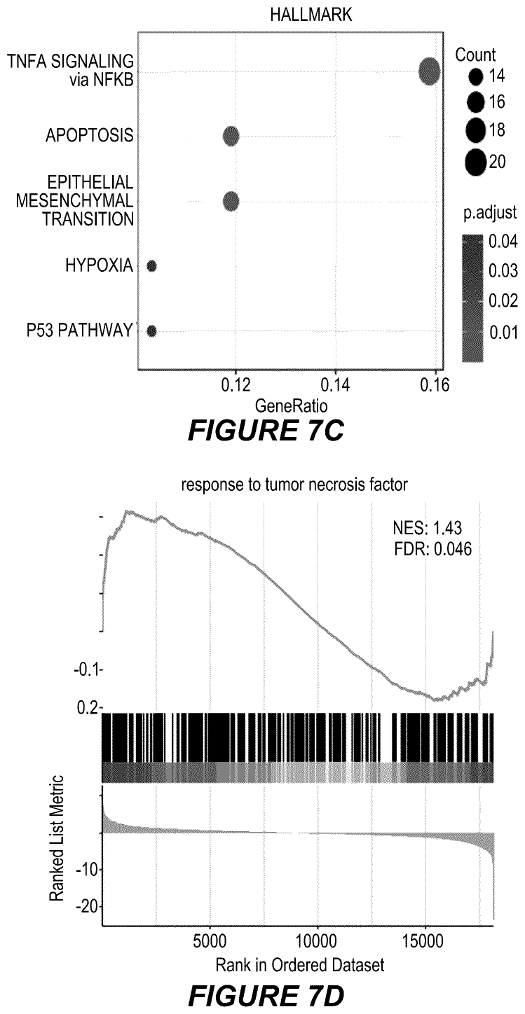

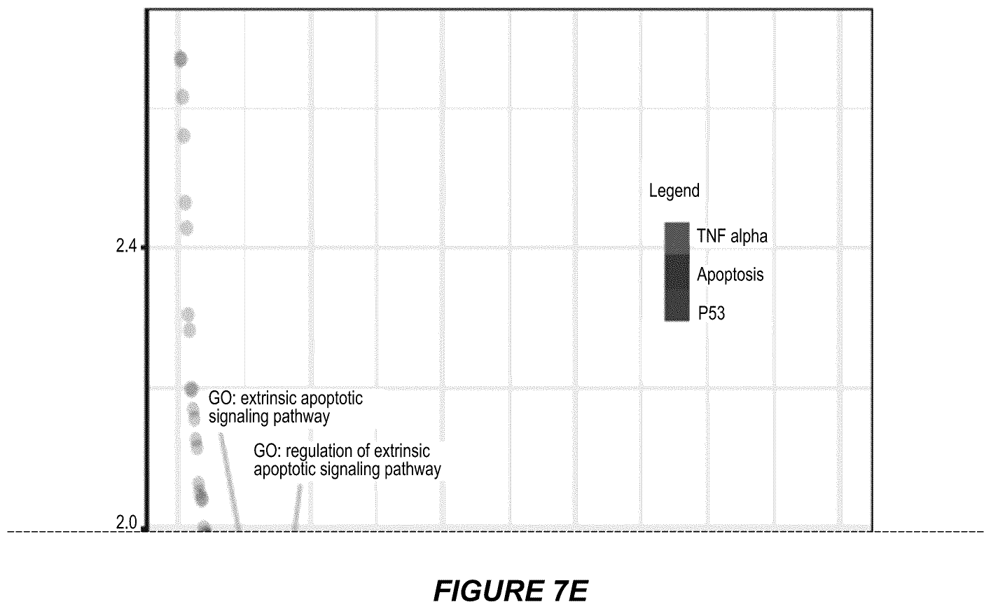

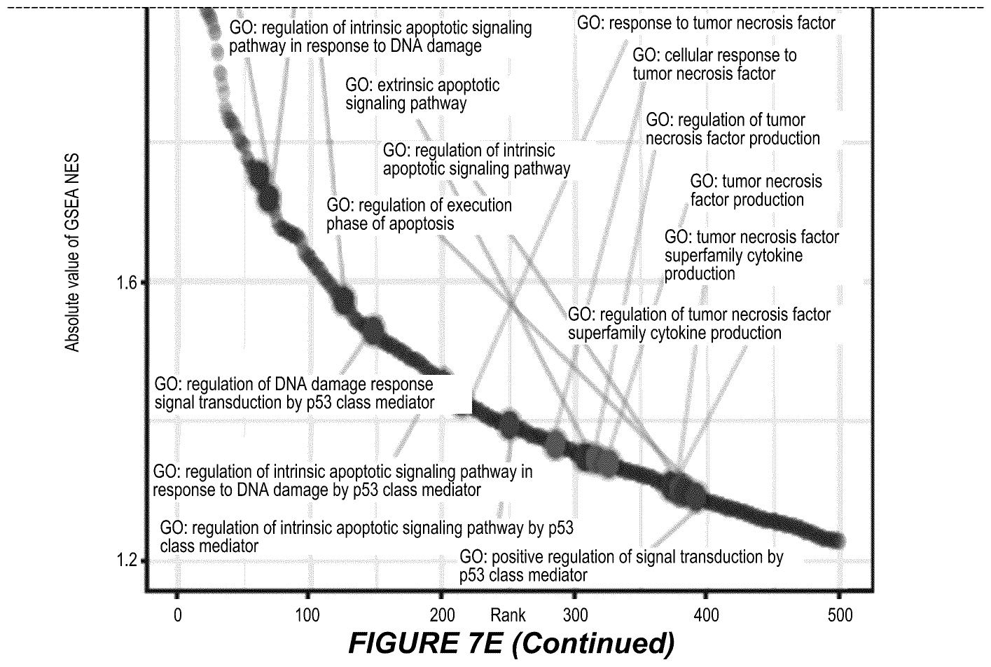

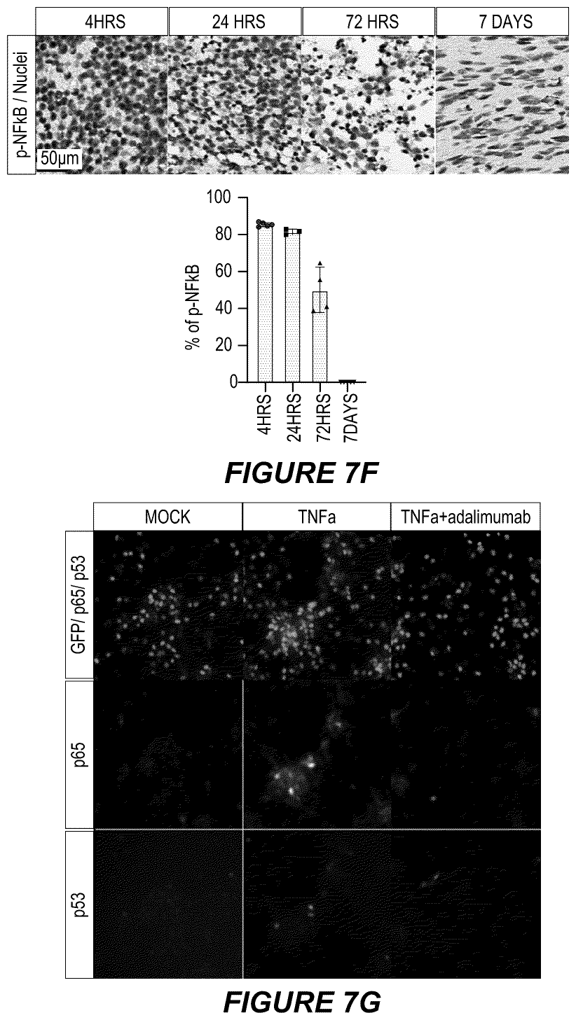

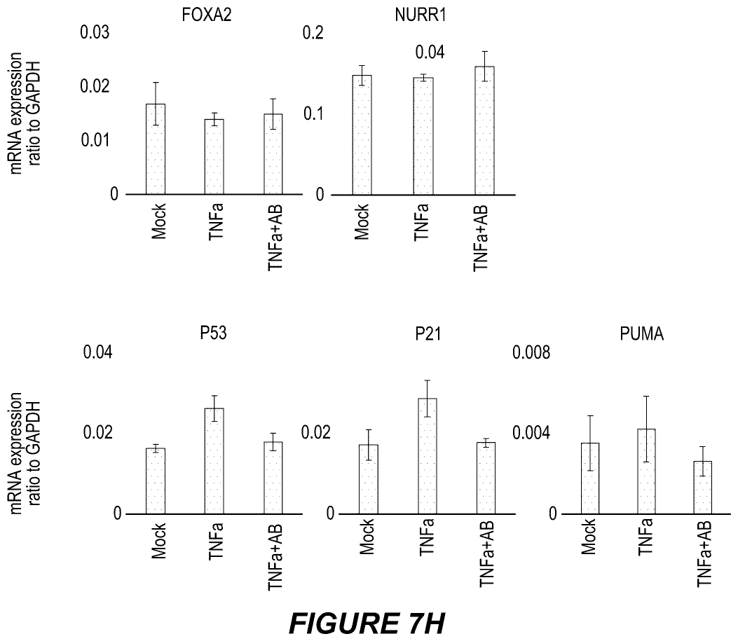

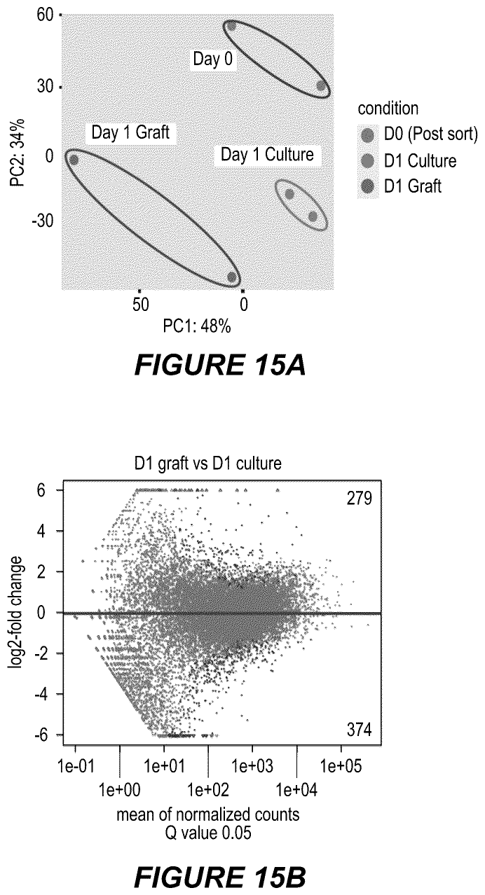

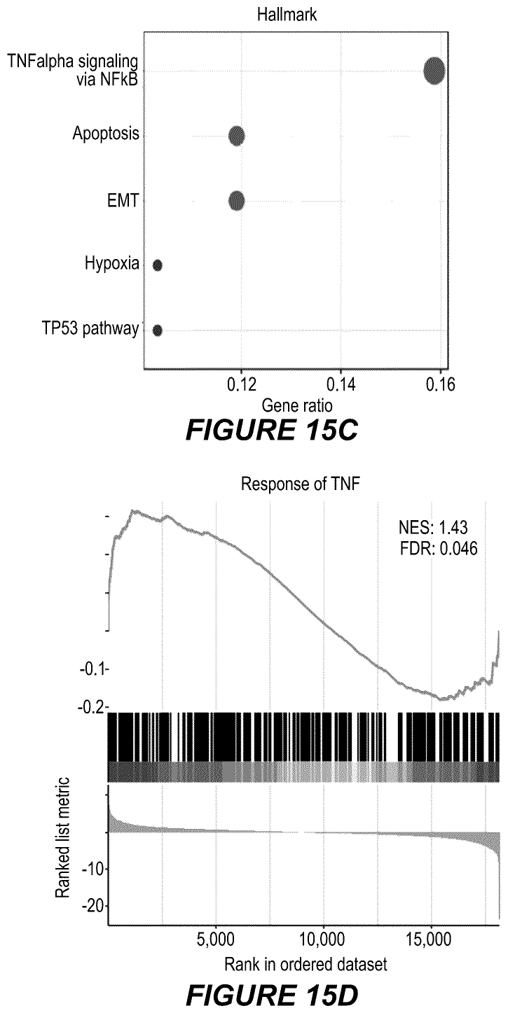

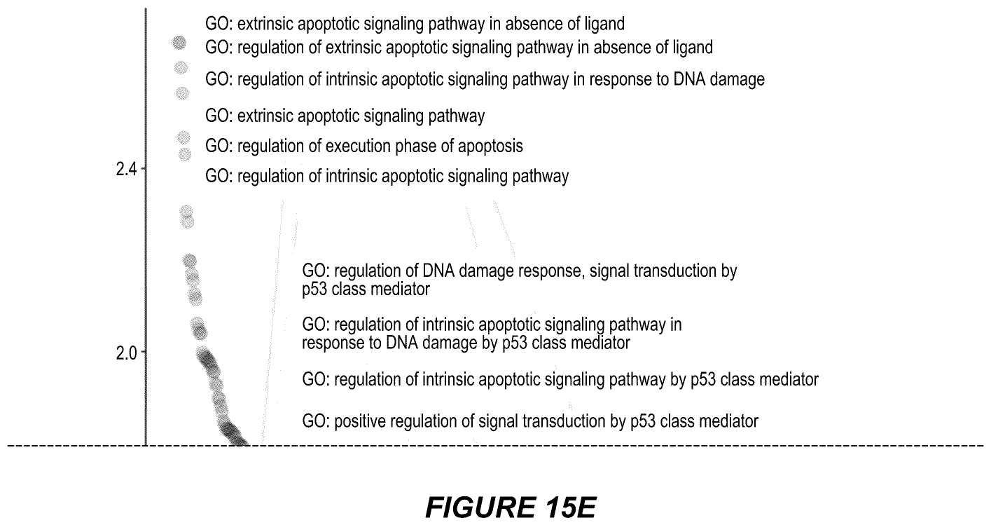

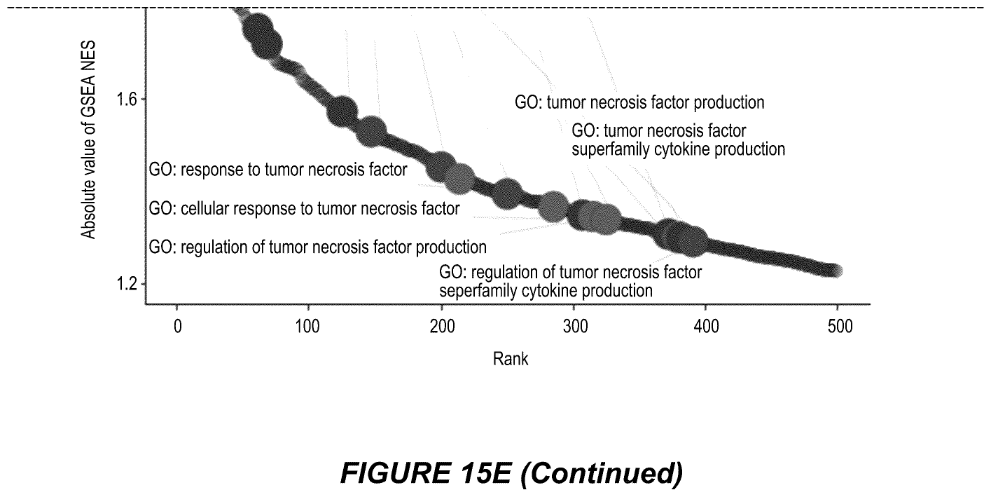

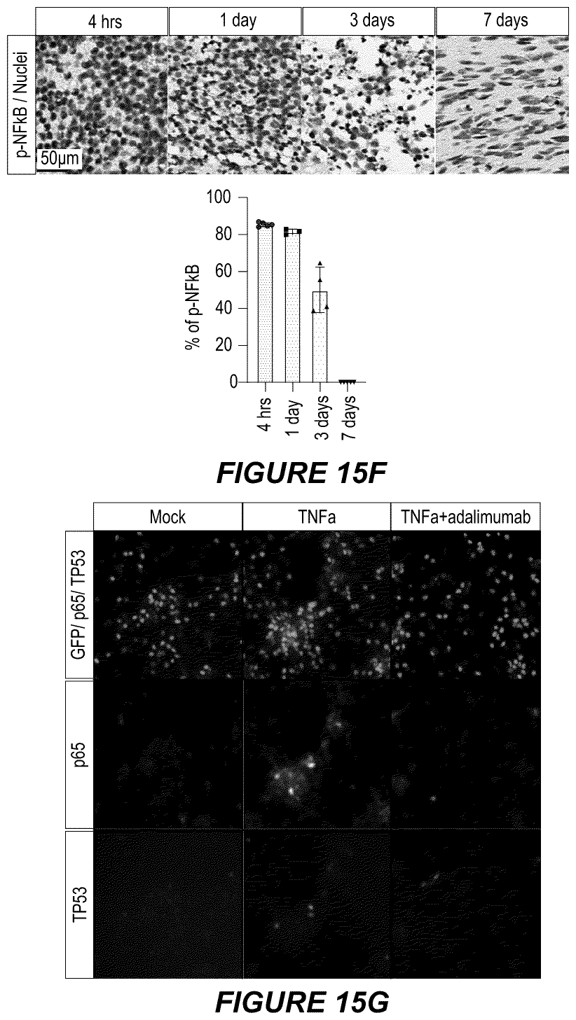

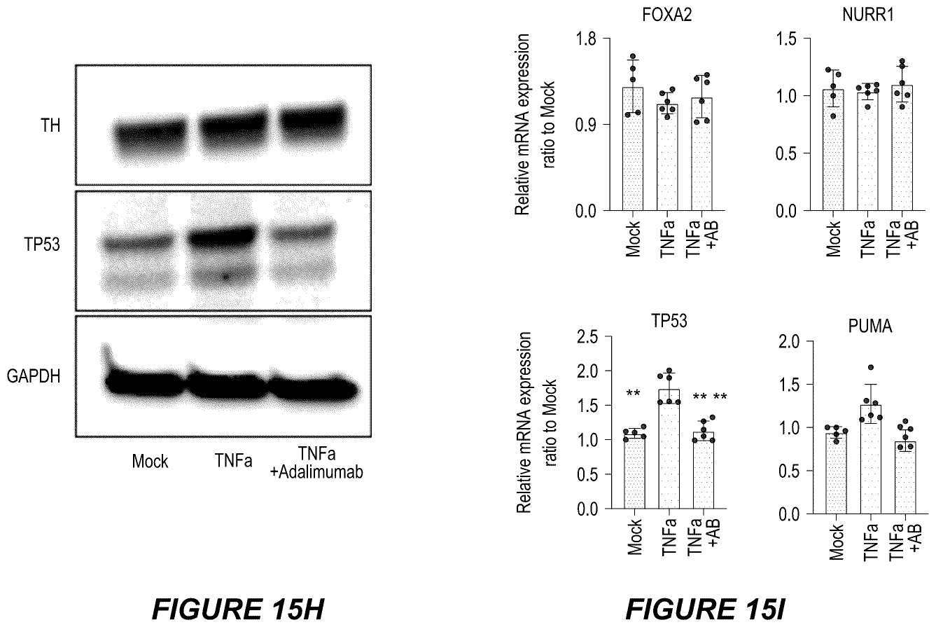

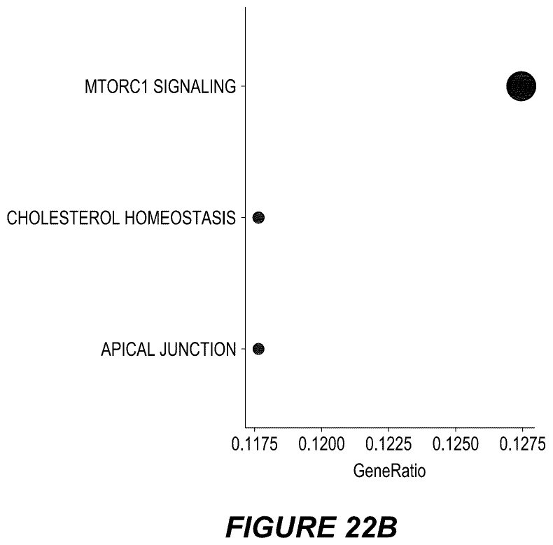

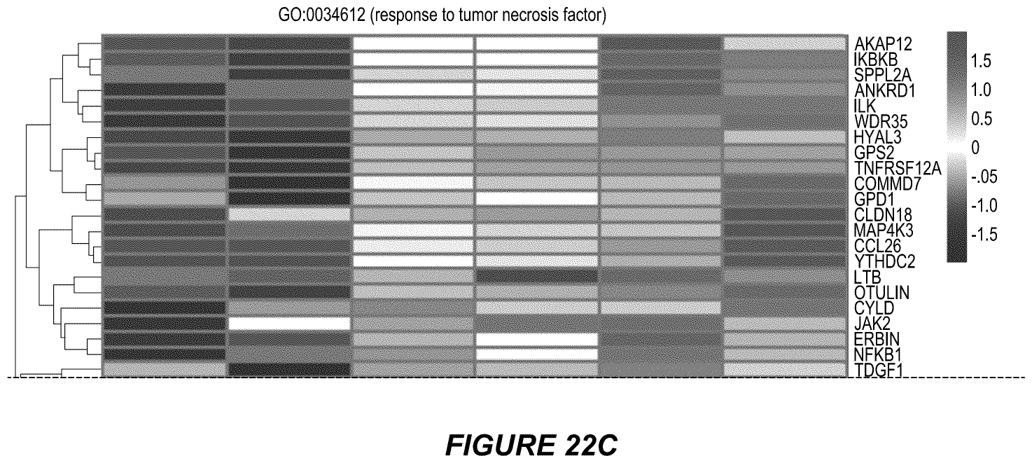

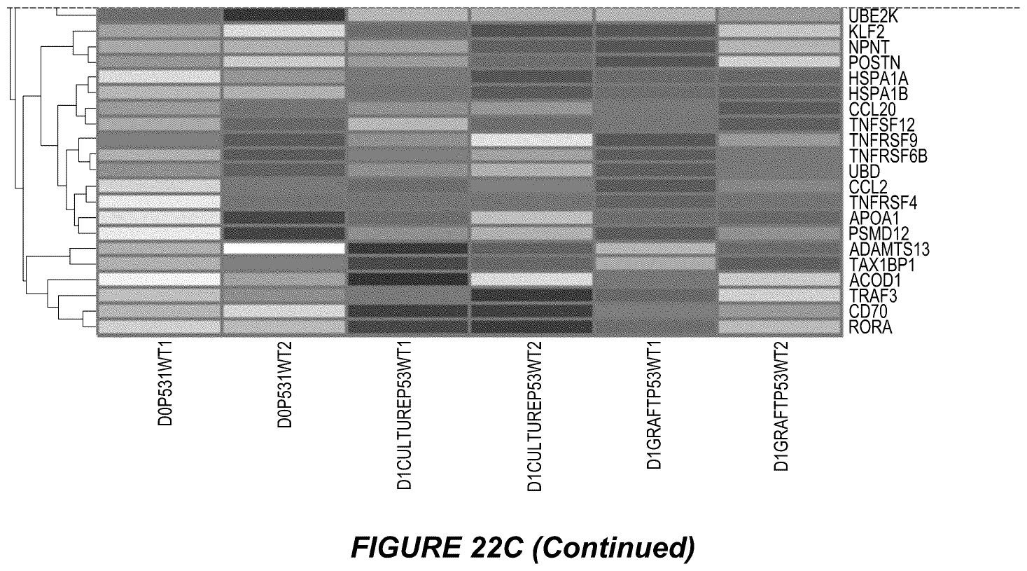

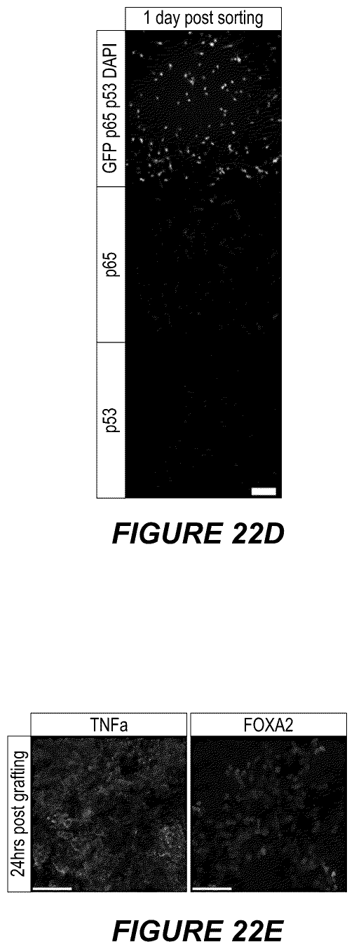

TNFα-NFκB pathway was an upstream regulator triggering TP53-dependent dopamine neuron death in the graft. Given a well-known p53 role as a tumor suppressor and high induction of p53 in the transplant at 1 dpt, it was sought to identify upstream factors of p53 induction. To gain a molecular mechanistic insight on the potential trigger of p53, a bulk RNA seq was performed to compare grafted neurons that were isolated 1 dpt from mouse brain with FACS-based sorted neurons at day 0 and in vitro culture neurons at 1-day post sorting. Principal component analysis (PCA) and dendrogram demonstrated that the grafted dopamine neurons exhibited a distinct transcriptional pattern compared to either sorted or in vitro cultured dopamine neurons (FIG. 7A; FIG. 15A). In particular, gene ontology analysis of the 279 differentially expressed genes (DEG) upregulated in 1 day in vivo grafted neurons versus 1-day in vitro cultured neurons showed that TNF alpha signaling via NF kappa B, apoptosis, hypoxia, and p53 pathways were significantly upregulated in the grafted neurons (FIGS. 7B and 7C; FIGS. 15B and 15C) whereas the 374 DEG downregulated in the in vivo grafted versus cultured neurons were associated with apical junction, mTORC1 signaling, and cholesterol homeostasis (FIG. 22B). Gene Set Enrichment Analysis (GSEA) of enriched genes in day 1 grafted neurons vs. day 1 in vitro plated neurons further confirmed increased signatures, including apoptosis and tumor necrosis factor signaling pathways (FIGS. 7D and 7E; FIGS. 15D and 15E).

Next, the phosphorylated form of nuclear factor kappa B (pNFκB) as an indication of active form of NFκB under TNF stimulation during the time course upon transplantation was examined. Time-course immunofluorescence assay during the transplantation indicated that pNFκB expression was heavily increased within the grafted neurons at 4 hours and 1 dpt, marking nearly 80% of the grafted neurons, and such induction was decreased within the graft beyond 3 dpt (FIG. 7F; FIG. 15F). At 1 day post plating, nuclear NFκB expression was not induced in dopamine neurons replaced in vitro (FIG. 22D). This result demonstrated that TNFα-NFκB signaling cascade immediately occurred after dopamine neuron engraftment prior to the p53-dependent apoptosis, suggesting a potential upstream role of TNFα-NFκB pathway on p53.

To validate the causal link of TNFα-mediated NFκB and p53 induction, treatments with a TNFα-blocking monoclonal antibody called adalimumab were administered at day 25 NURR1::H2B-GFP sorted neurons for 24 hours (FIG. 7G; FIG. 15G). Upon TNFα treatment, dopamine neurons increased p53 expression, which exhibited co-positive with NFκB-p65. In addition, p53 and p53 downstream genes, such as p21 and PUMA mRNA were upregulated in the TNFα exposure conditions while co-treatment with TNFα and adalimumab abolished such inductions (FIGS. 7G and 7H; FIG. 15G-15I), confirming a TNFα-NFκB-p53 axis in this established in vitro model system.

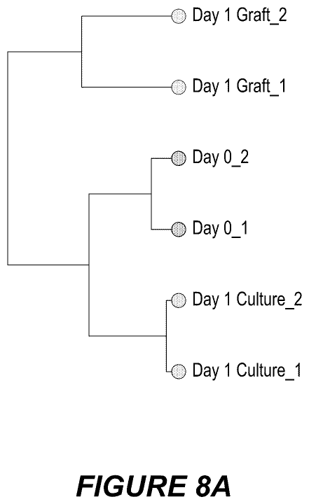

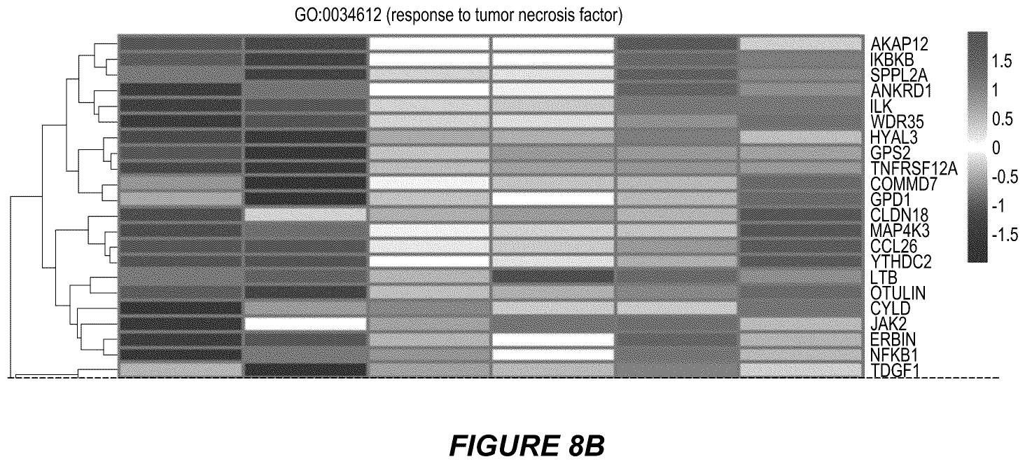

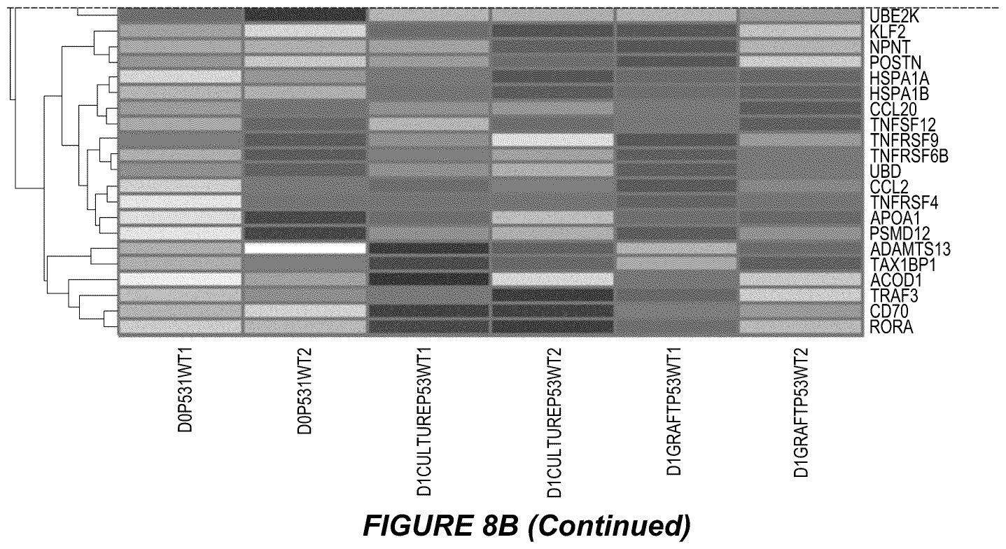

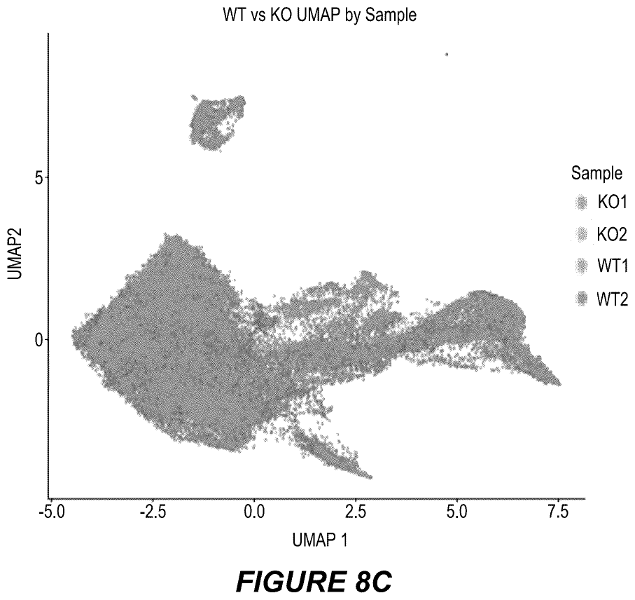

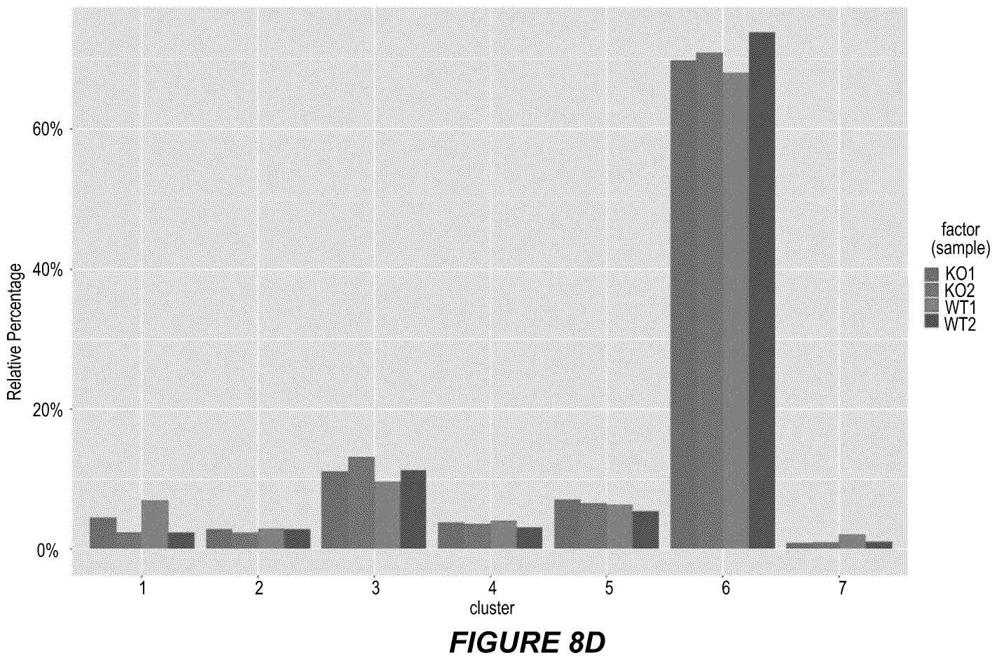

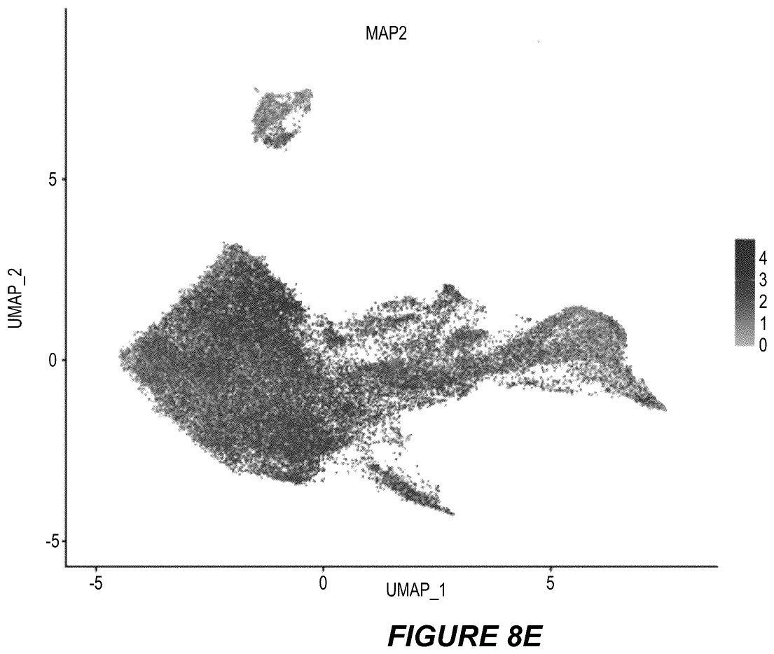

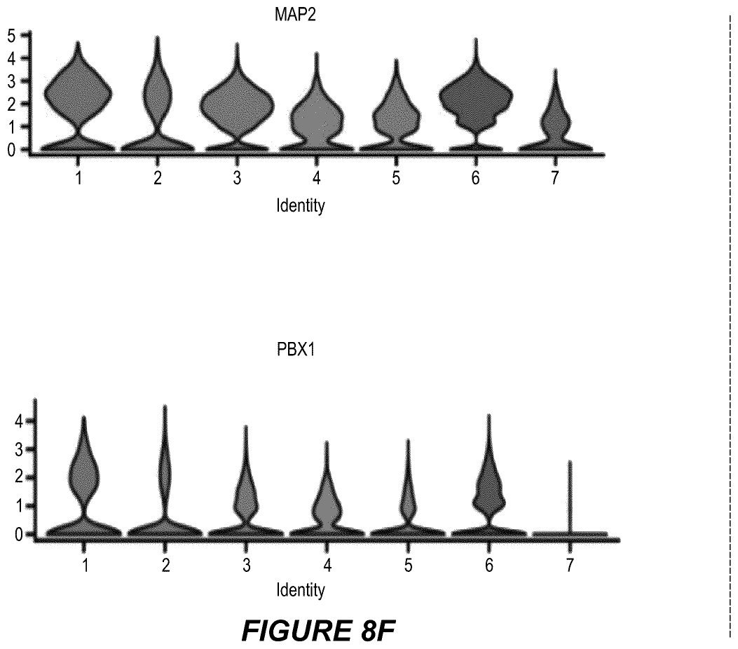

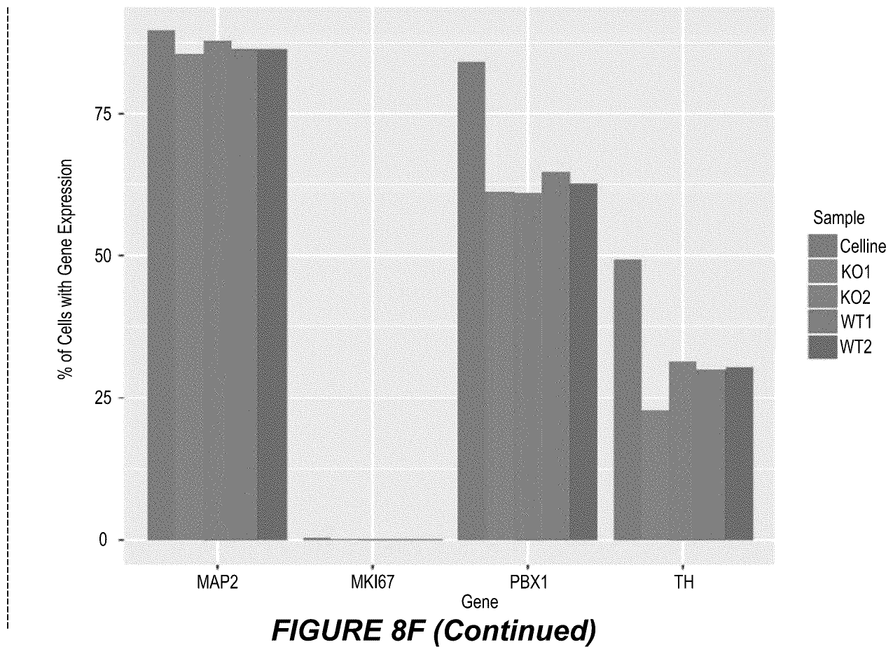

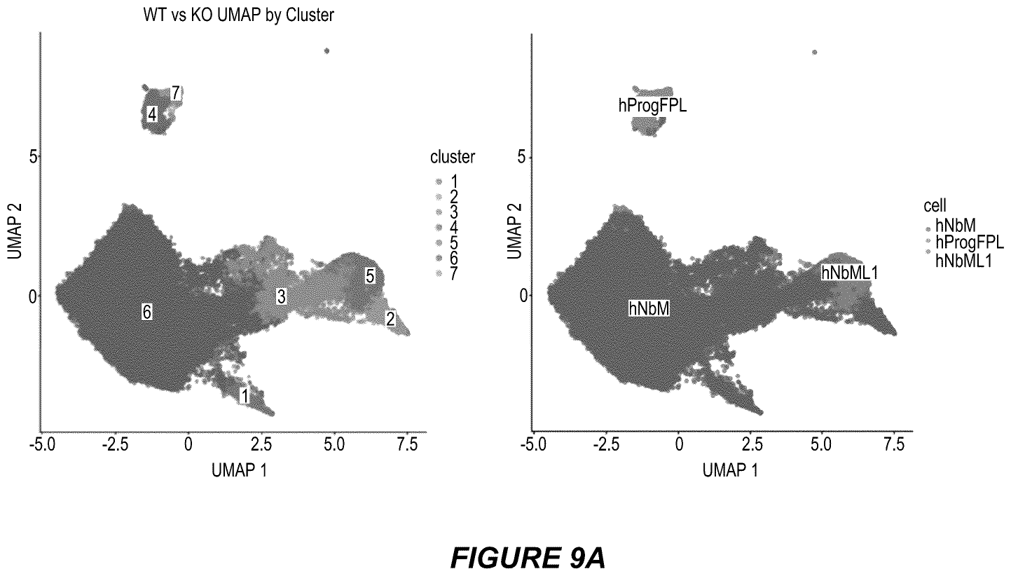

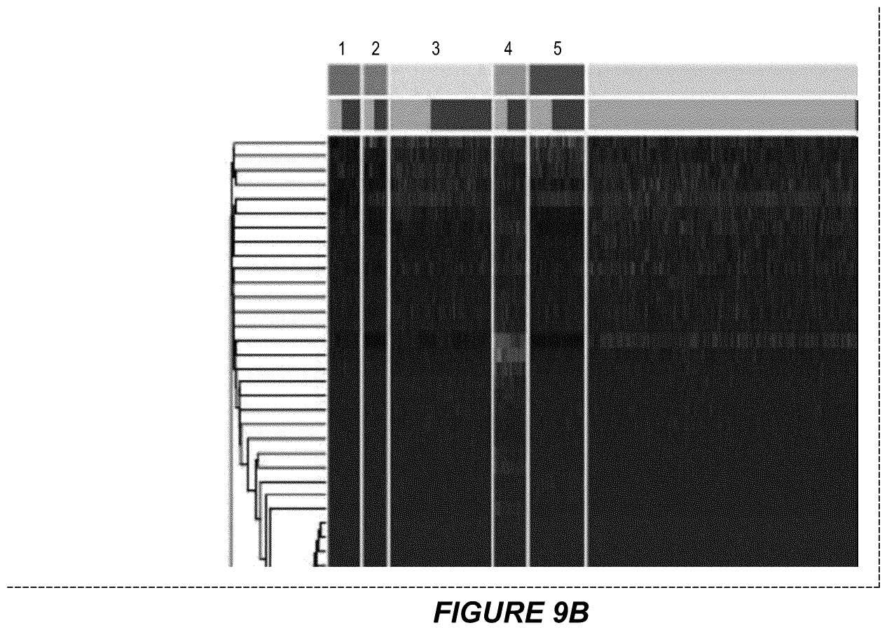

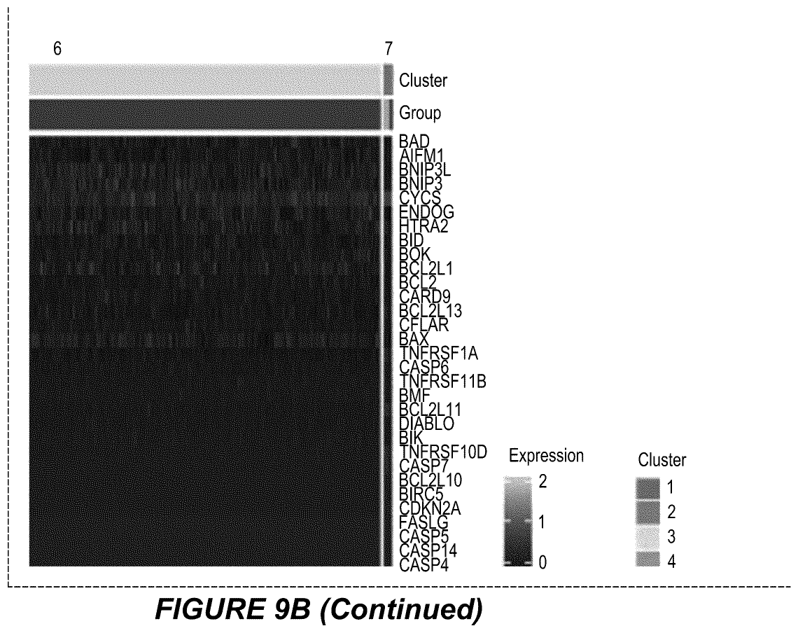

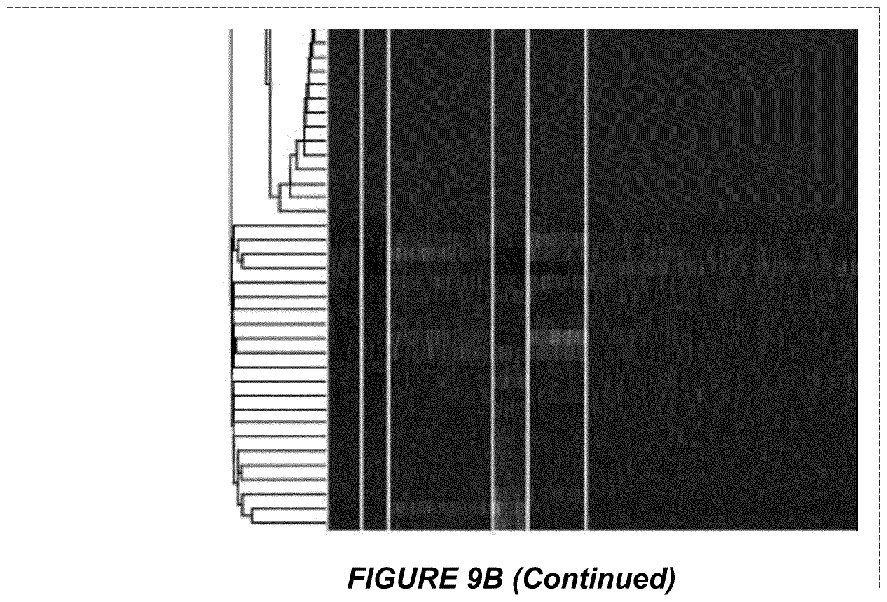

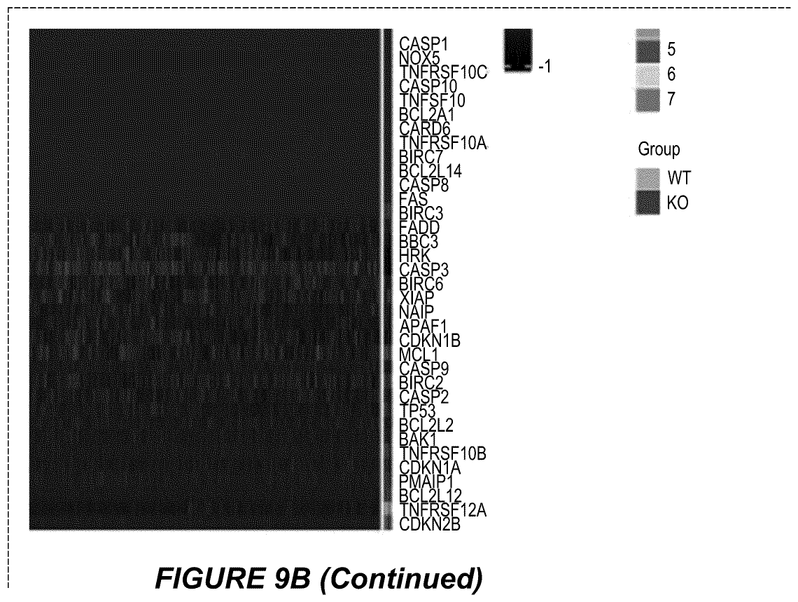

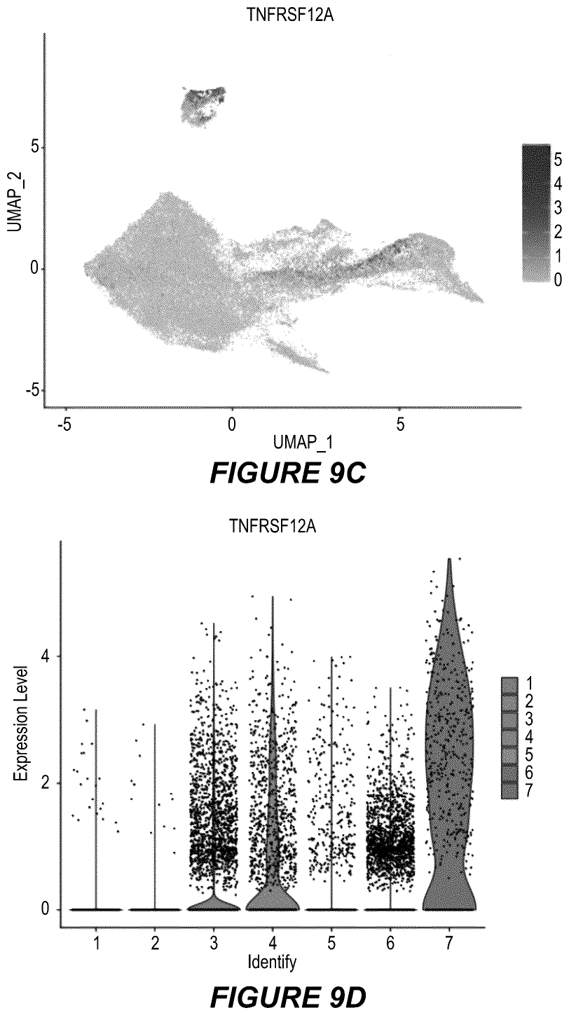

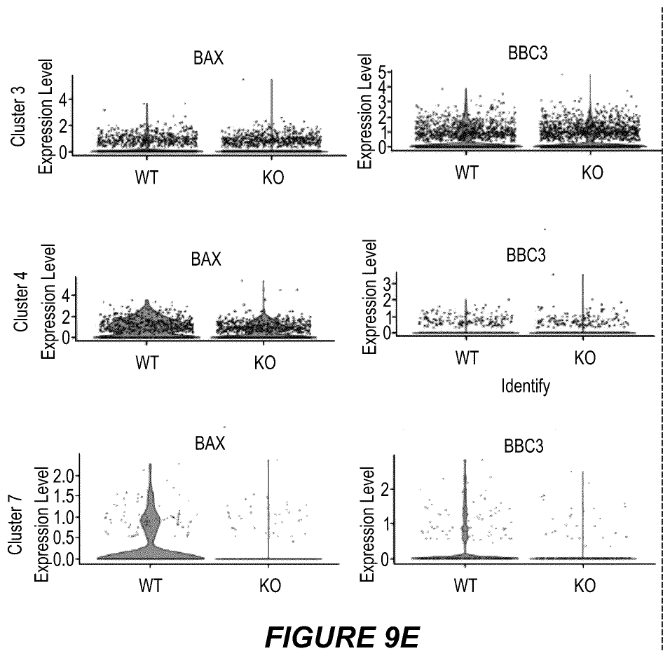

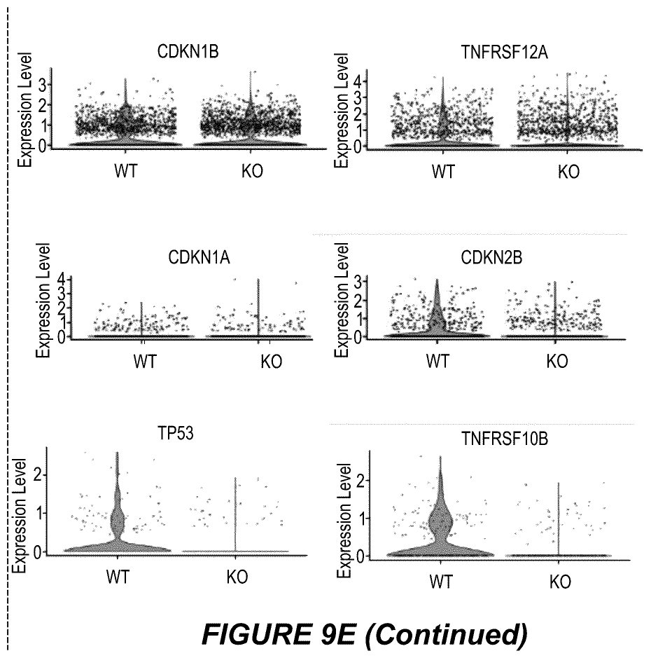

Single-cell RNA sequencing of grafted neurons revealed p53-BAX axis is a key driver for neuronal death during transplantation. In order to gain a molecular landscape of grafted dopamine neurons at a single cell level, a single cell mRNA sequencing from p53 WT and KO grafted neurons from the mouse brain at 1 dpt was performed. Uniform Manifold Approximation and Projection for Dimension Reduction (UMAP) analysis of wild-type and p53 knock-out (KO) samples showed a highly overlapping clustering distribution (FIGS. 8B and 8C). Based on PBX1 for a dopamine-specific marker and MAP2 expression for neurons, >90% of the cells exhibited post-mitotic dopamine neuron identity with very low expression of the proliferation marker, MK167, in both p53 WT and KO neurons (FIGS. 8D and 8E). By comparing these data to an available human fetus dataset, the majority of these cells, which occupy one large cluster, were annotated as neuroblasts, which indicated NURR1 expressing stage, and a small cluster as a floor plate progenitor (FIG. 9A).

Then, it was sought to determine whether any specific clusters showed molecular signatures of neuronal death. Heatmap from apoptotic cell-death related genes identified that clusters 3, 4, and 7 (FIGS. 9B-9D) showed increased genes, such as BAX, BAD, TNFRSF1A, TNFRSF12A, and TNFRSF10B. Violin plots of these genes from each cluster further demonstrated that these genes were more significantly expressed in WT than p53 KO neurons, supporting that TNF induced p53-BAX genes triggering p53 dependent apoptosis pathway in the graft (FIG. 9E).

Questions remain regarding the source of the TNFα ligands. This dataset points out that grafted dopamine neurons expressed TNFα superfamily ligands, potentially hinting that dying neurons secreted TNFα themselves upon acute post-traumatic injury (data not shown).

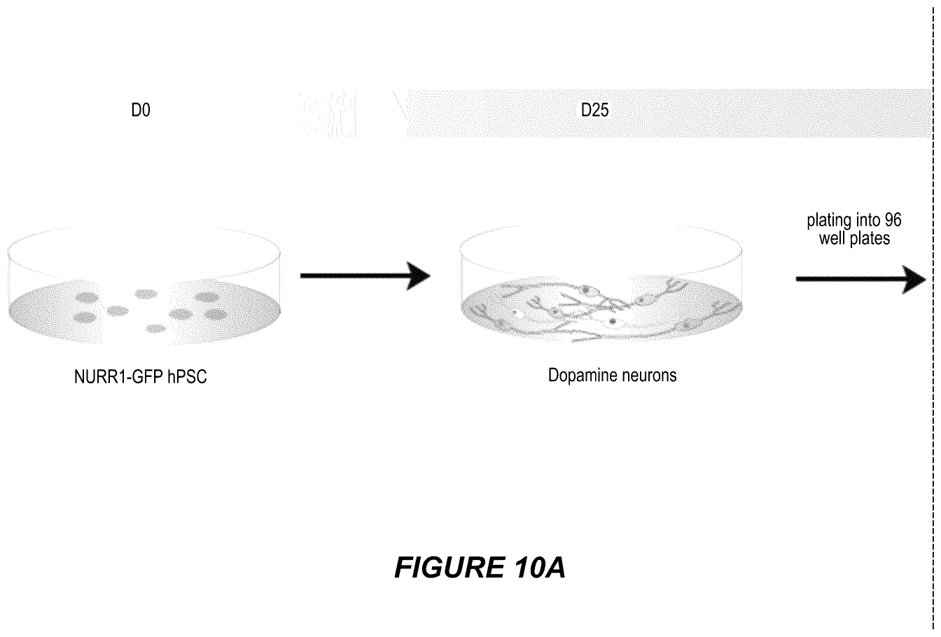

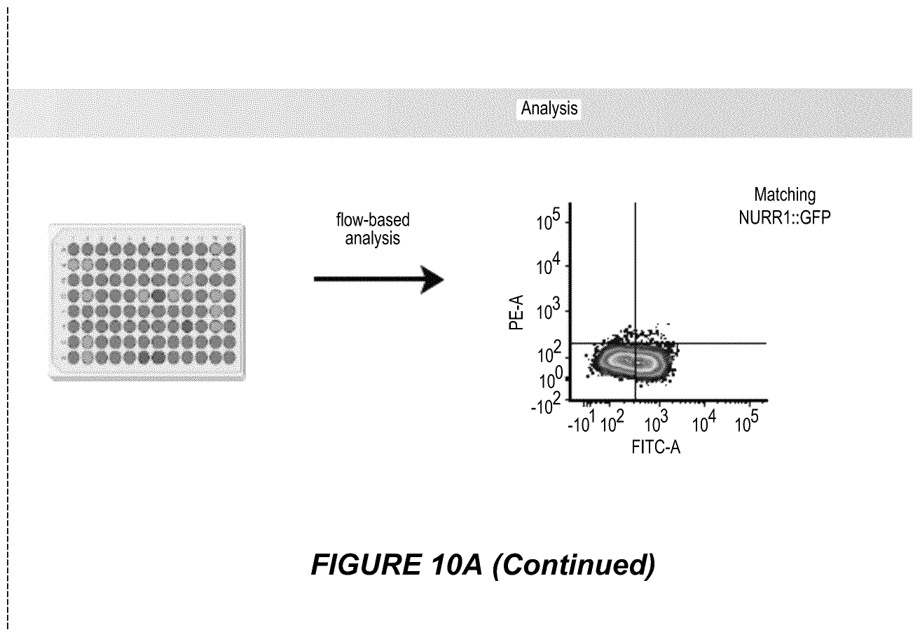

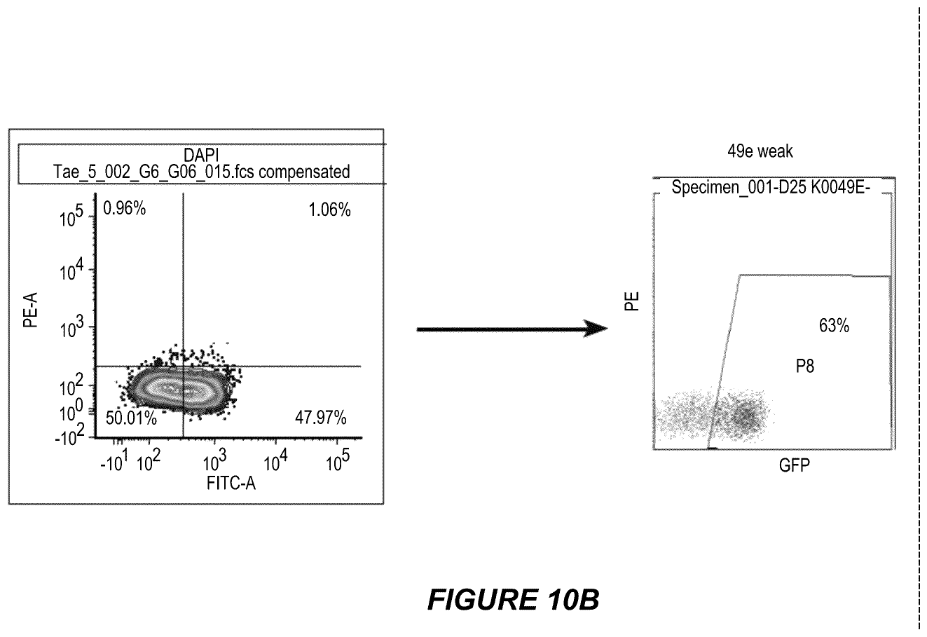

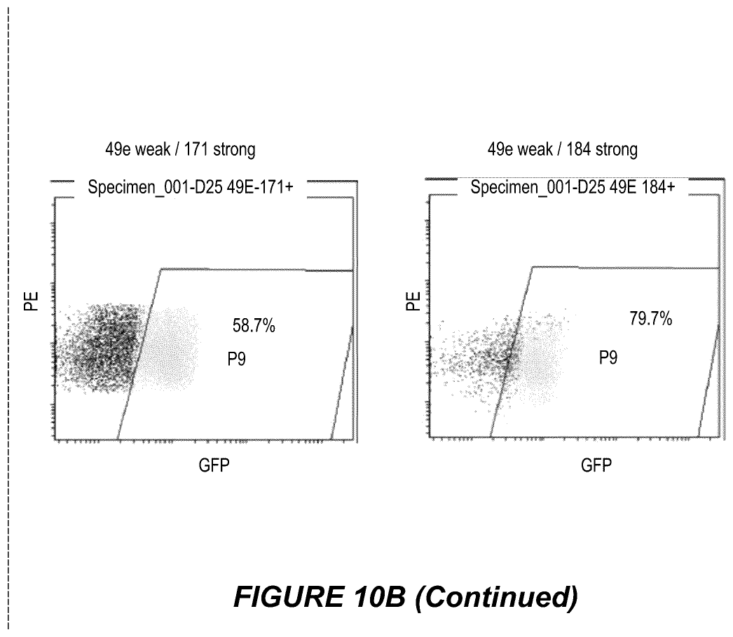

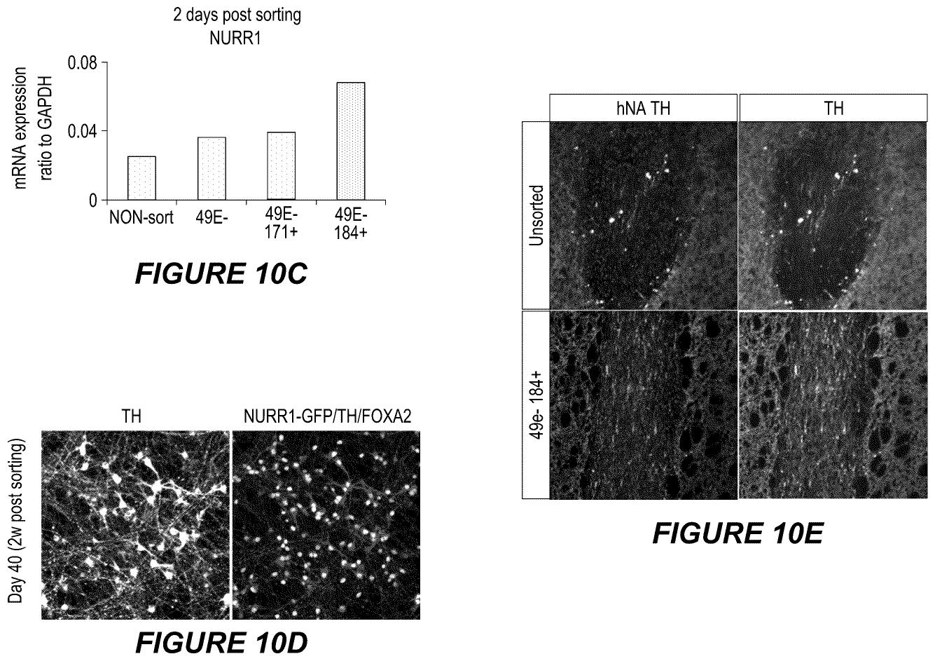

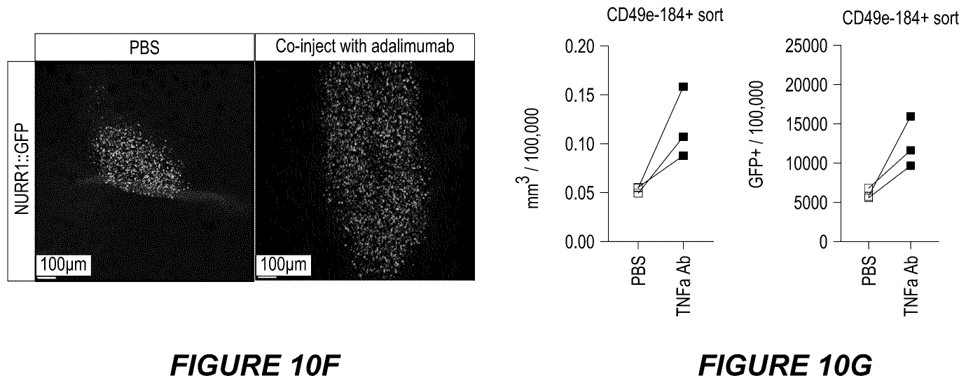

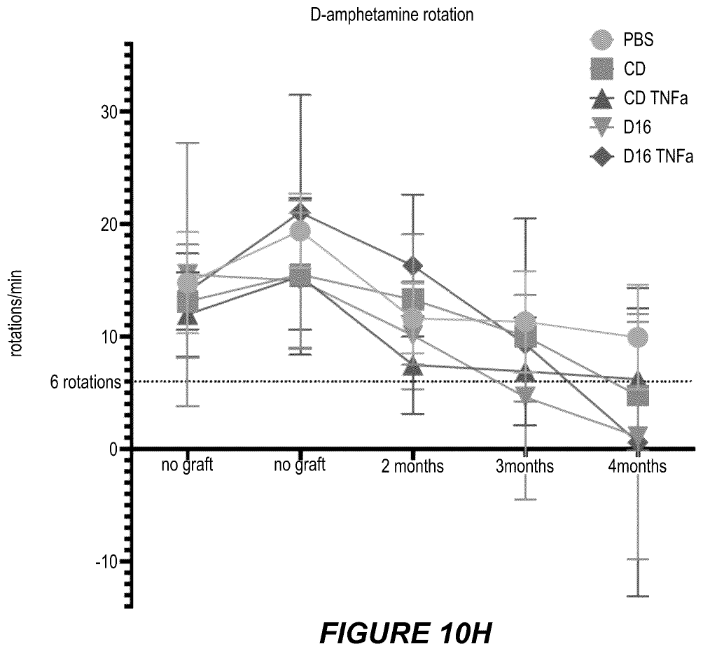

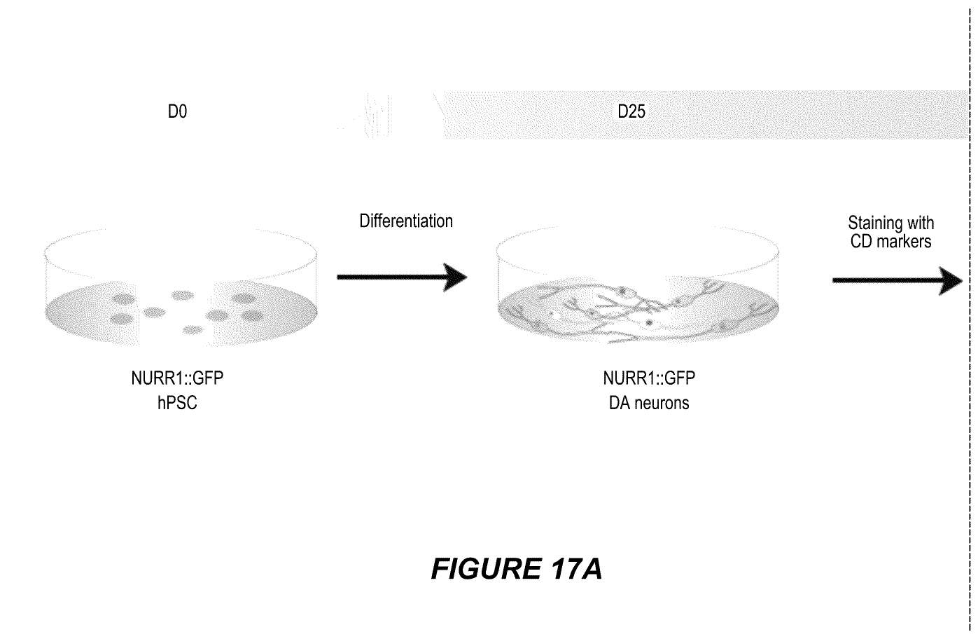

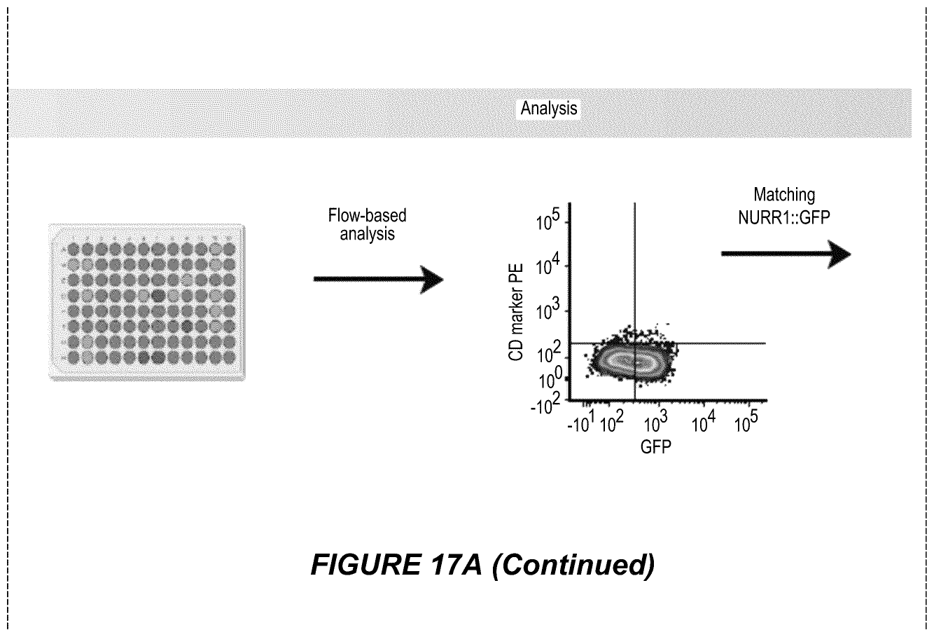

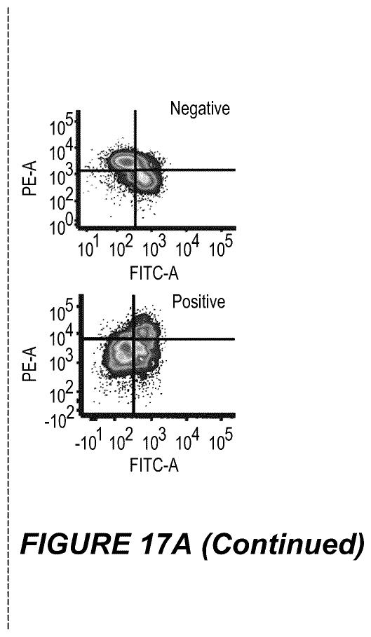

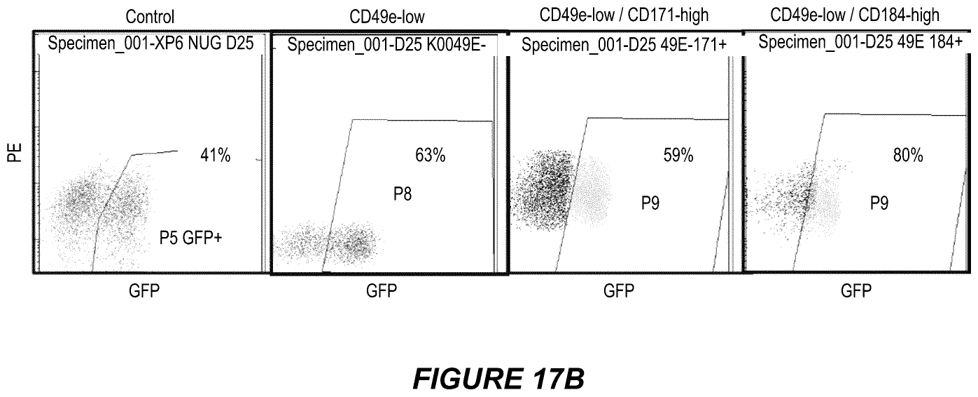

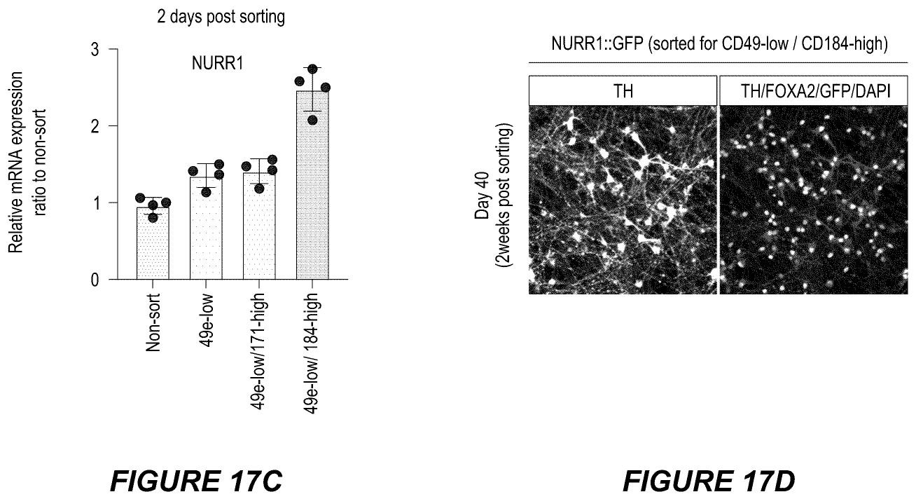

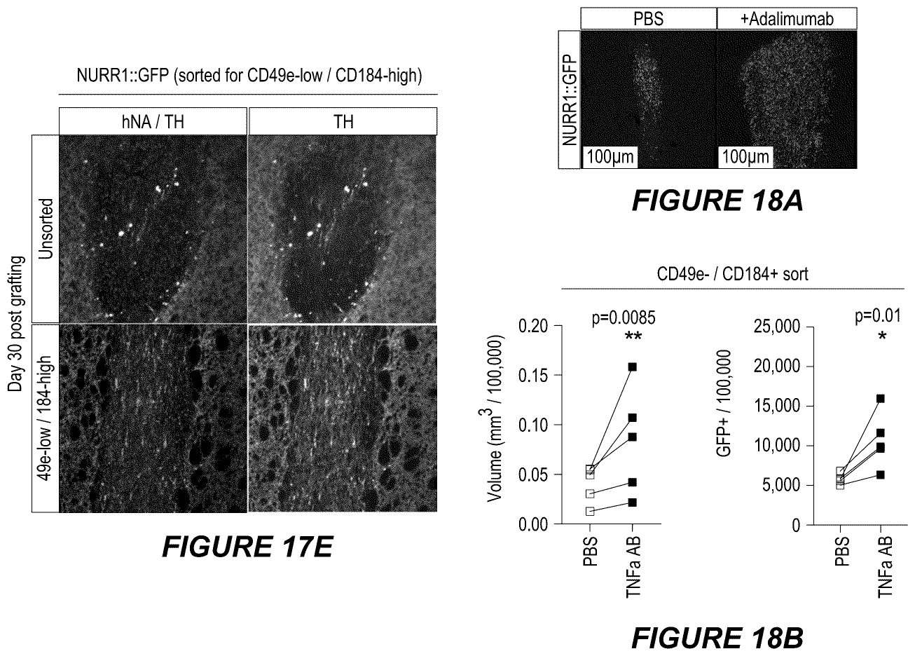

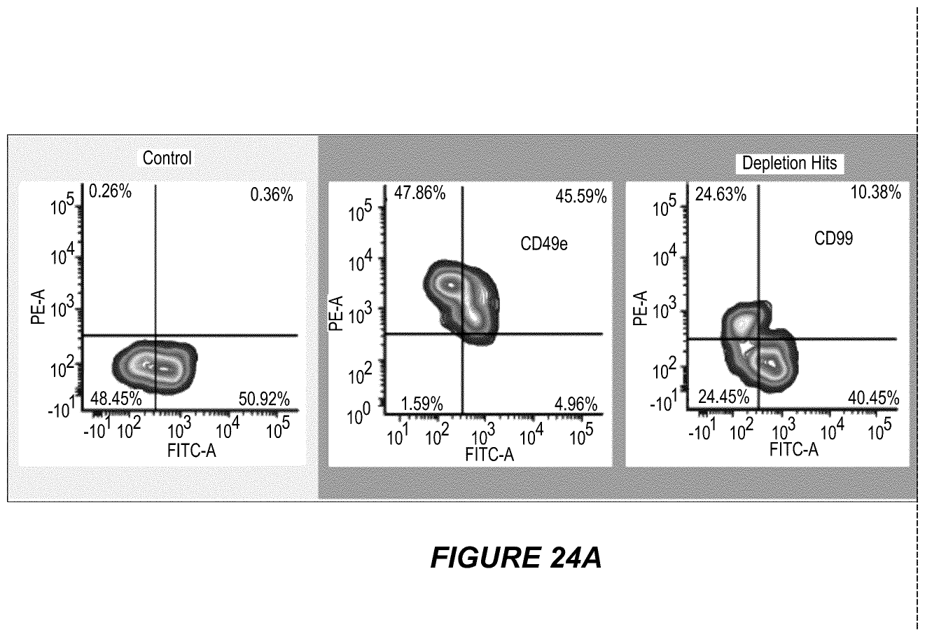

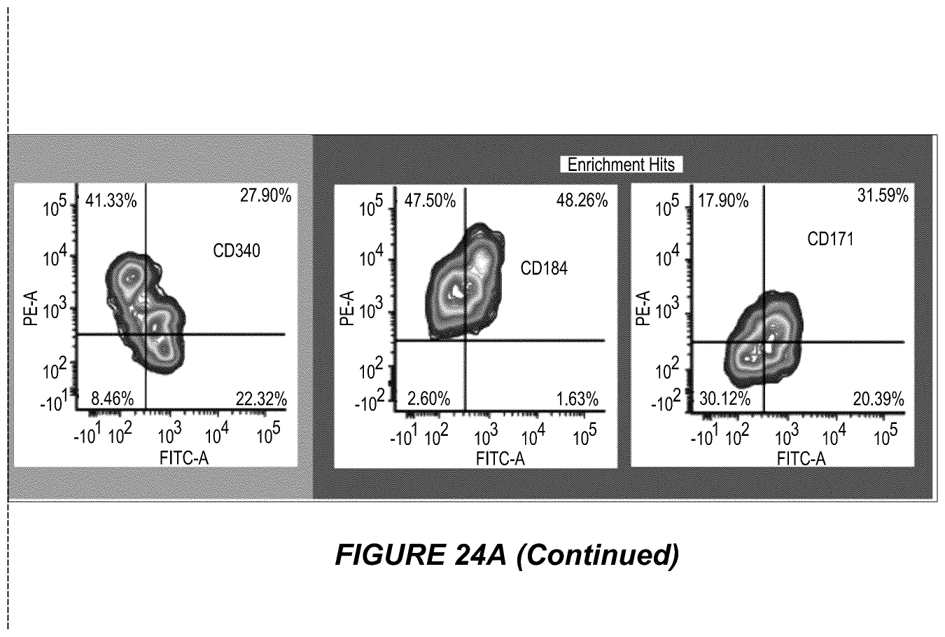

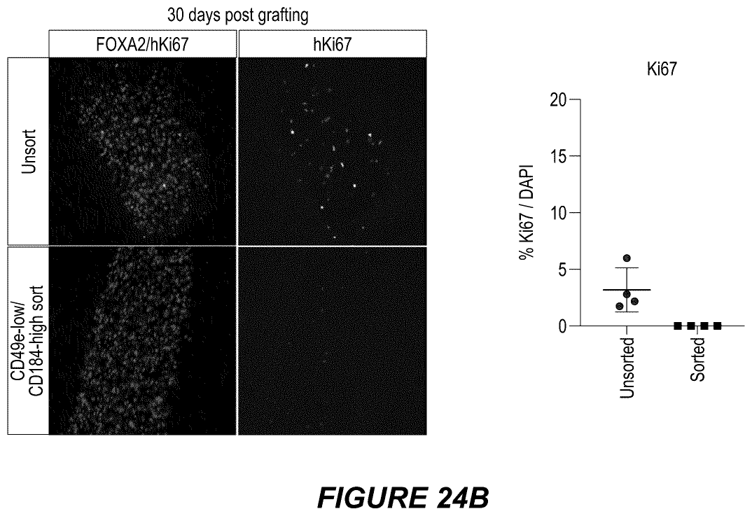

High throughput cell surface marker screen defined novel CD markers to enrich post-mitotic dopamine neurons. Given the complication of using genetically engineered hPSC for translational application, it was sought to define surface markers to capture post-mitotic dopamine neurons obviating the need for genetic selection. A high-throughput flow-based cell surface marker screen with 385 validated antibodies identified the candidate CD markers with three depletion hits (CD49c, CD99, CD340) and two enrichment hits (CD184, CD171) to match the GFP signal from NURR1::H2B-GFP hPSC derived dopamine neurons at day 25 (FIGS. 6 and 10A; FIGS. 21D and 17A). After extensively characterizing sorted cells either using a single and/or double CD markers via flow analysis, qRT-PCR, and immunofluorescence assay to examine dopamine neuron identity, it was found that CD49e low and CXCR4/CD184 high expressing cell showed the most highly enriched pure post-mitotic dopamine neurons, expressing FOXA2, NURR1::GFP, and TH 2 weeks post sorting in vitro (FIGS. 10B-10D; FIGS. 17B-17D). Moreover, the short-term histology analysis at 1-month post-transplantation demonstrated that CD-sorted dopamine neurons using this novel double sorting strategy exhibited graft survival expressing TH homogeneously without any human Ki67 proliferating cells, emphasizing on translation benefits to use enriched post-mitotic dopamine neurons via the novel CD marker strategy for transplantation (FIGS. 10E and 11B; FIGS. 17E and 24B).

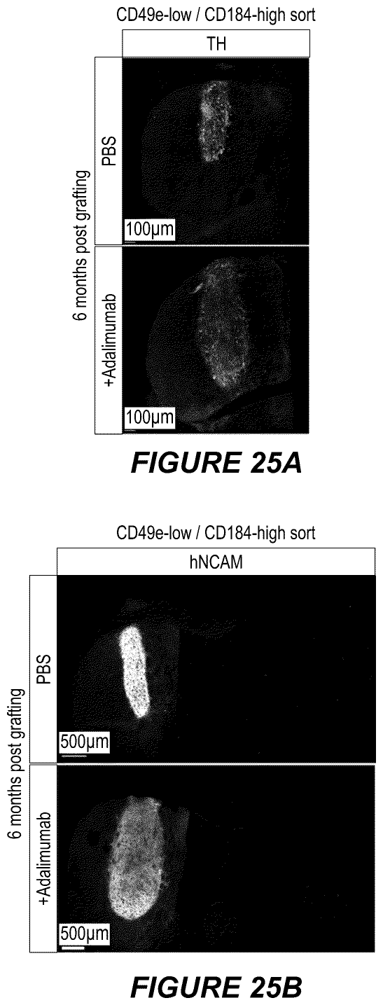

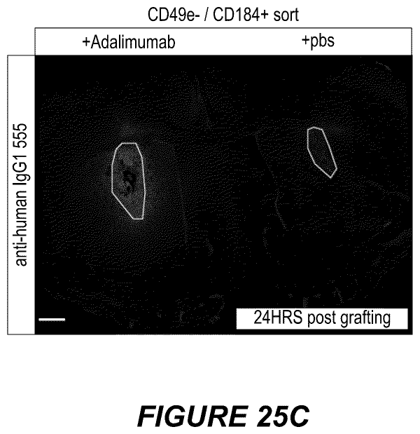

TNFα neutralizing monoclonal antibodies improved the survival of CD marker sorted post-mitotic dopamine neurons in the graft. Next, it was examined whether a publicly available TNFα neutralizing antibody called adalimumab (Humira) could have functional significance on CD-sorted dopamine neuron survival. The adalimumab has been reported to bind to soluble and transmembrane bound TNFα (McCoy and Tansey, 2008) and is widely used to treat arthritis and encephalitis to subdue the inflammation. Co-injection with adalimumab significantly improved the survival of CD marker sorted dopamine neurons in vivo post-transplantation (FIGS. 10F and 10G; FIGS. 18A and 18B). Stereological counts of NURR1::GFP+ cells at 1 month post grafting found 12,423 (mean)+1,859 (S.E.M) in adalimumab treated grafts per 100,000 cells injected versus 6,057 (mean)±378 (S.E.M) in PBS treated neurons (p=0.01, paired t-test, n=5) (FIG. 18B). The volume of the adalimumab treated graft was 0.1175 mm³ (mean)±0.003 mm³ (S.E.M) per 100,000 cells in PBS-treated grafts (p=0.0085, paired t-test, n=5). Furthermore, it was examined whether CD sorted postmitotic dopamine neurons, with or without adalimumab treatment, are functional in rescuing amphetamine-induced rotation behaviors in 6-OHDA treated mice, which represents a widely used preclinical model for treating PD motor symptoms. Robust functional recovery, regardless of adalimumab exposure, was observed at 6 months post grafting. Adalimumab-co-injected mDA neurons showed a trend towards an earlier and more complete functional rescue (FIG. 10H; FIG. 18C). Histological analyses showed that both groups give rise to highly enriched neuronal population in vivo at 6 months post implantation (FIGS. 25A-25B). However, TNFα inhibition via adalimumab resulted in significantly increased total numbers of surviving dopamine neurons as well as overall graft size (FIGS. 18D and 18E). Similar to the results with p53 KO cells, we observed that adalimumab treatment results in significant increase in the proportion of ALDH1A1+A9 mDA neuron subtype without affecting the fraction of CALB1 expressing A10 dopamine neuron subtype (FIGS. 18F and 18G). These results implied the CD marker sorting and TNFα neutralizing antibody strategies could be directly applied for clinical translation.

### Discussion

The main gene identified in presently disclosed in vivo Crispr screen for dopamine neuron survival was TP53. TP53 is a master regulator of diverse cellular processes ranging from tumor suppression to serving as a guardian of cell fate identity and reprogramming to sensing cellular stresses related to DNA damage, oxidative stress, or ischemic injury among others. TP53 has been recently implicated as a candidate factor in driving the vulnerability of human substantia nigra dopamine neurons during PD pathogenesis based on selective expression patterns by single cell analysis. Furthermore, there has been interest in the role of TP53 as a general signaling hub of neuronal cell death across neurodegenerative disorders such as the p53-mediated regulation of C9ORF72-mediated neuronal death in ALS (Maor-Nof et al., 2021). The presently disclosed initial screen identified several genes in addition to TP53 as limiting in vivo dopamine neuron survival including TNFRSF11B, BBC3, BCL2L11, CASP2, CASP9, genes that are all linked to the TNFα/TP53/apoptotic pathway. Alternative hits such as SLC7A11, IL18 or TLR4 are associated with either ferroptosis or neuroinflammatory responses respectively.

The present disclosure focused on cell intrinsic factors driving dopamine neuron death by screening for genes limiting survival directly in purified dopamine neurons and without manipulating any host-related responses. Histological analyses were used to describe a cascade of host cell-related responses at the graft site including the recruitment of neutrophils, microglial and vascular cells that enter the graft core by 3 dpt, and the activation of inflammatory programs in both astrocytes and microglia. The presently disclosed temporal analysis suggests that host responses occur in response to rather than mediating dopamine neuron death. The grafting-related dopamine neuron death had subsided completely by 7 dpt.

The presently disclosed gene expression data demonstrates that TNFα is produced by grafted dopamine neurons post transplantation, possibly in response to injury-related damage. TNFα is known as an inflammatory cytokine secreted in response to hemorrhagic, ischemic or traumatic injury (Tuttolomondo et al., 2014), events commonly associated with cell transplantation.

The use of a genetic reporter line to purify postmitotic dopamine neurons allowed to address the survival without any confounding factors related to cell proliferation or non-autonomous factors. This approach also sets the stage for grafting postmitotic dopamine neurons for translational applications. The presently disclosed CD surface marker-based strategy enables dopamine neuron enrichment at the NURR1 stage without the need for establishing genetic reporter lines. Therefore, this strategy is compatible for translational applications for dopamine neuron replacement therapy in PD. It can also facilitate the use of purified dopamine neuron preparations in human iPSC-based disease modeling given the challenge in reliably generating specific neuron subtypes across many cell lines and laboratories when modeling neural disorders in a dish.

The presently disclosed study demonstrates that co-injection of a TNF neutralizing antibody can greatly reduce neuronal death post grafting when combined with the CD marker strategy to enrich for post-mitotic dopamine neurons. This technology could result in cell therapy approach that maximizes safety for PD patients by avoiding the transplantation of dopamine neuron precursors that retain at least a short-term potential for in vivo cell proliferation (Kim et al., 2021, Cell Stem Cell February 4; 28 (2): 343-355.c5; Kirkeby et al., 2017, Cell Stem Cell 20, 135-148.; Piao et al., 2021, Cell Stem Cell 28, 217-229 e217; Schweitzer et al., 2020, N Engl J Med 382, 1926-1932; Takahashi, Neuron. 2017 Sep. 13; 95 (6): 1395-1405.e3).

### Example 2—Single Cell RNA Sequencing of Day 1 Grafted Neurons Identifies Molecular Signature of Surviving Dopamine Neurons and De-Differentiation Signature in Apoptotic Dopamine Neurons

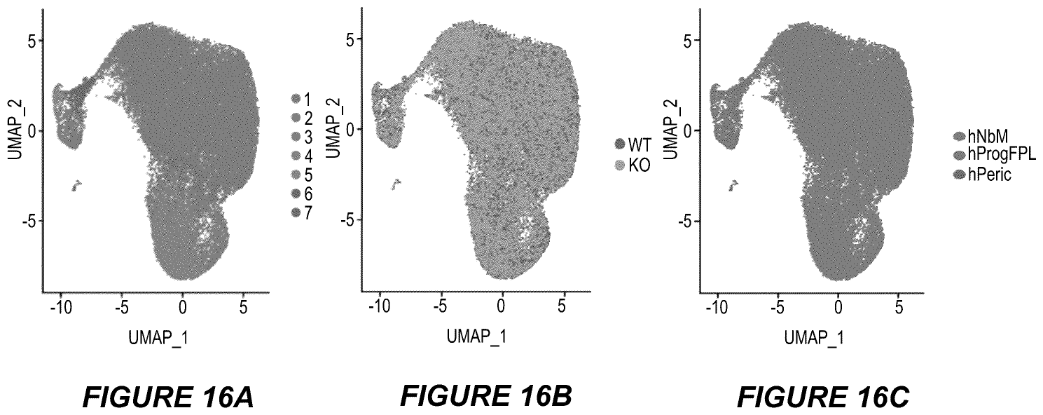

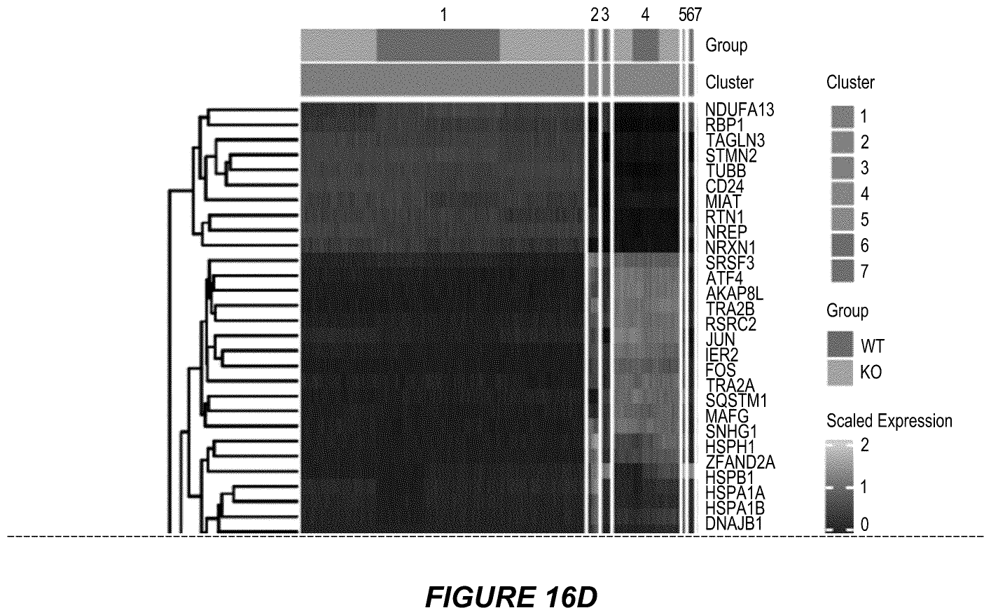

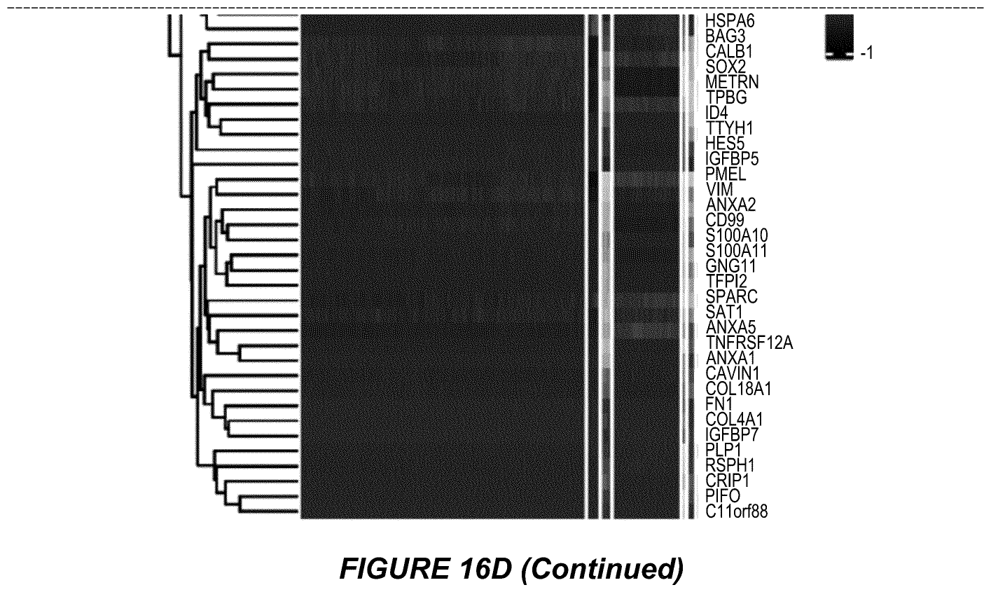

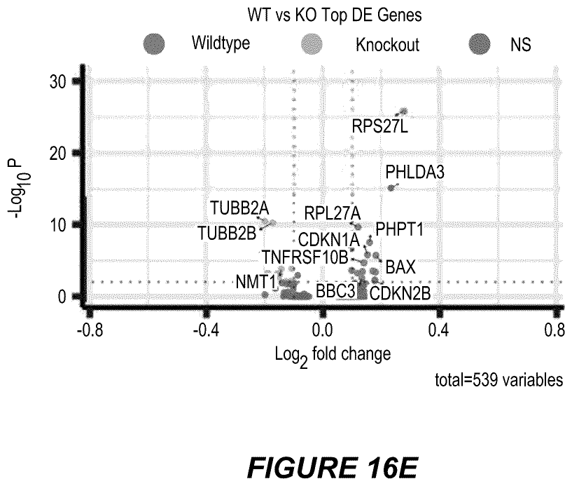

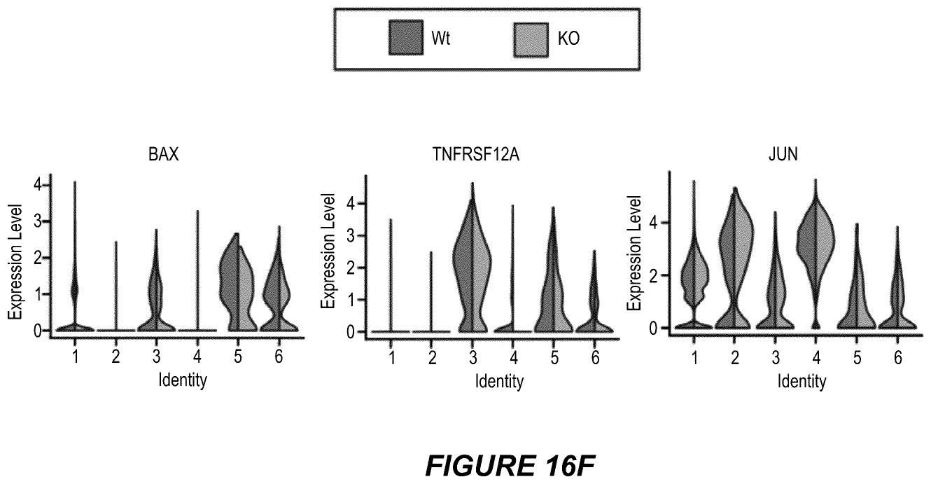

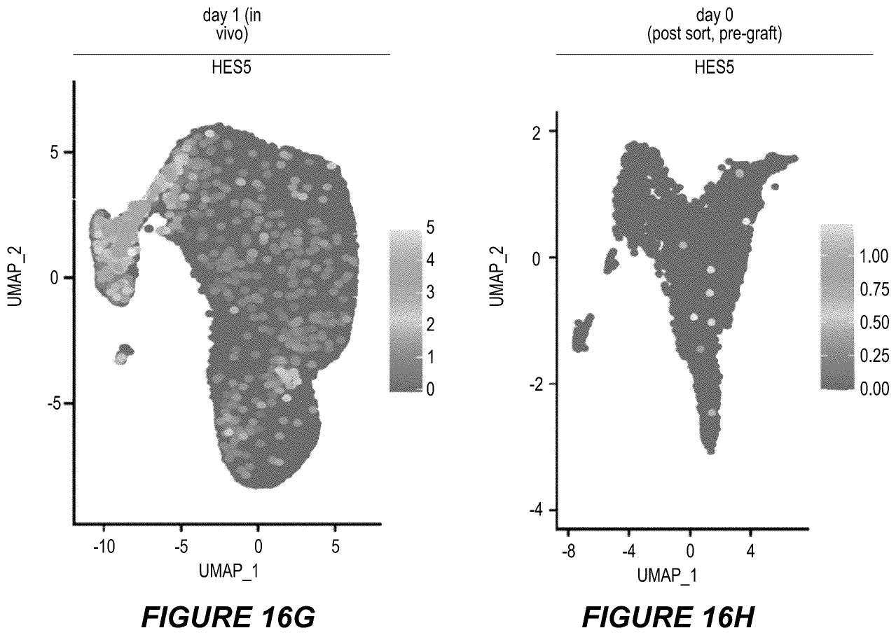

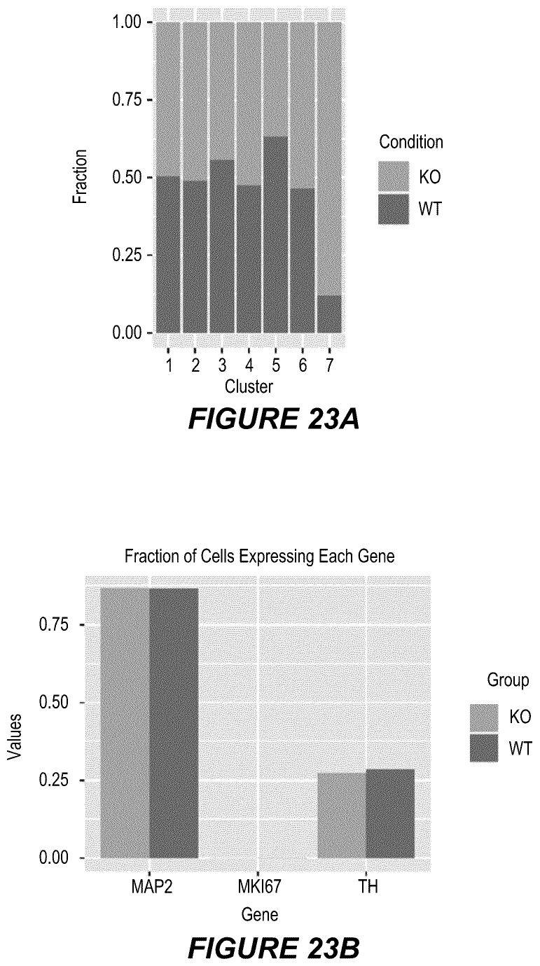

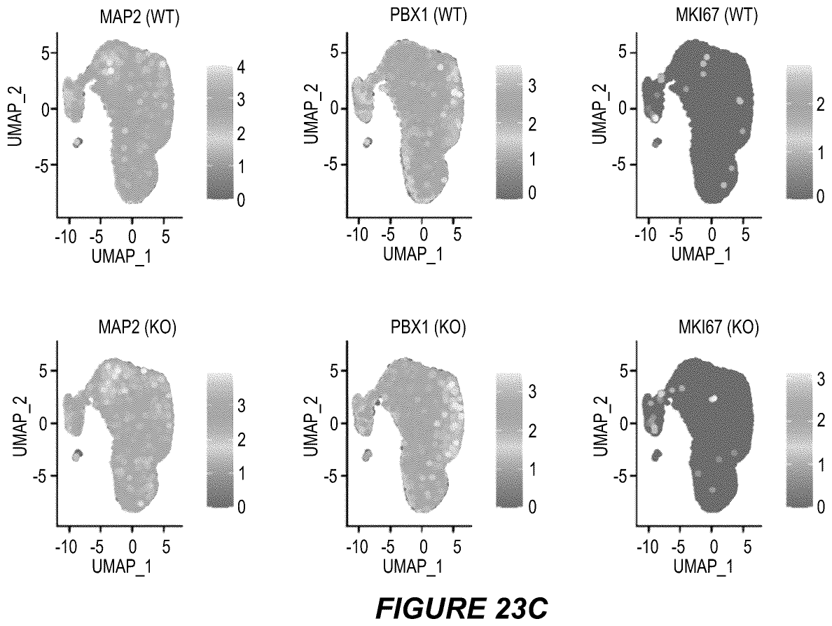

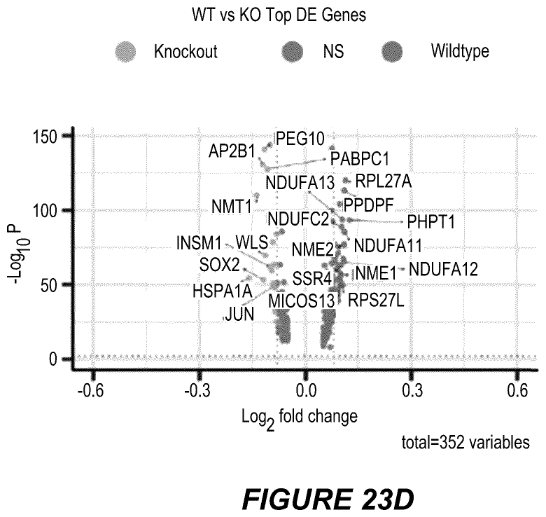

To further define the transcriptional landscape of grafted dopamine neurons, single cell mRNA sequencing was performed from p53 wild-type (WT) and p53 knock-out (KO) grafted neurons, re-isolated from the mouse brain at 1 dpt. Combined clustering of p53 WT and p53 KO cells showed highly overlapping distribution of clusters, implying that the cell state is not changed in dopamine neuron upon p53 KO (FIGS. 16A and 16B; FIG. 23A-23C). Based on PBX1, a marker particularly highly expressed in dopamine neurons, and on the expression of the neuronal marker MAP2, >90% of the cells exhibited features of postmitotic, dopamine neuron identity with very low expression of the proliferation marker, MKI67, in both p53 WT and KO neurons (FIG. 23B). By comparing our data to an available dataset from human fetal dopamine neuron development, most of the cells isolated at 1dpt were annotated as neuroblasts, compatible with early NURR1 positive identity while a small fraction of the cells was annotated as floor plate progenitors (FIG. 16C).

Clusters were identified that displayed molecular signatures of apoptosis or survival. Differential analysis of p53 WT versus p53 KO identified clusters 3, 5, and 6 with significantly increased expression of p53 downstream genes, such as BAX, BBC3, CDKNIA, and PHPT1 (FIGS. 16D-16F). In these clusters, TNFRSF12A was more robustly expressed in both p53WT and p53KO cells relative to the other clusters, supporting our findings that TNFα mediates a TP53-dependent apoptosis pathway in the graft (FIG. 16F). Interestingly, apoptosing clusters showed expression of SOX2 and HES5 and were annotated as floor plate progenitors. However, evidence of proliferation based on MK167 expression was not observed, suggesting that this cluster may reflect a dedifferentiation state rather than a bona fide, proliferating floor plate progenitor signature (FIGS. 16G and 16H). Furthermore, the progenitor signature was specific to immediately grafted neurons (1 dpt) and not observed in matched cells immediately pre-engraftment cells (FIG. 16H). These data argue that the progenitor-like phenotype is induced in those clusters within 24 hours post-engraftment and likely does not represent a contamination from imperfect sorting.

Conversely, clusters 1, 2, and 4 show high expression of survival signature characterized by the expression of ATF4, JUN, FOS, HSPA6, HSPA1A, HSPA1B, and DNAJB1 (FIG. 16D), highlighting that hPSC-derived dopamine neurons are under high cellular stress during engraftment procedure such as due to axotomy, endoplasmic reticulum stress, or DNA damage. Differential analysis between p53 WT and KO identified the survival marker JUN as significantly upregulated upon p53 KO (FIGS. 16F and 23D), emphasizing that blocking p53 expression imparts survival benefits to grafted dopamine neurons.

Questions remain regarding the source of the TNFα ligands. This dataset points out that grafted dopamine neurons show increased expression of TNFα superfamily ligands such as TNFSF12 and TNFSF10 (FIG. 22E) compared to their in vitro counterpart.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the present disclosures of which are incorporated herein by reference in their entireties for all purposes.

What is claimed is:

1. A method for treating a subject, comprising administering to the subject one or more midbrain dopamine (mDA) neurons, wherein p53-mediated apoptosis of the one or more mDA neurons is suppressed.

2. The method of claim 1, wherein the subject suffers from a neurodegenerative disorder and/or neurodegeneration of midbrain dopamine neurons, wherein the neurodegenerative disorder is selected from the group consisting of Parkinson's disease, Huntington's disease, Alzheimer's disease, multiple sclerosis, amyotrophic lateral sclerosis (ALS), frontotemporal dementia, and combinations thereof.

3. A method of improving in vivo survival of one or more midbrain dopamine (mDA) neurons, comprising suppressing p53-mediated apoptosis of the one or more mDA neurons.

4. The method of claim 3, wherein the suppression of p53-mediated apoptosis comprises contacting the one or more mDA neurons with a compound selected from the group consisting of TNFα inhibitors, NFκB inhibitors, p53 inhibitors, and combinations thereof, or wherein the suppression of p53-mediated apoptosis comprises inhibition of TNFα signaling, inhibition of NFκB signaling, inhibition of p53 signaling, or a combination of the foregoing.

5. The method of claim 4, wherein the TNFα inhibitor is selected from the group consisting of anti-TNFα antibodies, TNFα decoy receptors, chemical compounds, nucleic acid inhibitors, small molecule inhibitors, receptor biologic inhibitors, inactive TNF fragments, TNFα circulating receptor fusion protein, xanthine derivatives, 5-HT_{2A} agonist, and combinations thereof, wherein the anti-TNFα antibody is selected from the group consisting of adalimumab, adalimumab-adbm, adalimumab-adaz, adalimumab-atto, certolizumab pegol, golimumab, infliximab, infliximab-abda, infliximab-dyyb, remtolumab, afelimomab, nerelimomab, ozoralizumab, placulumab, and combinations thereof.

6. The method of claim 4, wherein the NFκB inhibitor is selected from the group consisting of upstream inhibitors of NFκB, inhibitors of IKK activity, inhibitors of IκB phosphorylation, inhibitors of IκB degradation, proteasome inhibitors, protease inhibitors, inhibitors of NFκB nuclear translocation and expression, NFκB DNA-binding inhibitors, and NFκB transactivation inhibitors, inhibitors of NFκB directed gene transactivation, antioxidants, and combinations thereof.

7. The method of claim 4, wherein the p53 inhibitor is selected from the group consisting of JNK inhibitors, p38 MAPK inhibitors, caspase inhibitors, puma/BBC3 inhibitors, BAX inhibitors, CDK inhibitors, MDM2 and MDMX activators, and combinations thereof.

8. The method of claim 3, wherein the suppression of p53-mediated apoptosis comprises knocking out or knocking down TP53 gene in the one or more mDA.

9. The method of claim 3, wherein the one or more mDA neurons express a marker selected from the group consisting of EN1, OTX2, TH, NURR1, FOXA2, LMX1A, PITX3, LM03, SNCA, ADCAP1, CHRNA4, ALDH1A1, SOX6, WNT1, DAT, VMAT2, GIRK2, SATB1, CALB1, CALB2, SNCG, PBX1, and combinations thereof.

10. The method of claim 3, wherein the one or more mDA neurons are post-mitotic mDA neurons.

11. The method of claim 3, wherein the one or more mDA neurons are in vitro differentiated from one or more stem cells, wherein the one or more stem cells are selected from the group consisting of embryonic stem cells, induced pluripotent stem cells, human stem cells, nonhuman primate stem cells, rodent nonembryonic stem cells, human embryonic stem cells, nonhuman primate embryonic stem cells, rodent embryonic stem cells, human induced pluripotent stem cells, nonhuman primate induced pluripotent stem cells, rodent induced pluripotent stem cells, and human recombinant pluripotent cells, nonhuman primate recombinant pluripotent cells, rodent recombinant pluripotent cells, and combinations thereof.

12. The method of claim 11, wherein the in vitro differentiation comprises contacting the one or more stem cells with at least one inhibitor of Small Mothers Against Decapentaplegic (SMAD) signaling, at least one activator of Sonic hedgehog (SHH) signaling, and at least one activator of wingless (Wnt) signaling.

13. The method of claim 12, wherein the concentration of the at least one activator of Wnt signaling that is contacted with the cells is increased between about 2 days and about 6 days from the initial contact of the cells with the at least one activator of Wnt signaling, wherein the concentration of the at least one activator of Wnt signaling that is contacted with the cells is increased by between about 250% and about 1800% of the initial concentration of the at least one activator of Wnt signaling contacted with the cells.

14. The method of claim 12, wherein the at least one activator of Wnt signaling is selected from the group consisting of an inhibitor of glycogen synthase kinase 3p (GSK3B) signaling, CHIR99021, CHIR98014, AMBMP hydrochloride, LP 922056, Lithium, deoxycholic acid, BIO, SB-216763, Wnt3A, Wnt1, Wnt5a, derivatives thereof, and combinations thereof; and the at least one inhibitor of SMAD signaling comprises an inhibitor of TGFβ/Activin-Nodal signaling, an inhibitor of bone morphogenetic protein (BMP) signaling, or a combination of the foregoing.

15. The method of claim 14, wherein the at least one inhibitor of TGFβ/Activin-Nodal signaling is selected from the group consisting of SB431542, derivatives of SB431542, and combinations thereof, and the at least one inhibitor of BMP signaling is selected from the group consisting of LDN193189, Noggin, dorsomorphin, derivatives of LDN193189, derivatives of Noggin, derivatives of dorsomorphin, and combinations thereof.

16. The method of claim 12, wherein the at least one activator of SHH signaling is selected from the group consisting of SHH proteins, recombinant SHHs, modified N-terminal SHHs, SHH C25II, purmorphamine, Smoothened agonists (SAG), and combinations thereof.

17. The method of claim 12, wherein the in vitro differentiation further comprises contacting the one or more stem cells with at least one activator of fibroblast growth factor (FGF) signaling, wherein the at least one activator of FGF signaling is selected from the group consisting of FGF18, FGF17, FGF8a, FGF8b, FGF4, FGF2, and combination thereof.

18. The method of claim 12, wherein the in vitro differentiation further comprises contacting the one or more stem cells with at least one inhibitor of Wnt signaling, wherein the at least one inhibitor of Wnt signaling is selected from the group consisting of IWP2, IWR1-endo, XAV939, IWP-01, Wnt-C59, IWP-L6, and ICG-001, and combinations thereof.

19. The method of claim 3, wherein the one or more mDA neurons express a detectable level of CD184 and do not express a detectable level of CD49e.

20. A composition comprising:
(a) one or more midbrain dopamine (mDA) neurons; and
(b) at least one compound selected from the group consisting of TNFα inhibitors, NFκB inhibitors, p53 inhibitors, and combinations thereof.
